SEQUENCE LISTING (7 sequences)

SEQ ID NO: 1 - RNA, 77 nt, Bacillus subtilis
uaaucgcgcugauuuaaccguauugcaagcgcgugauaaauguagcuaaaaaggggaugcccaguuuagcuggguuu

SEQ ID NO: 2 - RNA, 77 nt, Bacillus subtilis; identical to SEQ ID NO: 1

SEQ ID NO: 3 - DNA, 30 bp, Bacteriophage T7
gcgcgcgaattctaatacgactcactatag

SEQ ID NO: 4 - DNA, 41 bp, Hepatitis D virus
gttgtacgaagccgtaccgcttaccctggagatctgacacg

SEQ ID NO: 5 - RNA, 53 nt, Bacillus subtilis
gcgcggcgauuuaaccguauuagcagucgcgugauaaauguagcuaaaaagga

SEQ ID NO: 6 - RNA, 52 nt, Bacillus subtilis
ucgcgcugauuuaaccguauugcaagccgcugauaaauguagcuaaaaaggg

SEQ ID NO: 7 - RNA, 51 nt, Bacillus subtilis
gcgcggcgauuuaaccguauugcagucgcgugauaaauguagcuaaaagga

(19) United States
(12) Patent Application Publication
(10) Pub. No.: US 2011/0124713 A1
(43) Pub. Date: May 26, 2011

(54) SAM-II RIBOSWITCH AND USES THEREOF

(75) Inventors: Robert Batey, Boulder, CO (US); Sunny Gilbert, Boulder, CO (US)

(73) Assignee: The Regents of the University of Colorado, A body Corporate

(21) Appl. No.: 12/676,764

(22) PCT Filed: Sep. 5, 2008
(86) PCT No.: PCT/US08/10426

§ 371 Date: Dec. 7, 2010

Related U.S. Application Data
(60) Provisional application No. 60/967,603, filed on Sep. 6, 2007.

(51) Int. Cl. A61K 31/7088; C40B 30/02; C12N 1/20; C07H 21/02

(52) U.S. Cl. 514/44 R; 506/8; 435/252.1; 536/23.1

(57) ABSTRACT

Embodiments of the present invention provide for SAM-II riboswitches and analogs thereof, and methods for using the same. In certain embodiments of the present invention, test compounds are identified that associate with SAM-II riboswitches. In other embodiments, test compounds found to associate with SAM II can be used to increase or decrease gene expression of Gram-negative bacterial organisms.

This application claims priority from U.S. provisional application 60/967,603, filed Sep. 6, 2007, the contents of which are herein incorporated by reference.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant No. R-01 GM073850-01 awarded by the National Institutes of Health.

### FIELD OF THE INVENTION

The present invention relates to compositions and methods of use thereof related to SAM-II riboswitch.

### BACKGROUND OF THE INVENTION

Riboswitches are regulatory elements found within the 5′-untranslated regions (5′-UTRs) of many bacterial mRNAs. Riboswitches control gene expression in a cis-fashion through their ability to directly bind a specific small molecule metabolite. Ligand recognition is effected by the first domain of the riboswitch, termed the aptamer domain while the second, the expression platform, transduces the binding event into a regulatory switch. The switch includes an RNA element that can adapt to one of two mutually exclusive secondary structures. One of these structures is a signal for gene expression to be “on” and the other conformation turns the gene “off” (example in FIG. 1).). In Bacillus subtilis and other gram positive bacteria, it is believed riboswitches control greater than 2% of all genes, many of which are important for key pathways controlling the amino acid, nucleotide and cofactor metabolism.

Riboswitch aptamer domains are controlled by a diverse set of metabolites. In one example bacteria, amino acid metabolism in various Bacillus species is controlled by three known riboswitches: glycine, lysine and S-adenosylmethionine (SAM). Each has a distinct aptamer domain that has evolved to specifically recognize a specific ligand. Currently, there are three known distinct SAM riboswitches, one of which is dominant in gram positive bacteria, SAM-I, and one dominant in gram negative alpha-proteobacteria, SAM-II, and the third in lactobacteria, SAM-III. The SAM-II riboswitch is a cis-regulatory element found predominantly in alpha-proteobacteria that binds S-adenosyl methionine (SAM). Its structure and sequence appear to be unrelated to the SAM I riboswitch found in Gram-positive bacteria. This SAM II riboswitch is located upstream of the metA and metC genes in Agrobacterium tumefaciens, and other methionine and SAM biosynthesis genes in other alpha-proteobacteria. The SAM-II riboswitch is short with less than 70 nucleotides and is structurally relatively simple being composed of a single hairpin and a pseudoknot.

A need exist to better control bacterial growth, such as Gram negative bacterial growth, and generate effective treatments against bacterial infections. Embodiments of the present invention fulfill this need.

### SUMMARY OF THE INVENTION

One aspect of the present invention provides for methods of identifying a compound that associates with a SAM-II riboswitch including modeling at least a portion of the atomic structure depicted in FIGS. 4A and 4B with a test compound; and determining the interaction between the test compound and the SAM-II riboswitch structure.

Certain embodiments herein concern crystalline atomic structures of SAM-II riboswitches. In accordance with the methods, the structures may also be used for modeling and assessing the interaction of a riboswitch with a binding ligand.

In other embodiments herein, a compound may be identified that associates with the SAM-II riboswitch and reduces bacterial gene expression or associates with the SAM-II riboswitch and induces bacterial gene expression. In a more particular embodiment, a bacteria can be a gram negative bacteria. In accordance with these embodiments, atomic coordinates of the atomic structure can include at least a portion of the atomic coordinates listed in Table 1 for atoms depicted in FIGS. 4A and 4B wherein said association determination step can include determining a minimum interaction energy, a binding constant, a dissociation constant, or a combination thereof, for the test compound in the model of the SAM-II riboswitch. In some particular embodiments, an association determination step can include determining the interaction of the test compound with a nucleotide of SAM-II riboswitch including U10, U11, U12, U20, U21, G22, U44, A45, A46, A47 or a combination thereof. In other embodiments, an association determination step can include determining the interaction of the test compound with an S-adenosyl-methionine moiety including a ribose sugar, a methionine side chain, a sulfur moiety, an adenine moiety or combination thereof. Alternatively, in a more particular embodiment, the association determination step can include determining the interaction of the test compound with a nucleotide of SAM-II riboswitch depicted in FIGS. 4A and 4B including U10, U12, U20, G22, U44, A46, A47 or a combination thereof. Other embodiments contemplated herein include an association determination step of determining the interaction of the test compound with a P2b helix region of the SAM-II riboswitch. Yet other embodiments contemplated herein can include an association determination step including determining the interaction of the test compound within a pocket created in a major groove of the SAM-II riboswitch. Further embodiments concern an association determination step including determining the interaction of the test compound with a major groove of a P2b helix of the SAM-II riboswitch.

Bacterial cells contemplated of use in the methods and compositions herein include, but are not limited to, Gram negative species, for example, proteobacteria including Escherichia coli, Salmonella, and other Enterobacteriaceae, Pseudomonas, Moraxella, Helicobacter, Stenotrophomonas, Bdellovibrio, acetic acid bacteria, Legionella and many others. Other groups of Gram-negative bacteria include the cyanobacteria, spirochaetes, green sulfur and green non-sulfur bacteria. Medically relevant Gram-negative cocci include three organisms, which cause a sexually transmitted disease (Neisseria gonorrhoeae), a meningitis (Neisseria meningitidis), and respiratory symptoms (Moraxella catarrhalis). Medically relevant Gram-negative bacilli include, but are not limited to those that primarily cause respiratory problems (Hemophilus influenzae, Klebsiella pneumoniae, Legionella pneumophila, Pseudomonas aeruginosa), principally urinary problems (Escherichia coli, Proteus mirabilis, Enterobacter cloacae, Serratia marcescens), and usually gastrointestinal problems (Helicobacter pylori, Salmonella enteritidis, Salmonella typhi). Nosocomial gram negative bacteria can include Acinetobacter baumanii, which cause bacteremia, secondary meningitis, and ventilator-associated pneumonia. Medically relevant coccoid bacteria known to contain the SAM-II riboswitch include, but are not limited to, Bortedella pertusis and Bortedella bronchiseptica that causes whopping cough. In one embodiment, a bacterial organism can be medically relevant facultative intracellular bacteria known to contain the SAM-II riboswitch include, but are not limited to, Brucella melitenisis, which causes brucellosis in many areas of the world, and has been classified by the U.S. Center for Disease Control and Prevention as a potential agent in biological warfare. A related organism, Brucella suis, that also contains a SAM-II riboswitch, was the first pathogenic organism to be weaponized by the U.S. military in the 1950's, and thus represents a potential bioterrorism threat.

In certain embodiments, a SAM-II riboswitch disclosed herein can include one or more of the nucleotides listed in “Tertiary contacts” section of Table 2 where the nucleotide can be modified. In certain embodiments, the one or more modified nucleotides are selected from the group consisting of U10, U12, U20, G22, U44, A46, A47 or a combination thereof. In particular embodiments, the modified nucleotide of the SAM-II riboswitch can increase gene expression in a bacterial cell. For example, a test compound that contains a modified nucleotide may induce expression of a gene that is deleterious to a bacterial cell. In other embodiments, the modified nucleotide can decrease gene expression in a cell. For example, a test compound that contains a modified nucleotide may reduce expression of a gene that is necessary for survival of a bacterial cell. In certain particular embodiments, the modified nucleotide decreases sulfur production in a cell.

Embodiments of the present invention concern a test compound that associates with at least a portion of the SAM-II riboswitch atomic structure depicted in at least one of FIG. 4A or FIG. 4B. In accordance with these embodiments, the association can include association with at least one of nucleotides U10, U12, U20, G22, U44, A46, A47 or a combination thereof, wherein the composition is capable of modifying the SAM-II riboswitch activity of a bacterial organism by either inducing or reducing gene expression.

Certain embodiments concern compositions including, all of the 80 percent or more conserved nucleotides of the SAM-II riboswitch depicted in FIG. 1 (left) and 80% or greater, or 90% or greater or 95% or greater of the nucleotides depicted outside of the conserved region. One particular embodiment includes a composition of all 80 percent or more conserved nucleotides of the SAM-II riboswitch depicted in FIG. 1 (left) and all of the nucleotides depicted outside of the conserved region.

In one embodiment, the atomic coordinates of the atomic structure comprise the atomic coordinates listed in Table 1 for atoms depicted in FIGS. 4A and 4B.

Yet in another embodiment, the interaction determination step can include determining a minimum interaction energy, a binding constant, a dissociation constant, or a combination thereof, for the test compound in the model of the SAM-II riboswitch.

Still in other embodiments, the interaction determination step and test compound identification can include determining the interaction of the test compound with a nucleotide of SAM-II riboswitch comprising U10, U12, U20, G22, U44, A46, A47 or a combination thereof. Within this embodiment, the interaction determination step can include determining the interaction of the test compound with a nucleotide of SAM-II riboswitch comprising U10, U12, U20, G22, U44, A46, A47 or a combination thereof. In addition, the test compound that effectively interacts with one or more of the above mentioned nucleotides can be identified and expanded for use in targeting bacterial organisms disclosed herein.

Another aspect of the present invention provides, a method of regulating a gene in a cell by modulating an mRNA, said method comprising administering a SAM-II riboswitch modulating compound to the cell to modulate the SAM-II riboswitch activity of the mRNA. In certain embodiments, the gene expression is stimulated, while in other embodiments the gene expression is inhibited. Within certain embodiments where the gene expression is inhibited, the SAM-II riboswitch modulating compound forms a complex with the SAM-II riboswitch, thereby preventing the mRNA from forming an antiterminator element.

Certain embodiments include a compound that associates with one or more of the contact nucleotides and modulates the activity of the SAM-II riboswitch. In one particular embodiment, a compound capable of associating with one or more of the contact nucleotides may be capable of reducing sulfur metabolism in an organism having a SAM-II or SAM-II like riboswitch. In accordance with these embodiments, compounds of the present invention may be used to reduce infection caused by, or as a treatment for infection caused by an organism contemplated herein. In certain embodiments target organisms include bacteria. Bacteria contemplated herein include, but are not limited to Gram-negative bacterial organisms.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1 represents a schematic of a secondary structural switching in the Env12 metX mRNA (SEQ ID NO:1). In high intracellular concentrations of SAM, the effector ligand binds to the aptamer domain (dark grey box, left) incorporating a switching sequence (grey shaded area) into this domain, forcing the formation of a downstream rho-independent transcriptional terminator stem-loop in the expression platform (light grey box, middle). In the absence of SAM, the switching sequence is free to be incorporated into a more stable antiterminator element, allowing for transcription to proceed (SEQ ID NO: 2).

FIGS. 2A-2C represents an exemplary schematic of (A) secondary structure of the Env12 metX SAM-2 riboswitch with base pairing reflecting the tertiary structure of the SAM-bound RNA (SEQ ID NO:5); (B) an exemplary schematic of the global structure of the RNA; and (C) 90° rotation of the perspective shown in (B).

FIG. 3A-3E represents a schematic of (3A) details of the interactions between L3 (magenta) and the P1 helix (blue) emphasizing the role of four stacked adenosine residues in cementing the loop to the minor groove. FIG. 3B represents a schematic of a binding pocket of SAM (salmon) with the P2b helix. FIG. 3C represents a schematic of a hydrogen bonding interactions involving the adenine moiety of SAM. FIG. 3D represents a schematic of hydrogen bonding and electrostatic interactions involving the positively charged sulfur moiety and the methyl group of SAM. FIG. 3E represents a schematic of interactions between the RNA and the main chain atoms of the methionine residue of SAM.

FIGS. 4A (SEQ ID NO:6) and 4B (SEQ ID NO:7) represent schematics of exemplary sequences and secondary structures of the SAM-binding mRNA pseudoknot from Env12 and an exemplary sequence of the crystallized RNA construct with changes made to the sequence shaded in grey.

FIGS. 5A and 5B represent a schematic of electron density maps around the SAM binding site promoter A of the SAM-II riboswitch contoured at 1σ (orange cage). The final model is superimposed upon the density (green, RNA; magenta, SAM). (A) Solvent flattened experimental electron density map. (B) Final 2Fₒ-F_{c} electron density map.

FIGS. 6A and 6B. represent a schematic of superposition of the three protomers in the asymmetric unit that were built and refined individually. The standard pairwise r.m.s.d. for all atoms in the RNA and SAM is 1.26 | and the maximum likelihood r.m.s.d. for all atoms is 0.19 Å, as calculated using the program THESEUS. The two perspectives correspond to (A) FIGS. 4A and (B) FIG. 4B (FIG. 4B). Colors correspond to: red, molecule A; blue, molecule B; defined in the PDB coordinate file.

FIG. 7 represents a schematic of a side-by-side comparisons of the pseudoknot from human telomerase RNA (hTR, left) and SAM-II/SAM complex (right). The colors reflect the secondary structures of the RNA (blue, P1; green, P2; orange, L1; magenta, L3); the coloring pattern of SAM-II is slightly different from FIGS. 4A and 4B to make a clearer comparison between the two RNAs. The hTR structure shown is model 1 from the family of structures derived from NMR constraints (PDB ID 1YMO).

### DEFINITIONS

As used herein, “a” or “an” may mean one or more than one of an item.

### DETAILED DESCRIPTION

In the following sections, various exemplary compositions and methods are described in order to detail various embodiments of the invention. It will be obvious to one skilled in the art that practicing the various embodiments does not require the employment of all or even some of the specific details outlined herein, but rather that molecules, test compounds, samples, concentrations, times and other specific details may be modified through routine experimentation. In some cases, well known methods or components have not been included in the description.

Embodiments of the present invention provide for compositions and methods concerning SAM-II riboswitch and SAM-II riboswitch-like molecules.

Riboswitch aptamer domains are controlled by a diverse set of metabolites. Amino acid metabolism in various Bacillus species is controlled by three known riboswitches: glycine, lysine and S-adenosylmethionine (SAM). Each has a distinct aptamer domain that has evolved to specifically recognize a specific ligand. Currently, there are three distinct SAM riboswitches, one of which is dominant in gram positive bacteria, SAM-I, and one dominant in gram negative alpha-proteobacteria, SAM-II, and the third in lactobacteria, SAM-III. Characterization of the binding of SAM analogs has indicated that this RNA recognizes every feature of the ligand, although the reactive methyl group indirectly. To further understand how this extreme degree of discrimination between SAM and closely related compounds can be achieved by this mRNA element, a crystal structure in complex with SAM has been solved.

Certain embodiments herein concern compositions and methods for selecting and identifying compounds that can activate, deactivate or block SAM-II riboswitch. Activation or deactivation of a SAM-II riboswitch refers to the change in state of the riboswitch upon binding of the compound of interest, a test compound. The term trigger molecule is used herein to refer to molecules and compounds that can activate the SAM-II riboswitch.

Deactivation of a riboswitch refers to the change in state of the riboswitch when the trigger molecule is not bound. A riboswitch can be deactivated by binding of compounds other than the trigger molecule and in ways other than removal of the trigger molecule. Blocking of a riboswitch refers to a condition or state of the riboswitch where the presence of the trigger molecule does not activate the riboswitch.

In certain particular embodiments, methods of identifying a compound that interact with a SAM-II riboswitch include modeling the atomic structure of the SAM-II riboswitch with a test compound and determining if the test compound interacts with the SAM-II riboswitch. In accordance with these embodiments, the atomic contacts of the SAM-II riboswitch and test compound can be determined by means known in the art. Further, analogs of a compound known to interact with a SAM-II riboswitch can be generated by analyzing the atomic contacts for example the contacts that interact with ligand binding, then optimizing the atomic structure of the analog to maximize interaction. In certain embodiments, these methods can be used in a high throughput screen.

Other embodiments concern methods for identifying compounds that block a riboswitch. For example, an assay can be performed for assessing the induction or inhibition of SAM-II riboswitch in the presence of a test compound.

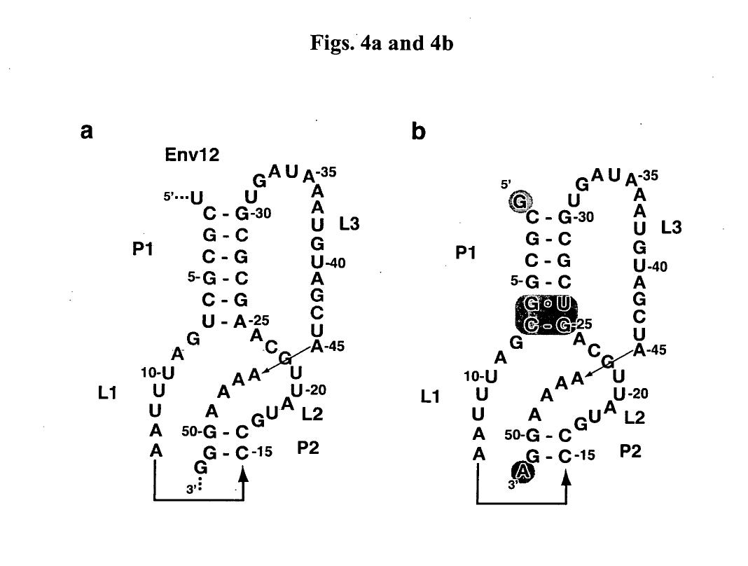

Some embodiments herein concern compositions and methods for identifying a test compound for significantly reducing the activity or inactivating a SAM-II riboswitch by binding the test compound to at least a portion of the atomic structure represented in FIGS. 4A and 4B. In accordance with these embodiments, activity of the SAM-II riboswitch can be measured by any methods known in the art. For example, the activity of the riboswitch can be measured in the presence or absence of a test compound in order to identify the efficiency of the test compound to reduce the activity of or inactivate the SAM-II riboswitch. Inactivation of a riboswitch in this manner can result from, for example, an alteration that prevents an S-adenosylmethionine molecule from binding; that prevents the change in state of the SAM-II riboswitch upon binding of S-adenosylmethionine; or the binding of the test compound interferes with ligand interaction or prevents the change in state of the SAM riboswitch.

In other embodiments, a test compound that activates a SAM-II riboswitch can be identified. For example, test compounds that activate a riboswitch can be identified by bringing into contact a test compound and a SAM-II riboswitch including at least a portion of the SAM-II riboswitch of FIG. 4A and FIG. 4B and assessing activation of the riboswitch. Activation of a SAM-II riboswitch can be assessed in any suitable manner. For example, activation of the SAM-II riboswitch can be measured by expression level of or modification of the expression level of a reporter gene in the presence or absence of the test compound. Examples of a reporter gene include, but are not limited to, beta-galactosidase, luciferase or green-fluorescence protein.

The SAM-II riboswitch is known to regulate multiple operons in a number of bacteria. Example bacteria contemplated herein include, but are not limited to, Gram negative species, for example, proteobacteria including Escherichia coli, Salmonella, and other Enterobacteriaceae, Pseudomonas, Moraxella, Helicobacter, Stenotrophomonas, Bdellovibrio, acetic acid bacteria, Legionella and many others. Other groups of Gram-negative bacteria include the cyanobacteria, spirochaetes, green sulfur and green non-sulfur bacteria. Medically relevant Gram-negative cocci include three organisms, which cause a sexually transmitted disease (Neisseria gonorrhoeae), a meningitis (Neisseria meningitidis), and respiratory symptoms (Moraxella catarrhalis). Medically relevant Gram-negative bacilli include, but are not limited to those that primarily cause respiratory problems (Hemophilus influenzae, Klebsiella pneumoniae, Legionella pneumophila, Pseudomonas aeruginosa), principally urinary problems (Escherichia coli, Proteus mirabilis, Enterobacter cloacae, Serratia marcescens), and usually gastrointestinal problems (Helicobacter pylori, Salmonella enteritidis, Salmonella typhi). Nosocomial gram negative bacteria can include Acinetobacter baumanii, which cause bacteremia, secondary meningitis, and ventilator-associated pneumonia. Other medically relevant coccoid bacteria known to contain the SAM-II riboswitch include, but are not limited to, Bortedella pertusis and Bortedella bronchiseptica that causes whopping cough. In other embodiments, medically relevant facultative intracellular bacteria known to contain the SAM-II riboswitch include Brucella melitenisis, which causes brucellosis in many areas of the world, and has been classified by the US Center for Disease Control and Prevention as a potential agent in biological warfare. In addition, contemplated in methods herein, a related organism, Brucella suis, that also contains a SAM-II riboswitch, was the first pathogenic organism to be weaponized by the U.S. military in the 1950's, and thus represents a potential bioterrorism threat.

### Organization of Riboswitch RNAs

Structural probing studies demonstrate that bacterial riboswitch elements are composed of two domains: a natural aptamer that serves as the ligand-binding domain, and an ‘expression platform’ that interfaces with RNA elements that are involved in gene expression. Structural probing investigations suggest that the aptamer domain of most riboswitches adopts a particular secondary- and tertiary-structure fold when examined independently, that is essentially identical to the aptamer structure when examined in the context of the entire 5′ leader RNA. This implies that, in many cases, the aptamer domain is a modular unit that folds independently of the expression platform.

The ligand-bound or unbound status of the aptamer domain is interpreted through the expression platform, which is responsible for exerting an influence upon gene expression. The aptamer domains are highly conserved amongst various organisms, whereas the expression platform varies in sequence, structure, and in the mechanism by which expression of the appended open reading frame is controlled.

Aptamer domains for riboswitch RNAs typically range from ˜70 to 170 nucleotides in length. Some aptamer domains, when isolated from the appended expression platform, exhibit improved affinity for the target ligand over that of the intact riboswitch. (˜10 to 100-fold). Presumably, there is an energetic cost in sampling the multiple distinct RNA conformations required by a fully intact riboswitch RNA, which is reflected by a loss in ligand affinity. Since the aptamer domain must serve as a molecular switch, this might also add to the functional demands on natural aptamers that might help rationalize their more sophisticated structures.

### Riboswitch Regulation

Bacteria primarily use two methods for termination of transcription. Certain genes incorporate a termination signal that is dependent upon the Rho protein, while others make use of Rho-independent terminators (intrinsic terminators) to destabilize the transcription elongation complex. The latter RNA elements are composed of a GC-rich stem-loop followed by a stretch of 6-9 uridyl residues. Intrinsic terminators are widespread throughout bacterial genomes, and are typically located at the 3′-termini of genes or operons. Interestingly, an increasing number of examples are being observed for intrinsic terminators located within 5″-UTRs.

In certain examples, RNA polymerase responds to a termination signal within the 5″-UTR in a regulated fashion. Under certain conditions, the RNA polymerase complex is directed by external signals either to perceive or to ignore the termination signal. Although transcription initiation might occur without regulation, control over mRNA synthesis (and of gene expression) is ultimately dictated by regulation of the intrinsic terminator. Presumably, one of at least two mutually exclusive mRNA conformations results in the formation or disruption of the RNA structure that signals transcription termination. A trans-acting factor, which in some instances an RNA is generally required for receiving a particular intracellular signal and subsequently stabilizing one of the RNA conformations. Riboswitches offer a direct link between RNA structure modulation and the metabolite signals that are interpreted by the genetic control machinery.

Certain mRNAs involved in thiamine biosynthesis bind to thiamine (vitamin B₁) or its bioactive pyrophosphate derivative (TPP) without the participation of protein factors. The mRNA-effector complex adopts a distinct structure that sequesters the ribosome-binding site and leads to a reduction in gene expression. This metabolite-sensing mRNA system provides an example of a genetic “riboswitch” (referred to herein as a riboswitch) whose origin might predate the evolutionary emergence of proteins. It has been discovered that the mRNA leader sequence of the btuB gene of Escherichia coli can bind coenzyme B₁₂ selectively, and that this binding event brings about a structural change in the RNA that is important for genetic control. It was also discovered that mRNAs that encode thiamine biosynthetic proteins also employ a riboswitch mechanism.

Although certain specific natural riboswitches such as SAM-I riboswitch was one of the first examples of mRNA elements that control genetic expression by metabolite binding, it is suspected that this genetic control strategy may be widespread in biology. If these metabolites were being biosynthesized and used before the advent of proteins, then certain riboswitches might be modern examples of the most ancient form of genetic control. A search of genomic sequence databases has revealed that sequences corresponding to the TPP aptamer exist in organisms from bacteria, archaea and eukarya-largely without major alteration. Although new metabolite-binding mRNAs are likely to emerge as evolution progresses, it is possible that the known riboswitches are molecular fossils from the RNA world.

In certain embodiments, it is contemplated that a SAM-II Reporter system can be used to assess whether a test compound activates or inactivates the SAM-II riboswitch. In certain particular embodiments, an in vitro selection protocol can be designed for example to assess whether a test compound activates or deactivates the SAM-II riboswitch. In one particular embodiment, binding of the ligand can be monitored by a mobility-shift assay, known in the art, to discern free and bound RNA, providing a basis for selection of binding-competent RNAs. Ligand binding to the RNA can cause a conformational and/or secondary structural change in the RNA that can result in a change in its migration in a native polyacrylamide gel.

In certain embodiments, a detectible tag can be incorporated into the SAM-II riboswitch. In accordance with these embodiments, a test compound can be placed in contact with the SAM-II riboswitch and the interaction of the test compound and the SAM-II riboswitch assessed by measuring the presence or absence of a detectible tag. In certain particular examples, a detectible tag may be undetectable in the presence of a test compound thereby quenching the signal. This mechanism can be adapted to existing SAM-II riboswitches, as this method can take advantage of assessing a ligand-mediated interaction of the SAM-II riboswitch. In certain particular embodiments, a detectible tag can be placed within the ligand interaction region. In more particular embodiments, a detectible tag can be placed on any one of ligand binding nucleic acids, including but not limited to, U10, U11, U12, U20, U21, G22, U44, A45, A46, A47, or a combination thereof of FIG. 4A or FIG. 4B of the SAM-II riboswitch. In these examples, a test compound can be combined with a SAM-II riboswitch depicted FIG. 4A or FIG. 4B and a detectible signal on the SAM-II riboswitch quenched when the test compound binds to at least one of the ligand-binding nucleic acids indicated above. In one particular example, a florescent tag molecule can be positioned in RNA adjacent to the binding site of SAM and binding can be monitored via a change in fluorescence of a reporter gene.

In other embodiments, control compounds can be used to assess interaction of the test compound compared to a known compound that interacts with a SAM-II riboswitch. To use riboswitches to report ligand binding by analyzing for a detectible tag, the appropriate construct can be determined empirically. The optimum length and composition of a test compound and its binding site on the riboswitch can be assessed systematically to result in the highest ligand binding region interaction possible. The validity of the assay can be determined by comparing apparent relative binding affinities of different SAM-II analogs, SAM-II antibodies or other SAM-II binding agents to a particular test compound (determined by the presence or level of detectible signal generation of the tag) to the binding constants determined by standard in-line probing.

In other embodiments, interaction of a test compound with at least a portion of the atomic structures depicted in FIG. 4A or FIG. 4B may be assessed by measuring uptake and/or synthesis of methionine and/or synthesis of SAM in a bacterial test cell system (e.g., cultures of B. subtilus). In accordance with these embodiments, a test compound confirmed to interact with at least a portion of the atomic structures depicted in FIG. 4A or 4B can be synthesized and/or purified for future use. In one example use, the test compound may be placed in contact with SAM-II riboswitch and the uptake and/or synthesis of methionine and/or synthesis of SAM can be measured. If a test compound is found to effectively block these functions, the test compound may be a candidate for use in inhibiting bacterial expansion or eliminating bacteria within a subject or a system.

In one example method, the structure depicted in FIG. 4A or 4B indicates that the RNA does not recognize the methyl group attached to the sulfur moiety, providing a place to build additional functionality that would be recognized by the RNA. Additionally, the positive charge on the sulfur is also recognized but not the sulfur atom itself, indicating that this region can be altered to ensure stability of the compound. Potential compounds could be computationally built and fit into the structure in place of SAM to determine if they would fit in the binding pocket of the riboswitch. Novel compounds can be synthesized by established chemistries and tested using a fluorescence or foot printing type assay to ensure that they are recognized by the RNA.

It is contemplated herein that test compounds capable of associating with the atomic structures depicted in FIG. 4A or 4B may be a nucleic acid molecule, a small molecule, an antibody, a pharmaceutical agent, small peptide, peptide mimetic, nucleic acid mimetic, modified saccharide or aminoglycoside. Preferred test compound compositions would be small molecule mimetics of SAM or nucleic acid mimetics that build off of the adenosine moiety of SAM.

### Kits

In still further embodiments, kits for methods and compositions described herein are contemplated. In one embodiment, the kits have a point-of care application, for example, the kits may have portability for use at a site of suspected bacterial outbreak. In another embodiment, a kit for treatment of a subject having a bacterial-induced infection is contemplated. In accordance with this embodiment, the kit may be used to reduce the bacterial infection of a subject.

The kits may include a container means. Any of the kits will generally include at least one vial, test tube, flask, bottle, syringe or other container means, into which the testing agent, may be preferably and/or suitably aliquoted. Kits herein may also include a means for comparing the results such as a suitable control sample such as a positive and/or negative control.

### Nucleic Acids

In various embodiments, isolated nucleic acids may be used as test compounds for binding the atomic structure depicted in FIG. 4A or 4B. The isolated nucleic acid may be derived from genomic RNA or complementary DNA (cDNA). In other embodiments, isolated nucleic acids, such as chemically or enzymatically synthesized DNA, may be of use for capture probes, primers and/or labeled detection oligonucleotides.

A “nucleic acid” includes single-stranded and double-stranded molecules, as well as DNA, RNA, chemically modified nucleic acids and nucleic acid analogs. It is contemplated that a nucleic acid may be of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000 or greater nucleotide residues in length, up to a full length protein encoding or regulatory genetic element.

### Construction of Nucleic Acids

Isolated nucleic acids may be made by any method known in the art, for example using standard recombinant methods, synthetic techniques, or combinations thereof. In some embodiments, the nucleic acids may be cloned, amplified, or otherwise constructed.

The nucleic acids may conveniently comprise sequences in addition to a portion of a SAM-II riboswitch. For example, a multi-cloning site comprising one or more endonuclease restriction sites may be added. A nucleic acid may be attached to a vector, adapter, or linker for cloning of a nucleic acid. Additional sequences may be added to such cloning and sequences to optimize their function, to aid in isolation of the nucleic acid, or to improve the introduction of the nucleic acid into a cell. Use of cloning vectors, expression vectors, adapters, and linkers is well known in the art.

### Recombinant Methods for Constructing Nucleic Acids

Isolated nucleic acids may be obtained from bacterial or other sources using any number of cloning methodologies known in the art. In some embodiments, oligonucleotide probes which selectively hybridize, under stringent conditions, to the nucleic acids of a bacterial organism. Methods for construction of nucleic acid libraries are known and any such known methods may be used.

### Nucleic Acid Screening and Isolation

Bacterial RNA or cDNA may be screened for the presence of an identified genetic element of interest using a probe based upon one or more sequences. Various degrees of stringency of hybridization may be employed in the assay. As the conditions for hybridization become more stringent, there must be a greater degree of complementarity between the probe and the target for duplex formation to occur. The degree of stringency may be controlled by temperature, ionic strength, pH and/or the presence of a partially denaturing solvent such as formamide. For example, the stringency of hybridization is conveniently varied by changing the concentration of formamide within the range up to and about 50%. The degree of complementarity (sequence identity) required for detectable binding will vary in accordance with the stringency of the hybridization medium and/or wash medium. In certain embodiments, the degree of complementarity can optimally be about 100 percent; but in other embodiments, sequence variations in the RNA may result in <100% complementarity, <90% complimentarity probes, <80% complimentarity probes, <70% complimentarity probes or lower depending upon the conditions. In certain examples, primers may be compensated for by reducing the stringency of the hybridization and/or wash medium.

High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. Other exemplary conditions are disclosed in the following Examples. It is understood that the temperature and ionic strength of a desired stringency are determined in part by the length of the particular nucleic acid(s), the length and nucleotide content of the target sequence(s), the charge composition of the nucleic acid(s), and by the presence or concentration of formamide, tetramethylammonium chloride or other solvent(s) in a hybridization mixture. Nucleic acids may be completely complementary to a target sequence or may exhibit one or more mismatches.

### Nucleic Acid Amplification

Nucleic acids of interest may also be amplified using a variety of known amplification techniques. For instance, polymerase chain reaction (PCR) technology may be used to amplify target sequences directly from bacterial RNA or cDNA. PCR and other in vitro amplification methods may also be useful, for example, to clone nucleic acid sequences, to make nucleic acids to use as probes for detecting the presence of a target nucleic acid in samples, for nucleic acid sequencing, or for other purposes.

### Synthetic Methods for Constructing Nucleic Acids

Isolated nucleic acids may be prepared by direct chemical synthesis by methods such as the phosphotriester method, or using an automated synthesizer. Chemical synthesis generally produces a single stranded oligonucleotide. This may be converted into double stranded DNA by hybridization with a complementary sequence or by polymerization with a DNA polymerase using the single strand as a template. While chemical synthesis of DNA is best employed for sequences of about 100 bases or less, longer sequences may be obtained by the ligation of shorter sequences.

### Covalent Modification of Nucleic Acids

A variety of cross-linking agents, alkylating agents and radical generating species may be used to bind, label, detect, and/or cleave nucleic acids. In addition, covalent crosslinking to a target nucleotide using an alkylating agent complementary to the single-stranded target nucleotide sequence can be used. A photoactivated crosslinking to single-stranded oligonucleotides mediated by psoralen can be used. Use of N4, N4-ethanocytosine as an alkylating agent to crosslink to single-stranded oligonucleotides has also been disclosed. Various compounds to bind, detect, label, and/or cleave nucleic acids are known in the art.

### Nucleic Acid Labeling

In various embodiments, tag nucleic acids may be labeled with one or more detectable labels to facilitate identification of a target nucleic acid sequence bound to a capture probe on the surface of a microchip. A number of different labels may be used, such as fluorophores, chromophores, radio-isotopes, enzymatic tags, antibodies, chemiluminescent, electroluminescent, affinity labels, etc. One of skill in the art will recognize that these and other label moieties not mentioned herein can be used. Examples of enzymatic tags include urease, alkaline phosphatase or peroxidase. Colorimetric indicator substrates can be employed with such enzymes to provide a detection means visible to the human eye or spectrophotometrically. A well-known example of a chemiluminescent label is the luciferin/luciferase combination.

In preferred embodiments, the label may be a fluorescent, phosphorescent or chemiluminescent label. Exemplary photodetectable labels may be selected from the group consisting of Alexa 350, Alexa 430, AMCA, aminoacridine, BODIPY 630/650, BODIPY 650/665, BODIPY-FL, BODIPY-R6G, BODIPY-TMR, BODIPY-TRX, 5-carboxy-4′,5′-dichloro-2′,7′-dimethoxy fluorescein, 5-carboxy-2′,4′,5′,7′-tetrachlorofluorescein, 5′-carboxyfluorescein, 5-carboxyrhodamine, 6-carboxyrhodamine, 6-carboxytetramethyl amino, Cascade Blue, Cy2, Cy3, Cy5,6-FAM, dansyl chloride, Fluorescein, HEX, 6-JOE, NBD (7-nitrobenz-2-oxa-1,3-diazole), Oregon Green 488, Oregon Green 500, Oregon Green 514, Pacific Blue, phthalic acid, terephthalic acid, isophthalic acid, cresyl fast violet, cresyl blue violet, brilliant cresyl blue, para-aminobenzoic acid, erythrosine, phthalocyanines, azomethines, cyanines, xanthines, succinylfluoresceins, rare earth metal cryptates, europium trisbipyridine diamine, a europium cryptate or chelate, diamine, dicyanins, La Jolla blue dye, allopycocyanin, allococyanin B, phycocyanin C, phycocyanin R, thiamine, phycoerythrocyanin, phycoerythrin R, REG, Rhodamine Green, rhodamine isothiocyanate, Rhodamine Red, ROX, TAMRA, TET, TRIT (tetramethyl rhodamine isothiol), Tetramethylrhodamine, and Texas Red. These and other labels are available from commercial sources, such as Molecular Probes (Eugene, OR).

Solid Supports

Solid supports are solid-state substrates or supports with which molecules (such as trigger molecules, e.g., SAM) and riboswitches (or other components used in, or produced by, the disclosed methods) can be associated. Riboswitches and other molecules can be associated with solid supports directly or indirectly. For example, analytes (e.g., trigger molecules, test compounds) can be bound to the surface of a solid support or associated with capture agents (e.g., compounds or molecules that bind an analyte) immobilized on solid supports. As another example, riboswitches can be bound to the surface of a solid support or associated with probes immobilized on solid supports. An array is a solid support to which multiple riboswitches, probes or other molecules have been associated in an array, grid, or other organized pattern.

In some embodiments, a solid-state substrate may be used. Solid supports contemplated of use can include any solid material with which components can be associated, directly or indirectly. These material include but are not limited to acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multi-well glass slide can be employed.

In certain embodiments, an array can include a plurality of riboswitches, trigger molecules, other molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of riboswitches, trigger molecules, other molecules, compounds and/or probes can be distributed over any number of solid supports. For example, in some embodiments, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, riboswitches, trigger molecules, or other molecules) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances. In accordance with these examples, components can be associated or immobilized on a solid support at any density. Components can be immobilized to the solid support at a density exceeding 400 different components per cubic centimeter. Arrays of components can have any number of components depending on the circumstances.

### Pharmaceutical Compositions

In certain embodiments, compositions of identified test compounds may be generated for use in a subject having a bacterial infection in order to reduce or eliminate the infection in the subject. In accordance with these embodiments, the compositions can be administered in a subject in a biologically compatible form suitable for pharmaceutical administration in vivo. By “biologically compatible form suitable for administration in vivo” is meant a form of the active agent (e.g., pharmaceutical chemical, protein, gene, antibody etc of the embodiments) to be administered in which any toxic effects are outweighed by the therapeutic effects of the active agent. Administration of a therapeutically active amount of the therapeutic compositions is defined as an amount effective, at dosages and for periods of time necessary to achieve the desired result. For example, a therapeutically effective amount of an antibody or nucleic acid molecule reactive with at least a portion of SAM-II riboswitch depicted in FIG. 4A or FIG. 4B may vary according to factors such as the disease state, age, sex, and weight of the individual, and the ability of antibody to elicit a desired response in the individual. Dosage regimens may be adjusted to provide the optimum therapeutic response. For example, several divided doses may be administered daily or the dose may be proportionally reduced as indicated by the exigencies of the therapeutic situation.

In one embodiment, the compound (e.g., pharmaceutical chemical, nucleic acid molecule, gene, protein, antibody etc of the embodiments) may be administered in a convenient manner such as by injection such as subcutaneous, intravenous, by oral administration, inhalation, transdermal application, intravaginal application, topical application, intranasal or rectal administration. Depending on the route of administration, the active compound may be coated in a material to protect the compound from the degradation by enzymes, acids and other natural conditions that may inactivate the compound. In a preferred embodiment, the compound may be orally administered. In another preferred embodiment, the compound may be inhaled in order to make the compound bioavailable to the lung.

A compound may be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. To administer a compound that stimulates or inhibits a SAM-II riboswitch by other than parenteral administration, it may be necessary to coat the compound with, or co-administer the compound with, a material to prevent its inactivation. Enzyme inhibitors include pancreatic trypsin inhibitor, diisopropylfluorophosphate (DEP) and trasylol. Liposomes include water-in-oil-in-water emulsions as well as conventional liposomes. The active agent may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of microorganisms can be achieved by various antibacterial and antifungal agents (i.e., parabens, chlorobutanol, phenol, ascorbic acid, thimerosal, and the like). In many cases, it will be preferable to include isotonic agents, for example, sugars, polyalcohols such as manitol, sorbitol, sodium chloride in the composition. A compound such as aluminum monostearate and gelatin can be included to prolong absorption of the injectable compositions.

Sterile injectable solutions can be prepared by incorporating active compound (e.g., a chemical that modulates the SAM-II riboswitch) in the required amount in an appropriate solvent with one or a combination of ingredients enumerated above, as required, followed by filtered sterilization. Generally, dispersions are prepared by incorporating the active compound into a sterile vehicle that contains a dispersion medium and other required ingredients from those enumerated above. In the case of sterile powders for the preparation of sterile injectable solutions, the preferred methods of preparation are vacuum drying and freeze-drying which yields a powder of the active ingredient (i.e., a chemical agent, antibody etc.) plus any additional desired ingredient from a previously sterile-filtered solution thereof.

When the active agent is suitably protected, as described above, the composition may be orally administered (or otherwise indicated), for example, with an inert diluent or an assimilable edible carrier. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active agent and the particular therapeutic effect to be achieved, and (b) the limitations inherent an active agent for the therapeutic treatment of individuals.

### EXAMPLES

The following examples are included to illustrate various embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that many changes may be made in the specific embodiments which are disclosed and still obtain a like or similar result without departing from the spirit and scope of the invention.

Riboswitches act as genetic regulatory elements through the interplay of two distinct domains in the 5′-untranslated region (5′-UTR) of an mRNA: the aptamer domain that directly binds a specific cellular metabolite and a downstream expression platform containing a secondary structural switch that determines whether the gene will be expressed. Among the known mRNA elements that specifically bind small molecules in vivo, there are now at least three characterized S-adenosylmethionine (SAM)-responsive riboswitches: SAM-I, SAM-II (SAM-α), and SAM-III (SAM-MK), underscoring the importance of riboregulation of sulfur metabolism by SAM. Each riboswitch is unique in its primary or secondary structure and appears to be exclusive within a particular bacterial genome. SAM-I, the only SAM riboswitch whose tertiary structure has been solved.

In one exemplary method, to further illuminate how mRNAs respond to SAM, the structure of a second class of SAM-responsive RNA: SAM-II was determined. To find a SAM-II mRNA that yielded diffraction-quality crystals, 13 phylogenetic variants were examined that differed in the lengths of its two primary helices in the predicted secondary structure. Crystals that diffracted X-rays to 2.8 Å resolution were obtained in the presence of SAM of an RNA derived from a Sargasso Sea environmental sequence (Env12) upstream of the metX gene (homoserine acetyltransferase). Phasing by SIRAS was achieved using a cesium atom bound to a G•U phasing module engineered into a non-conserved region of the P1 helix. All of the nucleotides in the RNA as well as SAM were found in three slightly different RNA-ligand complexes in the asymmetric unit were built and refined without NCS constraints to yield a final model with Rxtal=20.6% and Rfree=26.3% (Table 1).

The global architecture of the SAM-II riboswitch comprises a classic (H-type) pseudoknot8 (FIGS. 4A and 4B). The secondary structure consists of two Watson-Crick paired helices (P1 and P2a) and two loop regions (L1 and L3). A third helical element, not predicted from sequence alignment, is formed from highly conserved sequences in L2 and L3 to form P2b (FIG. 4, FIG. 4A). The loops L1 and L3 interact with the major and minor grooves of P2a/b and P1, respectively, to form an intricate tertiary structure with the SAM binding pocket located in the center of P2b (FIG. 4B). Each of the helical segments stack upon the others without distortions creating a nearly straight structure.

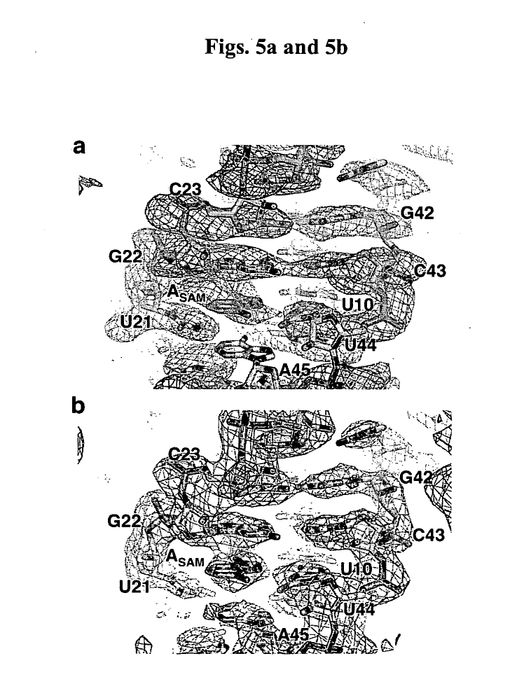

Consistent with the sequence conservation patterns of classic pseudoknots, L1 is uracil rich while L3 is adenine rich. Bases in L3 do not form planar triples with the minor groove of P1, but rather are skewed at an ˜70° angle with respect to P1 forming hydrogen bonding interactions with two successive base pairs (FIG. 5A). A stack of four adenosines in L3 (A33, A35-37) rotate clockwise (as viewed from A33) along P1, such that A33 interacts via its Hoogsteen face, while A37 uses its sugar face in a fashion akin to a type-1 A-minor groove triple9. As a result, residues of L3 form extensive hydrogen bonding interactions with the minor groove side of every base pair of P1 except the C2-G30 base pair at the very top (FIG. 5A).

The SAM binding pocket is created by the formation of an extended triplex between L1 and the major groove of P2b. The beginning of the P2b-L1 interaction is defined by a single minor groove triple between A24-U40•A41 (FIG. 4A), followed by a series of major groove triples formed by nucleotides G8-U12 in L1 with the face of P2b. The Watson-Crick face of each nucleotide in L1 interacts with the Hoogsteen face of nucleotides in 3′-strand of the P2b helix (nucleotides 42-46). This near-perfect triplex is terminated by a sheared A19•A47 pair. Below this pair, the P2b triplex transitions into the P2a helix, defining the lower boundary of the SAM binding pocket. An isolated A13•(U18-A48) triple and a sheared G17•A49 pair comprise this transition. The final nucleotide of L1, which is not conserved in phylogeny, is flipped out and makes no contacts with the two-base-pair helix of P2a that corresponds to the second helix of the classic pseudoknot fold.

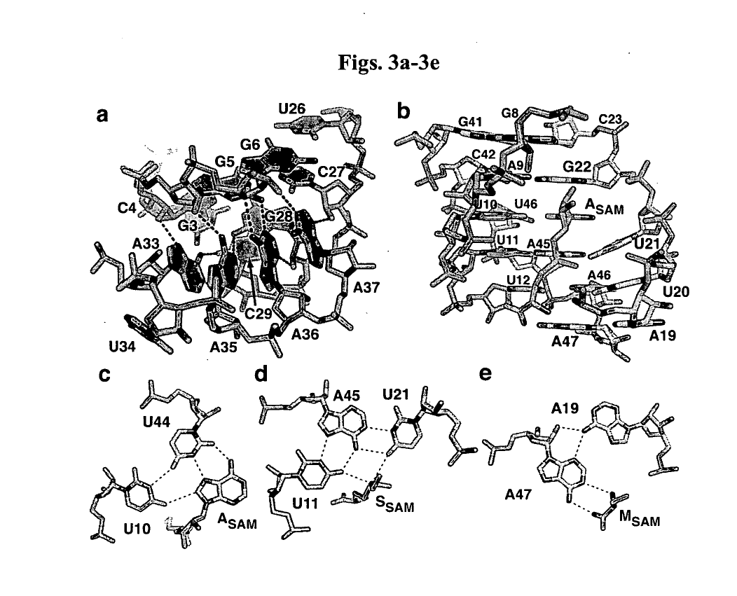

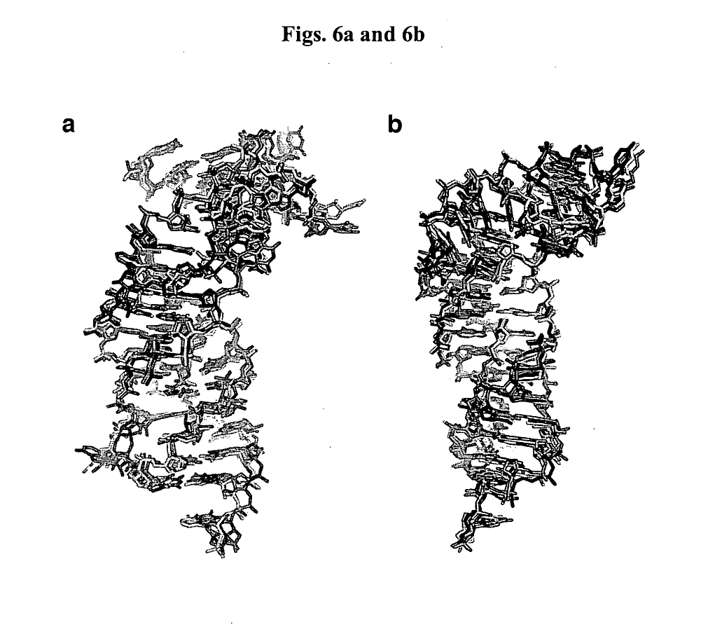

SAM binds in an extended trans-configuration along the major groove face of the P2b/L1 triplex forming direct contacts with five successive base pairs and triples (FIG. 3B, C). The adenine moiety of SAM (ASAM) participates in a base triple between U10 and U44 using its Hoogsteen face to pair with U44 (FIGS. 5A and 5B) similar to that observed in the SAM-I structure. Interestingly, this site appears to be created by the deletion of a single residue between U21 and G22; this “hole” thus requires adenine to be threaded through the helix sideways such that its Watson-Crick face is solvent exposed on the minor groove face of the triplex. The positively charged sulfur moiety and activated methyl group are recognized by the carbonyl oxygens of U11 and U21 (FIG. 3D) explaining the ability of SAM-II to discriminate between SAM and S-adenosylhomocysteine (SAH). Again, this is strikingly similar to discrimination by SAM-I, which uses the minor groove carbonyl moieties of two universally conserved A-U pairs to interact electrostatically with the positive charge. The main chain atoms of methionine (carboxylate and amino groups) are positioned in the major groove adjacent to the sheared A19•A47 pair (FIG. 3E). However, these groups are in different configurations in the three protomers in the asymmetric unit (FIGS. 6A and 6B). Molecules A and C place the amino and carboxylate groups along the Watson-Crick face of A47 (FIG. 3E), while molecule B does not place the amino group in hydrogen bonding contact with the RNA. Recognition of the main chain atoms by the Watson-Crick face of an adenine base is similar to that observed in SAM-I, in which the Watson-Crick face of a guanine forms a salt bridge with the carboxylate moiety. Thus, all of the available functional groups in SAM appear to be directly or indirectly recognized by the mRNA, consistent with an investigation of the binding of SAM analogs to the SAM-II riboswitch. Despite the extensive recognition of SAM, the ligand is not nearly as extensively buried within the RNA (64% solvent inaccessible) as observed in other riboswitch-small molecule complexes.

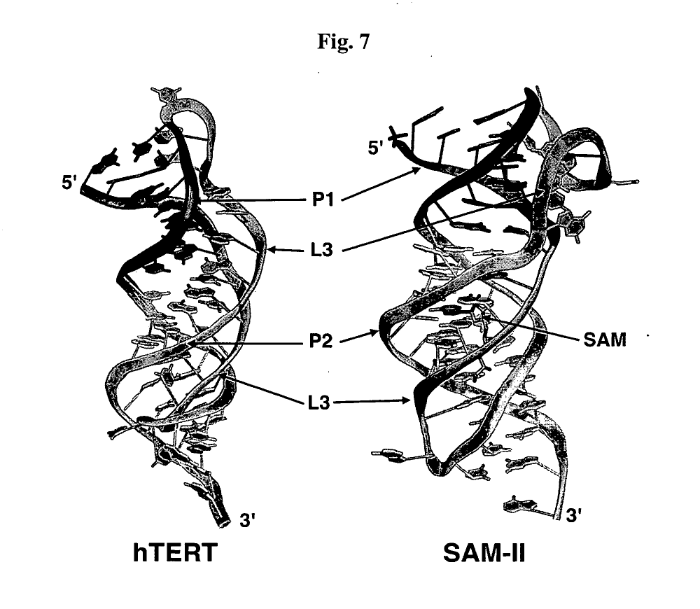

RNA pseudoknot motifs are ubiquitous throughout all classes of structured RNAs stabilizing local tertiary structure and acting as protein recognition elements. They function in diverse pathways from translational regulation like those found in viral genomes to their role in telomerase RNA (hTR) as a key element of TERT repeat addition processivity. SAM-II has an architecture consistent with other H-type pseudoknots, most notably the hTR pseudoknot core that is involved in protein recognition (FIGS. 7A and 7B). Both the hTR RNA and SAM-II contain triplexes at the junction between P1 and P2a/b that have a strong preference for A•U base pairs in this region. The hTR core is a five-base-pair triplex broken only by a non-canonical A•U Hoogsteen base pair in the center. Similarly, the SAM-II core is a five-base-pair triplex, containing the ASAM motif in the center that forms a Hoogsteen base pair with U44. Thus, the SAM-II riboswitch employs a ligand-independent structure with which to scaffold the binding pocket; deletion of a single critical residue in the middle of the triplex destabilizes the tertiary structure such that SAM is required to fully form the pseudoknot.

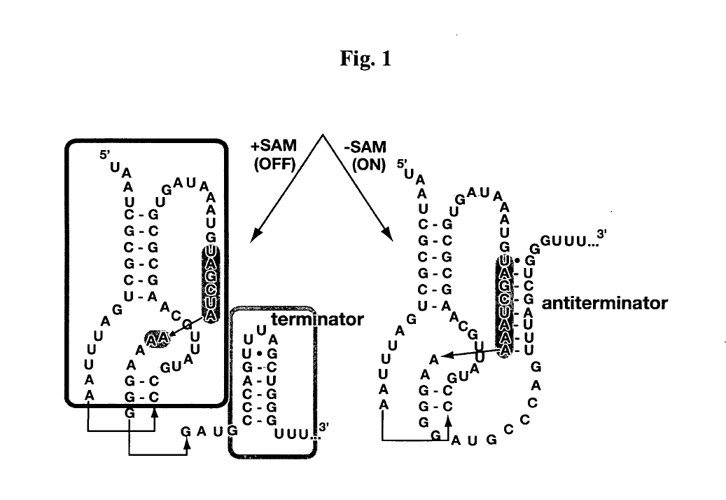

Most riboswitches contain a switching sequence within the 3′-side of the P1 helix that is either incorporated within the aptamer domain or forms part of a secondary structure in the expression platform, depending upon whether the aptamer domain is ligand-bound14. Typically, the tertiary architecture of the aptamer domain in the presence of ligand stabilizes formation of the P1 helix. The SAM-II riboswitch differs from most other riboswitches in that its pseudoknot architecture prevents pairing of the 5′- and 3′-ends of the aptamer domain (FIG. 1). Despite this difference, the switching sequence (nucleotides 40-47) remains localized to the 3′-side of the aptamer domain, residing in P2b. In-line probing of the riboswitch in the absence and presence of SAM reveals strong ligand-dependent protections corresponding to nucleotides G8-U12 in L14. SAM binding to P2b, stabilizes the formation of key tertiary interactions between L1 and the 3′-side of P2b that serve to cement the switching sequence into the aptamer domain and thereby fating the secondary structure of the downstream expression platform. Thus, this structure lends support to a general mechanism of riboswitch action in which ligand binding is communicated to the expression platform via ligand-induced tertiary interactions with a switching sequence.

### Methods and Materials.

RNA library synthesis and purification. A series of RNAs corresponding to secondary structure and sequence variations of the SAM-II RNA observed across phylogeny was constructed according to the length of the P1 and P2 helices of the minimal riboswitch (FIG. 4). These helices vary between 5-8 base pairs and 2-6 base pairs, respectively. Combinations of different helix length resulted in an initial library containing 13 representative RNAs that included the metA RNA previously characterized. RNAs were constructed by standard PCR methods using overlapping DNA oligonucleotides (Integrated DNA Technologies). The resulting DNA fragment, which contained EcoRI and NcoI restriction sites at the 5′ and 3′ ends, respectively, as well as sequences coding for a T7 RNA polymerase promoter and an 3′ HδV ribozyme was ligated into pRAV12. The resulting plasmid was verified by sequence analysis. Template DNA for in vitro transcription was generated by PCR from each individual plasmid using primers directed against the T7 RNA polymerase promoter at the 5′ end and the 3′ side HdV ribozyme (5′GCGCGCGAATTCTAATACGACTCACTATAG (SEQ ID NO: 3); 3′ GCACAGTCTAGAGGTCCCATTCGCCATGCCGAAGCATGTTG (SEQ ID NO: 4)). RNA was transcribed in a 12.5 mL reactions containing 30 mM Tris-HCl (pH 8.0), 10 mM DTT, 0.1% Triton X-100, 0.1 mM spermidine-HCl, 6 mM of both ATP and GTP, 4 mM of both UTP and CTP, 36 mM MgCl₂, 25 mg/mL T7 RNA polymerase, 1 mL of ˜0.5 mM template³, and 1 unit/mL inorganic pyrophosphatase. The reaction was incubated for 2 hr at 37 ° C. RNA was precipitated in 70% EtOH, pelleted, and resuspended in load buffer containing 4 M urea, 100 mM Na-EDTA, pH 8.0, 25% formamide, xylene cyanol, and bromophenol blue, and purified by denaturing 12% PAGE. Gel slices containing target RNA were excised from the gel, electroeluted in 1× TBE buffer, collected, exchanged against 3×15 ml aliquots of buffer containing 10 mM K⁺-HEPES, pH 7.5 using a 10,000 MWCO centrifugal filter device (Amicon, Ultra-15), and concentrated to ˜500 μL. RNA concentrations were determined from the magnitude of the UV absorbance at 260 nm and the calculated extinction coefficient (556,400 M⁻¹ cm⁻¹) of the individual RNA's base composition.

RNA crystallization. SAM-II riboswitch crystals were obtained by the hanging drop/vapor diffusion method in which the RNA solution was mixed in a 1:1 ratio with mother liquor. The initial library of RNAs was screened versus the PEG-Ion, Crystal Screen, Natrix, and Nucleic Acid Mini-screen (Hampton Research). RNAs and promising conditions were further refined based on crystal morphology and size, as well as diffraction quality and space group. The final RNA construct was further refined to contain a heavy-ion binding phasing module in the P1 helix that did not alter any of the residues that are conserved across phylogeny (FIG. 4). The final conditions that yielded diffractions quality crystals contained 8 mM cobalt hexammine chloride, 640 mM ammonium acetate, 10% PEG 1K, 10 mM barium chloride, 50 mM Na⁺-cacodylate, pH 6.0, 25 ° C. Single crystal growth required cat whisker micro-seeding from a solution containing microcrystals from previously grown, but polymorphic, SAM-II crystals suspended in mother liquor plus 8% (2R,3R)-(−)-2,3-butanediol. Crystals reached their maximum size (-200 mM, cube-like) in 3-5 days, and were subsequently backsoaked in 30 μl one of two heavy-atom derivative solutions. Crystals designated for isomoprphous replacement were backsoaked in mother liquor containing 2 mM magnesium chloride, 10 mM SAM, and 200 mM cesium chloride for ˜10 minutes. Crystals designated as native were backsoaked in mother liquor that only contained 320 mM ammonium acetate (all other components remaining the same) with the addition of 2 mM magnesium chloride, 10 mM SAM, and 600 mM lithium chloride also for ˜10 minutes. This was followed by a 10 minute exchange into the same solutions containing the addition of 8% (2R,3R)-(−)-2,3-butanediol. Crystals were looped and flash-frozen in liquid nitrogen. Diffraction data was collected on a home X-ray source (Rigaku MSC) using CuK_{α} radiation. Anomalous diffraction data was collected by an inverse-beam experiment and was integrated, indexed, and scaled using HKL2000.

Phasing and structure determination. Phases were determined using a single wavelength isomorphous replacement with anomalous scattering (SIRAS) experiment and diffraction data extending to 2.5 Å resolution for that native data set and 2.8 Å resolution for the derivative set. In this experiment, cesium was treated as the heavy atom derivative, yielding anomalous signal at CuK_{α} wavelength (f′=1.16, f″=8.80), while a lithium backsoaked crystal was treated as the native data set. A phasing solution containing four heavy atoms (FOM=0.27, Z-score=17) was generated in SOLVE. Following density modification in CNS, the figure of merit improved to 0.35. An experimental electron density map was of sufficient quality to follow the trace of the phosphate backbone and identify regions of base-pairing for two of the three molecules in the asymmetric unit. Using a combination of model and experimental phases, a final density modified electron density map was calculated (FOM=0.74) and the location of the third protomer was identified. All nucleotides in the three RNAs, as well as the locations of SAM, and three Cs atoms were eventually built through subsequent rounds of iterative model building in Pymol and refinement in CNS while following the improvement of R_{free}. Solvent molecules were added using two rounds of the water-picking in CNS. The 5′- and 3′-ends of the RNAs contained functional groups that required additional refinement at the end of the building process. The addition of a 5′ppGTP and a 2′-3′-cyclic phosphate at the RNA termini were supported by the electron density in these regions. A Mg²⁺ ion was also identified in the region where protomers A and B stack 5′ to 5′ and 3′ to 3′ causing the two 5′-diphosphate groups to come into close proximity. The final R and R_{free} are 20.6% and 26.3%, respectively.

FIG. 1 represents an exemplary schematic of secondary structural switching in the Env12 metX mRNA. In high intracellular concentrations of SAM, the effector ligand binds to the aptamer domain (dark grey box, left) incorporating a switching sequence (grey shaded area) into this domain, forcing the formation of a downstream rho-independent transcriptional terminator stem-loop in the expression platform (light grey box). In the absence of SAM, the switching sequence is free to be incorporated into a more stable antiterminator element, allowing for transcription to proceed.

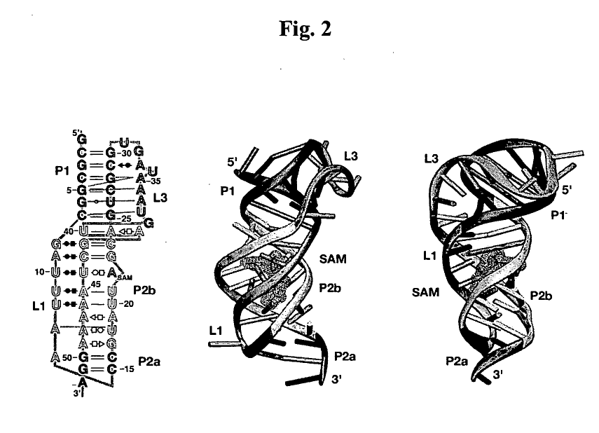

FIG. 2. represents an exemplary schematic of (A) secondary structure of the Env12 metX SAM-2 riboswitch with base pairing reflects the tertiary structure of the SAM-bound RNA. Base interactions are shown using the notation of Neocoles and Westhof. Circles indicate an interaction involving the Watson-Crick face, squares the Hoogsteen face, and triangles the sugar edge; black symbols denote a parallel arrangement while open symbols denote an antiparallel arrangement. Dashed lines denote hydrogen bonding interactions that cannot be described as one of the standard pairing interactions. Colors of the bases reflect their position in the tertiary structure (blue, P1 or P2a; green, P2b; magenta, L3; orange, L1). (B) represents an exemplary schematic of the global structure of the RNA; colors are consistent with the secondary structure. The red dots represent the van der Waals surface of S-adenosylmethionine. (C) 90° rotation of the perspective shown in (B).

FIG. 3A-3E represents in (A) details of the interactions between L3 (magenta) and the P1 helix (blue) emphasizing the role of four stacked adenosine residues in cementing the loop to the minor groove. (B) represents a schematic of a binding pocket of SAM (salmon) with the P2b helix. The adenine base of SAM is accommodated by an opening in the 5′-strand of P2b between U21 and G22, while the ribose sugar and methionine residue reside in the narrow major groove of the triplex. (C) represents hydrogen bonding interactions involving the adenine moiety of SAM. (D) represents hydrogen bonding and electrostatic interactions involving the positively charged sulfur moiety and the methyl group of SAM. (E) represents interactions between the RNA and the main chain atoms of the methionine residue of SAM.

FIG. 4 represents in (A) Sequence and secondary structure of the SAM-binding mRNA pseudoknot from Env12. The nomenclature for the stems and loops (P1-P2 and L1-L3, respectively) is derived from standard naming of H-type pseudoknots. Light grey nucleotides are those whose identity is >95% conserved and dark grey nucleotides corresponds to >80% conservation of identity. (B) represents a schematic of sequence of the crystallized RNA construct with changes made to the sequence shaded in grey. Each change was made for a specific purpose: U1G for efficient transcriptional initiation by T7 RNA polymerase, G52A for efficient 3′-end processing by a 3′-HδV ribozyme, and (C6G,U7C,A25G,G26U) to add a cesium/iridium hexammine ion binding site for phasing.

FIG. 5 represents electron density maps around the SAM binding site protomer A of the SAM-II riboswitch contoured at 1σ (orange cage). The final model is superimposed upon the density (green, RNA; magenta, SAM). (A) Solvent flattened experimental electron density map. (B) Final 2Fₒ-F_{c} electron density map.

FIG. 6 represents an exemplary schematic of superposition of the three protomers in the asymmetric unit that were built and refined individually. The standard pairwise r.m.s.d. for all atoms in the RNA and SAM is 1.26 Å and the maximum likelihood r.m.s.d. for all atoms is 0.19 Å, as calculated using in one example, the program THESEUS. The two perspectives correspond to (A) FIGS. 6B and (b) FIG. 6C (FIGS. 6B and C represent protomer A). Colors correspond to: red, molecule A; blue, molecule B; green, molecule C, as defined in the PDB coordinate file.

FIG. 7 represents an exemplary schematic of side-by-side comparisons of the pseudoknot from human telomerase RNA (hTR, left) and SAM-II/SAM complex (right). The colors reflect secondary structures of the RNA (blue, P1; green, P2; orange, L1; magenta, L3); the coloring pattern of SAM-II is slightly different from FIG. 4 to make a clearer comparison between the two RNAs. The hTR structure shown is model 1 from the family of structures derived from NMR constraints (PDB ID 1YMO).

TABLE 1
Data collection, phasing, and refinement statistics (SIRAS)

| | Native | | Derivative | |
|---|---|---|---|---|
| Data collection | | | | |
| Space group | C2 | | C2 | |
| Cell dimensions | | | | |
| a, b, c (Å) | 115.26, 48.10, 109.62 | | 115.64, 48.29, 109.51 | |
| α, β, γ (°) | 90, 108.26, 90 | | 90, 108.20, 90 | |
| Resolution (Å) | 20-2.6 | (2.69-2.60) | 20-2.8 | (2.90-2.80)¹ |
| R_{merge} | 0.053 | (0.368) | 0.061 | (0.349) |
| I/σI | 28.5 | (2.5) | 31.9 | (5.0) |
| Completeness (%) | 89.6 | (54.4) | 97.9 | (94.7) |
| Redundancy | 3.6 | (2.8) | 7.8 | (7.7) |

| | |
|---|---|
| Refinement | |
| Resolution (Å) | 20-2.8 |
| No. reflections | 26,866 |
| R_{work}/R_{free} | 20.6/26.3 |
| No. atoms | |
| RNA | 3,375 |
| Ligand/ion | 84 |
| Water | 154 |
| B-factors | |
| RNA | 64.90 |
| Ligand/ion | 64.87 |
| Water | 45.50 |
| R. m. s. deviations | |
| Bond lengths (Å) | 0.0059 |
| Bond angles (°) | 1.017 |

¹Reflections obtained from a single crystal. Values in parentheses are for highest-resolution shell.

TABLE 2

| | | | |
|---|---|---|---|
| REMARK | 3 | | |
| REMARK | 3 | REFINEMENT. | |
| REMARK | 3 | PROGRAM | : CNS 1.2 |
| REMARK | 3 | AUTHORS | : BRUNGER, ADAMS, CLORE, DELANO, |
| REMARK | 3 | | GROS, GROSSE-KUNSTLEVE, JIANG, |
| REMARK | 3 | | KUSZEWSKI, NILGES, PANNU, READ, |
| REMARK | 3 | | RICE, SIMONSON, WARREN |
| REMARK | 3 | | |

| REMARK | 3 | DATA USED IN REFINEMENT. | | |
|---|---|---|---|---|
| REMARK | 3 | RESOLUTION RANGE HIGH | (ANGSTROMS) | : 2.80 |
| REMARK | 3 | RESOLUTION RANGE LOW | (ANGSTROMS) | : 19.90 |
| REMARK | 3 | DATA CUTOFF | (SIGMA(F)) | : 0.0 |
| REMARK | 3 | DATA CUTOFF HIGH | (ABS(F)) | : 1022121.53 |
| REMARK | 3 | DATA CUTOFF LOW | (ABS(F)) | : 0.000000 |

| | | | | |
|---|---|---|---|---|
| REMARK | 3 | COMPLETENESS (WORKING + TEST) | (%) | : 97.9 |
| REMARK | 3 | NUMBER OF REFLECTIONS | | : 26866 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING SET) | : 0.206 |
| REMARK | 3 | FREE R VALUE | | : 0.263 |

| | | | | |
|---|---|---|---|---|
| REMARK | 3 | FREE R VALUE TEST SET SIZE | (%) | : 7.2 |

| REMARK | 3 | FREE R VALUE TEST SET COUNT | : 1924 | |
|---|---|---|---|---|
| REMARK | 3 | ESTIMATED ERROR OF FREE R VALUE | : 0.006 | |
| REMARK | 3 | | | |
| REMARK | 3 | FIT IN THE HIGHEST RESOLUTION BIN. | | |
| REMARK | 3 | TOTAL NUMBER OF BINS USED | | : 6 |
| REMARK | 3 | BIN RESOLUTION RANGE HIGH | (A) | : 2.80 |
| REMARK | 3 | BIN RESOLUTION RANGE LOW | (A) | : 2.97 |
| REMARK | 3 | BIN COMPLETENESS (WORKING + TEST) | (%) | : 94.8 |

| | | | | |
|---|---|---|---|---|
| REMARK | 3 | REFLECTIONS IN BIN | (WORKING SET) | : 4033 |
| REMARK | 3 | BIN R VALUE | (WORKING SET) | : 0.407 |
| REMARK | 3 | BIN FREE R VALUE | | : 0.408 |

| | | | | |
|---|---|---|---|---|
| REMARK | 3 | BIN FREE R VALUE TEST SET SIZE | (%) | : 7.4 |

| | | | |
|---|---|---|---|
| REMARK | 3 | BIN FREE R VALUE TEST SET COUNT | : 324 |
| REMARK | 3 | ESTIMATED ERROR OF BIN FREE R VALUE | : 0.023 |
| REMARK | 3 | | |

| REMARK | 3 | NUMBER OF NON-HYDROGEN ATOMS USED IN REFINEMENT. | |
|---|---|---|---|
| REMARK | 3 | PROTEIN ATOMS | : 0 |
| REMARK | 3 | NUCLEIC ACID ATOMS | : 1050 |
| REMARK | 3 | HETEROGEN ATOMS | : 81 |
| REMARK | 3 | SOLVENT ATOMS | : 154 |
| REMARK | 3 | | |
| REMARK | 3 | B VALUES. | |

| | | | | |
|---|---|---|---|---|
| REMARK | 3 | FROM WILSON PLOT | (A**2) | :129.5 |
| REMARK | 3 | MEAN B VALUE | (OVERALL, A**2) | : 65.6 |

| REMARK | 3 | OVERALL ANISOTROPIC B VALUE. | |
|---|---|---|---|
| REMARK | 3 | B11 (A**2) : 34.88 | |
| REMARK | 3 | B22 (A**2) : −5.03 | |
| REMARK | 3 | B33 (A**2) : −29.85 | |
| REMARK | 3 | B12 (A**2) : 0.00 | |
| REMARK | 3 | B13 (A**2) : 23.73 | |
| REMARK | 3 | B23 (A**2) : 0.00 | |
| REMARK | 3 | | |
| REMARK | 3 | BULK SOLVENT MODELING. | |
| REMARK | 3 | METHOD USED | : FLAT MODEL |
| REMARK | 3 | KSOL | : 0.25 |
| REMARK | 3 | BSOL | : 9.21319 (A**2) |

| REMARK | 3 | | | |
|---|---|---|---|---|
| REMARK | 3 | ESTIMATED COORDINATE ERROR. | | |
| REMARK | 3 | ESD FROM LUZZATI PLOT | (A) : | 0.40 |
| REMARK | 3 | ESD FROM SIGMAA | (A) : | 0.62 |
| REMARK | 3 | LOW RESOLUTION CUTOFF | (A) : | 5.00 |
| REMARK | 3 | | | |

| REMARK | 3 | CROSS-VALIDATED ESTIMATED COORDINATE ERROR. | | |
|---|---|---|---|---|
| REMARK | 3 | ESD FROM C-V LUZZATI PLOT | (A) : | 0.50 |
| REMARK | 3 | ESD FROM C-V SIGMAA | (A) : | 0.62 |
| REMARK | 3 | | | |

| REMARK | 3 | RMS DEVIATIONS FROM IDEAL VALUES. | | |
|---|---|---|---|---|
| REMARK | 3 | BOND LENGTHS | (A) : | 0.006 |
| REMARK | 3 | BOND ANGLES | (DEGREES) : | 1.0 |
| REMARK | 3 | DIHEDRAL ANGLES | (DEGREES) : | 21.5 |
| REMARK | 3 | IMPROPER ANGLES | (DEGREES) : | 1.40 |
| REMARK | 3 | | | |

| REMARK | 3 | ISOTROPIC THERMAL MODEL : RESTRAINED | | |
|---|---|---|---|---|
| REMARK | 3 | | | |
| REMARK | 3 | ISOTROPIC THERMAL FACTOR RESTRAINTS. | RMS | SIGMA |

| | | | | | |
|---|---|---|---|---|---|
| REMARK | 3 | MAIN-CHAIN BOND | (A**2) : | NULL ; | NULL |
| REMARK | 3 | MAIN-CHAIN ANGLE | (A**2) : | NULL ; | NULL |
| REMARK | 3 | SIDE-CHAIN BOND | (A**2) : | NULL ; | NULL |
| REMARK | 3 | SIDE-CHAIN ANGLE | (A**2) : | NULL ; | NULL |
| REMARK | 3 | | | | |
| REMARK | 3 | NCS MODEL : NONE | | | |
| REMARK | 3 | | | | |

| | | | | |
|---|---|---|---|---|
| REMARK | 3 | NCS RESTRAINTS. | RMS | SIGMA/WEIGHT |

| | | | | | |
|---|---|---|---|---|---|
| REMARK | 3 | GROUP 1 POSITIONAL | (A) : | NULL ; | NULL |
| REMARK | 3 | GROUP 1 B-FACTOR | (A**2) : | NULL ; | NULL |
| REMARK | 3 | | | | |

| | | | |
|---|---|---|---|
| REMARK | 3 | PARAMETER FILE 1 | : CNS_TOPPAR/protein_rep.param |
| REMARK | 3 | PARAMETER FILE 2 | : dna-rna_rep_revise4.param |
| REMARK | 3 | PARAMETER FILE 3 | : CNS_TOPPAR/water_rep.param |
| REMARK | 3 | PARAMETER FILE 4 | : CNS_TOPPAR/ion.param |
| REMARK | 3 | PARAMETER FILE 5 | : sam7.param |
| REMARK | 3 | TOPOLOGY FILE 1 | : CNS_TOPPAR/protein.top |
| REMARK | 3 | TOPOLOGY FILE 2 | : dna-rna_rep_revise4.top |
| REMARK | 3 | TOPOLOGY FILE 3 | : CNS_TOPPAR/water_rep.top |
| REMARK | 3 | TOPOLOGY FILE 4 | : CNS_TOPPAR/ion.top |
| REMARK | 3 | TOPOLOGY FILE 5 | : sam7.top |
| REMARK | 3 | | |

| | | | | | | | |
|---|---|---|---|---|---|---|---|
| REMARK | 3 | OTHER | REFINEMENT | REMARKS : | BULK | SOLVENT | MODEL USED |

| | | | | | | | | | | | | | | | | |
|---|---|---|---|---|---|---|---|---|---|---|---|---|---|---|---|---|
| SEQRES | 1 | A | 52 | GUA | CYT | GUA | CYT | GUA | GUA | CYT | GUA | ADE | URI | URI | URI | ADE |
| SEQRES | 2 | A | 52 | ADE | CYT | CYT | GUA | URI | ADE | URI | URI | GUA | CYT | ADE | GUA | URI |
| SEQRES | 3 | A | 52 | CYT | GUA | CYT | GUA | URI | GUA | ADE | URI | ADE | ADE | ADE | URI | GUA |
| SEQRES | 4 | A | 52 | URI | ADE | GUA | CYT | URI | ADE | ADE | ADE | ADE | ADE | GUA | GUA | ADE |
| SEQRES | 1 | B | 52 | GUA | CYT | GUA | CYT | GUA | GUA | CYT | GUA | ADE | URI | URI | URI | ADE |
| SEQRES | 2 | B | 52 | ADE | CYT | CYT | GUA | URI | ADE | URI | URI | GUA | CYT | ADE | GUA | URI |
| SEQRES | 3 | B | 52 | CYT | GUA | CYT | GUA | URI | GUA | ADE | URI | ADE | ADE | ADE | URI | GUA |
| SEQRES | 4 | B | 52 | URI | ADE | GUA | CYT | URI | ADE | ADE | ADE | ADE | ADE | GUA | GUA | ADE |
| SEQRES | 1 | C | 52 | GUA | CYT | GUA | CYT | GUA | GUA | CYT | GUA | ADE | URI | URI | URI | ADE |
| SEQRES | 2 | C | 52 | ADE | CYT | CYT | GUA | URI | ADE | URI | URI | GUA | CYT | ADE | GUA | URI |
| SEQRES | 3 | C | 52 | CYT | GUA | CYT | GUA | URI | GUA | ADE | URI | ADE | ADE | ADE | URI | GUA |
| SEQRES | 4 | C | 52 | URI | ADE | GUA | CYT | URI | ADE | ADE | ADE | ADE | ADE | GUA | GUA | ADE |

| | | | | | | | |
|---|---|---|---|---|---|---|---|
| CRYST1 | 115.258 | 48.095 | 109.617 | 90.00 | 108.25 | 90.00 C 2 | 12 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.008676 | 0.000000 | 0.002862 | 0.00000 |
| SCALE2 | 0.000000 | 0.020792 | 0.000000 | 0.00000 |
| SCALE3 | 0.000000 | 0.000000 | 0.009606 | 0.00000 |

| | | | | | | | | | | | |
|---|---|---|---|---|---|---|---|---|---|---|---|
| ATOM | 1 | O6P | GUA | A | 1 | −3.122 | 22.893 | 35.812 | 1.00 | 79.01 | A |
| ATOM | 2 | O3P | GUA | A | 1 | −2.183 | 21.111 | 37.439 | 1.00 | 75.49 | A |
| ATOM | 3 | O4P | GUA | A | 1 | −1.328 | 21.287 | 35.020 | 1.00 | 77.61 | A |
| ATOM | 4 | O5P | GUA | A | 1 | −0.763 | 23.120 | 36.591 | 1.00 | 81.95 | A |
| ATOM | 5 | P2 | GUA | A | 1 | −1.851 | 22.131 | 36.189 | 1.00 | 81.46 | A |
| ATOM | 6 | P | GUA | A | 1 | −0.796 | 20.349 | 37.787 | 1.00 | 70.43 | A |
| ATOM | 7 | O1P | GUA | A | 1 | 0.242 | 21.364 | 38.237 | 1.00 | 69.31 | A |
| ATOM | 8 | O2P | GUA | A | 1 | −0.373 | 19.586 | 36.525 | 1.00 | 67.03 | A |
| ATOM | 9 | O5′ | GUA | A | 1 | −0.900 | 19.278 | 39.005 | 1.00 | 62.45 | A |
| ATOM | 10 | C5′ | GUA | A | 1 | 0.300 | 18.834 | 39.669 | 1.00 | 58.69 | A |
| ATOM | 11 | C4′ | GUA | A | 1 | 0.890 | 17.581 | 39.014 | 1.00 | 53.75 | A |
| ATOM | 12 | O4′ | GUA | A | 1 | 1.235 | 17.789 | 37.609 | 1.00 | 53.70 | A |
| ATOM | 13 | C1′ | GUA | A | 1 | 2.550 | 17.294 | 37.340 | 1.00 | 52.22 | A |
| ATOM | 14 | N9 | GUA | A | 1 | 3.421 | 18.451 | 37.118 | 1.00 | 53.59 | A |
| ATOM | 15 | C4 | GUA | A | 1 | 4.803 | 18.541 | 37.218 | 1.00 | 53.64 | A |
| ATOM | 16 | N3 | GUA | A | 1 | 5.653 | 17.553 | 37.565 | 1.00 | 53.63 | A |
| ATOM | 17 | C2 | GUA | A | 1 | 6.917 | 17.975 | 37.595 | 1.00 | 53.12 | A |
| ATOM | 18 | N2 | GUA | A | 1 | 7.905 | 17.149 | 37.950 | 1.00 | 51.55 | A |
| ATOM | 19 | N1 | GUA | A | 1 | 7.309 | 19.248 | 37.290 | 1.00 | 53.64 | A |
| ATOM | 20 | C6 | GUA | A | 1 | 6.459 | 20.280 | 36.926 | 1.00 | 53.32 | A |
| ATOM | 21 | O6 | GUA | A | 1 | 6.922 | 21.401 | 36.675 | 1.00 | 50.63 | A |
| ATOM | 22 | C5 | GUA | A | 1 | 5.104 | 19.855 | 36.904 | 1.00 | 54.13 | A |
| ATOM | 23 | N7 | GUA | A | 1 | 3.951 | 20.571 | 36.615 | 1.00 | 54.92 | A |
| ATOM | 24 | C8 | GUA | A | 1 | 2.986 | 19.704 | 36.753 | 1.00 | 55.27 | A |
| ATOM | 25 | C2′ | GUA | A | 1 | 2.921 | 16.420 | 38.535 | 1.00 | 51.79 | A |
| ATOM | 26 | O2′ | GUA | A | 1 | 2.437 | 15.113 | 38.346 | 1.00 | 51.21 | A |
| ATOM | 27 | C3′ | GUA | A | 1 | 2.199 | 17.146 | 39.660 | 1.00 | 51.46 | A |
| ATOM | 28 | O3′ | GUA | A | 1 | 1.933 | 16.312 | 40.761 | 1.00 | 46.95 | A |
| ATOM | 29 | P | CYT | A | 2 | 2.157 | 16.875 | 42.244 | 1.00 | 48.57 | A |
| ATOM | 30 | O1P | CYT | A | 2 | 1.929 | 18.348 | 42.278 | 1.00 | 42.96 | A |
| ATOM | 31 | O2P | CYT | A | 2 | 1.391 | 15.995 | 43.167 | 1.00 | 46.56 | A |
| ATOM | 32 | O5′ | CYT | A | 2 | 3.706 | 16.597 | 42.479 | 1.00 | 47.77 | A |
| ATOM | 33 | C5′ | CYT | A | 2 | 4.204 | 15.277 | 42.381 | 1.00 | 45.44 | A |
| ATOM | 34 | C4′ | CYT | A | 2 | 5.702 | 15.280 | 42.403 | 1.00 | 45.59 | A |
| ATOM | 35 | O4′ | CYT | A | 2 | 6.215 | 15.773 | 41.143 | 1.00 | 46.82 | A |
| ATOM | 36 | C1′ | CYT | A | 2 | 7.449 | 16.434 | 41.369 | 1.00 | 47.01 | A |
| ATOM | 37 | N1 | CYT | A | 2 | 7.263 | 17.843 | 41.054 | 1.00 | 47.95 | A |
| ATOM | 38 | C6 | CYT | A | 2 | 6.035 | 18.339 | 40.736 | 1.00 | 47.93 | A |
| ATOM | 39 | C2 | CYT | A | 2 | 8.361 | 18.671 | 41.107 | 1.00 | 48.84 | A |
| ATOM | 40 | O2 | CYT | A | 2 | 9.458 | 18.170 | 41.388 | 1.00 | 52.02 | A |
| ATOM | 41 | N3 | CYT | A | 2 | 8.215 | 19.990 | 40.851 | 1.00 | 48.53 | A |
| ATOM | 42 | C4 | CYT | A | 2 | 7.016 | 20.470 | 40.531 | 1.00 | 48.44 | A |
| ATOM | 43 | N4 | CYT | A | 2 | 6.913 | 21.769 | 40.247 | 1.00 | 48.35 | A |
| ATOM | 44 | C5 | CYT | A | 2 | 5.868 | 19.636 | 40.471 | 1.00 | 49.66 | A |
| ATOM | 45 | C2′ | CYT | A | 2 | 7.763 | 16.289 | 42.859 | 1.00 | 46.37 | A |
| ATOM | 46 | O2′ | CYT | A | 2 | 8.562 | 15.149 | 43.091 | 1.00 | 45.81 | A |
| ATOM | 47 | C3′ | CYT | A | 2 | 6.364 | 16.166 | 43.439 | 1.00 | 44.45 | A |
| ATOM | 48 | O3′ | CYT | A | 2 | 6.412 | 15.484 | 44.675 | 1.00 | 41.89 | A |
| ATOM | 49 | P | GUA | A | 3 | 6.252 | 16.299 | 46.043 | 1.00 | 42.10 | A |
| ATOM | 50 | O1P | GUA | A | 3 | 5.280 | 17.407 | 45.892 | 1.00 | 40.61 | A |
| ATOM | 51 | O2P | GUA | A | 3 | 6.050 | 15.280 | 47.092 | 1.00 | 39.17 | A |
| ATOM | 52 | O5′ | GUA | A | 3 | 7.692 | 16.926 | 46.261 | 1.00 | 44.58 | A |
| ATOM | 53 | C5′ | GUA | A | 3 | 8.831 | 16.078 | 46.324 | 1.00 | 42.59 | A |
| ATOM | 54 | C4′ | GUA | A | 3 | 10.078 | 16.903 | 46.297 | 1.00 | 42.78 | A |
| ATOM | 55 | O4′ | GUA | A | 3 | 10.222 | 17.528 | 44.995 | 1.00 | 44.47 | A |
| ATOM | 56 | C1′ | GUA | A | 3 | 10.876 | 18.778 | 45.148 | 1.00 | 45.39 | A |
| ATOM | 57 | N9 | GUA | A | 3 | 9.983 | 19.828 | 44.670 | 1.00 | 48.03 | A |
| ATOM | 58 | C4 | GUA | A | 3 | 10.288 | 21.163 | 44.574 | 1.00 | 49.13 | A |
| ATOM | 59 | N3 | GUA | A | 3 | 11.468 | 21.727 | 44.896 | 1.00 | 50.26 | A |
| ATOM | 60 | C2 | GUA | A | 3 | 11.459 | 23.035 | 44.705 | 1.00 | 51.54 | A |
| ATOM | 61 | N2 | GUA | A | 3 | 12.545 | 23.757 | 44.991 | 1.00 | 54.67 | A |
| ATOM | 62 | N1 | GUA | A | 3 | 10.385 | 23.732 | 44.223 | 1.00 | 51.22 | A |
| ATOM | 63 | C6 | GUA | A | 3 | 9.162 | 23.176 | 43.880 | 1.00 | 50.02 | A |
| ATOM | 64 | O6 | GUA | A | 3 | 8.267 | 23.902 | 43.447 | 1.00 | 50.31 | A |
| ATOM | 65 | C5 | GUA | A | 3 | 9.148 | 21.767 | 44.095 | 1.00 | 49.23 | A |
| ATOM | 66 | N7 | GUA | A | 3 | 8.143 | 20.830 | 43.893 | 1.00 | 48.38 | A |
| ATOM | 67 | C8 | GUA | A | 3 | 8.683 | 19.694 | 44.245 | 1.00 | 47.42 | A |
| ATOM | 68 | C2′ | GUA | A | 3 | 11.186 | 18.924 | 46.639 | 1.00 | 43.47 | A |
| ATOM | 69 | O2′ | GUA | A | 3 | 12.459 | 18.377 | 46.910 | 1.00 | 45.99 | A |
| ATOM | 70 | C3′ | GUA | A | 3 | 10.077 | 18.083 | 47.243 | 1.00 | 42.30 | A |
| ATOM | 71 | O3′ | GUA | A | 3 | 10.414 | 17.644 | 48.543 | 1.00 | 43.04 | A |
| ATOM | 72 | P | CYT | A | 4 | 9.640 | 18.244 | 49.810 | 1.00 | 45.36 | A |
| ATOM | 73 | O1P | CYT | A | 4 | 8.240 | 18.530 | 49.414 | 1.00 | 46.28 | A |
| ATOM | 74 | O2P | CYT | A | 4 | 9.919 | 17.323 | 50.937 | 1.00 | 43.82 | A |
| ATOM | 75 | O5′ | CYT | A | 4 | 10.345 | 19.649 | 50.068 | 1.00 | 43.71 | A |
| ATOM | 76 | C5′ | CYT | A | 4 | 11.712 | 19.831 | 49.737 | 1.00 | 42.38 | A |
| ATOM | 77 | C4′ | CYT | A | 4 | 12.118 | 21.257 | 49.960 | 1.00 | 40.12 | A |
| ATOM | 78 | O4′ | CYT | A | 4 | 12.112 | 21.977 | 48.706 | 1.00 | 40.61 | A |
| ATOM | 79 | C1′ | CYT | A | 4 | 11.708 | 23.313 | 48.930 | 1.00 | 41.97 | A |
| ATOM | 80 | N1 | CYT | A | 4 | 10.437 | 23.527 | 48.227 | 1.00 | 44.61 | A |
| ATOM | 81 | C6 | CYT | A | 4 | 9.659 | 22.471 | 47.856 | 1.00 | 46.63 | A |
| ATOM | 82 | C2 | CYT | A | 4 | 10.033 | 24.820 | 47.950 | 1.00 | 44.55 | A |
| ATOM | 83 | O2 | CYT | A | 4 | 10.754 | 25.743 | 48.291 | 1.00 | 46.38 | A |
| ATOM | 84 | N3 | CYT | A | 4 | 8.862 | 25.031 | 47.319 | 1.00 | 45.19 | A |
| ATOM | 85 | C4 | CYT | A | 4 | 8.102 | 23.996 | 46.969 | 1.00 | 44.95 | A |
| ATOM | 86 | N4 | CYT | A | 4 | 6.953 | 24.242 | 46.367 | 1.00 | 45.12 | A |
| ATOM | 87 | C5 | CYT | A | 4 | 8.491 | 22.662 | 47.228 | 1.00 | 47.16 | A |
| ATOM | 88 | C2′ | CYT | A | 4 | 11.590 | 23.475 | 50.444 | 1.00 | 40.74 | A |
| ATOM | 89 | O2′ | CYT | A | 4 | 12.850 | 23.840 | 50.938 | 1.00 | 42.41 | A |
| ATOM | 90 | C3′ | CYT | A | 4 | 11.217 | 22.064 | 50.867 | 1.00 | 39.44 | A |
| ATOM | 91 | O3′ | CYT | A | 4 | 11.634 | 21.823 | 52.196 | 1.00 | 39.48 | A |
| ATOM | 92 | P | GUA | A | 5 | 10.570 | 21.822 | 53.397 | 1.00 | 40.65 | A |
| ATOM | 93 | O1P | GUA | A | 5 | 9.225 | 22.097 | 52.851 | 1.00 | 41.86 | A |
| ATOM | 94 | O2P | GUA | A | 5 | 10.792 | 20.615 | 54.220 | 1.00 | 38.51 | A |
| ATOM | 95 | O5′ | GUA | A | 5 | 11.010 | 23.078 | 54.260 | 1.00 | 40.18 | A |
| ATOM | 96 | C5′ | GUA | A | 5 | 10.119 | 24.153 | 54.485 | 1.00 | 39.67 | A |
| ATOM | 97 | C4′ | GUA | A | 5 | 10.754 | 25.446 | 54.064 | 1.00 | 38.08 | A |
| ATOM | 98 | O4′ | GUA | A | 5 | 11.037 | 25.373 | 52.646 | 1.00 | 38.21 | A |
| ATOM | 99 | C1′ | GUA | A | 5 | 10.629 | 26.579 | 52.018 | 1.00 | 41.62 | A |
| ATOM | 100 | N9 | GUA | A | 5 | 9.439 | 26.289 | 51.222 | 1.00 | 42.26 | A |
| ATOM | 101 | C4 | GUA | A | 5 | 8.684 | 27.173 | 50.489 | 1.00 | 43.07 | A |
| ATOM | 102 | N3 | GUA | A | 5 | 8.883 | 28.501 | 50.392 | 1.00 | 45.16 | A |
| ATOM | 103 | C2 | GUA | A | 5 | 7.985 | 29.083 | 49.613 | 1.00 | 43.59 | A |
| ATOM | 104 | N2 | GUA | A | 5 | 8.014 | 30.407 | 49.437 | 1.00 | 39.55 | A |
| ATOM | 105 | N1 | GUA | A | 5 | 6.993 | 28.408 | 48.963 | 1.00 | 44.54 | A |
| ATOM | 106 | C6 | GUA | A | 5 | 6.782 | 27.037 | 49.043 | 1.00 | 46.16 | A |
| ATOM | 107 | O6 | GUA | A | 5 | 5.867 | 26.515 | 48.401 | 1.00 | 51.42 | A |
| ATOM | 108 | C5 | GUA | A | 5 | 7.713 | 26.413 | 49.892 | 1.00 | 43.96 | A |
| ATOM | 109 | N7 | GUA | A | 5 | 7.838 | 25.086 | 50.251 | 1.00 | 44.24 | A |
| ATOM | 110 | C8 | GUA | A | 5 | 8.868 | 25.061 | 51.044 | 1.00 | 43.14 | A |
| ATOM | 111 | C2′ | GUA | A | 5 | 10.392 | 27.578 | 53.146 | 1.00 | 40.55 | A |
| ATOM | 112 | O2′ | GUA | A | 5 | 11.615 | 28.116 | 53.575 | 1.00 | 40.58 | A |
| ATOM | 113 | C3′ | GUA | A | 5 | 9.859 | 26.661 | 54.221 | 1.00 | 39.47 | A |
| ATOM | 114 | O3′ | GUA | A | 5 | 10.051 | 27.260 | 55.484 | 1.00 | 39.23 | A |
| ATOM | 115 | P | GUA | A | 6 | 8.814 | 27.981 | 56.202 | 1.00 | 39.81 | A |
| ATOM | 116 | O1P | GUA | A | 6 | 7.644 | 27.061 | 56.084 | 1.00 | 34.66 | A |
| ATOM | 117 | O2P | GUA | A | 6 | 9.244 | 28.441 | 57.539 | 1.00 | 38.23 | A |
| ATOM | 118 | O5′ | GUA | A | 6 | 8.606 | 29.286 | 55.316 | 1.00 | 37.57 | A |
| ATOM | 119 | C5′ | GUA | A | 6 | 9.549 | 30.343 | 55.374 | 1.00 | 35.30 | A |
| ATOM | 120 | C4′ | GUA | A | 6 | 9.070 | 31.533 | 54.575 | 1.00 | 35.94 | A |
| ATOM | 121 | O4′ | GUA | A | 6 | 8.934 | 31.175 | 53.178 | 1.00 | 36.31 | A |
| ATOM | 122 | C1′ | GUA | A | 6 | 7.826 | 31.865 | 52.619 | 1.00 | 37.34 | A |
| ATOM | 123 | N9 | GUA | A | 6 | 6.814 | 30.888 | 52.222 | 1.00 | 38.69 | A |
| ATOM | 124 | C4 | GUA | A | 6 | 5.667 | 31.157 | 51.531 | 1.00 | 39.43 | A |
| ATOM | 125 | N3 | GUA | A | 6 | 5.244 | 32.379 | 51.154 | 1.00 | 39.49 | A |
| ATOM | 126 | C2 | GUA | A | 6 | 4.118 | 32.327 | 50.467 | 1.00 | 39.64 | A |
| ATOM | 127 | N2 | GUA | A | 6 | 3.564 | 33.461 | 50.032 | 1.00 | 38.79 | A |
| ATOM | 128 | N1 | GUA | A | 6 | 3.456 | 31.160 | 50.161 | 1.00 | 39.77 | A |
| ATOM | 129 | C6 | GUA | A | 6 | 3.864 | 29.881 | 50.541 | 1.00 | 40.75 | A |
| ATOM | 130 | O6 | GUA | A | 6 | 3.190 | 28.886 | 50.209 | 1.00 | 36.84 | A |
| ATOM | 131 | C5 | GUA | A | 6 | 5.079 | 29.929 | 51.300 | 1.00 | 41.36 | A |
| ATOM | 132 | N7 | GUA | A | 6 | 5.824 | 28.910 | 51.883 | 1.00 | 38.94 | A |
| ATOM | 133 | C8 | GUA | A | 6 | 6.836 | 29.527 | 52.427 | 1.00 | 38.93 | A |
| ATOM | 134 | C2′ | GUA | A | 6 | 7.332 | 32.845 | 53.679 | 1.00 | 35.62 | A |
| ATOM | 135 | O2′ | GUA | A | 6 | 8.027 | 34.065 | 53.563 | 1.00 | 35.89 | A |
| ATOM | 136 | C3′ | GUA | A | 6 | 7.718 | 32.115 | 54.948 | 1.00 | 34.49 | A |
| ATOM | 137 | O3′ | GUA | A | 6 | 7.848 | 33.067 | 55.974 | 1.00 | 33.17 | A |
| ATOM | 138 | P | CYT | A | 7 | 6.760 | 33.100 | 57.135 | 1.00 | 36.95 | A |
| ATOM | 139 | O1P | CYT | A | 7 | 6.435 | 31.689 | 57.467 | 1.00 | 36.80 | A |
| ATOM | 140 | O2P | CYT | A | 7 | 7.223 | 34.013 | 58.202 | 1.00 | 37.25 | A |
| ATOM | 141 | O5′ | CYT | A | 7 | 5.510 | 33.776 | 56.427 | 1.00 | 37.87 | A |
| ATOM | 142 | C5′ | CYT | A | 7 | 5.579 | 35.130 | 56.015 | 1.00 | 39.08 | A |
| ATOM | 143 | C4′ | CYT | A | 7 | 4.321 | 35.514 | 55.296 | 1.00 | 41.04 | A |
| ATOM | 144 | O4′ | CYT | A | 7 | 4.262 | 34.828 | 54.017 | 1.00 | 41.75 | A |
| ATOM | 145 | C1′ | CYT | A | 7 | 2.927 | 34.445 | 53.751 | 1.00 | 42.97 | A |
| ATOM | 146 | N1 | CYT | A | 7 | 2.856 | 32.986 | 53.794 | 1.00 | 44.72 | A |
| ATOM | 147 | C6 | CYT | A | 7 | 3.633 | 32.261 | 54.651 | 1.00 | 45.48 | A |
| ATOM | 148 | C2 | CYT | A | 7 | 1.986 | 32.351 | 52.935 | 1.00 | 44.06 | A |
| ATOM | 149 | O2 | CYT | A | 7 | 1.282 | 33.047 | 52.206 | 1.00 | 46.39 | A |
| ATOM | 150 | N3 | CYT | A | 7 | 1.923 | 31.003 | 52.920 | 1.00 | 42.20 | A |
| ATOM | 151 | C4 | CYT | A | 7 | 2.679 | 30.299 | 53.754 | 1.00 | 43.31 | A |
| ATOM | 152 | N4 | CYT | A | 7 | 2.577 | 28.971 | 53.721 | 1.00 | 43.90 | A |
| ATOM | 153 | C5 | CYT | A | 7 | 3.574 | 30.926 | 54.666 | 1.00 | 46.03 | A |
| ATOM | 154 | C2′ | CYT | A | 7 | 2.076 | 35.118 | 54.820 | 1.00 | 43.20 | A |
| ATOM | 155 | O2′ | CYT | A | 7 | 1.826 | 36.454 | 54.434 | 1.00 | 47.51 | A |
| ATOM | 156 | C3′ | CYT | A | 7 | 3.038 | 35.108 | 55.987 | 1.00 | 40.73 | A |
| ATOM | 157 | O3′ | CYT | A | 7 | 2.664 | 36.118 | 56.886 | 1.00 | 38.58 | A |
| ATOM | 158 | P | GUA | A | 8 | 1.936 | 35.720 | 58.247 | 1.00 | 42.00 | A |
| ATOM | 159 | O1P | GUA | A | 8 | 2.563 | 34.485 | 58.780 | 1.00 | 41.25 | A |
| ATOM | 160 | O2P | GUA | A | 8 | 1.927 | 36.956 | 59.061 | 1.00 | 38.47 | A |
| ATOM | 161 | O5′ | GUA | A | 8 | 0.458 | 35.310 | 57.814 | 1.00 | 43.41 | A |
| ATOM | 162 | C5′ | GUA | A | 8 | −0.427 | 36.243 | 57.189 | 1.00 | 45.59 | A |
| ATOM | 163 | C4′ | GUA | A | 8 | −1.864 | 35.893 | 57.503 | 1.00 | 47.15 | A |
| ATOM | 164 | O4′ | GUA | A | 8 | −2.222 | 34.610 | 56.904 | 1.00 | 46.35 | A |
| ATOM | 165 | C1′ | GUA | A | 8 | −2.960 | 33.871 | 57.845 | 1.00 | 47.37 | A |
| ATOM | 166 | N9 | GUA | A | 8 | −2.819 | 32.449 | 57.568 | 1.00 | 50.30 | A |
| ATOM | 167 | C4 | GUA | A | 8 | −3.739 | 31.651 | 56.951 | 1.00 | 52.71 | A |
| ATOM | 168 | N3 | GUA | A | 8 | −4.914 | 32.058 | 56.430 | 1.00 | 53.07 | A |
| ATOM | 169 | C2 | GUA | A | 8 | −5.611 | 31.051 | 55.942 | 1.00 | 53.51 | A |
| ATOM | 170 | N2 | GUA | A | 8 | −6.804 | 31.279 | 55.392 | 1.00 | 55.21 | A |
| ATOM | 171 | N1 | GUA | A | 8 | −5.189 | 29.744 | 55.960 | 1.00 | 55.13 | A |
| ATOM | 172 | C6 | GUA | A | 8 | −3.975 | 29.298 | 56.485 | 1.00 | 55.81 | A |
| ATOM | 173 | O6 | GUA | A | 8 | −3.683 | 28.085 | 56.453 | 1.00 | 56.49 | A |
| ATOM | 174 | C5 | GUA | A | 8 | −3.216 | 30.374 | 57.012 | 1.00 | 53.87 | A |
| ATOM | 175 | N7 | GUA | A | 8 | −1.969 | 30.380 | 57.615 | 1.00 | 51.04 | A |
| ATOM | 176 | C8 | GUA | A | 8 | −1.770 | 31.633 | 57.919 | 1.00 | 52.05 | A |
| ATOM | 177 | C2′ | GUA | A | 8 | −2.329 | 34.255 | 59.174 | 1.00 | 46.97 | A |
| ATOM | 178 | O2′ | GUA | A | 8 | −3.150 | 33.861 | 60.245 | 1.00 | 43.61 | A |
| ATOM | 179 | C3′ | GUA | A | 8 | −2.172 | 35.760 | 58.994 | 1.00 | 48.46 | A |
| ATOM | 180 | O3′ | GUA | A | 8 | −3.372 | 36.455 | 59.313 | 1.00 | 51.14 | A |
| ATOM | 181 | P | ADE | A | 9 | −3.597 | 37.012 | 60.803 | 1.00 | 54.42 | A |
| ATOM | 182 | O1P | ADE | A | 9 | −3.394 | 38.489 | 60.798 | 1.00 | 52.70 | A |
| ATOM | 183 | O2P | ADE | A | 9 | −2.835 | 36.144 | 61.759 | 1.00 | 51.84 | A |
| ATOM | 184 | O5′ | ADE | A | 9 | −5.147 | 36.795 | 61.060 | 1.00 | 55.36 | A |
| ATOM | 185 | C5′ | ADE | A | 9 | −5.675 | 35.492 | 61.258 | 1.00 | 54.25 | A |
| ATOM | 186 | C4′ | ADE | A | 9 | −7.003 | 35.374 | 60.562 | 1.00 | 52.00 | A |
| ATOM | 187 | O4′ | ADE | A | 9 | −6.781 | 34.888 | 59.210 | 1.00 | 49.91 | A |
| ATOM | 188 | C1′ | ADE | A | 9 | −7.716 | 33.866 | 58.919 | 1.00 | 49.59 | A |
| ATOM | 189 | N9 | ADE | A | 9 | −7.032 | 32.582 | 59.020 | 1.00 | 46.92 | A |
| ATOM | 190 | C4 | ADE | A | 9 | −7.519 | 31.391 | 58.552 | 1.00 | 46.44 | A |
| ATOM | 191 | N3 | ADE | A | 9 | −8.686 | 31.187 | 57.925 | 1.00 | 45.85 | A |
| ATOM | 192 | C2 | ADE | A | 9 | −8.834 | 29.901 | 57.630 | 1.00 | 46.76 | A |
| ATOM | 193 | N1 | ADE | A | 9 | −8.009 | 28.877 | 57.867 | 1.00 | 46.92 | A |
| ATOM | 194 | C6 | ADE | A | 9 | −6.837 | 29.122 | 58.492 | 1.00 | 46.05 | A |
| ATOM | 195 | N6 | ADE | A | 9 | −6.009 | 28.100 | 58.715 | 1.00 | 46.20 | A |
| ATOM | 196 | C5 | ADE | A | 9 | −6.565 | 30.444 | 58.865 | 1.00 | 44.50 | A |
| ATOM | 197 | N7 | ADE | A | 9 | −5.493 | 31.028 | 59.515 | 1.00 | 44.66 | A |
| ATOM | 198 | C8 | ADE | A | 9 | −5.818 | 32.296 | 59.582 | 1.00 | 45.49 | A |
| ATOM | 199 | C2′ | ADE | A | 9 | −8.808 | 34.000 | 59.975 | 1.00 | 51.40 | A |
| ATOM | 200 | O2′ | ADE | A | 9 | −9.692 | 35.040 | 59.615 | 1.00 | 54.37 | A |
| ATOM | 201 | C3′ | ADE | A | 9 | −7.974 | 34.382 | 61.182 | 1.00 | 51.00 | A |
| ATOM | 202 | O3′ | ADE | A | 9 | −8.768 | 35.020 | 62.172 | 1.00 | 49.06 | A |
| ATOM | 203 | P | URI | A | 10 | −9.231 | 34.196 | 63.471 | 1.00 | 51.70 | A |
| ATOM | 204 | O1P | URI | A | 10 | −8.107 | 33.313 | 63.874 | 1.00 | 50.55 | A |
| ATOM | 205 | O2P | URI | A | 10 | −9.786 | 35.166 | 64.454 | 1.00 | 48.53 | A |
| ATOM | 206 | O5′ | URI | A | 10 | −10.403 | 33.261 | 62.918 | 1.00 | 51.67 | A |
| ATOM | 207 | C5′ | URI | A | 10 | −11.358 | 33.775 | 61.994 | 1.00 | 52.81 | A |
| ATOM | 208 | C4′ | URI | A | 10 | −12.176 | 32.661 | 61.394 | 1.00 | 54.72 | A |
| ATOM | 209 | O4′ | URI | A | 10 | −11.443 | 31.990 | 60.339 | 1.00 | 54.38 | A |
| ATOM | 210 | C1′ | URI | A | 10 | −11.921 | 30.661 | 60.223 | 1.00 | 54.28 | A |
| ATOM | 211 | N1 | URI | A | 10 | −10.816 | 29.747 | 60.502 | 1.00 | 54.62 | A |
| ATOM | 212 | C6 | URI | A | 10 | −9.672 | 30.161 | 61.130 | 1.00 | 54.70 | A |
| ATOM | 213 | C2 | URI | A | 10 | −10.988 | 28.448 | 60.125 | 1.00 | 55.57 | A |
| ATOM | 214 | O2 | URI | A | 10 | −11.989 | 28.064 | 59.553 | 1.00 | 55.75 | A |
| ATOM | 215 | N3 | URI | A | 10 | −9.951 | 27.613 | 60.440 | 1.00 | 56.18 | A |
| ATOM | 216 | C4 | URI | A | 10 | −8.785 | 27.952 | 61.079 | 1.00 | 55.37 | A |
| ATOM | 217 | O4 | URI | A | 10 | −7.964 | 27.073 | 61.337 | 1.00 | 58.05 | A |
| ATOM | 218 | C5 | URI | A | 10 | −8.676 | 29.330 | 61.425 | 1.00 | 54.29 | A |
| ATOM | 219 | C2′ | URI | A | 10 | −12.991 | 30.472 | 61.295 | 1.00 | 54.86 | A |
| ATOM | 220 | O2′ | URI | A | 10 | −14.268 | 30.721 | 60.757 | 1.00 | 56.63 | A |
| ATOM | 221 | C3′ | URI | A | 10 | −12.577 | 31.521 | 62.312 | 1.00 | 55.55 | A |
| ATOM | 222 | O3′ | URI | A | 10 | −13.695 | 31.899 | 63.096 | 1.00 | 58.29 | A |
| ATOM | 223 | P | URI | A | 11 | −14.115 | 31.020 | 64.373 | 1.00 | 59.94 | A |
| ATOM | 224 | O1P | URI | A | 11 | −12.956 | 30.853 | 65.283 | 1.00 | 59.36 | A |
| ATOM | 225 | O2P | URI | A | 11 | −15.352 | 31.662 | 64.888 | 1.00 | 60.92 | A |
| ATOM | 226 | O5′ | URI | A | 11 | −14.431 | 29.576 | 63.768 | 1.00 | 59.83 | A |
| ATOM | 227 | C5′ | URI | A | 11 | −15.507 | 29.360 | 62.854 | 1.00 | 59.21 | A |
| ATOM | 228 | C4′ | URI | A | 11 | −15.684 | 27.878 | 62.593 | 1.00 | 59.51 | A |
| ATOM | 229 | O4′ | URI | A | 11 | −14.571 | 27.380 | 61.805 | 1.00 | 59.78 | A |
| ATOM | 230 | C1′ | URI | A | 11 | −14.284 | 26.036 | 62.175 | 1.00 | 59.78 | A |
| ATOM | 231 | N1 | URI | A | 11 | −12.899 | 25.949 | 62.662 | 1.00 | 59.00 | A |
| ATOM | 232 | C6 | URI | A | 11 | −12.236 | 27.040 | 63.169 | 1.00 | 57.75 | A |
| ATOM | 233 | C2 | URI | A | 11 | −12.291 | 24.707 | 62.615 | 1.00 | 58.51 | A |
| ATOM | 234 | O2 | URI | A | 11 | −12.834 | 23.726 | 62.144 | 1.00 | 59.28 | A |
| ATOM | 235 | N3 | URI | A | 11 | −11.027 | 24.658 | 63.137 | 1.00 | 56.77 | A |
| ATOM | 236 | C4 | URI | A | 11 | −10.319 | 25.699 | 63.680 | 1.00 | 57.51 | A |
| ATOM | 237 | O4 | URI | A | 11 | −9.224 | 25.475 | 64.189 | 1.00 | 58.27 | A |
| ATOM | 238 | C5 | URI | A | 11 | −11.000 | 26.961 | 63.666 | 1.00 | 57.92 | A |
| ATOM | 239 | C2′ | URI | A | 11 | −15.266 | 25.639 | 63.276 | 1.00 | 59.82 | A |
| ATOM | 240 | O2′ | URI | A | 11 | −16.313 | 24.858 | 62.743 | 1.00 | 58.65 | A |
| ATOM | 241 | C3′ | URI | A | 11 | −15.673 | 27.001 | 63.833 | 1.00 | 60.24 | A |
| ATOM | 242 | O3′ | URI | A | 11 | −16.953 | 26.967 | 64.437 | 1.00 | 63.21 | A |
| ATOM | 243 | P | URI | A | 12 | −17.085 | 26.618 | 65.999 | 1.00 | 66.40 | A |
| ATOM | 244 | O1P | URI | A | 12 | −16.143 | 27.477 | 66.754 | 1.00 | 66.07 | A |
| ATOM | 245 | O2P | URI | A | 12 | −18.527 | 26.661 | 66.331 | 1.00 | 68.45 | A |
| ATOM | 246 | O5′ | URI | A | 12 | −16.586 | 25.107 | 66.094 | 1.00 | 65.97 | A |
| ATOM | 247 | C5′ | URI | A | 12 | −17.182 | 24.093 | 65.296 | 1.00 | 67.36 | A |
| ATOM | 248 | C4′ | URI | A | 12 | −16.479 | 22.772 | 65.505 | 1.00 | 69.81 | A |
| ATOM | 249 | O4′ | URI | A | 12 | −15.173 | 22.795 | 64.879 | 1.00 | 70.39 | A |
| ATOM | 250 | C1′ | URI | A | 12 | −14.329 | 21.861 | 65.529 | 1.00 | 69.88 | A |
| ATOM | 251 | N1 | URI | A | 12 | −13.093 | 22.547 | 65.926 | 1.00 | 70.58 | A |
| ATOM | 252 | C6 | URI | A | 12 | −13.009 | 23.915 | 65.928 | 1.00 | 70.52 | A |
| ATOM | 253 | C2 | URI | A | 12 | −12.019 | 21.765 | 66.322 | 1.00 | 70.85 | A |
| ATOM | 254 | O2 | URI | A | 12 | −12.042 | 20.550 | 66.310 | 1.00 | 72.51 | A |
| ATOM | 255 | N3 | URI | A | 12 | −10.916 | 22.462 | 66.734 | 1.00 | 69.65 | A |
| ATOM | 256 | C4 | URI | A | 12 | −10.779 | 23.828 | 66.789 | 1.00 | 70.03 | A |
| ATOM | 257 | O4 | URI | A | 12 | −9.773 | 24.304 | 67.296 | 1.00 | 70.62 | A |
| ATOM | 258 | C5 | URI | A | 12 | −11.917 | 24.566 | 66.335 | 1.00 | 70.95 | A |
| ATOM | 259 | C2′ | URI | A | 12 | −15.089 | 21.334 | 66.747 | 1.00 | 70.55 | A |
| ATOM | 260 | O2′ | URI | A | 12 | −15.628 | 20.071 | 66.444 | 1.00 | 71.94 | A |
| ATOM | 261 | C3′ | URI | A | 12 | −16.158 | 22.402 | 66.939 | 1.00 | 70.69 | A |
| ATOM | 262 | O3′ | URI | A | 12 | −17.305 | 21.860 | 67.579 | 1.00 | 72.76 | A |
| ATOM | 263 | P | ADE | A | 13 | −17.510 | 22.068 | 69.158 | 1.00 | 74.36 | A |
| ATOM | 264 | O1P | ADE | A | 13 | −18.547 | 21.100 | 69.565 | 1.00 | 73.45 | A |
| ATOM | 265 | O2P | ADE | A | 13 | −17.701 | 23.513 | 69.441 | 1.00 | 72.68 | A |
| ATOM | 266 | O5′ | ADE | A | 13 | −16.133 | 21.590 | 69.797 | 1.00 | 76.15 | A |
| ATOM | 267 | C5′ | ADE | A | 13 | −15.971 | 20.254 | 70.263 | 1.00 | 80.22 | A |
| ATOM | 268 | C4′ | ADE | A | 13 | −15.731 | 20.261 | 71.749 | 1.00 | 83.46 | A |
| ATOM | 269 | O4′ | ADE | A | 13 | −14.414 | 20.806 | 71.974 | 1.00 | 83.69 | A |
| ATOM | 270 | C1′ | ADE | A | 13 | −14.387 | 21.437 | 73.233 | 1.00 | 84.72 | A |
| ATOM | 271 | N9 | ADE | A | 13 | −13.487 | 22.587 | 73.160 | 1.00 | 83.53 | A |
| ATOM | 272 | C4 | ADE | A | 13 | −12.161 | 22.593 | 73.518 | 1.00 | 83.42 | A |
| ATOM | 273 | N3 | ADE | A | 13 | −11.430 | 21.552 | 73.955 | 1.00 | 83.67 | A |
| ATOM | 274 | C2 | ADE | A | 13 | −10.186 | 21.936 | 74.234 | 1.00 | 83.32 | A |
| ATOM | 275 | N1 | ADE | A | 13 | −9.634 | 23.151 | 74.139 | 1.00 | 83.45 | A |
| ATOM | 276 | C6 | ADE | A | 13 | −10.399 | 24.177 | 73.701 | 1.00 | 84.07 | A |
| ATOM | 277 | N6 | ADE | A | 13 | −9.855 | 25.395 | 73.624 | 1.00 | 85.24 | A |
| ATOM | 278 | C5 | ADE | A | 13 | −11.735 | 23.898 | 73.358 | 1.00 | 83.50 | A |
| ATOM | 279 | N7 | ADE | A | 13 | −12.765 | 24.696 | 72.874 | 1.00 | 82.37 | A |
| ATOM | 280 | C8 | ADE | A | 13 | −13.776 | 23.872 | 72.768 | 1.00 | 82.92 | A |
| ATOM | 281 | C2′ | ADE | A | 13 | −15.818 | 21.829 | 73.620 | 1.00 | 85.51 | A |
| ATOM | 282 | O2′ | ADE | A | 13 | −16.107 | 21.444 | 74.949 | 1.00 | 86.56 | A |
| ATOM | 283 | C3′ | ADE | A | 13 | −16.675 | 21.184 | 72.518 | 1.00 | 85.91 | A |
| ATOM | 284 | O3′ | ADE | A | 13 | −17.914 | 20.556 | 72.906 | 1.00 | 87.87 | A |
| ATOM | 285 | P | ADE | A | 14 | −17.976 | 19.507 | 74.134 | 1.00 | 88.89 | A |
| ATOM | 286 | O1P | ADE | A | 14 | −16.620 | 19.050 | 74.536 | 1.00 | 88.89 | A |
| ATOM | 287 | O2P | ADE | A | 14 | −18.998 | 18.501 | 73.756 | 1.00 | 89.39 | A |
| ATOM | 288 | O5′ | ADE | A | 14 | −18.566 | 20.387 | 75.314 | 1.00 | 88.88 | A |
| ATOM | 289 | C5′ | ADE | A | 14 | −18.936 | 21.735 | 75.070 | 1.00 | 91.70 | A |
| ATOM | 290 | C4′ | ADE | A | 14 | −20.142 | 22.101 | 75.891 | 1.00 | 94.04 | A |
| ATOM | 291 | O4′ | ADE | A | 14 | −21.326 | 22.017 | 75.082 | 1.00 | 95.65 | A |
| ATOM | 292 | C1′ | ADE | A | 14 | −22.438 | 21.956 | 75.937 | 1.00 | 97.65 | A |
| ATOM | 293 | N9 | ADE | A | 14 | −23.456 | 21.153 | 75.272 | 1.00 | 101.14 | A |
| ATOM | 294 | C4 | ADE | A | 14 | −24.067 | 21.476 | 74.086 | 1.00 | 103.55 | A |
| ATOM | 295 | N3 | ADE | A | 14 | −23.868 | 22.580 | 73.342 | 1.00 | 104.35 | A |
| ATOM | 296 | C2 | ADE | A | 14 | −24.625 | 22.549 | 72.244 | 1.00 | 104.89 | A |
| ATOM | 297 | N1 | ADE | A | 14 | −25.490 | 21.609 | 71.836 | 1.00 | 104.95 | A |
| ATOM | 298 | C6 | ADE | A | 14 | −25.664 | 20.510 | 72.608 | 1.00 | 104.89 | A |
| ATOM | 299 | N6 | ADE | A | 14 | −26.516 | 19.566 | 72.198 | 1.00 | 104.41 | A |
| ATOM | 300 | C5 | ADE | A | 14 | −24.924 | 20.428 | 73.805 | 1.00 | 104.57 | A |
| ATOM | 301 | N7 | ADE | A | 14 | −24.871 | 19.469 | 74.808 | 1.00 | 104.45 | A |
| ATOM | 302 | C8 | ADE | A | 14 | −23.990 | 19.947 | 75.654 | 1.00 | 103.05 | A |
| ATOM | 303 | C2′ | ADE | A | 14 | −21.959 | 21.406 | 77.289 | 1.00 | 95.73 | A |
| ATOM | 304 | O2′ | ADE | A | 14 | −22.285 | 22.320 | 78.317 | 1.00 | 96.76 | A |
| ATOM | 305 | C3′ | ADE | A | 14 | −20.450 | 21.191 | 77.068 | 1.00 | 93.55 | A |
| ATOM | 306 | O3′ | ADE | A | 14 | −19.615 | 21.602 | 78.153 | 1.00 | 89.70 | A |
| ATOM | 307 | P | CYT | A | 15 | −19.810 | 20.980 | 79.624 | 1.00 | 85.69 | A |
| ATOM | 308 | O1P | CYT | A | 15 | −18.605 | 20.172 | 79.932 | 1.00 | 84.11 | A |
| ATOM | 309 | O2P | CYT | A | 15 | −21.155 | 20.352 | 79.716 | 1.00 | 86.79 | A |
| ATOM | 310 | O5′ | CYT | A | 15 | −19.795 | 22.277 | 80.554 | 1.00 | 83.02 | A |
| ATOM | 311 | C5′ | CYT | A | 15 | −20.789 | 23.293 | 80.395 | 1.00 | 78.80 | A |
| ATOM | 312 | C4′ | CYT | A | 15 | −20.213 | 24.670 | 80.663 | 1.00 | 75.64 | A |
| ATOM | 313 | O4′ | CYT | A | 15 | −19.728 | 24.752 | 82.029 | 1.00 | 75.02 | A |
| ATOM | 314 | C1′ | CYT | A | 15 | −18.542 | 25.525 | 82.071 | 1.00 | 72.30 | A |
| ATOM | 315 | N1 | CYT | A | 15 | −17.419 | 24.625 | 82.362 | 1.00 | 71.80 | A |
| ATOM | 316 | C6 | CYT | A | 15 | −17.508 | 23.282 | 82.117 | 1.00 | 70.65 | A |
| ATOM | 317 | C2 | CYT | A | 15 | −16.246 | 25.171 | 82.878 | 1.00 | 71.69 | A |
| ATOM | 318 | O2 | CYT | A | 15 | −16.209 | 26.387 | 83.120 | 1.00 | 72.35 | A |
| ATOM | 319 | N3 | CYT | A | 15 | −15.185 | 24.364 | 83.102 | 1.00 | 70.96 | A |
| ATOM | 320 | C4 | CYT | A | 15 | −15.272 | 23.060 | 82.838 | 1.00 | 69.95 | A |
| ATOM | 321 | N4 | CYT | A | 15 | −14.194 | 22.315 | 83.047 | 1.00 | 68.69 | A |
| ATOM | 322 | C5 | CYT | A | 15 | −16.467 | 22.470 | 82.339 | 1.00 | 70.20 | A |
| ATOM | 323 | C2′ | CYT | A | 15 | −18.407 | 26.168 | 80.696 | 1.00 | 72.73 | A |
| ATOM | 324 | O2′ | CYT | A | 15 | −19.148 | 27.365 | 80.658 | 1.00 | 71.72 | A |
| ATOM | 325 | C3′ | CYT | A | 15 | −19.026 | 25.095 | 79.821 | 1.00 | 73.99 | A |
| ATOM | 326 | O3′ | CYT | A | 15 | −19.470 | 25.667 | 78.607 | 1.00 | 74.25 | A |
| ATOM | 327 | P | CYT | A | 16 | −18.436 | 25.845 | 77.399 | 1.00 | 72.99 | A |
| ATOM | 328 | O1P | CYT | A | 16 | −17.774 | 24.535 | 77.181 | 1.00 | 72.15 | A |
| ATOM | 329 | O2P | CYT | A | 16 | −19.150 | 26.508 | 76.283 | 1.00 | 73.64 | A |
| ATOM | 330 | O5′ | CYT | A | 16 | −17.369 | 26.872 | 77.971 | 1.00 | 71.54 | A |
| ATOM | 331 | C5′ | CYT | A | 16 | −17.678 | 28.252 | 78.047 | 1.00 | 70.37 | A |
| ATOM | 332 | C4′ | CYT | A | 16 | −16.510 | 29.015 | 78.622 | 1.00 | 70.94 | A |
| ATOM | 333 | O4′ | CYT | A | 16 | −16.071 | 28.393 | 79.858 | 1.00 | 70.27 | A |
| ATOM | 334 | C1′ | CYT | A | 16 | −14.679 | 28.598 | 80.016 | 1.00 | 69.83 | A |
| ATOM | 335 | N1 | CYT | A | 16 | −14.026 | 27.287 | 80.081 | 1.00 | 70.24 | A |
| ATOM | 336 | C6 | CYT | A | 16 | −14.719 | 26.140 | 79.812 | 1.00 | 70.01 | A |
| ATOM | 337 | C2 | CYT | A | 16 | −12.676 | 27.232 | 80.424 | 1.00 | 70.90 | A |
| ATOM | 338 | O2 | CYT | A | 16 | −12.073 | 28.290 | 80.649 | 1.00 | 71.93 | A |
| ATOM | 339 | N3 | CYT | A | 16 | −12.061 | 26.033 | 80.502 | 1.00 | 71.06 | A |
| ATOM | 340 | C4 | CYT | A | 16 | −12.745 | 24.918 | 80.250 | 1.00 | 71.12 | A |
| ATOM | 341 | N4 | CYT | A | 16 | −12.098 | 23.759 | 80.356 | 1.00 | 72.78 | A |
| ATOM | 342 | C5 | CYT | A | 16 | −14.123 | 24.944 | 79.883 | 1.00 | 70.62 | A |
| ATOM | 343 | C2′ | CYT | A | 16 | −14.212 | 29.422 | 78.815 | 1.00 | 70.28 | A |
| ATOM | 344 | O2′ | CYT | A | 16 | −14.284 | 30.798 | 79.118 | 1.00 | 72.82 | A |
| ATOM | 345 | C3′ | CYT | A | 16 | −15.249 | 29.041 | 77.777 | 1.00 | 70.11 | A |
| ATOM | 346 | O3′ | CYT | A | 16 | −15.331 | 30.049 | 76.783 | 1.00 | 68.85 | A |
| ATOM | 347 | P | GUA | A | 17 | −14.822 | 29.729 | 75.302 | 1.00 | 70.04 | A |
| ATOM | 348 | O1P | GUA | A | 17 | −15.539 | 28.515 | 74.834 | 1.00 | 68.06 | A |
| ATOM | 349 | O2P | GUA | A | 17 | −14.890 | 30.975 | 74.499 | 1.00 | 68.97 | A |
| ATOM | 350 | O5′ | GUA | A | 17 | −13.292 | 29.349 | 75.528 | 1.00 | 70.16 | A |
| ATOM | 351 | C5′ | GUA | A | 17 | −12.336 | 30.346 | 75.879 | 1.00 | 69.19 | A |
| ATOM | 352 | C4′ | GUA | A | 17 | −11.021 | 29.706 | 76.258 | 1.00 | 69.17 | A |
| ATOM | 353 | O4′ | GUA | A | 17 | −11.239 | 28.790 | 77.365 | 1.00 | 69.13 | A |
| ATOM | 354 | C1′ | GUA | A | 17 | −10.382 | 27.666 | 77.235 | 1.00 | 69.09 | A |
| ATOM | 355 | N9 | GUA | A | 17 | −11.205 | 26.467 | 77.085 | 1.00 | 67.10 | A |
| ATOM | 356 | C4 | GUA | A | 17 | −10.803 | 25.158 | 77.245 | 1.00 | 65.67 | A |
| ATOM | 357 | N3 | GUA | A | 17 | −9.569 | 24.740 | 77.593 | 1.00 | 66.51 | A |
| ATOM | 358 | C2 | GUA | A | 17 | −9.485 | 23.415 | 77.633 | 1.00 | 66.53 | A |
| ATOM | 359 | N2 | GUA | A | 17 | −8.323 | 22.819 | 77.952 | 1.00 | 66.24 | A |
| ATOM | 360 | N1 | GUA | A | 17 | −10.530 | 22.577 | 77.359 | 1.00 | 65.61 | A |
| ATOM | 361 | C6 | GUA | A | 17 | −11.810 | 22.988 | 77.003 | 1.00 | 66.05 | A |
| ATOM | 362 | O6 | GUA | A | 17 | −12.686 | 22.143 | 76.771 | 1.00 | 67.69 | A |
| ATOM | 363 | C5 | GUA | A | 17 | −11.913 | 24.399 | 76.955 | 1.00 | 65.35 | A |
| ATOM | 364 | N7 | GUA | A | 17 | −12.996 | 25.207 | 76.645 | 1.00 | 65.91 | A |
| ATOM | 365 | C8 | GUA | A | 17 | −12.532 | 26.422 | 76.738 | 1.00 | 66.71 | A |
| ATOM | 366 | C2′ | GUA | A | 17 | −9.467 | 27.952 | 76.040 | 1.00 | 69.82 | A |
| ATOM | 367 | O2′ | GUA | A | 17 | −8.322 | 28.647 | 76.492 | 1.00 | 68.53 | A |
| ATOM | 368 | C3′ | GUA | A | 17 | −10.351 | 28.854 | 75.190 | 1.00 | 69.79 | A |
| ATOM | 369 | O3′ | GUA | A | 17 | −9.536 | 29.659 | 74.347 | 1.00 | 71.64 | A |
| ATOM | 370 | P | URI | A | 18 | −9.841 | 29.732 | 72.773 | 1.00 | 75.29 | A |
| ATOM | 371 | O1P | URI | A | 18 | −10.045 | 28.347 | 72.291 | 1.00 | 75.45 | A |
| ATOM | 372 | O2P | URI | A | 18 | −10.896 | 30.747 | 72.545 | 1.00 | 74.13 | A |
| ATOM | 373 | O5′ | URI | A | 18 | −8.478 | 30.266 | 72.138 | 1.00 | 78.88 | A |
| ATOM | 374 | C5′ | URI | A | 18 | −8.001 | 31.590 | 72.399 | 1.00 | 84.21 | A |
| ATOM | 375 | C4′ | URI | A | 18 | −6.671 | 31.537 | 73.125 | 1.00 | 87.36 | A |
| ATOM | 376 | O4′ | URI | A | 18 | −6.874 | 30.864 | 74.406 | 1.00 | 92.07 | A |
| ATOM | 377 | C1′ | URI | A | 18 | −5.734 | 30.059 | 74.718 | 1.00 | 94.03 | A |
| ATOM | 378 | N1 | URI | A | 18 | −6.098 | 28.632 | 74.613 | 1.00 | 98.01 | A |
| ATOM | 379 | C6 | URI | A | 18 | −6.878 | 28.149 | 73.574 | 1.00 | 99.76 | A |
| ATOM | 380 | C2 | URI | A | 18 | −5.611 | 27.772 | 75.590 | 1.00 | 99.81 | A |
| ATOM | 381 | O2 | URI | A | 18 | −4.936 | 28.154 | 76.531 | 1.00 | 99.86 | A |
| ATOM | 382 | N3 | URI | A | 18 | −5.946 | 26.447 | 75.423 | 1.00 | 101.21 | A |
| ATOM | 383 | C4 | URI | A | 18 | −6.707 | 25.898 | 74.406 | 1.00 | 101.59 | A |
| ATOM | 384 | O4 | URI | A | 18 | −6.922 | 24.682 | 74.399 | 1.00 | 101.64 | A |
| ATOM | 385 | C5 | URI | A | 18 | −7.186 | 26.848 | 73.439 | 1.00 | 101.34 | A |
| ATOM | 386 | C2′ | URI | A | 18 | −4.684 | 30.427 | 73.679 | 1.00 | 90.65 | A |
| ATOM | 387 | O2′ | URI | A | 18 | −3.971 | 31.589 | 74.065 | 1.00 | 91.21 | A |
| ATOM | 388 | C3′ | URI | A | 18 | −5.588 | 30.677 | 72.493 | 1.00 | 87.49 | A |
| ATOM | 389 | O3′ | URI | A | 18 | −4.851 | 31.276 | 71.460 | 1.00 | 83.08 | A |
| ATOM | 390 | P | ADE | A | 19 | −3.932 | 30.345 | 70.538 | 1.00 | 81.25 | A |
| ATOM | 391 | O1P | ADE | A | 19 | −3.088 | 31.243 | 69.704 | 1.00 | 80.29 | A |
| ATOM | 392 | O2P | ADE | A | 19 | −4.832 | 29.356 | 69.885 | 1.00 | 80.09 | A |
| ATOM | 393 | O5′ | ADE | A | 19 | −2.994 | 29.564 | 71.567 | 1.00 | 79.28 | A |
| ATOM | 394 | C5′ | ADE | A | 19 | −1.630 | 29.937 | 71.742 | 1.00 | 77.82 | A |
| ATOM | 395 | C4′ | ADE | A | 19 | −0.795 | 28.745 | 72.158 | 1.00 | 77.72 | A |
| ATOM | 396 | O4′ | ADE | A | 19 | −1.556 | 27.877 | 73.031 | 1.00 | 77.57 | A |
| ATOM | 397 | C1′ | ADE | A | 19 | −1.016 | 26.564 | 72.971 | 1.00 | 77.68 | A |
| ATOM | 398 | N9 | ADE | A | 19 | −2.106 | 25.618 | 72.713 | 1.00 | 78.65 | A |
| ATOM | 399 | C4 | ADE | A | 19 | −2.044 | 24.245 | 72.774 | 1.00 | 78.79 | A |
| ATOM | 400 | N3 | ADE | A | 19 | −0.979 | 23.488 | 73.085 | 1.00 | 79.18 | A |
| ATOM | 401 | C2 | ADE | A | 19 | −1.292 | 22.193 | 73.032 | 1.00 | 79.24 | A |
| ATOM | 402 | N1 | ADE | A | 19 | −2.462 | 21.612 | 72.727 | 1.00 | 78.75 | A |
| ATOM | 403 | C6 | ADE | A | 19 | −3.515 | 22.399 | 72.424 | 1.00 | 78.55 | A |
| ATOM | 404 | N6 | ADE | A | 19 | −4.681 | 21.818 | 72.123 | 1.00 | 76.43 | A |
| ATOM | 405 | C5 | ADE | A | 19 | −3.313 | 23.793 | 72.444 | 1.00 | 79.04 | A |
| ATOM | 406 | N7 | ADE | A | 19 | −4.162 | 24.861 | 72.185 | 1.00 | 79.73 | A |
| ATOM | 407 | C8 | ADE | A | 19 | −3.401 | 25.916 | 72.359 | 1.00 | 80.36 | A |
| ATOM | 408 | C2′ | ADE | A | 19 | 0.063 | 26.576 | 71.886 | 1.00 | 77.49 | A |
| ATOM | 409 | O2′ | ADE | A | 19 | 1.327 | 26.762 | 72.486 | 1.00 | 76.48 | A |
| ATOM | 410 | C3′ | ADE | A | 19 | −0.343 | 27.792 | 71.064 | 1.00 | 78.16 | A |
| ATOM | 411 | O3′ | ADE | A | 19 | 0.795 | 28.312 | 70.379 | 1.00 | 79.19 | A |
| ATOM | 412 | P | URI | A | 20 | 1.119 | 27.836 | 68.871 | 1.00 | 79.14 | A |
| ATOM | 413 | O1P | URI | A | 20 | 0.046 | 28.324 | 67.964 | 1.00 | 76.77 | A |
| ATOM | 414 | O2P | URI | A | 20 | 2.536 | 28.205 | 68.611 | 1.00 | 78.22 | A |
| ATOM | 415 | O5′ | URI | A | 20 | 1.015 | 26.249 | 68.908 | 1.00 | 75.01 | A |
| ATOM | 416 | C5′ | URI | A | 20 | 2.060 | 25.466 | 69.454 | 1.00 | 72.40 | A |
| ATOM | 417 | C4′ | URI | A | 20 | 1.807 | 24.016 | 69.163 | 1.00 | 71.99 | A |
| ATOM | 418 | O4′ | URI | A | 20 | 0.764 | 23.525 | 70.046 | 1.00 | 71.40 | A |
| ATOM | 419 | C1′ | URI | A | 20 | −0.064 | 22.610 | 69.348 | 1.00 | 70.50 | A |
| ATOM | 420 | N1 | URI | A | 20 | −1.419 | 23.173 | 69.278 | 1.00 | 69.99 | A |
| ATOM | 421 | C6 | URI | A | 20 | −1.641 | 24.526 | 69.396 | 1.00 | 69.65 | A |
| ATOM | 422 | C2 | URI | A | 20 | −2.470 | 22.296 | 69.079 | 1.00 | 69.79 | A |
| ATOM | 423 | O2 | URI | A | 20 | −2.315 | 21.091 | 68.976 | 1.00 | 69.83 | A |
| ATOM | 424 | N3 | URI | A | 20 | −3.711 | 22.883 | 69.006 | 1.00 | 68.93 | A |
| ATOM | 425 | C4 | URI | A | 20 | −4.003 | 24.223 | 69.116 | 1.00 | 68.00 | A |
| ATOM | 426 | O4 | URI | A | 20 | −5.179 | 24.586 | 69.103 | 1.00 | 66.76 | A |
| ATOM | 427 | C5 | URI | A | 20 | −2.863 | 25.066 | 69.320 | 1.00 | 68.94 | A |
| ATOM | 428 | C2′ | URI | A | 20 | 0.552 | 22.421 | 67.960 | 1.00 | 71.39 | A |
| ATOM | 429 | O2′ | URI | A | 20 | 1.424 | 21.310 | 67.946 | 1.00 | 69.58 | A |
| ATOM | 430 | C3′ | URI | A | 20 | 1.267 | 23.750 | 67.771 | 1.00 | 72.13 | A |
| ATOM | 431 | O3′ | URI | A | 20 | 2.338 | 23.633 | 66.851 | 1.00 | 74.59 | A |
| ATOM | 432 | P | URI | A | 21 | 2.098 | 23.975 | 65.301 | 1.00 | 75.64 | A |
| ATOM | 433 | O1P | URI | A | 21 | 3.447 | 24.101 | 64.692 | 1.00 | 75.59 | A |
| ATOM | 434 | O2P | URI | A | 21 | 1.146 | 25.116 | 65.209 | 1.00 | 76.07 | A |
| ATOM | 435 | O5′ | URI | A | 21 | 1.388 | 22.660 | 64.745 | 1.00 | 72.90 | A |
| ATOM | 436 | C5′ | URI | A | 21 | 2.021 | 21.390 | 64.850 | 1.00 | 69.19 | A |
| ATOM | 437 | C4′ | URI | A | 21 | 1.095 | 20.303 | 64.371 | 1.00 | 67.68 | A |
| ATOM | 438 | O4′ | URI | A | 21 | 0.085 | 20.078 | 65.387 | 1.00 | 67.43 | A |
| ATOM | 439 | C1′ | URI | A | 21 | −1.146 | 19.748 | 64.772 | 1.00 | 68.79 | A |
| ATOM | 440 | N1 | URI | A | 21 | −2.134 | 20.782 | 65.113 | 1.00 | 70.91 | A |
| ATOM | 441 | C6 | URI | A | 21 | −1.751 | 22.047 | 65.500 | 1.00 | 70.89 | A |
| ATOM | 442 | C2 | URI | A | 21 | −3.479 | 20.439 | 65.021 | 1.00 | 72.02 | A |
| ATOM | 443 | O2 | URI | A | 21 | −3.860 | 19.327 | 64.684 | 1.00 | 72.32 | A |
| ATOM | 444 | N3 | URI | A | 21 | −4.362 | 21.450 | 65.333 | 1.00 | 72.47 | A |
| ATOM | 445 | C4 | URI | A | 21 | −4.048 | 22.740 | 65.716 | 1.00 | 72.29 | A |
| ATOM | 446 | O4 | URI | A | 21 | −4.958 | 23.549 | 65.917 | 1.00 | 70.84 | A |
| ATOM | 447 | C5 | URI | A | 21 | −2.637 | 23.013 | 65.796 | 1.00 | 71.91 | A |
| ATOM | 448 | C2′ | URI | A | 21 | −0.888 | 19.673 | 63.267 | 1.00 | 68.38 | A |
| ATOM | 449 | O2′ | URI | A | 21 | −0.583 | 18.348 | 62.893 | 1.00 | 67.60 | A |
| ATOM | 450 | C3′ | URI | A | 21 | 0.293 | 20.617 | 63.119 | 1.00 | 67.38 | A |
| ATOM | 451 | O3′ | URI | A | 21 | 1.015 | 20.314 | 61.935 | 1.00 | 66.83 | A |
| ATOM | 452 | P | GUA | A | 22 | 0.452 | 20.816 | 60.513 | 1.00 | 69.29 | A |
| ATOM | 453 | O1P | GUA | A | 22 | −0.119 | 22.178 | 60.708 | 1.00 | 67.61 | A |
| ATOM | 454 | O2P | GUA | A | 22 | 1.497 | 20.595 | 59.478 | 1.00 | 66.54 | A |
| ATOM | 455 | O5′ | GUA | A | 22 | −0.771 | 19.845 | 60.199 | 1.00 | 67.90 | A |
| ATOM | 456 | C5′ | GUA | A | 22 | −0.616 | 18.743 | 59.316 | 1.00 | 67.34 | A |
| ATOM | 457 | C4′ | GUA | A | 22 | −1.951 | 18.338 | 58.728 | 1.00 | 67.34 | A |
| ATOM | 458 | O4′ | GUA | A | 22 | −3.052 | 18.853 | 59.524 | 1.00 | 67.07 | A |
| ATOM | 459 | C1′ | GUA | A | 22 | −4.156 | 19.137 | 58.674 | 1.00 | 65.78 | A |
| ATOM | 460 | N9 | GUA | A | 22 | −4.409 | 20.570 | 58.714 | 1.00 | 63.69 | A |
| ATOM | 461 | C4 | GUA | A | 22 | −5.519 | 21.196 | 58.234 | 1.00 | 64.17 | A |
| ATOM | 462 | N3 | GUA | A | 22 | −6.578 | 20.590 | 57.669 | 1.00 | 66.48 | A |
| ATOM | 463 | C2 | GUA | A | 22 | −7.508 | 21.456 | 57.316 | 1.00 | 66.72 | A |
| ATOM | 464 | N2 | GUA | A | 22 | −8.640 | 21.019 | 56.741 | 1.00 | 67.37 | A |
| ATOM | 465 | N1 | GUA | A | 22 | −7.400 | 22.810 | 57.504 | 1.00 | 64.80 | A |
| ATOM | 466 | C6 | GUA | A | 22 | −6.319 | 23.449 | 58.092 | 1.00 | 62.77 | A |
| ATOM | 467 | O6 | GUA | A | 22 | −6.336 | 24.670 | 58.235 | 1.00 | 63.28 | A |
| ATOM | 468 | C5 | GUA | A | 22 | −5.317 | 22.535 | 58.467 | 1.00 | 63.01 | A |
| ATOM | 469 | N7 | GUA | A | 22 | −4.098 | 22.745 | 59.086 | 1.00 | 62.71 | A |
| ATOM | 470 | C8 | GUA | A | 22 | −3.594 | 21.551 | 59.213 | 1.00 | 62.98 | A |
| ATOM | 471 | C2′ | GUA | A | 22 | −3.750 | 18.723 | 57.262 | 1.00 | 67.09 | A |
| ATOM | 472 | O2′ | GUA | A | 22 | −4.164 | 17.403 | 56.976 | 1.00 | 66.74 | A |
| ATOM | 473 | C3′ | GUA | A | 22 | −2.242 | 18.887 | 57.346 | 1.00 | 67.87 | A |
| ATOM | 474 | O3′ | GUA | A | 22 | −1.577 | 18.129 | 56.355 | 1.00 | 68.73 | A |
| ATOM | 475 | P | CYT | A | 23 | −0.934 | 18.878 | 55.097 | 1.00 | 69.00 | A |
| ATOM | 476 | O1P | CYT | A | 23 | −0.475 | 20.227 | 55.551 | 1.00 | 66.88 | A |
| ATOM | 477 | O2P | CYT | A | 23 | 0.034 | 17.929 | 54.486 | 1.00 | 69.69 | A |
| ATOM | 478 | O5′ | CYT | A | 23 | −2.168 | 19.061 | 54.112 | 1.00 | 66.23 | A |
| ATOM | 479 | C5′ | CYT | A | 23 | −3.205 | 18.095 | 54.073 | 1.00 | 61.77 | A |
| ATOM | 480 | C4′ | CYT | A | 23 | −4.466 | 18.709 | 53.528 | 1.00 | 59.16 | A |
| ATOM | 481 | O4′ | CYT | A | 23 | −5.143 | 19.424 | 54.589 | 1.00 | 57.70 | A |
| ATOM | 482 | C1′ | CYT | A | 23 | −5.882 | 20.493 | 54.027 | 1.00 | 57.19 | A |
| ATOM | 483 | N1 | CYT | A | 23 | −5.376 | 21.740 | 54.583 | 1.00 | 55.24 | A |
| ATOM | 484 | C6 | CYT | A | 23 | −4.177 | 21.791 | 55.232 | 1.00 | 54.51 | A |
| ATOM | 485 | C2 | CYT | A | 23 | −6.128 | 22.881 | 54.404 | 1.00 | 55.15 | A |
| ATOM | 486 | O2 | CYT | A | 23 | −7.221 | 22.783 | 53.826 | 1.00 | 53.58 | A |
| ATOM | 487 | N3 | CYT | A | 23 | −5.652 | 24.063 | 54.851 | 1.00 | 55.42 | A |
| ATOM | 488 | C4 | CYT | A | 23 | −4.464 | 24.116 | 55.450 | 1.00 | 54.64 | A |
| ATOM | 489 | N4 | CYT | A | 23 | −4.011 | 25.308 | 55.831 | 1.00 | 55.93 | A |
| ATOM | 490 | C5 | CYT | A | 23 | −3.683 | 22.951 | 55.672 | 1.00 | 54.66 | A |
| ATOM | 491 | C2′ | CYT | A | 23 | −5.636 | 20.472 | 52.517 | 1.00 | 57.73 | A |
| ATOM | 492 | O2′ | CYT | A | 23 | −6.674 | 19.773 | 51.861 | 1.00 | 58.26 | A |
| ATOM | 493 | C3′ | CYT | A | 23 | −4.292 | 19.765 | 52.451 | 1.00 | 57.38 | A |
| ATOM | 494 | O3′ | CYT | A | 23 | −4.104 | 19.165 | 51.181 | 1.00 | 57.87 | A |
| ATOM | 495 | P | ADE | A | 24 | −3.341 | 19.973 | 50.014 | 1.00 | 61.30 | A |
| ATOM | 496 | O1P | ADE | A | 24 | −3.432 | 19.164 | 48.772 | 1.00 | 59.15 | A |
| ATOM | 497 | O2P | ADE | A | 24 | −2.010 | 20.417 | 50.511 | 1.00 | 60.39 | A |
| ATOM | 498 | O5′ | ADE | A | 24 | −4.235 | 21.263 | 49.770 | 1.00 | 58.67 | A |
| ATOM | 499 | C5′ | ADE | A | 24 | −5.394 | 21.182 | 48.973 | 1.00 | 55.21 | A |
| ATOM | 500 | C4′ | ADE | A | 24 | −6.204 | 22.429 | 49.134 | 1.00 | 54.44 | A |
| ATOM | 501 | O4′ | ADE | A | 24 | −6.327 | 22.758 | 50.543 | 1.00 | 55.41 | A |
| ATOM | 502 | C1′ | ADE | A | 24 | −6.383 | 24.170 | 50.691 | 1.00 | 54.19 | A |
| ATOM | 503 | N9 | ADE | A | 24 | −5.209 | 24.575 | 51.446 | 1.00 | 54.73 | A |
| ATOM | 504 | C4 | ADE | A | 24 | −4.890 | 25.849 | 51.828 | 1.00 | 55.04 | A |
| ATOM | 505 | N3 | ADE | A | 24 | −5.585 | 26.968 | 51.577 | 1.00 | 55.11 | A |
| ATOM | 506 | C2 | ADE | A | 24 | −4.980 | 28.021 | 52.120 | 1.00 | 56.89 | A |
| ATOM | 507 | N1 | ADE | A | 24 | −3.843 | 28.074 | 52.836 | 1.00 | 56.19 | A |
| ATOM | 508 | C6 | ADE | A | 24 | −3.176 | 26.924 | 53.069 | 1.00 | 56.00 | A |
| ATOM | 509 | N6 | ADE | A | 24 | −2.056 | 26.967 | 53.791 | 1.00 | 57.98 | A |
| ATOM | 510 | C5 | ADE | A | 24 | −3.710 | 25.744 | 52.541 | 1.00 | 55.31 | A |
| ATOM | 511 | N7 | ADE | A | 24 | −3.287 | 24.425 | 52.596 | 1.00 | 55.37 | A |
| ATOM | 512 | C8 | ADE | A | 24 | −4.207 | 23.774 | 51.929 | 1.00 | 55.10 | A |
| ATOM | 513 | C2′ | ADE | A | 24 | −6.361 | 24.744 | 49.279 | 1.00 | 53.40 | A |
| ATOM | 514 | O2′ | ADE | A | 24 | −7.683 | 24.807 | 48.799 | 1.00 | 53.18 | A |
| ATOM | 515 | C3′ | ADE | A | 24 | −5.562 | 23.671 | 48.570 | 1.00 | 52.64 | A |
| ATOM | 516 | O3′ | ADE | A | 24 | −5.743 | 23.711 | 47.178 | 1.00 | 50.69 | A |
| ATOM | 517 | P | GUA | A | 25 | −4.634 | 24.416 | 46.271 | 1.00 | 51.67 | A |
| ATOM | 518 | O1P | GUA | A | 25 | −3.300 | 23.875 | 46.655 | 1.00 | 47.23 | A |
| ATOM | 519 | O2P | GUA | A | 25 | −5.078 | 24.335 | 44.861 | 1.00 | 49.51 | A |
| ATOM | 520 | O5′ | GUA | A | 25 | −4.740 | 25.936 | 46.718 | 1.00 | 48.34 | A |
| ATOM | 521 | C5′ | GUA | A | 25 | −5.596 | 26.822 | 46.023 | 1.00 | 46.65 | A |
| ATOM | 522 | C4′ | GUA | A | 25 | −5.610 | 28.158 | 46.701 | 1.00 | 45.25 | A |
| ATOM | 523 | O4′ | GUA | A | 25 | −5.440 | 27.968 | 48.132 | 1.00 | 46.00 | A |
| ATOM | 524 | C1′ | GUA | A | 25 | −4.661 | 29.028 | 48.666 | 1.00 | 46.72 | A |
| ATOM | 525 | N9 | GUA | A | 25 | −3.430 | 28.456 | 49.219 | 1.00 | 49.02 | A |
| ATOM | 526 | C4 | GUA | A | 25 | −2.443 | 29.116 | 49.914 | 1.00 | 47.40 | A |
| ATOM | 527 | N3 | GUA | A | 25 | −2.422 | 30.427 | 50.202 | 1.00 | 47.74 | A |
| ATOM | 528 | C2 | GUA | A | 25 | −1.357 | 30.759 | 50.909 | 1.00 | 48.49 | A |
| ATOM | 529 | N2 | GUA | A | 25 | −1.178 | 32.029 | 51.298 | 1.00 | 48.42 | A |
| ATOM | 530 | N1 | GUA | A | 25 | −0.387 | 29.875 | 51.290 | 1.00 | 48.24 | A |
| ATOM | 531 | C6 | GUA | A | 25 | −0.384 | 28.520 | 50.998 | 1.00 | 49.17 | A |
| ATOM | 532 | O6 | GUA | A | 25 | 0.547 | 27.806 | 51.392 | 1.00 | 51.70 | A |
| ATOM | 533 | C5 | GUA | A | 25 | −1.522 | 28.149 | 50.251 | 1.00 | 48.43 | A |
| ATOM | 534 | N7 | GUA | A | 25 | −1.910 | 26.908 | 49.770 | 1.00 | 48.09 | A |
| ATOM | 535 | C8 | GUA | A | 25 | −3.040 | 27.138 | 49.160 | 1.00 | 49.59 | A |
| ATOM | 536 | C2′ | GUA | A | 25 | −4.446 | 30.015 | 47.517 | 1.00 | 45.58 | A |
| ATOM | 537 | O2′ | GUA | A | 25 | −5.520 | 30.921 | 47.503 | 1.00 | 45.79 | A |
| ATOM | 538 | C3′ | GUA | A | 25 | −4.475 | 29.081 | 46.318 | 1.00 | 43.20 | A |
| ATOM | 539 | O3′ | GUA | A | 25 | −4.781 | 29.760 | 45.115 | 1.00 | 40.74 | A |
| ATOM | 540 | P | URI | A | 26 | −3.717 | 29.749 | 43.908 | 1.00 | 43.39 | A |
| ATOM | 541 | O1P | URI | A | 26 | −3.032 | 28.438 | 43.967 | 1.00 | 39.68 | A |
| ATOM | 542 | O2P | URI | A | 26 | −4.351 | 30.177 | 42.633 | 1.00 | 40.27 | A |
| ATOM | 543 | O5′ | URI | A | 26 | −2.687 | 30.886 | 44.340 | 1.00 | 42.45 | A |
| ATOM | 544 | C5′ | URI | A | 26 | −3.136 | 32.211 | 44.602 | 1.00 | 42.90 | A |
| ATOM | 545 | C4′ | URI | A | 26 | −2.187 | 32.935 | 45.546 | 1.00 | 43.91 | A |
| ATOM | 546 | O4′ | URI | A | 26 | −2.250 | 32.368 | 46.882 | 1.00 | 43.17 | A |
| ATOM | 547 | C1′ | URI | A | 26 | −0.958 | 32.404 | 47.469 | 1.00 | 42.74 | A |
| ATOM | 548 | N1 | URI | A | 26 | −0.447 | 31.033 | 47.527 | 1.00 | 41.76 | A |
| ATOM | 549 | C6 | URI | A | 26 | −1.034 | 30.006 | 46.830 | 1.00 | 40.65 | A |
| ATOM | 550 | C2 | URI | A | 26 | 0.645 | 30.820 | 48.319 | 1.00 | 38.14 | A |
| ATOM | 551 | O2 | URI | A | 26 | 1.221 | 31.726 | 48.877 | 1.00 | 34.19 | A |
| ATOM | 552 | N3 | URI | A | 26 | 1.050 | 29.517 | 48.409 | 1.00 | 36.80 | A |
| ATOM | 553 | C4 | URI | A | 26 | 0.507 | 28.443 | 47.754 | 1.00 | 37.46 | A |
| ATOM | 554 | O4 | URI | A | 26 | 0.959 | 27.329 | 47.962 | 1.00 | 38.00 | A |
| ATOM | 555 | C5 | URI | A | 26 | −0.597 | 28.753 | 46.905 | 1.00 | 39.39 | A |
| ATOM | 556 | C2′ | URI | A | 26 | −0.083 | 33.246 | 46.551 | 1.00 | 43.16 | A |
| ATOM | 557 | O2′ | URI | A | 26 | −0.193 | 34.611 | 46.897 | 1.00 | 43.72 | A |
| ATOM | 558 | C3′ | URI | A | 26 | −0.705 | 32.916 | 45.209 | 1.00 | 43.08 | A |
| ATOM | 559 | O3′ | URI | A | 26 | −0.386 | 33.922 | 44.284 | 1.00 | 42.91 | A |
| ATOM | 560 | P | CYT | A | 27 | 0.781 | 33.656 | 43.243 | 1.00 | 47.08 | A |
| ATOM | 561 | O1P | CYT | A | 27 | 0.666 | 32.223 | 42.869 | 1.00 | 43.13 | A |
| ATOM | 562 | O2P | CYT | A | 27 | 0.747 | 34.699 | 42.184 | 1.00 | 45.54 | A |
| ATOM | 563 | O5′ | CYT | A | 27 | 2.092 | 33.854 | 44.129 | 1.00 | 49.14 | A |
| ATOM | 564 | C5′ | CYT | A | 27 | 2.469 | 35.144 | 44.602 | 1.00 | 47.17 | A |
| ATOM | 565 | C4′ | CYT | A | 27 | 3.765 | 35.055 | 45.362 | 1.00 | 46.81 | A |
| ATOM | 566 | O4′ | CYT | A | 27 | 3.558 | 34.217 | 46.522 | 1.00 | 48.01 | A |
| ATOM | 567 | C1′ | CYT | A | 27 | 4.764 | 33.560 | 46.856 | 1.00 | 46.91 | A |
| ATOM | 568 | N1 | CYT | A | 27 | 4.514 | 32.118 | 46.875 | 1.00 | 46.48 | A |
| ATOM | 569 | C6 | CYT | A | 27 | 3.504 | 31.568 | 46.147 | 1.00 | 45.80 | A |
| ATOM | 570 | C2 | CYT | A | 27 | 5.331 | 31.320 | 47.646 | 1.00 | 47.12 | A |
| ATOM | 571 | O2 | CYT | A | 27 | 6.233 | 31.854 | 48.289 | 1.00 | 49.30 | A |
| ATOM | 572 | N3 | CYT | A | 27 | 5.127 | 29.990 | 47.676 | 1.00 | 46.54 | A |
| ATOM | 573 | C4 | CYT | A | 27 | 4.144 | 29.458 | 46.961 | 1.00 | 44.47 | A |
| ATOM | 574 | N4 | CYT | A | 27 | 3.979 | 28.144 | 47.013 | 1.00 | 44.09 | A |
| ATOM | 575 | C5 | CYT | A | 27 | 3.287 | 30.251 | 46.159 | 1.00 | 45.18 | A |
| ATOM | 576 | C2′ | CYT | A | 27 | 5.794 | 33.956 | 45.803 | 1.00 | 48.09 | A |
| ATOM | 577 | O2′ | CYT | A | 27 | 6.562 | 35.031 | 46.282 | 1.00 | 49.01 | A |
| ATOM | 578 | C3′ | CYT | A | 27 | 4.900 | 34.362 | 44.643 | 1.00 | 46.40 | A |
| ATOM | 579 | O3′ | CYT | A | 27 | 5.589 | 35.277 | 43.816 | 1.00 | 46.57 | A |
| ATOM | 580 | P | GUA | A | 28 | 5.737 | 34.965 | 42.253 | 1.00 | 47.65 | A |
| ATOM | 581 | O1P | GUA | A | 28 | 4.565 | 34.146 | 41.873 | 1.00 | 48.71 | A |
| ATOM | 582 | O2P | GUA | A | 28 | 6.029 | 36.244 | 41.550 | 1.00 | 44.75 | A |
| ATOM | 583 | O5′ | GUA | A | 28 | 6.988 | 33.995 | 42.133 | 1.00 | 48.15 | A |
| ATOM | 584 | C5′ | GUA | A | 28 | 8.304 | 34.513 | 42.003 | 1.00 | 45.36 | A |
| ATOM | 585 | C4′ | GUA | A | 28 | 9.135 | 34.087 | 43.181 | 1.00 | 42.58 | A |
| ATOM | 586 | O4′ | GUA | A | 28 | 8.258 | 33.510 | 44.180 | 1.00 | 40.98 | A |
| ATOM | 587 | C1′ | GUA | A | 28 | 8.963 | 32.528 | 44.906 | 1.00 | 40.82 | A |
| ATOM | 588 | N9 | GUA | A | 28 | 8.238 | 31.274 | 44.785 | 1.00 | 41.22 | A |
| ATOM | 589 | C4 | GUA | A | 28 | 8.577 | 30.088 | 45.376 | 1.00 | 42.88 | A |
| ATOM | 590 | N3 | GUA | A | 28 | 9.650 | 29.876 | 46.163 | 1.00 | 42.86 | A |
| ATOM | 591 | C2 | GUA | A | 28 | 9.701 | 28.629 | 46.600 | 1.00 | 44.96 | A |
| ATOM | 592 | N2 | GUA | A | 28 | 10.691 | 28.242 | 47.405 | 1.00 | 44.64 | A |
| ATOM | 593 | N1 | GUA | A | 28 | 8.780 | 27.666 | 46.283 | 1.00 | 45.93 | A |
| ATOM | 594 | C6 | GUA | A | 28 | 7.674 | 27.859 | 45.470 | 1.00 | 43.79 | A |
| ATOM | 595 | O6 | GUA | A | 28 | 6.918 | 26.919 | 45.245 | 1.00 | 44.25 | A |
| ATOM | 596 | C5 | GUA | A | 28 | 7.599 | 29.197 | 44.999 | 1.00 | 43.04 | A |
| ATOM | 597 | N7 | GUA | A | 28 | 6.667 | 29.812 | 44.180 | 1.00 | 42.58 | A |
| ATOM | 598 | C8 | GUA | A | 28 | 7.091 | 31.044 | 44.078 | 1.00 | 40.68 | A |
| ATOM | 599 | C2′ | GUA | A | 28 | 10.374 | 32.465 | 44.326 | 1.00 | 40.07 | A |
| ATOM | 600 | O2′ | GUA | A | 28 | 11.213 | 33.316 | 45.079 | 1.00 | 37.21 | A |
| ATOM | 601 | C3′ | GUA | A | 28 | 10.128 | 32.974 | 42.917 | 1.00 | 40.00 | A |
| ATOM | 602 | O3′ | GUA | A | 28 | 11.306 | 33.508 | 42.343 | 1.00 | 38.81 | A |
| ATOM | 603 | P | CYT | A | 29 | 11.983 | 32.776 | 41.088 | 1.00 | 40.37 | A |
| ATOM | 604 | O1P | CYT | A | 29 | 10.945 | 32.136 | 40.247 | 1.00 | 43.72 | A |
| ATOM | 605 | O2P | CYT | A | 29 | 12.870 | 33.786 | 40.482 | 1.00 | 38.14 | A |
| ATOM | 606 | O5′ | CYT | A | 29 | 12.817 | 31.590 | 41.759 | 1.00 | 38.45 | A |
| ATOM | 607 | C5′ | CYT | A | 29 | 13.936 | 31.870 | 42.589 | 1.00 | 36.61 | A |
| ATOM | 608 | C4′ | CYT | A | 29 | 14.295 | 30.674 | 43.441 | 1.00 | 36.86 | A |
| ATOM | 609 | O4′ | CYT | A | 29 | 13.190 | 30.316 | 44.311 | 1.00 | 37.88 | A |
| ATOM | 610 | C1′ | CYT | A | 29 | 13.239 | 28.917 | 44.576 | 1.00 | 40.99 | A |
| ATOM | 611 | N1 | CYT | A | 29 | 12.051 | 28.285 | 43.971 | 1.00 | 44.33 | A |
| ATOM | 612 | C6 | CYT | A | 29 | 11.208 | 28.999 | 43.167 | 1.00 | 46.13 | A |
| ATOM | 613 | C2 | CYT | A | 29 | 11.796 | 26.930 | 44.233 | 1.00 | 47.42 | A |
| ATOM | 614 | O2 | CYT | A | 29 | 12.582 | 26.302 | 44.964 | 1.00 | 48.50 | A |
| ATOM | 615 | N3 | CYT | A | 29 | 10.698 | 26.342 | 43.682 | 1.00 | 48.76 | A |
| ATOM | 616 | C4 | CYT | A | 29 | 9.877 | 27.059 | 42.908 | 1.00 | 47.66 | A |
| ATOM | 617 | N4 | CYT | A | 29 | 8.802 | 26.457 | 42.403 | 1.00 | 47.18 | A |
| ATOM | 618 | C5 | CYT | A | 29 | 10.121 | 28.431 | 42.622 | 1.00 | 47.63 | A |
| ATOM | 619 | C2′ | CYT | A | 29 | 14.525 | 28.412 | 43.923 | 1.00 | 38.38 | A |
| ATOM | 620 | O2′ | CYT | A | 29 | 15.633 | 28.573 | 44.781 | 1.00 | 36.10 | A |
| ATOM | 621 | C3′ | CYT | A | 29 | 14.615 | 29.359 | 42.752 | 1.00 | 36.23 | A |
| ATOM | 622 | O3′ | CYT | A | 29 | 15.919 | 29.304 | 42.233 | 1.00 | 34.41 | A |
| ATOM | 623 | P | GUA | A | 30 | 16.152 | 28.661 | 40.790 | 1.00 | 38.05 | A |
| ATOM | 624 | O1P | GUA | A | 30 | 14.826 | 28.589 | 40.130 | 1.00 | 39.90 | A |
| ATOM | 625 | O2P | GUA | A | 30 | 17.252 | 29.385 | 40.126 | 1.00 | 33.67 | A |
| ATOM | 626 | O5′ | GUA | A | 30 | 16.582 | 27.160 | 41.107 | 1.00 | 38.02 | A |
| ATOM | 627 | C5′ | GUA | A | 30 | 16.835 | 26.749 | 42.437 | 1.00 | 40.68 | A |
| ATOM | 628 | C4′ | GUA | A | 30 | 16.851 | 25.248 | 42.523 | 1.00 | 43.03 | A |
| ATOM | 629 | O4′ | GUA | A | 30 | 15.496 | 24.779 | 42.704 | 1.00 | 45.49 | A |
| ATOM | 630 | C1′ | GUA | A | 30 | 15.149 | 23.912 | 41.646 | 1.00 | 46.19 | A |
| ATOM | 631 | N9 | GUA | A | 30 | 13.761 | 24.171 | 41.287 | 1.00 | 46.68 | A |
| ATOM | 632 | C4 | GUA | A | 30 | 12.741 | 23.263 | 41.260 | 1.00 | 46.15 | A |
| ATOM | 633 | N3 | GUA | A | 30 | 12.830 | 21.966 | 41.595 | 1.00 | 46.81 | A |
| ATOM | 634 | C2 | GUA | A | 30 | 11.677 | 21.337 | 41.433 | 1.00 | 47.98 | A |
| ATOM | 635 | N2 | GUA | A | 30 | 11.573 | 20.030 | 41.724 | 1.00 | 47.46 | A |
| ATOM | 636 | N1 | GUA | A | 30 | 10.537 | 21.940 | 40.974 | 1.00 | 48.25 | A |
| ATOM | 637 | C6 | GUA | A | 30 | 10.430 | 23.278 | 40.624 | 1.00 | 48.04 | A |
| ATOM | 638 | O6 | GUA | A | 30 | 9.359 | 23.723 | 40.214 | 1.00 | 48.66 | A |
| ATOM | 639 | C5 | GUA | A | 30 | 11.648 | 23.958 | 40.801 | 1.00 | 47.58 | A |
| ATOM | 640 | N7 | GUA | A | 30 | 11.967 | 25.283 | 40.575 | 1.00 | 48.54 | A |
| ATOM | 641 | C8 | GUA | A | 30 | 13.230 | 25.365 | 40.881 | 1.00 | 47.36 | A |
| ATOM | 642 | C2′ | GUA | A | 30 | 16.131 | 24.219 | 40.514 | 1.00 | 45.05 | A |
| ATOM | 643 | O2′ | GUA | A | 30 | 16.324 | 23.065 | 39.711 | 1.00 | 42.26 | A |
| ATOM | 644 | C3′ | GUA | A | 30 | 17.387 | 24.513 | 41.306 | 1.00 | 44.67 | A |
| ATOM | 645 | O3′ | GUA | A | 30 | 17.875 | 23.241 | 41.698 | 1.00 | 47.60 | A |
| ATOM | 646 | P | URI | A | 31 | 19.368 | 23.092 | 42.242 | 1.00 | 46.90 | A |
| ATOM | 647 | O1P | URI | A | 31 | 19.788 | 21.707 | 41.922 | 1.00 | 43.72 | A |
| ATOM | 648 | O2P | URI | A | 31 | 20.164 | 24.243 | 41.749 | 1.00 | 42.41 | A |
| ATOM | 649 | O5′ | URI | A | 31 | 19.165 | 23.178 | 43.810 | 1.00 | 44.16 | A |
| ATOM | 650 | C5′ | URI | A | 31 | 18.437 | 22.169 | 44.475 | 1.00 | 47.25 | A |
| ATOM | 651 | C4′ | URI | A | 31 | 19.073 | 21.875 | 45.800 | 1.00 | 50.83 | A |
| ATOM | 652 | O4′ | URI | A | 31 | 19.933 | 20.714 | 45.670 | 1.00 | 51.79 | A |
| ATOM | 653 | C1′ | URI | A | 31 | 21.260 | 21.063 | 46.011 | 1.00 | 53.42 | A |
| ATOM | 654 | N1 | URI | A | 31 | 22.159 | 20.294 | 45.141 | 1.00 | 54.61 | A |
| ATOM | 655 | C6 | URI | A | 31 | 22.280 | 20.580 | 43.797 | 1.00 | 55.01 | A |
| ATOM | 656 | C2 | URI | A | 31 | 22.864 | 19.255 | 45.711 | 1.00 | 53.45 | A |
| ATOM | 657 | O2 | URI | A | 31 | 22.796 | 18.992 | 46.901 | 1.00 | 54.06 | A |
| ATOM | 658 | N3 | URI | A | 31 | 23.644 | 18.533 | 44.834 | 1.00 | 51.58 | A |
| ATOM | 659 | C4 | URI | A | 31 | 23.787 | 18.749 | 43.473 | 1.00 | 51.47 | A |
| ATOM | 660 | O4 | URI | A | 31 | 24.429 | 17.943 | 42.792 | 1.00 | 52.23 | A |
| ATOM | 661 | C5 | URI | A | 31 | 23.050 | 19.861 | 42.969 | 1.00 | 52.57 | A |
| ATOM | 662 | C2′ | URI | A | 31 | 21.341 | 22.579 | 45.844 | 1.00 | 53.16 | A |
| ATOM | 663 | O2′ | URI | A | 31 | 22.354 | 23.115 | 46.667 | 1.00 | 55.10 | A |
| ATOM | 664 | C3′ | URI | A | 31 | 19.958 | 22.995 | 46.323 | 1.00 | 51.91 | A |
| ATOM | 665 | O3′ | URI | A | 31 | 19.921 | 22.992 | 47.737 | 1.00 | 51.68 | A |
| ATOM | 666 | P | GUA | A | 32 | 19.570 | 24.336 | 48.516 | 1.00 | 52.63 | A |
| ATOM | 667 | O1P | GUA | A | 32 | 20.544 | 24.456 | 49.629 | 1.00 | 52.90 | A |
| ATOM | 668 | O2P | GUA | A | 32 | 19.402 | 25.444 | 47.546 | 1.00 | 54.44 | A |
| ATOM | 669 | O5′ | GUA | A | 32 | 18.151 | 24.043 | 49.147 | 1.00 | 52.92 | A |
| ATOM | 670 | C5′ | GUA | A | 32 | 18.056 | 23.544 | 50.461 | 1.00 | 50.79 | A |
| ATOM | 671 | C4′ | GUA | A | 32 | 16.887 | 22.616 | 50.572 | 1.00 | 49.82 | A |
| ATOM | 672 | O4′ | GUA | A | 32 | 16.685 | 21.897 | 49.326 | 1.00 | 46.72 | A |
| ATOM | 673 | C1′ | GUA | A | 32 | 16.347 | 20.554 | 49.606 | 1.00 | 48.10 | A |
| ATOM | 674 | N9 | GUA | A | 32 | 17.438 | 19.710 | 49.133 | 1.00 | 49.09 | A |
| ATOM | 675 | C4 | GUA | A | 32 | 17.638 | 19.232 | 47.851 | 1.00 | 49.10 | A |
| ATOM | 676 | N3 | GUA | A | 32 | 16.845 | 19.452 | 46.782 | 1.00 | 49.00 | A |
| ATOM | 677 | C2 | GUA | A | 32 | 17.325 | 18.878 | 45.688 | 1.00 | 48.99 | A |
| ATOM | 678 | N2 | GUA | A | 32 | 16.690 | 19.006 | 44.518 | 1.00 | 48.27 | A |
| ATOM | 679 | N1 | GUA | A | 32 | 18.473 | 18.136 | 45.653 | 1.00 | 50.19 | A |
| ATOM | 680 | C6 | GUA | A | 32 | 19.297 | 17.892 | 46.740 | 1.00 | 48.64 | A |
| ATOM | 681 | O6 | GUA | A | 32 | 20.311 | 17.205 | 46.595 | 1.00 | 49.28 | A |
| ATOM | 682 | C5 | GUA | A | 32 | 18.808 | 18.510 | 47.913 | 1.00 | 48.50 | A |
| ATOM | 683 | N7 | GUA | A | 32 | 19.324 | 18.518 | 49.197 | 1.00 | 50.10 | A |
| ATOM | 684 | C8 | GUA | A | 32 | 18.481 | 19.238 | 49.884 | 1.00 | 49.69 | A |
| ATOM | 685 | C2′ | GUA | A | 32 | 16.142 | 20.468 | 51.120 | 1.00 | 48.46 | A |
| ATOM | 686 | O2′ | GUA | A | 32 | 14.815 | 20.805 | 51.432 | 1.00 | 50.02 | A |
| ATOM | 687 | C3′ | GUA | A | 32 | 17.087 | 21.547 | 51.615 | 1.00 | 49.56 | A |
| ATOM | 688 | O3′ | GUA | A | 32 | 16.674 | 22.088 | 52.855 | 1.00 | 51.72 | A |
| ATOM | 689 | P | ADE | A | 33 | 17.734 | 22.865 | 53.778 | 1.00 | 54.75 | A |
| ATOM | 690 | O1P | ADE | A | 33 | 17.193 | 22.727 | 55.153 | 1.00 | 53.71 | A |
| ATOM | 691 | O2P | ADE | A | 33 | 19.089 | 22.361 | 53.463 | 1.00 | 54.42 | A |
| ATOM | 692 | O5′ | ADE | A | 33 | 17.655 | 24.403 | 53.346 | 1.00 | 54.90 | A |
| ATOM | 693 | C5′ | ADE | A | 33 | 18.840 | 25.165 | 53.082 | 1.00 | 56.73 | A |
| ATOM | 694 | C4′ | ADE | A | 33 | 18.598 | 26.656 | 53.296 | 1.00 | 58.69 | A |
| ATOM | 695 | O4′ | ADE | A | 33 | 18.004 | 27.270 | 52.123 | 1.00 | 55.47 | A |
| ATOM | 696 | C1′ | ADE | A | 33 | 16.732 | 27.800 | 52.448 | 1.00 | 57.59 | A |
| ATOM | 697 | N9 | ADE | A | 33 | 15.796 | 27.377 | 51.407 | 1.00 | 55.80 | A |
| ATOM | 698 | C4 | ADE | A | 33 | 15.851 | 27.674 | 50.066 | 1.00 | 54.67 | A |
| ATOM | 699 | N3 | ADE | A | 33 | 16.754 | 28.443 | 49.434 | 1.00 | 54.07 | A |
| ATOM | 700 | C2 | ADE | A | 33 | 16.509 | 28.473 | 48.126 | 1.00 | 53.39 | A |
| ATOM | 701 | N1 | ADE | A | 33 | 15.532 | 27.875 | 47.435 | 1.00 | 54.37 | A |
| ATOM | 702 | C6 | ADE | A | 33 | 14.636 | 27.117 | 48.107 | 1.00 | 53.78 | A |
| ATOM | 703 | N6 | ADE | A | 33 | 13.654 | 26.522 | 47.427 | 1.00 | 52.21 | A |
| ATOM | 704 | C5 | ADE | A | 33 | 14.790 | 26.999 | 49.490 | 1.00 | 53.97 | A |
| ATOM | 705 | N7 | ADE | A | 33 | 14.066 | 26.310 | 50.450 | 1.00 | 54.45 | A |
| ATOM | 706 | C8 | ADE | A | 33 | 14.693 | 26.573 | 51.567 | 1.00 | 54.16 | A |
| ATOM | 707 | C2′ | ADE | A | 33 | 16.349 | 27.215 | 53.810 | 1.00 | 60.60 | A |
| ATOM | 708 | O2′ | ADE | A | 33 | 15.509 | 28.085 | 54.546 | 1.00 | 61.50 | A |
| ATOM | 709 | C3′ | ADE | A | 33 | 17.706 | 27.046 | 54.462 | 1.00 | 61.94 | A |
| ATOM | 710 | O3′ | ADE | A | 33 | 18.122 | 28.286 | 55.015 | 1.00 | 68.98 | A |
| ATOM | 711 | P | URI | A | 34 | 19.075 | 28.289 | 56.309 | 1.00 | 74.21 | A |
| ATOM | 712 | O1P | URI | A | 34 | 18.592 | 27.226 | 57.235 | 1.00 | 71.88 | A |
| ATOM | 713 | O2P | URI | A | 34 | 20.478 | 28.258 | 55.795 | 1.00 | 72.15 | A |
| ATOM | 714 | O5′ | URI | A | 34 | 18.811 | 29.688 | 57.024 | 1.00 | 73.89 | A |
| ATOM | 715 | C5′ | URI | A | 34 | 17.508 | 30.253 | 57.099 | 1.00 | 74.78 | A |
| ATOM | 716 | C4′ | URI | A | 34 | 17.553 | 31.691 | 56.640 | 1.00 | 76.05 | A |
| ATOM | 717 | O4′ | URI | A | 34 | 18.494 | 32.435 | 57.460 | 1.00 | 78.89 | A |
| ATOM | 718 | C1′ | URI | A | 34 | 19.610 | 32.847 | 56.688 | 1.00 | 81.15 | A |
| ATOM | 719 | N1 | URI | A | 34 | 20.836 | 32.426 | 57.390 | 1.00 | 87.35 | A |
| ATOM | 720 | C6 | URI | A | 34 | 20.975 | 31.133 | 57.855 | 1.00 | 89.21 | A |
| ATOM | 721 | C2 | URI | A | 34 | 21.849 | 33.365 | 57.573 | 1.00 | 89.29 | A |
| ATOM | 722 | O2 | URI | A | 34 | 21.775 | 34.523 | 57.169 | 1.00 | 89.92 | A |
| ATOM | 723 | N3 | URI | A | 34 | 22.955 | 32.893 | 58.245 | 1.00 | 91.09 | A |
| ATOM | 724 | C4 | URI | A | 34 | 23.152 | 31.608 | 58.734 | 1.00 | 92.28 | A |
| ATOM | 725 | O4 | URI | A | 34 | 24.214 | 31.326 | 59.303 | 1.00 | 93.85 | A |
| ATOM | 726 | C5 | URI | A | 34 | 22.064 | 30.705 | 58.500 | 1.00 | 91.36 | A |
| ATOM | 727 | C2′ | URI | A | 34 | 19.489 | 32.244 | 55.289 | 1.00 | 76.83 | A |
| ATOM | 728 | O2′ | URI | A | 34 | 20.020 | 33.134 | 54.327 | 1.00 | 73.41 | A |
| ATOM | 729 | C3′ | URI | A | 34 | 17.996 | 31.874 | 55.196 | 1.00 | 75.09 | A |
| ATOM | 730 | O3′ | URI | A | 34 | 17.057 | 32.711 | 54.511 | 1.00 | 68.53 | A |
| ATOM | 731 | P | ADE | A | 35 | 17.398 | 34.241 | 54.188 | 1.00 | 64.01 | A |
| ATOM | 732 | O1P | ADE | A | 35 | 16.110 | 34.964 | 54.265 | 1.00 | 63.53 | A |
| ATOM | 733 | O2P | ADE | A | 35 | 18.545 | 34.712 | 54.999 | 1.00 | 66.80 | A |
| ATOM | 734 | O5′ | ADE | A | 35 | 17.868 | 34.190 | 52.667 | 1.00 | 59.77 | A |
| ATOM | 735 | C5′ | ADE | A | 35 | 18.506 | 33.031 | 52.150 | 1.00 | 53.12 | A |
| ATOM | 736 | C4′ | ADE | A | 35 | 18.690 | 33.158 | 50.666 | 1.00 | 50.24 | A |
| ATOM | 737 | O4′ | ADE | A | 35 | 18.497 | 31.850 | 50.068 | 1.00 | 50.66 | A |
| ATOM | 738 | C1′ | ADE | A | 35 | 17.427 | 31.904 | 49.149 | 1.00 | 48.04 | A |
| ATOM | 739 | N9 | ADE | A | 35 | 16.228 | 31.385 | 49.815 | 1.00 | 47.05 | A |
| ATOM | 740 | C4 | ADE | A | 35 | 15.054 | 30.994 | 49.220 | 1.00 | 46.40 | A |
| ATOM | 741 | N3 | ADE | A | 35 | 14.750 | 31.027 | 47.914 | 1.00 | 47.60 | A |
| ATOM | 742 | C2 | ADE | A | 35 | 13.540 | 30.532 | 47.707 | 1.00 | 44.79 | A |
| ATOM | 743 | N1 | ADE | A | 35 | 12.664 | 30.052 | 48.586 | 1.00 | 43.36 | A |
| ATOM | 744 | C6 | ADE | A | 35 | 12.993 | 30.042 | 49.887 | 1.00 | 43.86 | A |
| ATOM | 745 | N6 | ADE | A | 35 | 12.105 | 29.577 | 50.766 | 1.00 | 42.72 | A |
| ATOM | 746 | C5 | ADE | A | 35 | 14.251 | 30.529 | 50.241 | 1.00 | 45.10 | A |
| ATOM | 747 | N7 | ADE | A | 35 | 14.892 | 30.643 | 51.461 | 1.00 | 45.15 | A |
| ATOM | 748 | C8 | ADE | A | 35 | 16.055 | 31.161 | 51.154 | 1.00 | 47.04 | A |
| ATOM | 749 | C2′ | ADE | A | 35 | 17.350 | 33.361 | 48.702 | 1.00 | 46.80 | A |
| ATOM | 750 | O2′ | ADE | A | 35 | 18.306 | 33.616 | 47.701 | 1.00 | 41.13 | A |
| ATOM | 751 | C3′ | ADE | A | 35 | 17.699 | 34.097 | 49.989 | 1.00 | 47.70 | A |
| ATOM | 752 | O3′ | ADE | A | 35 | 18.352 | 35.324 | 49.695 | 1.00 | 46.15 | A |
| ATOM | 753 | P | ADE | A | 36 | 17.500 | 36.579 | 49.176 | 1.00 | 44.91 | A |
| ATOM | 754 | O1P | ADE | A | 36 | 18.409 | 37.475 | 48.418 | 1.00 | 45.54 | A |
| ATOM | 755 | O2P | ADE | A | 36 | 16.717 | 37.121 | 50.316 | 1.00 | 42.81 | A |
| ATOM | 756 | O5′ | ADE | A | 36 | 16.511 | 35.947 | 48.103 | 1.00 | 44.04 | A |
| ATOM | 757 | C5′ | ADE | A | 36 | 16.849 | 35.905 | 46.722 | 1.00 | 39.76 | A |
| ATOM | 758 | C4′ | ADE | A | 36 | 15.618 | 35.594 | 45.904 | 1.00 | 40.10 | A |
| ATOM | 759 | O4′ | ADE | A | 36 | 15.126 | 34.280 | 46.278 | 1.00 | 38.85 | A |
| ATOM | 760 | C1′ | ADE | A | 36 | 13.731 | 34.317 | 46.453 | 1.00 | 38.26 | A |
| ATOM | 761 | N9 | ADE | A | 36 | 13.432 | 34.093 | 47.861 | 1.00 | 39.33 | A |
| ATOM | 762 | C4 | ADE | A | 36 | 12.309 | 33.474 | 48.356 | 1.00 | 41.34 | A |
| ATOM | 763 | N3 | ADE | A | 36 | 11.279 | 32.972 | 47.655 | 1.00 | 41.49 | A |
| ATOM | 764 | C2 | ADE | A | 36 | 10.382 | 32.433 | 48.467 | 1.00 | 40.53 | A |
| ATOM | 765 | N1 | ADE | A | 36 | 10.390 | 32.345 | 49.796 | 1.00 | 39.89 | A |
| ATOM | 766 | C6 | ADE | A | 36 | 11.432 | 32.860 | 50.471 | 1.00 | 40.76 | A |
| ATOM | 767 | N6 | ADE | A | 36 | 11.428 | 32.775 | 51.804 | 1.00 | 41.52 | A |
| ATOM | 768 | C5 | ADE | A | 36 | 12.459 | 33.458 | 49.724 | 1.00 | 40.32 | A |
| ATOM | 769 | N7 | ADE | A | 36 | 13.650 | 34.057 | 50.086 | 1.00 | 41.48 | A |
| ATOM | 770 | C8 | ADE | A | 36 | 14.183 | 34.421 | 48.944 | 1.00 | 39.27 | A |
| ATOM | 771 | C2′ | ADE | A | 36 | 13.248 | 35.653 | 45.919 | 1.00 | 38.75 | A |
| ATOM | 772 | O2′ | ADE | A | 36 | 13.033 | 35.501 | 44.544 | 1.00 | 42.97 | A |
| ATOM | 773 | C3′ | ADE | A | 36 | 14.455 | 36.537 | 46.164 | 1.00 | 40.00 | A |
| ATOM | 774 | O3′ | ADE | A | 36 | 14.470 | 37.597 | 45.220 | 1.00 | 37.24 | A |
| ATOM | 775 | P | ADE | A | 37 | 13.703 | 38.959 | 45.564 | 1.00 | 40.82 | A |
| ATOM | 776 | O1P | ADE | A | 37 | 14.010 | 39.984 | 44.531 | 1.00 | 36.22 | A |
| ATOM | 777 | O2P | ADE | A | 37 | 13.933 | 39.254 | 47.005 | 1.00 | 36.06 | A |
| ATOM | 778 | O5′ | ADE | A | 37 | 12.162 | 38.587 | 45.420 | 1.00 | 42.41 | A |
| ATOM | 779 | C5′ | ADE | A | 37 | 11.607 | 38.229 | 44.170 | 1.00 | 43.07 | A |
| ATOM | 780 | C4′ | ADE | A | 37 | 10.234 | 37.659 | 44.380 | 1.00 | 45.84 | A |
| ATOM | 781 | O4′ | ADE | A | 37 | 10.341 | 36.414 | 45.126 | 1.00 | 47.43 | A |
| ATOM | 782 | C1′ | ADE | A | 37 | 9.225 | 36.284 | 45.991 | 1.00 | 47.98 | A |
| ATOM | 783 | N9 | ADE | A | 37 | 9.703 | 36.337 | 47.369 | 1.00 | 48.10 | A |
| ATOM | 784 | C4 | ADE | A | 37 | 9.166 | 35.679 | 48.446 | 1.00 | 48.11 | A |
| ATOM | 785 | N3 | ADE | A | 37 | 8.126 | 34.837 | 48.446 | 1.00 | 47.34 | A |
| ATOM | 786 | C2 | ADE | A | 37 | 7.867 | 34.417 | 49.683 | 1.00 | 48.62 | A |
| ATOM | 787 | N1 | ADE | A | 37 | 8.485 | 34.719 | 50.832 | 1.00 | 47.87 | A |
| ATOM | 788 | C6 | ADE | A | 37 | 9.528 | 35.569 | 50.794 | 1.00 | 48.60 | A |
| ATOM | 789 | N6 | ADE | A | 37 | 10.138 | 35.876 | 51.934 | 1.00 | 48.53 | A |
| ATOM | 790 | C5 | ADE | A | 37 | 9.904 | 36.084 | 49.544 | 1.00 | 48.72 | A |
| ATOM | 791 | N7 | ADE | A | 37 | 10.904 | 36.961 | 49.163 | 1.00 | 49.65 | A |
| ATOM | 792 | C8 | ADE | A | 37 | 10.748 | 37.071 | 47.865 | 1.00 | 50.20 | A |
| ATOM | 793 | C2′ | ADE | A | 37 | 8.299 | 37.459 | 45.667 | 1.00 | 47.59 | A |
| ATOM | 794 | O2′ | ADE | A | 37 | 7.452 | 37.118 | 44.584 | 1.00 | 51.37 | A |
| ATOM | 795 | C3′ | ADE | A | 37 | 9.306 | 38.505 | 45.235 | 1.00 | 45.40 | A |
| ATOM | 796 | O3′ | ADE | A | 37 | 8.660 | 39.477 | 44.430 | 1.00 | 42.84 | A |
| ATOM | 797 | P | URI | A | 38 | 7.845 | 40.673 | 45.122 | 1.00 | 42.60 | A |
| ATOM | 798 | O1P | URI | A | 38 | 8.528 | 41.081 | 46.379 | 1.00 | 42.82 | A |
| ATOM | 799 | O2P | URI | A | 38 | 7.641 | 41.675 | 44.052 | 1.00 | 36.14 | A |
| ATOM | 800 | O5′ | URI | A | 38 | 6.462 | 39.999 | 45.548 | 1.00 | 42.36 | A |
| ATOM | 801 | C5′ | URI | A | 38 | 5.891 | 40.240 | 46.841 | 1.00 | 44.35 | A |
| ATOM | 802 | C4′ | URI | A | 38 | 4.952 | 39.115 | 47.235 | 1.00 | 46.75 | A |
| ATOM | 803 | O4′ | URI | A | 38 | 5.725 | 37.988 | 47.741 | 1.00 | 47.80 | A |
| ATOM | 804 | C1′ | URI | A | 38 | 4.998 | 37.325 | 48.765 | 1.00 | 48.04 | A |
| ATOM | 805 | N1 | URI | A | 38 | 5.771 | 37.389 | 50.011 | 1.00 | 47.05 | A |
| ATOM | 806 | C6 | URI | A | 38 | 6.835 | 38.239 | 50.144 | 1.00 | 46.72 | A |
| ATOM | 807 | C2 | URI | A | 38 | 5.371 | 36.579 | 51.062 | 1.00 | 46.91 | A |
| ATOM | 808 | O2 | URI | A | 38 | 4.473 | 35.766 | 50.969 | 1.00 | 46.67 | A |
| ATOM | 809 | N3 | URI | A | 38 | 6.078 | 36.747 | 52.224 | 1.00 | 46.31 | A |
| ATOM | 810 | C4 | URI | A | 38 | 7.142 | 37.594 | 52.429 | 1.00 | 46.35 | A |
| ATOM | 811 | O4 | URI | A | 38 | 7.685 | 37.627 | 53.533 | 1.00 | 45.78 | A |
| ATOM | 812 | C5 | URI | A | 38 | 7.518 | 38.365 | 51.285 | 1.00 | 47.05 | A |
| ATOM | 813 | C2′ | URI | A | 38 | 3.660 | 38.050 | 48.911 | 1.00 | 48.80 | A |
| ATOM | 814 | O2′ | URI | A | 38 | 2.661 | 37.414 | 48.141 | 1.00 | 52.16 | A |
| ATOM | 815 | C3′ | URI | A | 38 | 4.004 | 39.434 | 48.383 | 1.00 | 48.58 | A |
| ATOM | 816 | O3′ | URI | A | 38 | 2.825 | 40.090 | 47.936 | 1.00 | 48.56 | A |
| ATOM | 817 | P | GUA | A | 39 | 2.254 | 41.336 | 48.767 | 1.00 | 48.44 | A |
| ATOM | 818 | O1P | GUA | A | 39 | 3.327 | 42.356 | 48.824 | 1.00 | 43.94 | A |
| ATOM | 819 | O2P | GUA | A | 39 | 0.950 | 41.673 | 48.150 | 1.00 | 50.57 | A |
| ATOM | 820 | O5′ | GUA | A | 39 | 2.001 | 40.763 | 50.237 | 1.00 | 49.08 | A |
| ATOM | 821 | C5′ | GUA | A | 39 | 0.842 | 39.975 | 50.537 | 1.00 | 52.34 | A |
| ATOM | 822 | C4′ | GUA | A | 39 | 0.884 | 39.493 | 51.974 | 1.00 | 52.64 | A |
| ATOM | 823 | O4′ | GUA | A | 39 | 2.200 | 38.950 | 52.198 | 1.00 | 53.74 | A |
| ATOM | 824 | C1′ | GUA | A | 39 | 2.677 | 39.322 | 53.472 | 1.00 | 55.12 | A |
| ATOM | 825 | N9 | GUA | A | 39 | 4.040 | 39.832 | 53.331 | 1.00 | 53.40 | A |
| ATOM | 826 | C4 | GUA | A | 39 | 5.030 | 39.793 | 54.280 | 1.00 | 53.51 | A |
| ATOM | 827 | N3 | GUA | A | 39 | 4.921 | 39.272 | 55.521 | 1.00 | 55.12 | A |
| ATOM | 828 | C2 | GUA | A | 39 | 6.055 | 39.368 | 56.203 | 1.00 | 56.07 | A |
| ATOM | 829 | N2 | GUA | A | 39 | 6.133 | 38.880 | 57.444 | 1.00 | 55.26 | A |
| ATOM | 830 | N1 | GUA | A | 39 | 7.198 | 39.945 | 55.713 | 1.00 | 57.90 | A |
| ATOM | 831 | C6 | GUA | A | 39 | 7.329 | 40.498 | 54.442 | 1.00 | 57.91 | A |
| ATOM | 832 | O6 | GUA | A | 39 | 8.405 | 41.009 | 54.100 | 1.00 | 61.79 | A |
| ATOM | 833 | C5 | GUA | A | 39 | 6.122 | 40.385 | 53.693 | 1.00 | 55.10 | A |
| ATOM | 834 | N7 | GUA | A | 39 | 5.827 | 40.789 | 52.403 | 1.00 | 53.11 | A |
| ATOM | 835 | C8 | GUA | A | 39 | 4.584 | 40.441 | 52.233 | 1.00 | 52.96 | A |
| ATOM | 836 | C2′ | GUA | A | 39 | 1.702 | 40.302 | 54.127 | 1.00 | 54.78 | A |
| ATOM | 837 | O2′ | GUA | A | 39 | 1.187 | 39.738 | 55.314 | 1.00 | 54.03 | A |
| ATOM | 838 | C3′ | GUA | A | 39 | 0.674 | 40.577 | 53.025 | 1.00 | 55.19 | A |
| ATOM | 839 | O3′ | GUA | A | 39 | −0.706 | 40.787 | 53.445 | 1.00 | 59.20 | A |
| ATOM | 840 | P | URI | A | 40 | −1.599 | 39.602 | 54.126 | 1.00 | 56.47 | A |
| ATOM | 841 | O1P | URI | A | 40 | −1.160 | 39.317 | 55.525 | 1.00 | 54.80 | A |
| ATOM | 842 | O2P | URI | A | 40 | −3.004 | 39.999 | 53.874 | 1.00 | 58.19 | A |
| ATOM | 843 | O5′ | URI | A | 40 | −1.371 | 38.302 | 53.245 | 1.00 | 55.95 | A |
| ATOM | 844 | C5′ | URI | A | 40 | −1.812 | 37.034 | 53.718 | 1.00 | 55.43 | A |
| ATOM | 845 | C4′ | URI | A | 40 | −2.982 | 36.549 | 52.905 | 1.00 | 54.71 | A |
| ATOM | 846 | O4′ | URI | A | 40 | −2.742 | 35.169 | 52.519 | 1.00 | 54.93 | A |
| ATOM | 847 | C1′ | URI | A | 40 | −3.665 | 34.317 | 53.174 | 1.00 | 54.07 | A |
| ATOM | 848 | N1 | URI | A | 40 | −2.937 | 33.133 | 53.656 | 1.00 | 54.14 | A |
| ATOM | 849 | C6 | URI | A | 40 | −1.741 | 33.250 | 54.316 | 1.00 | 53.97 | A |
| ATOM | 850 | C2 | URI | A | 40 | −3.485 | 31.892 | 53.401 | 1.00 | 51.99 | A |
| ATOM | 851 | O2 | URI | A | 40 | −4.562 | 31.753 | 52.881 | 1.00 | 52.24 | A |
| ATOM | 852 | N3 | URI | A | 40 | −2.724 | 30.824 | 53.795 | 1.00 | 51.15 | A |
| ATOM | 853 | C4 | URI | A | 40 | −1.504 | 30.871 | 54.432 | 1.00 | 52.19 | A |
| ATOM | 854 | O4 | URI | A | 40 | −0.904 | 29.822 | 54.676 | 1.00 | 52.40 | A |
| ATOM | 855 | C5 | URI | A | 40 | −1.028 | 32.190 | 54.706 | 1.00 | 52.91 | A |
| ATOM | 856 | C2′ | URI | A | 40 | −4.307 | 35.144 | 54.280 | 1.00 | 53.49 | A |
| ATOM | 857 | O2′ | URI | A | 40 | −5.602 | 34.648 | 54.520 | 1.00 | 53.75 | A |
| ATOM | 858 | C3′ | URI | A | 40 | −4.307 | 36.529 | 53.647 | 1.00 | 54.99 | A |
| ATOM | 859 | O3′ | URI | A | 40 | −5.363 | 36.652 | 52.705 | 1.00 | 57.21 | A |
| ATOM | 860 | P | ADE | A | 41 | −6.710 | 37.431 | 53.104 | 1.00 | 58.89 | A |
| ATOM | 861 | O1P | ADE | A | 41 | −6.562 | 38.826 | 52.614 | 1.00 | 58.50 | A |
| ATOM | 862 | O2P | ADE | A | 41 | −7.038 | 37.180 | 54.546 | 1.00 | 55.76 | A |
| ATOM | 863 | O5′ | ADE | A | 41 | −7.800 | 36.713 | 52.199 | 1.00 | 58.92 | A |
| ATOM | 864 | C5′ | ADE | A | 41 | −7.650 | 35.342 | 51.838 | 1.00 | 60.17 | A |
| ATOM | 865 | C4′ | ADE | A | 41 | −8.829 | 34.888 | 51.014 | 1.00 | 59.95 | A |
| ATOM | 866 | O4′ | ADE | A | 41 | −8.375 | 34.035 | 49.934 | 1.00 | 60.38 | A |
| ATOM | 867 | C1′ | ADE | A | 41 | −9.373 | 33.076 | 49.636 | 1.00 | 61.63 | A |
| ATOM | 868 | N9 | ADE | A | 41 | −8.821 | 31.756 | 49.905 | 1.00 | 62.90 | A |
| ATOM | 869 | C4 | ADE | A | 41 | −9.190 | 30.583 | 49.294 | 1.00 | 64.74 | A |
| ATOM | 870 | N3 | ADE | A | 41 | −10.063 | 30.430 | 48.284 | 1.00 | 65.58 | A |
| ATOM | 871 | C2 | ADE | A | 41 | −10.204 | 29.139 | 47.975 | 1.00 | 66.23 | A |
| ATOM | 872 | N1 | ADE | A | 41 | −9.627 | 28.066 | 48.531 | 1.00 | 66.78 | A |
| ATOM | 873 | C6 | ADE | A | 41 | −8.763 | 28.256 | 49.556 | 1.00 | 66.47 | A |
| ATOM | 874 | N6 | ADE | A | 41 | −8.218 | 27.183 | 50.141 | 1.00 | 66.76 | A |
| ATOM | 875 | C5 | ADE | A | 41 | −8.503 | 29.583 | 49.957 | 1.00 | 65.70 | A |
| ATOM | 876 | N7 | ADE | A | 41 | −7.669 | 30.123 | 50.926 | 1.00 | 65.96 | A |
| ATOM | 877 | C8 | ADE | A | 41 | −7.888 | 31.414 | 50.846 | 1.00 | 63.92 | A |
| ATOM | 878 | C2′ | ADE | A | 41 | −10.552 | 33.358 | 50.573 | 1.00 | 60.59 | A |
| ATOM | 879 | O2′ | ADE | A | 41 | −11.506 | 34.185 | 49.940 | 1.00 | 59.58 | A |
| ATOM | 880 | C3′ | ADE | A | 41 | −9.840 | 34.027 | 51.741 | 1.00 | 60.33 | A |
| ATOM | 881 | O3′ | ADE | A | 41 | −10.708 | 34.839 | 52.517 | 1.00 | 60.73 | A |
| ATOM | 882 | P | GUA | A | 42 | −11.635 | 34.165 | 53.642 | 1.00 | 63.04 | A |
| ATOM | 883 | O1P | GUA | A | 42 | −10.778 | 33.611 | 54.722 | 1.00 | 60.30 | A |
| ATOM | 884 | O2P | GUA | A | 42 | −12.698 | 35.154 | 53.979 | 1.00 | 61.99 | A |
| ATOM | 885 | O5′ | GUA | A | 42 | −12.294 | 32.926 | 52.888 | 1.00 | 60.43 | A |
| ATOM | 886 | C5′ | GUA | A | 42 | −12.827 | 31.843 | 53.623 | 1.00 | 58.01 | A |
| ATOM | 887 | C4′ | GUA | A | 42 | −13.024 | 30.658 | 52.724 | 1.00 | 57.39 | A |
| ATOM | 888 | O4′ | GUA | A | 42 | −11.830 | 30.476 | 51.921 | 1.00 | 56.25 | A |
| ATOM | 889 | C1′ | GUA | A | 42 | −11.422 | 29.117 | 51.969 | 1.00 | 57.45 | A |
| ATOM | 890 | N9 | GUA | A | 42 | −10.237 | 29.022 | 52.821 | 1.00 | 55.08 | A |
| ATOM | 891 | C4 | GUA | A | 42 | −9.535 | 27.884 | 53.138 | 1.00 | 53.31 | A |
| ATOM | 892 | N3 | GUA | A | 42 | −9.812 | 26.635 | 52.714 | 1.00 | 53.57 | A |
| ATOM | 893 | C2 | GUA | A | 42 | −8.959 | 25.742 | 53.213 | 1.00 | 55.55 | A |
| ATOM | 894 | N2 | GUA | A | 42 | −9.082 | 24.441 | 52.911 | 1.00 | 56.87 | A |
| ATOM | 895 | N1 | GUA | A | 42 | −7.920 | 26.055 | 54.047 | 1.00 | 55.96 | A |
| ATOM | 896 | C6 | GUA | A | 42 | −7.606 | 27.335 | 54.486 | 1.00 | 55.98 | A |
| ATOM | 897 | O6 | GUA | A | 42 | −6.626 | 27.508 | 55.224 | 1.00 | 56.71 | A |
| ATOM | 898 | C5 | GUA | A | 42 | −8.520 | 28.301 | 53.973 | 1.00 | 54.30 | A |
| ATOM | 899 | N7 | GUA | A | 42 | −8.581 | 29.671 | 54.173 | 1.00 | 53.33 | A |
| ATOM | 900 | C8 | GUA | A | 42 | −9.610 | 30.056 | 53.472 | 1.00 | 53.69 | A |
| ATOM | 901 | C2′ | GUA | A | 42 | −12.616 | 28.332 | 52.510 | 1.00 | 58.44 | A |
| ATOM | 902 | O2′ | GUA | A | 42 | −13.472 | 27.964 | 51.450 | 1.00 | 58.26 | A |
| ATOM | 903 | C3′ | GUA | A | 42 | −13.221 | 29.349 | 53.470 | 1.00 | 58.81 | A |
| ATOM | 904 | O3′ | GUA | A | 42 | −14.605 | 29.138 | 53.722 | 1.00 | 61.50 | A |
| ATOM | 905 | P | CYT | A | 43 | −15.070 | 28.243 | 54.977 | 1.00 | 64.98 | A |
| ATOM | 906 | O1P | CYT | A | 43 | −14.485 | 28.761 | 56.250 | 1.00 | 59.74 | A |
| ATOM | 907 | O2P | CYT | A | 43 | −16.546 | 28.116 | 54.851 | 1.00 | 64.87 | A |
| ATOM | 908 | O5′ | CYT | A | 43 | −14.458 | 26.811 | 54.652 | 1.00 | 64.07 | A |
| ATOM | 909 | C5′ | CYT | A | 43 | −14.898 | 26.095 | 53.506 | 1.00 | 65.62 | A |
| ATOM | 910 | C4′ | CYT | A | 43 | −14.456 | 24.660 | 53.584 | 1.00 | 65.98 | A |
| ATOM | 911 | O4′ | CYT | A | 43 | −13.029 | 24.599 | 53.330 | 1.00 | 65.81 | A |
| ATOM | 912 | C1′ | CYT | A | 43 | −12.432 | 23.669 | 54.211 | 1.00 | 65.26 | A |
| ATOM | 913 | N1 | CYT | A | 43 | −11.549 | 24.400 | 55.135 | 1.00 | 63.82 | A |
| ATOM | 914 | C6 | CYT | A | 43 | −11.704 | 25.739 | 55.352 | 1.00 | 62.45 | A |
| ATOM | 915 | C2 | CYT | A | 43 | −10.531 | 23.698 | 55.780 | 1.00 | 64.02 | A |
| ATOM | 916 | O2 | CYT | A | 43 | −10.427 | 22.480 | 55.576 | 1.00 | 65.38 | A |
| ATOM | 917 | N3 | CYT | A | 43 | −9.688 | 24.356 | 56.604 | 1.00 | 62.61 | A |
| ATOM | 918 | C4 | CYT | A | 43 | −9.831 | 25.666 | 56.793 | 1.00 | 62.22 | A |
| ATOM | 919 | N4 | CYT | A | 43 | −8.954 | 26.279 | 57.593 | 1.00 | 60.19 | A |
| ATOM | 920 | C5 | CYT | A | 43 | −10.874 | 26.407 | 56.164 | 1.00 | 62.02 | A |
| ATOM | 921 | C2′ | CYT | A | 43 | −13.573 | 22.913 | 54.890 | 1.00 | 65.89 | A |
| ATOM | 922 | O2′ | CYT | A | 43 | −13.956 | 21.825 | 54.071 | 1.00 | 66.59 | A |
| ATOM | 923 | C3′ | CYT | A | 43 | −14.649 | 23.987 | 54.935 | 1.00 | 66.04 | A |
| ATOM | 924 | O3′ | CYT | A | 43 | −15.946 | 23.395 | 55.000 | 1.00 | 66.73 | A |
| ATOM | 925 | P | URI | A | 44 | −16.711 | 23.269 | 56.415 | 1.00 | 65.82 | A |
| ATOM | 926 | O1P | URI | A | 44 | −16.592 | 24.556 | 57.157 | 1.00 | 63.68 | A |
| ATOM | 927 | O2P | URI | A | 44 | −18.052 | 22.723 | 56.105 | 1.00 | 66.14 | A |
| ATOM | 928 | O5′ | URI | A | 44 | −15.917 | 22.137 | 57.199 | 1.00 | 63.62 | A |
| ATOM | 929 | C5′ | URI | A | 44 | −16.200 | 20.773 | 56.957 | 1.00 | 63.38 | A |
| ATOM | 930 | C4′ | URI | A | 44 | −15.136 | 19.904 | 57.572 | 1.00 | 65.62 | A |
| ATOM | 931 | O4′ | URI | A | 44 | −13.825 | 20.362 | 57.151 | 1.00 | 65.65 | A |
| ATOM | 932 | C1′ | URI | A | 44 | −12.880 | 20.075 | 58.172 | 1.00 | 65.84 | A |
| ATOM | 933 | N1 | URI | A | 44 | −12.292 | 21.338 | 58.638 | 1.00 | 66.30 | A |
| ATOM | 934 | C6 | URI | A | 44 | −12.758 | 22.562 | 58.215 | 1.00 | 65.36 | A |
| ATOM | 935 | C2 | URI | A | 44 | −11.233 | 21.246 | 59.514 | 1.00 | 65.77 | A |
| ATOM | 936 | O2 | URI | A | 44 | −10.826 | 20.185 | 59.927 | 1.00 | 67.70 | A |
| ATOM | 937 | N3 | URI | A | 44 | −10.678 | 22.438 | 59.891 | 1.00 | 64.76 | A |
| ATOM | 938 | C4 | URI | A | 44 | −11.075 | 23.692 | 59.494 | 1.00 | 66.40 | A |
| ATOM | 939 | O4 | URI | A | 44 | −10.453 | 24.674 | 59.898 | 1.00 | 67.76 | A |
| ATOM | 940 | C5 | URI | A | 44 | −12.201 | 23.713 | 58.602 | 1.00 | 65.92 | A |
| ATOM | 941 | C2′ | URI | A | 44 | −13.638 | 19.357 | 59.285 | 1.00 | 66.12 | A |
| ATOM | 942 | O2′ | URI | A | 44 | −13.566 | 17.958 | 59.087 | 1.00 | 66.69 | A |
| ATOM | 943 | C3′ | URI | A | 44 | −15.032 | 19.932 | 59.086 | 1.00 | 65.90 | A |
| ATOM | 944 | O3′ | URI | A | 44 | −16.017 | 19.111 | 59.690 | 1.00 | 67.53 | A |
| ATOM | 945 | P | ADE | A | 45 | −16.217 | 19.151 | 61.284 | 1.00 | 68.53 | A |
| ATOM | 946 | O1P | ADE | A | 45 | −16.924 | 17.900 | 61.656 | 1.00 | 68.32 | A |
| ATOM | 947 | O2P | ADE | A | 45 | −16.789 | 20.469 | 61.673 | 1.00 | 67.51 | A |
| ATOM | 948 | O5′ | ADE | A | 45 | −14.740 | 19.052 | 61.865 | 1.00 | 69.08 | A |
| ATOM | 949 | C5′ | ADE | A | 45 | −14.502 | 18.673 | 63.214 | 1.00 | 68.01 | A |
| ATOM | 950 | C4′ | ADE | A | 45 | −13.284 | 17.797 | 63.283 | 1.00 | 68.82 | A |
| ATOM | 951 | O4′ | ADE | A | 45 | −12.273 | 18.326 | 62.385 | 1.00 | 70.13 | A |
| ATOM | 952 | C1′ | ADE | A | 45 | −10.995 | 18.206 | 62.981 | 1.00 | 71.44 | A |
| ATOM | 953 | N9 | ADE | A | 45 | −10.492 | 19.550 | 63.260 | 1.00 | 73.11 | A |
| ATOM | 954 | C4 | ADE | A | 45 | −9.264 | 19.852 | 63.792 | 1.00 | 73.86 | A |
| ATOM | 955 | N3 | ADE | A | 45 | −8.291 | 18.988 | 64.129 | 1.00 | 74.54 | A |
| ATOM | 956 | C2 | ADE | A | 45 | −7.241 | 19.638 | 64.619 | 1.00 | 75.77 | A |
| ATOM | 957 | N1 | ADE | A | 45 | −7.066 | 20.955 | 64.797 | 1.00 | 75.85 | A |
| ATOM | 958 | C6 | ADE | A | 45 | −8.063 | 21.795 | 64.441 | 1.00 | 74.73 | A |
| ATOM | 959 | N6 | ADE | A | 45 | −7.881 | 23.107 | 64.606 | 1.00 | 74.84 | A |
| ATOM | 960 | C5 | ADE | A | 45 | −9.231 | 21.230 | 63.910 | 1.00 | 73.92 | A |
| ATOM | 961 | N7 | ADE | A | 45 | −10.412 | 21.791 | 63.448 | 1.00 | 73.89 | A |
| ATOM | 962 | C8 | ADE | A | 45 | −11.124 | 20.756 | 63.070 | 1.00 | 73.73 | A |
| ATOM | 963 | C2′ | ADE | A | 45 | −11.175 | 17.369 | 64.249 | 1.00 | 71.14 | A |
| ATOM | 964 | O2′ | ADE | A | 45 | −11.023 | 16.000 | 63.944 | 1.00 | 71.70 | A |
| ATOM | 965 | C3′ | ADE | A | 45 | −12.606 | 17.709 | 64.638 | 1.00 | 69.95 | A |
| ATOM | 966 | O3′ | ADE | A | 45 | −13.179 | 16.632 | 65.359 | 1.00 | 71.13 | A |
| ATOM | 967 | P | ADE | A | 46 | −13.171 | 16.646 | 66.961 | 1.00 | 72.38 | A |
| ATOM | 968 | O1P | ADE | A | 46 | −13.939 | 15.447 | 67.395 | 1.00 | 70.63 | A |
| ATOM | 969 | O2P | ADE | A | 46 | −13.583 | 17.997 | 67.434 | 1.00 | 71.11 | A |
| ATOM | 970 | O5′ | ADE | A | 46 | −11.638 | 16.438 | 67.330 | 1.00 | 71.76 | A |
| ATOM | 971 | C5′ | ADE | A | 46 | −10.956 | 15.253 | 66.964 | 1.00 | 72.17 | A |
| ATOM | 972 | C4′ | ADE | A | 46 | −9.555 | 15.292 | 67.496 | 1.00 | 73.44 | A |
| ATOM | 973 | O4′ | ADE | A | 46 | −8.795 | 16.246 | 66.714 | 1.00 | 73.28 | A |
| ATOM | 974 | C1′ | ADE | A | 46 | −7.798 | 16.844 | 67.532 | 1.00 | 73.77 | A |
| ATOM | 975 | N9 | ADE | A | 46 | −7.983 | 18.292 | 67.495 | 1.00 | 71.50 | A |
| ATOM | 976 | C4 | ADE | A | 46 | −7.068 | 19.231 | 67.896 | 1.00 | 70.59 | A |
| ATOM | 977 | N3 | ADE | A | 46 | −5.832 | 19.010 | 68.365 | 1.00 | 70.30 | A |
| ATOM | 978 | C2 | ADE | A | 46 | −5.240 | 20.156 | 68.673 | 1.00 | 70.11 | A |
| ATOM | 979 | N1 | ADE | A | 46 | −5.708 | 21.403 | 68.574 | 1.00 | 70.07 | A |
| ATOM | 980 | C6 | ADE | A | 46 | −6.954 | 21.589 | 68.100 | 1.00 | 69.89 | A |
| ATOM | 981 | N6 | ADE | A | 46 | −7.423 | 22.831 | 68.011 | 1.00 | 69.62 | A |
| ATOM | 982 | C5 | ADE | A | 46 | −7.685 | 20.455 | 67.732 | 1.00 | 70.34 | A |
| ATOM | 983 | N7 | ADE | A | 46 | −8.961 | 20.295 | 67.217 | 1.00 | 70.99 | A |
| ATOM | 984 | C8 | ADE | A | 46 | −9.087 | 18.996 | 67.092 | 1.00 | 71.76 | A |
| ATOM | 985 | C2′ | ADE | A | 46 | −7.983 | 16.299 | 68.952 | 1.00 | 75.60 | A |
| ATOM | 986 | O2′ | ADE | A | 46 | −7.045 | 15.278 | 69.248 | 1.00 | 76.78 | A |
| ATOM | 987 | C3′ | ADE | A | 46 | −9.434 | 15.830 | 68.910 | 1.00 | 75.50 | A |
| ATOM | 988 | O3′ | ADE | A | 46 | −9.700 | 14.831 | 69.883 | 1.00 | 77.29 | A |
| ATOM | 989 | P | ADE | A | 47 | −10.668 | 15.174 | 71.117 | 1.00 | 79.69 | A |
| ATOM | 990 | O1P | ADE | A | 47 | −10.647 | 14.026 | 72.061 | 1.00 | 79.57 | A |
| ATOM | 991 | O2P | ADE | A | 47 | −11.959 | 15.634 | 70.546 | 1.00 | 78.48 | A |
| ATOM | 992 | O5′ | ADE | A | 47 | −9.939 | 16.386 | 71.851 | 1.00 | 79.12 | A |
| ATOM | 993 | C5′ | ADE | A | 47 | −8.813 | 16.148 | 72.694 | 1.00 | 79.56 | A |
| ATOM | 994 | C4′ | ADE | A | 47 | −7.789 | 17.251 | 72.536 | 1.00 | 79.57 | A |
| ATOM | 995 | O4′ | ADE | A | 47 | −8.054 | 17.996 | 71.320 | 1.00 | 78.89 | A |
| ATOM | 996 | C1′ | ADE | A | 47 | −7.612 | 19.336 | 71.482 | 1.00 | 76.86 | A |
| ATOM | 997 | N9 | ADE | A | 47 | −8.699 | 20.239 | 71.089 | 1.00 | 73.28 | A |
| ATOM | 998 | C4 | ADE | A | 47 | −8.651 | 21.616 | 71.042 | 1.00 | 70.83 | A |
| ATOM | 999 | N3 | ADE | A | 47 | −7.618 | 22.412 | 71.369 | 1.00 | 69.61 | A |
| ATOM | 1000 | C2 | ADE | A | 47 | −7.935 | 23.696 | 71.184 | 1.00 | 68.39 | A |
| ATOM | 1001 | N1 | ADE | A | 47 | −9.075 | 24.231 | 70.734 | 1.00 | 66.31 | A |
| ATOM | 1002 | C6 | ADE | A | 47 | −10.090 | 23.404 | 70.403 | 1.00 | 67.06 | A |
| ATOM | 1003 | N6 | ADE | A | 47 | −11.218 | 23.937 | 69.931 | 1.00 | 65.64 | A |
| ATOM | 1004 | C5 | ADE | A | 47 | −9.889 | 22.020 | 70.569 | 1.00 | 68.98 | A |
| ATOM | 1005 | N7 | ADE | A | 47 | −10.710 | 20.923 | 70.341 | 1.00 | 69.48 | A |
| ATOM | 1006 | C8 | ADE | A | 47 | −9.963 | 19.896 | 70.669 | 1.00 | 71.21 | A |
| ATOM | 1007 | C2′ | ADE | A | 47 | −7.133 | 19.498 | 72.928 | 1.00 | 79.08 | A |
| ATOM | 1008 | O2′ | ADE | A | 47 | −5.720 | 19.415 | 72.987 | 1.00 | 79.49 | A |
| ATOM | 1009 | C3′ | ADE | A | 47 | −7.810 | 18.318 | 73.619 | 1.00 | 79.92 | A |
| ATOM | 1010 | O3′ | ADE | A | 47 | −7.040 | 17.900 | 74.735 | 1.00 | 81.30 | A |
| ATOM | 1011 | P | ADE | A | 48 | −7.584 | 18.174 | 76.218 | 1.00 | 82.37 | A |
| ATOM | 1012 | O1P | ADE | A | 48 | −8.374 | 16.983 | 76.624 | 1.00 | 81.64 | A |
| ATOM | 1013 | O2P | ADE | A | 48 | −8.214 | 19.516 | 76.241 | 1.00 | 81.56 | A |
| ATOM | 1014 | O5′ | ADE | A | 48 | −6.260 | 18.214 | 77.095 | 1.00 | 82.32 | A |
| ATOM | 1015 | C5′ | ADE | A | 48 | −5.365 | 17.113 | 77.094 | 1.00 | 83.46 | A |
| ATOM | 1016 | C4′ | ADE | A | 48 | −3.960 | 17.594 | 77.326 | 1.00 | 85.20 | A |
| ATOM | 1017 | O4′ | ADE | A | 48 | −3.499 | 18.305 | 76.143 | 1.00 | 84.90 | A |
| ATOM | 1018 | C1′ | ADE | A | 48 | −2.600 | 19.334 | 76.533 | 1.00 | 85.40 | A |
| ATOM | 1019 | N9 | ADE | A | 48 | −3.174 | 20.620 | 76.142 | 1.00 | 85.75 | A |
| ATOM | 1020 | C4 | ADE | A | 48 | −2.527 | 21.834 | 76.201 | 1.00 | 85.93 | A |
| ATOM | 1021 | N3 | ADE | A | 48 | −1.253 | 22.065 | 76.574 | 1.00 | 85.58 | A |
| ATOM | 1022 | C2 | ADE | A | 48 | −0.984 | 23.367 | 76.546 | 1.00 | 85.50 | A |
| ATOM | 1023 | N1 | ADE | A | 48 | −1.783 | 24.390 | 76.216 | 1.00 | 85.56 | A |
| ATOM | 1024 | C6 | ADE | A | 48 | −3.055 | 24.125 | 75.843 | 1.00 | 84.69 | A |
| ATOM | 1025 | N6 | ADE | A | 48 | −3.849 | 25.145 | 75.523 | 1.00 | 83.49 | A |
| ATOM | 1026 | C5 | ADE | A | 48 | −3.464 | 22.779 | 75.821 | 1.00 | 84.98 | A |
| ATOM | 1027 | N7 | ADE | A | 48 | −4.669 | 22.171 | 75.491 | 1.00 | 84.83 | A |
| ATOM | 1028 | C8 | ADE | A | 48 | −4.442 | 20.892 | 75.691 | 1.00 | 85.38 | A |
| ATOM | 1029 | C2′ | ADE | A | 48 | −2.463 | 19.256 | 78.059 | 1.00 | 85.92 | A |
| ATOM | 1030 | O2′ | ADE | A | 48 | −1.327 | 18.501 | 78.444 | 1.00 | 84.47 | A |
| ATOM | 1031 | C3′ | ADE | A | 48 | −3.796 | 18.622 | 78.435 | 1.00 | 85.80 | A |
| ATOM | 1032 | O3′ | ADE | A | 48 | −3.766 | 18.027 | 79.729 | 1.00 | 86.27 | A |
| ATOM | 1033 | P | ADE | A | 49 | −4.373 | 18.832 | 80.986 | 1.00 | 88.05 | A |
| ATOM | 1034 | O1P | ADE | A | 49 | −4.142 | 18.025 | 82.212 | 1.00 | 87.72 | A |
| ATOM | 1035 | O2P | ADE | A | 49 | −5.755 | 19.264 | 80.647 | 1.00 | 88.03 | A |
| ATOM | 1036 | O5′ | ADE | A | 49 | −3.455 | 20.131 | 81.084 | 1.00 | 85.69 | A |
| ATOM | 1037 | C5′ | ADE | A | 49 | −2.071 | 20.026 | 81.397 | 1.00 | 82.44 | A |
| ATOM | 1038 | C4′ | ADE | A | 49 | −1.389 | 21.358 | 81.192 | 1.00 | 81.27 | A |
| ATOM | 1039 | O4′ | ADE | A | 49 | −1.679 | 21.845 | 79.853 | 1.00 | 80.07 | A |
| ATOM | 1040 | C1′ | ADE | A | 49 | −1.745 | 23.264 | 79.861 | 1.00 | 77.99 | A |
| ATOM | 1041 | N9 | ADE | A | 49 | −3.084 | 23.658 | 79.430 | 1.00 | 76.14 | A |
| ATOM | 1042 | C4 | ADE | A | 49 | −3.522 | 24.942 | 79.227 | 1.00 | 75.30 | A |
| ATOM | 1043 | N3 | ADE | A | 49 | −2.812 | 26.072 | 79.362 | 1.00 | 74.58 | A |
| ATOM | 1044 | C2 | ADE | A | 49 | −3.569 | 27.135 | 79.106 | 1.00 | 74.96 | A |
| ATOM | 1045 | N1 | ADE | A | 49 | −4.860 | 27.192 | 78.758 | 1.00 | 75.05 | A |
| ATOM | 1046 | C6 | ADE | A | 49 | −5.546 | 26.036 | 78.631 | 1.00 | 75.57 | A |
| ATOM | 1047 | N6 | ADE | A | 49 | −6.834 | 26.093 | 78.292 | 1.00 | 75.44 | A |
| ATOM | 1048 | C5 | ADE | A | 49 | −4.853 | 24.836 | 78.871 | 1.00 | 75.74 | A |
| ATOM | 1049 | N7 | ADE | A | 49 | −5.247 | 23.506 | 78.834 | 1.00 | 75.97 | A |
| ATOM | 1050 | C8 | ADE | A | 49 | −4.162 | 22.849 | 79.167 | 1.00 | 76.26 | A |
| ATOM | 1051 | C2′ | ADE | A | 49 | −1.442 | 23.720 | 81.289 | 1.00 | 79.04 | A |
| ATOM | 1052 | O2′ | ADE | A | 49 | −0.075 | 24.059 | 81.411 | 1.00 | 77.88 | A |
| ATOM | 1053 | C3′ | ADE | A | 49 | −1.862 | 22.490 | 82.087 | 1.00 | 80.35 | A |
| ATOM | 1054 | O3′ | ADE | A | 49 | −1.261 | 22.428 | 83.375 | 1.00 | 80.33 | A |
| ATOM | 1055 | P | GUA | A | 50 | −2.185 | 22.582 | 84.679 | 1.00 | 81.00 | A |
| ATOM | 1056 | O1P | GUA | A | 50 | −3.300 | 21.604 | 84.529 | 1.00 | 80.20 | A |
| ATOM | 1057 | O2P | GUA | A | 50 | −1.341 | 22.560 | 85.906 | 1.00 | 79.72 | A |
| ATOM | 1058 | O5′ | GUA | A | 50 | −2.784 | 24.050 | 84.525 | 1.00 | 79.65 | A |
| ATOM | 1059 | C5′ | GUA | A | 50 | −1.921 | 25.173 | 84.372 | 1.00 | 77.16 | A |
| ATOM | 1060 | C4′ | GUA | A | 50 | −2.719 | 26.402 | 84.007 | 1.00 | 75.00 | A |
| ATOM | 1061 | O4′ | GUA | A | 50 | −3.208 | 26.286 | 82.638 | 1.00 | 73.43 | A |
| ATOM | 1062 | C1′ | GUA | A | 50 | −4.541 | 26.770 | 82.564 | 1.00 | 72.01 | A |
| ATOM | 1063 | N9 | GUA | A | 50 | −5.433 | 25.623 | 82.411 | 1.00 | 70.22 | A |
| ATOM | 1064 | C4 | GUA | A | 50 | −6.724 | 25.655 | 81.960 | 1.00 | 67.92 | A |
| ATOM | 1065 | N3 | GUA | A | 50 | −7.402 | 26.758 | 81.590 | 1.00 | 66.32 | A |
| ATOM | 1066 | C2 | GUA | A | 50 | −8.637 | 26.476 | 81.219 | 1.00 | 66.81 | A |
| ATOM | 1067 | N2 | GUA | A | 50 | −9.463 | 27.461 | 80.851 | 1.00 | 65.04 | A |
| ATOM | 1068 | N1 | GUA | A | 50 | −9.158 | 25.205 | 81.191 | 1.00 | 68.08 | A |
| ATOM | 1069 | C6 | GUA | A | 50 | −8.473 | 24.051 | 81.564 | 1.00 | 69.44 | A |
| ATOM | 1070 | O6 | GUA | A | 50 | −9.037 | 22.946 | 81.506 | 1.00 | 70.39 | A |
| ATOM | 1071 | C5 | GUA | A | 50 | −7.153 | 24.344 | 81.981 | 1.00 | 68.58 | A |
| ATOM | 1072 | N7 | GUA | A | 50 | −6.150 | 23.503 | 82.441 | 1.00 | 68.42 | A |
| ATOM | 1073 | C8 | GUA | A | 50 | −5.149 | 24.303 | 82.685 | 1.00 | 69.29 | A |
| ATOM | 1074 | C2′ | GUA | A | 50 | −4.770 | 27.520 | 83.872 | 1.00 | 72.81 | A |
| ATOM | 1075 | O2′ | GUA | A | 50 | −4.222 | 28.817 | 83.756 | 1.00 | 71.66 | A |
| ATOM | 1076 | C3′ | GUA | A | 50 | −3.977 | 26.639 | 84.822 | 1.00 | 73.60 | A |
| ATOM | 1077 | O3′ | GUA | A | 50 | −3.684 | 27.280 | 86.054 | 1.00 | 73.95 | A |
| ATOM | 1078 | P | GUA | A | 51 | −4.694 | 27.112 | 87.294 | 1.00 | 74.38 | A |
| ATOM | 1079 | O1P | GUA | A | 51 | −5.138 | 25.695 | 87.343 | 1.00 | 73.33 | A |
| ATOM | 1080 | O2P | GUA | A | 51 | −4.067 | 27.727 | 88.487 | 1.00 | 72.68 | A |
| ATOM | 1081 | O5′ | GUA | A | 51 | −5.952 | 27.991 | 86.870 | 1.00 | 74.71 | A |
| ATOM | 1082 | C5′ | GUA | A | 51 | −5.783 | 29.325 | 86.397 | 1.00 | 73.94 | A |
| ATOM | 1083 | C4′ | GUA | A | 51 | −7.125 | 29.948 | 86.118 | 1.00 | 73.27 | A |
| ATOM | 1084 | O4′ | GUA | A | 51 | −7.656 | 29.393 | 84.888 | 1.00 | 72.61 | A |
| ATOM | 1085 | C1′ | GUA | A | 51 | −9.060 | 29.227 | 85.005 | 1.00 | 72.51 | A |
| ATOM | 1086 | N9 | GUA | A | 51 | −9.354 | 27.805 | 84.898 | 1.00 | 71.49 | A |
| ATOM | 1087 | C4 | GUA | A | 51 | −10.535 | 27.252 | 84.485 | 1.00 | 69.81 | A |
| ATOM | 1088 | N3 | GUA | A | 51 | −11.648 | 27.931 | 84.147 | 1.00 | 68.51 | A |
| ATOM | 1089 | C2 | GUA | A | 51 | −12.622 | 27.123 | 83.780 | 1.00 | 68.74 | A |
| ATOM | 1090 | N2 | GUA | A | 51 | −13.810 | 27.628 | 83.424 | 1.00 | 68.49 | A |
| ATOM | 1091 | N1 | GUA | A | 51 | −12.505 | 25.757 | 83.737 | 1.00 | 69.64 | A |
| ATOM | 1092 | C6 | GUA | A | 51 | −11.365 | 25.039 | 84.076 | 1.00 | 70.00 | A |
| ATOM | 1093 | O6 | GUA | A | 51 | −11.362 | 23.804 | 83.990 | 1.00 | 71.28 | A |
| ATOM | 1094 | C5 | GUA | A | 51 | −10.322 | 25.892 | 84.486 | 1.00 | 69.35 | A |
| ATOM | 1095 | N7 | GUA | A | 51 | −9.040 | 25.595 | 84.918 | 1.00 | 70.02 | A |
| ATOM | 1096 | C8 | GUA | A | 51 | −8.505 | 26.759 | 85.160 | 1.00 | 70.88 | A |
| ATOM | 1097 | C2′ | GUA | A | 51 | −9.475 | 29.821 | 86.347 | 1.00 | 73.56 | A |
| ATOM | 1098 | O2′ | GUA | A | 51 | −9.867 | 31.166 | 86.164 | 1.00 | 74.00 | A |
| ATOM | 1099 | C3′ | GUA | A | 51 | −8.194 | 29.643 | 87.150 | 1.00 | 73.77 | A |
| ATOM | 1100 | O3′ | GUA | A | 51 | −8.114 | 30.545 | 88.238 | 1.00 | 74.52 | A |
| ATOM | 1101 | P | ADE | A | 52 | −8.773 | 30.142 | 89.642 | 1.00 | 76.30 | A |
| ATOM | 1102 | O1P | ADE | A | 52 | −8.331 | 28.768 | 90.007 | 1.00 | 73.86 | A |
| ATOM | 1103 | O2P | ADE | A | 52 | −8.551 | 31.261 | 90.586 | 1.00 | 76.17 | A |
| ATOM | 1104 | O5′ | ADE | A | 52 | −10.323 | 30.090 | 89.311 | 1.00 | 78.15 | A |
| ATOM | 1105 | PC | ADE | A | 52 | −13.399 | 30.333 | 93.693 | 1.00 | 89.30 | A |
| ATOM | 1106 | O1C | ADE | A | 52 | −12.966 | 29.470 | 94.833 | 1.00 | 88.46 | A |
| ATOM | 1107 | O2C | ADE | A | 52 | −13.966 | 31.592 | 94.237 | 1.00 | 89.76 | A |
| ATOM | 1108 | O3′ | ADE | A | 52 | −12.135 | 30.667 | 92.749 | 1.00 | 88.47 | A |
| ATOM | 1109 | C5′ | ADE | A | 52 | −11.120 | 29.037 | 89.802 | 1.00 | 82.75 | A |
| ATOM | 1110 | C4′ | ADE | A | 52 | −12.297 | 29.587 | 90.555 | 1.00 | 85.33 | A |
| ATOM | 1111 | O4′ | ADE | A | 52 | −13.454 | 28.844 | 90.104 | 1.00 | 86.70 | A |
| ATOM | 1112 | C1′ | ADE | A | 52 | −13.899 | 28.008 | 91.146 | 1.00 | 88.21 | A |
| ATOM | 1113 | N9 | ADE | A | 52 | −13.256 | 26.699 | 91.016 | 1.00 | 87.28 | A |
| ATOM | 1114 | C4 | ADE | A | 52 | −13.874 | 25.553 | 90.585 | 1.00 | 85.99 | A |
| ATOM | 1115 | N3 | ADE | A | 52 | −15.152 | 25.418 | 90.189 | 1.00 | 84.73 | A |
| ATOM | 1116 | C2 | ADE | A | 52 | −15.402 | 24.160 | 89.845 | 1.00 | 84.82 | A |
| ATOM | 1117 | N1 | ADE | A | 52 | −14.580 | 23.101 | 89.854 | 1.00 | 85.66 | A |
| ATOM | 1118 | C6 | ADE | A | 52 | −13.299 | 23.276 | 90.261 | 1.00 | 86.47 | A |
| ATOM | 1119 | N6 | ADE | A | 52 | −12.472 | 22.225 | 90.275 | 1.00 | 87.46 | A |
| ATOM | 1120 | C5 | ADE | A | 52 | −12.910 | 24.561 | 90.648 | 1.00 | 86.01 | A |
| ATOM | 1121 | N7 | ADE | A | 52 | −11.702 | 25.071 | 91.102 | 1.00 | 85.88 | A |
| ATOM | 1122 | C8 | ADE | A | 52 | −11.956 | 26.339 | 91.305 | 1.00 | 87.01 | A |
| ATOM | 1123 | C2′ | ADE | A | 52 | −13.529 | 28.719 | 92.445 | 1.00 | 88.72 | A |
| ATOM | 1124 | O2′ | ADE | A | 52 | −14.557 | 29.604 | 92.858 | 1.00 | 89.03 | A |
| ATOM | 1125 | C3′ | ADE | A | 52 | −12.218 | 29.415 | 92.075 | 1.00 | 86.83 | A |
| TER | | | | | | | | | | | |
| ATOM | 1126 | O6P | GUA | B | 201 | 0.475 | 29.070 | 36.643 | 1.00 | 78.07 | B |
| ATOM | 1127 | O3P | GUA | B | 201 | 2.504 | 29.382 | 35.176 | 1.00 | 72.28 | B |
| ATOM | 1128 | O4P | GUA | B | 201 | 1.151 | 27.209 | 35.096 | 1.00 | 78.40 | B |
| ATOM | 1129 | O5P | GUA | B | 201 | 0.153 | 29.309 | 34.172 | 1.00 | 77.17 | B |
| ATOM | 1130 | P2 | GUA | B | 201 | 1.035 | 28.724 | 35.273 | 1.00 | 78.53 | B |
| ATOM | 1131 | P | GUA | B | 201 | 3.207 | 29.078 | 33.730 | 1.00 | 67.30 | B |
| ATOM | 1132 | O1P | GUA | B | 201 | 2.397 | 29.678 | 32.604 | 1.00 | 68.58 | B |
| ATOM | 1133 | O2P | GUA | B | 201 | 3.397 | 27.584 | 33.493 | 1.00 | 65.14 | B |
| ATOM | 1134 | O5′ | GUA | B | 201 | 4.661 | 29.812 | 33.757 | 1.00 | 65.23 | B |
| ATOM | 1135 | C5′ | GUA | B | 201 | 4.779 | 31.168 | 34.260 | 1.00 | 59.17 | B |
| ATOM | 1136 | C4′ | GUA | B | 201 | 6.020 | 31.321 | 35.109 | 1.00 | 54.52 | B |
| ATOM | 1137 | O4′ | GUA | B | 201 | 5.753 | 30.983 | 36.494 | 1.00 | 52.68 | B |
| ATOM | 1138 | C1′ | GUA | B | 201 | 6.913 | 30.394 | 37.065 | 1.00 | 52.45 | B |
| ATOM | 1139 | N9 | GUA | B | 201 | 6.599 | 29.022 | 37.436 | 1.00 | 51.23 | B |
| ATOM | 1140 | C4 | GUA | B | 201 | 7.438 | 28.136 | 38.073 | 1.00 | 50.83 | B |
| ATOM | 1141 | N3 | GUA | B | 201 | 8.698 | 28.388 | 38.480 | 1.00 | 50.53 | B |
| ATOM | 1142 | C2 | GUA | B | 201 | 9.253 | 27.328 | 39.046 | 1.00 | 50.31 | B |
| ATOM | 1143 | N2 | GUA | B | 201 | 10.512 | 27.380 | 39.486 | 1.00 | 48.27 | B |
| ATOM | 1144 | N1 | GUA | B | 201 | 8.615 | 26.133 | 39.216 | 1.00 | 51.41 | B |
| ATOM | 1145 | C6 | GUA | B | 201 | 7.317 | 25.855 | 38.814 | 1.00 | 51.48 | B |
| ATOM | 1146 | O6 | GUA | B | 201 | 6.835 | 24.742 | 39.029 | 1.00 | 53.50 | B |
| ATOM | 1147 | C5 | GUA | B | 201 | 6.717 | 26.970 | 38.189 | 1.00 | 51.05 | B |
| ATOM | 1148 | N7 | GUA | B | 201 | 5.452 | 27.117 | 37.639 | 1.00 | 51.16 | B |
| ATOM | 1149 | C8 | GUA | B | 201 | 5.426 | 28.351 | 37.209 | 1.00 | 50.70 | B |
| ATOM | 1150 | C2′ | GUA | B | 201 | 7.999 | 30.409 | 35.993 | 1.00 | 52.40 | B |
| ATOM | 1151 | O2′ | GUA | B | 201 | 8.806 | 31.555 | 36.121 | 1.00 | 52.70 | B |
| ATOM | 1152 | C3′ | GUA | B | 201 | 7.161 | 30.406 | 34.729 | 1.00 | 52.42 | B |
| ATOM | 1153 | O3′ | GUA | B | 201 | 7.874 | 30.965 | 33.658 | 1.00 | 50.70 | B |
| ATOM | 1154 | P | CYT | B | 202 | 8.761 | 30.015 | 32.732 | 1.00 | 54.04 | B |
| ATOM | 1155 | O1P | CYT | B | 202 | 7.936 | 28.821 | 32.381 | 1.00 | 53.07 | B |
| ATOM | 1156 | O2P | CYT | B | 202 | 9.316 | 30.881 | 31.657 | 1.00 | 49.44 | B |
| ATOM | 1157 | O5′ | CYT | B | 202 | 9.943 | 29.532 | 33.689 | 1.00 | 51.35 | B |
| ATOM | 1158 | C5′ | CYT | B | 202 | 10.766 | 30.485 | 34.352 | 1.00 | 47.88 | B |
| ATOM | 1159 | C4′ | CYT | B | 202 | 11.962 | 29.817 | 34.972 | 1.00 | 44.57 | B |
| ATOM | 1160 | O4′ | CYT | B | 202 | 11.589 | 29.271 | 36.260 | 1.00 | 42.31 | B |
| ATOM | 1161 | C1′ | CYT | B | 202 | 12.237 | 28.030 | 36.451 | 1.00 | 41.89 | B |
| ATOM | 1162 | N1 | CYT | B | 202 | 11.233 | 26.976 | 36.380 | 1.00 | 41.10 | B |
| ATOM | 1163 | C6 | CYT | B | 202 | 9.994 | 27.211 | 35.848 | 1.00 | 43.60 | B |
| ATOM | 1164 | C2 | CYT | B | 202 | 11.570 | 25.720 | 36.840 | 1.00 | 40.20 | B |
| ATOM | 1165 | O2 | CYT | B | 202 | 12.674 | 25.559 | 37.350 | 1.00 | 38.30 | B |
| ATOM | 1166 | N3 | CYT | B | 202 | 10.680 | 24.708 | 36.725 | 1.00 | 43.53 | B |
| ATOM | 1167 | C4 | CYT | B | 202 | 9.477 | 24.934 | 36.194 | 1.00 | 43.15 | B |
| ATOM | 1168 | N4 | CYT | B | 202 | 8.637 | 23.907 | 36.097 | 1.00 | 43.58 | B |
| ATOM | 1169 | C5 | CYT | B | 202 | 9.090 | 26.225 | 35.740 | 1.00 | 43.56 | B |
| ATOM | 1170 | C2′ | CYT | B | 202 | 13.247 | 27.874 | 35.317 | 1.00 | 42.94 | B |
| ATOM | 1171 | O2′ | CYT | B | 202 | 14.469 | 28.448 | 35.705 | 1.00 | 43.96 | B |
| ATOM | 1172 | C3′ | CYT | B | 202 | 12.565 | 28.653 | 34.202 | 1.00 | 43.70 | B |
| ATOM | 1173 | O3′ | CYT | B | 202 | 13.531 | 29.144 | 33.290 | 1.00 | 43.33 | B |
| ATOM | 1174 | P | GUA | B | 203 | 13.858 | 28.332 | 31.949 | 1.00 | 46.72 | B |
| ATOM | 1175 | O1P | GUA | B | 203 | 12.593 | 27.844 | 31.370 | 1.00 | 48.52 | B |
| ATOM | 1176 | O2P | GUA | B | 203 | 14.725 | 29.223 | 31.147 | 1.00 | 46.78 | B |
| ATOM | 1177 | O5′ | GUA | B | 203 | 14.709 | 27.082 | 32.448 | 1.00 | 46.96 | B |
| ATOM | 1178 | C5′ | GUA | B | 203 | 15.833 | 27.282 | 33.296 | 1.00 | 46.67 | B |
| ATOM | 1179 | C4′ | GUA | B | 203 | 16.366 | 25.968 | 33.800 | 1.00 | 46.87 | B |
| ATOM | 1180 | O4′ | GUA | B | 203 | 15.544 | 25.479 | 34.885 | 1.00 | 46.06 | B |
| ATOM | 1181 | C1′ | GUA | B | 203 | 15.525 | 24.059 | 34.853 | 1.00 | 49.05 | B |
| ATOM | 1182 | N9 | GUA | B | 203 | 14.176 | 23.616 | 34.536 | 1.00 | 50.83 | B |
| ATOM | 1183 | C4 | GUA | B | 203 | 13.697 | 22.350 | 34.693 | 1.00 | 51.60 | B |
| ATOM | 1184 | N3 | GUA | B | 203 | 14.391 | 21.306 | 35.180 | 1.00 | 52.97 | B |
| ATOM | 1185 | C2 | GUA | B | 203 | 13.677 | 20.193 | 35.173 | 1.00 | 55.12 | B |
| ATOM | 1186 | N2 | GUA | B | 203 | 14.227 | 19.044 | 35.596 | 1.00 | 55.46 | B |
| ATOM | 1187 | N1 | GUA | B | 203 | 12.373 | 20.120 | 34.741 | 1.00 | 55.24 | B |
| ATOM | 1188 | C6 | GUA | B | 203 | 11.638 | 21.187 | 34.236 | 1.00 | 54.21 | B |
| ATOM | 1189 | O6 | GUA | B | 203 | 10.471 | 21.008 | 33.857 | 1.00 | 55.53 | B |
| ATOM | 1190 | C5 | GUA | B | 203 | 12.400 | 22.385 | 34.228 | 1.00 | 52.79 | B |
| ATOM | 1191 | N7 | GUA | B | 203 | 12.065 | 23.661 | 33.803 | 1.00 | 52.05 | B |
| ATOM | 1192 | C8 | GUA | B | 203 | 13.147 | 24.360 | 34.008 | 1.00 | 52.06 | B |
| ATOM | 1193 | C2′ | GUA | B | 203 | 16.491 | 23.627 | 33.754 | 1.00 | 49.39 | B |
| ATOM | 1194 | O2′ | GUA | B | 203 | 17.774 | 23.426 | 34.320 | 1.00 | 50.70 | B |
| ATOM | 1195 | C3′ | GUA | B | 203 | 16.394 | 24.818 | 32.813 | 1.00 | 48.50 | B |
| ATOM | 1196 | O3′ | GUA | B | 203 | 17.540 | 24.916 | 31.986 | 1.00 | 51.54 | B |
| ATOM | 1197 | P | CYT | B | 204 | 17.445 | 24.511 | 30.436 | 1.00 | 53.75 | B |
| ATOM | 1198 | O1P | CYT | B | 204 | 16.121 | 24.953 | 29.937 | 1.00 | 55.60 | B |
| ATOM | 1199 | O2P | CYT | B | 204 | 18.682 | 25.001 | 29.770 | 1.00 | 53.17 | B |
| ATOM | 1200 | O5′ | CYT | B | 204 | 17.457 | 22.922 | 30.460 | 1.00 | 51.80 | B |
| ATOM | 1201 | C5′ | CYT | B | 204 | 18.473 | 22.229 | 31.158 | 1.00 | 51.55 | B |
| ATOM | 1202 | C4′ | CYT | B | 204 | 18.146 | 20.765 | 31.227 | 1.00 | 52.80 | B |
| ATOM | 1203 | O4′ | CYT | B | 204 | 17.050 | 20.563 | 32.153 | 1.00 | 53.65 | B |
| ATOM | 1204 | C1′ | CYT | B | 204 | 16.323 | 19.404 | 31.769 | 1.00 | 51.69 | B |
| ATOM | 1205 | N1 | CYT | B | 204 | 14.946 | 19.803 | 31.490 | 1.00 | 50.29 | B |
| ATOM | 1206 | C6 | CYT | B | 204 | 14.635 | 21.098 | 31.189 | 1.00 | 51.07 | B |
| ATOM | 1207 | C2 | CYT | B | 204 | 13.956 | 18.837 | 31.544 | 1.00 | 51.03 | B |
| ATOM | 1208 | O2 | CYT | B | 204 | 14.279 | 17.677 | 31.819 | 1.00 | 50.84 | B |
| ATOM | 1209 | N3 | CYT | B | 204 | 12.671 | 19.187 | 31.302 | 1.00 | 51.56 | B |
| ATOM | 1210 | C4 | CYT | B | 204 | 12.370 | 20.456 | 31.026 | 1.00 | 51.67 | B |
| ATOM | 1211 | N4 | CYT | B | 204 | 11.091 | 20.764 | 30.824 | 1.00 | 51.58 | B |
| ATOM | 1212 | C5 | CYT | B | 204 | 13.369 | 21.467 | 30.953 | 1.00 | 51.72 | B |
| ATOM | 1213 | C2′ | CYT | B | 204 | 17.028 | 18.840 | 30.538 | 1.00 | 52.66 | B |
| ATOM | 1214 | O2′ | CYT | B | 204 | 18.028 | 17.937 | 30.975 | 1.00 | 50.99 | B |
| ATOM | 1215 | C3′ | CYT | B | 204 | 17.615 | 20.117 | 29.960 | 1.00 | 54.04 | B |
| ATOM | 1216 | O3′ | CYT | B | 204 | 18.650 | 19.862 | 29.017 | 1.00 | 57.06 | B |
| ATOM | 1217 | P | GUA | B | 205 | 18.527 | 20.459 | 27.539 | 1.00 | 57.66 | B |
| ATOM | 1218 | O1P | GUA | B | 205 | 17.497 | 21.519 | 27.638 | 1.00 | 59.12 | B |
| ATOM | 1219 | O2P | GUA | B | 205 | 19.878 | 20.793 | 27.025 | 1.00 | 55.89 | B |
| ATOM | 1220 | O5′ | GUA | B | 205 | 17.899 | 19.260 | 26.700 | 1.00 | 58.91 | B |
| ATOM | 1221 | C5′ | GUA | B | 205 | 18.625 | 18.050 | 26.478 | 1.00 | 58.65 | B |
| ATOM | 1222 | C4′ | GUA | B | 205 | 17.775 | 16.846 | 26.835 | 1.00 | 57.77 | B |
| ATOM | 1223 | O4′ | GUA | B | 205 | 17.003 | 17.156 | 28.024 | 1.00 | 58.08 | B |
| ATOM | 1224 | C1′ | GUA | B | 205 | 15.757 | 16.478 | 27.976 | 1.00 | 58.15 | B |
| ATOM | 1225 | N9 | GUA | B | 205 | 14.689 | 17.469 | 27.941 | 1.00 | 57.22 | B |
| ATOM | 1226 | C4 | GUA | B | 205 | 13.342 | 17.211 | 27.974 | 1.00 | 55.21 | B |
| ATOM | 1227 | N3 | GUA | B | 205 | 12.771 | 15.997 | 28.063 | 1.00 | 54.66 | B |
| ATOM | 1228 | C2 | GUA | B | 205 | 11.452 | 16.071 | 28.036 | 1.00 | 54.04 | B |
| ATOM | 1229 | N2 | GUA | B | 205 | 10.719 | 14.963 | 28.091 | 1.00 | 53.92 | B |
| ATOM | 1230 | N1 | GUA | B | 205 | 10.756 | 17.238 | 27.942 | 1.00 | 54.33 | B |
| ATOM | 1231 | C6 | GUA | B | 205 | 11.323 | 18.497 | 27.838 | 1.00 | 55.11 | B |
| ATOM | 1232 | O6 | GUA | B | 205 | 10.599 | 19.484 | 27.714 | 1.00 | 57.19 | B |
| ATOM | 1233 | C5 | GUA | B | 205 | 12.733 | 18.437 | 27.872 | 1.00 | 55.05 | B |
| ATOM | 1234 | N7 | GUA | B | 205 | 13.677 | 19.450 | 27.805 | 1.00 | 57.05 | B |
| ATOM | 1235 | C8 | GUA | B | 205 | 14.823 | 18.830 | 27.850 | 1.00 | 56.48 | B |
| ATOM | 1236 | C2′ | GUA | B | 205 | 15.775 | 15.615 | 26.718 | 1.00 | 57.78 | B |
| ATOM | 1237 | O2′ | GUA | B | 205 | 16.279 | 14.348 | 27.071 | 1.00 | 60.51 | B |
| ATOM | 1238 | C3′ | GUA | B | 205 | 16.720 | 16.411 | 25.828 | 1.00 | 57.96 | B |
| ATOM | 1239 | O3′ | GUA | B | 205 | 17.269 | 15.591 | 24.793 | 1.00 | 59.77 | B |
| ATOM | 1240 | P | GUA | B | 206 | 16.445 | 15.362 | 23.419 | 1.00 | 61.50 | B |
| ATOM | 1241 | O1P | GUA | B | 206 | 15.949 | 16.687 | 22.956 | 1.00 | 59.32 | B |
| ATOM | 1242 | O2P | GUA | B | 206 | 17.276 | 14.536 | 22.510 | 1.00 | 60.35 | B |
| ATOM | 1243 | O5′ | GUA | B | 206 | 15.191 | 14.472 | 23.844 | 1.00 | 60.21 | B |
| ATOM | 1244 | C5′ | GUA | B | 206 | 15.360 | 13.112 | 24.241 | 1.00 | 60.08 | B |
| ATOM | 1245 | C4′ | GUA | B | 206 | 14.035 | 12.378 | 24.209 | 1.00 | 60.45 | B |
| ATOM | 1246 | O4′ | GUA | B | 206 | 13.174 | 12.848 | 25.287 | 1.00 | 61.47 | B |
| ATOM | 1247 | C1′ | GUA | B | 206 | 11.829 | 12.928 | 24.835 | 1.00 | 61.03 | B |
| ATOM | 1248 | N9 | GUA | B | 206 | 11.441 | 14.339 | 24.794 | 1.00 | 59.51 | B |
| ATOM | 1249 | C4 | GUA | B | 206 | 10.167 | 14.845 | 24.683 | 1.00 | 57.68 | B |
| ATOM | 1250 | N3 | GUA | B | 206 | 9.038 | 14.124 | 24.594 | 1.00 | 57.86 | B |
| ATOM | 1251 | C2 | GUA | B | 206 | 7.973 | 14.892 | 24.488 | 1.00 | 57.96 | B |
| ATOM | 1252 | N2 | GUA | B | 206 | 6.762 | 14.332 | 24.390 | 1.00 | 60.81 | B |
| ATOM | 1253 | N1 | GUA | B | 206 | 8.010 | 16.265 | 24.470 | 1.00 | 57.03 | B |
| ATOM | 1254 | C6 | GUA | B | 206 | 9.161 | 17.035 | 24.555 | 1.00 | 55.93 | B |
| ATOM | 1255 | O6 | GUA | B | 206 | 9.083 | 18.275 | 24.518 | 1.00 | 49.77 | B |
| ATOM | 1256 | C5 | GUA | B | 206 | 10.316 | 16.219 | 24.673 | 1.00 | 57.07 | B |
| ATOM | 1257 | N7 | GUA | B | 206 | 11.651 | 16.568 | 24.781 | 1.00 | 58.68 | B |
| ATOM | 1258 | C8 | GUA | B | 206 | 12.280 | 15.425 | 24.851 | 1.00 | 60.02 | B |
| ATOM | 1259 | C2′ | GUA | B | 206 | 11.807 | 12.242 | 23.467 | 1.00 | 61.82 | B |
| ATOM | 1260 | O2′ | GUA | B | 206 | 11.614 | 10.848 | 23.619 | 1.00 | 63.71 | B |
| ATOM | 1261 | C3′ | GUA | B | 206 | 13.201 | 12.569 | 22.956 | 1.00 | 61.23 | B |
| ATOM | 1262 | O3′ | GUA | B | 206 | 13.593 | 11.654 | 21.946 | 1.00 | 60.82 | B |
| ATOM | 1263 | P | CYT | B | 207 | 13.254 | 11.977 | 20.411 | 1.00 | 62.09 | B |
| ATOM | 1264 | O1P | CYT | B | 207 | 13.802 | 13.316 | 20.065 | 1.00 | 59.83 | B |
| ATOM | 1265 | O2P | CYT | B | 207 | 13.687 | 10.794 | 19.646 | 1.00 | 62.58 | B |
| ATOM | 1266 | O5′ | CYT | B | 207 | 11.666 | 12.035 | 20.365 | 1.00 | 60.49 | B |
| ATOM | 1267 | C5′ | CYT | B | 207 | 10.910 | 10.862 | 20.580 | 1.00 | 61.91 | B |
| ATOM | 1268 | C4′ | CYT | B | 207 | 9.470 | 11.104 | 20.236 | 1.00 | 63.97 | B |
| ATOM | 1269 | O4′ | CYT | B | 207 | 8.838 | 11.868 | 21.297 | 1.00 | 64.87 | B |
| ATOM | 1270 | C1′ | CYT | B | 207 | 7.926 | 12.791 | 20.734 | 1.00 | 63.80 | B |
| ATOM | 1271 | N1 | CYT | B | 207 | 8.496 | 14.125 | 20.900 | 1.00 | 63.17 | B |
| ATOM | 1272 | C6 | CYT | B | 207 | 9.847 | 14.323 | 20.840 | 1.00 | 62.47 | B |
| ATOM | 1273 | C2 | CYT | B | 207 | 7.640 | 15.188 | 21.116 | 1.00 | 62.29 | B |
| ATOM | 1274 | O2 | CYT | B | 207 | 6.419 | 14.973 | 21.138 | 1.00 | 62.58 | B |
| ATOM | 1275 | N3 | CYT | B | 207 | 8.153 | 16.423 | 21.288 | 1.00 | 62.17 | B |
| ATOM | 1276 | C4 | CYT | B | 207 | 9.472 | 16.610 | 21.235 | 1.00 | 61.91 | B |
| ATOM | 1277 | N4 | CYT | B | 207 | 9.936 | 17.844 | 21.422 | 1.00 | 62.98 | B |
| ATOM | 1278 | C5 | CYT | B | 207 | 10.372 | 15.539 | 20.994 | 1.00 | 61.76 | B |
| ATOM | 1279 | C2′ | CYT | B | 207 | 7.802 | 12.407 | 19.263 | 1.00 | 64.14 | B |
| ATOM | 1280 | O2′ | CYT | B | 207 | 6.858 | 11.364 | 19.158 | 1.00 | 64.99 | B |
| ATOM | 1281 | C3′ | CYT | B | 207 | 9.217 | 11.932 | 18.996 | 1.00 | 64.31 | B |
| ATOM | 1282 | O3′ | CYT | B | 207 | 9.304 | 11.120 | 17.845 | 1.00 | 68.01 | B |
| ATOM | 1283 | P | GUA | B | 208 | 10.039 | 11.681 | 16.530 | 1.00 | 71.50 | B |
| ATOM | 1284 | O1P | GUA | B | 208 | 10.903 | 12.834 | 16.895 | 1.00 | 71.03 | B |
| ATOM | 1285 | O2P | GUA | B | 208 | 10.630 | 10.512 | 15.838 | 1.00 | 70.19 | B |
| ATOM | 1286 | O5′ | GUA | B | 208 | 8.831 | 12.216 | 15.654 | 1.00 | 71.24 | B |
| ATOM | 1287 | C5′ | GUA | B | 208 | 7.619 | 11.488 | 15.626 | 1.00 | 75.00 | B |
| ATOM | 1288 | C4′ | GUA | B | 208 | 6.593 | 12.222 | 14.819 | 1.00 | 77.63 | B |
| ATOM | 1289 | O4′ | GUA | B | 208 | 6.258 | 13.454 | 15.502 | 1.00 | 78.81 | B |
| ATOM | 1290 | C1′ | GUA | B | 208 | 5.958 | 14.454 | 14.546 | 1.00 | 77.89 | B |
| ATOM | 1291 | N9 | GUA | B | 208 | 6.908 | 15.543 | 14.725 | 1.00 | 76.63 | B |
| ATOM | 1292 | C4 | GUA | B | 208 | 6.596 | 16.873 | 14.757 | 1.00 | 76.06 | B |
| ATOM | 1293 | N3 | GUA | B | 208 | 5.363 | 17.394 | 14.605 | 1.00 | 75.43 | B |
| ATOM | 1294 | C2 | GUA | B | 208 | 5.371 | 18.710 | 14.688 | 1.00 | 75.88 | B |
| ATOM | 1295 | N2 | GUA | B | 208 | 4.225 | 19.386 | 14.555 | 1.00 | 76.45 | B |
| ATOM | 1296 | N1 | GUA | B | 208 | 6.503 | 19.462 | 14.907 | 1.00 | 75.46 | B |
| ATOM | 1297 | C6 | GUA | B | 208 | 7.786 | 18.948 | 15.071 | 1.00 | 76.04 | B |
| ATOM | 1298 | O6 | GUA | B | 208 | 8.739 | 19.716 | 15.276 | 1.00 | 75.23 | B |
| ATOM | 1299 | C5 | GUA | B | 208 | 7.792 | 17.526 | 14.976 | 1.00 | 76.48 | B |
| ATOM | 1300 | N7 | GUA | B | 208 | 8.839 | 16.616 | 15.068 | 1.00 | 75.92 | B |
| ATOM | 1301 | C8 | GUA | B | 208 | 8.268 | 15.453 | 14.909 | 1.00 | 75.95 | B |
| ATOM | 1302 | C2′ | GUA | B | 208 | 6.023 | 13.790 | 13.170 | 1.00 | 79.19 | B |
| ATOM | 1303 | O2′ | GUA | B | 208 | 4.734 | 13.291 | 12.840 | 1.00 | 80.56 | B |
| ATOM | 1304 | C3′ | GUA | B | 208 | 7.025 | 12.675 | 13.434 | 1.00 | 78.39 | B |
| ATOM | 1305 | O3′ | GUA | B | 208 | 6.853 | 11.614 | 12.505 | 1.00 | 78.55 | B |
| ATOM | 1306 | P | ADE | B | 209 | 7.557 | 11.694 | 11.066 | 1.00 | 79.91 | B |
| ATOM | 1307 | O1P | ADE | B | 209 | 7.729 | 10.295 | 10.601 | 1.00 | 79.05 | B |
| ATOM | 1308 | O2P | ADE | B | 209 | 8.743 | 12.592 | 11.172 | 1.00 | 79.16 | B |
| ATOM | 1309 | O5′ | ADE | B | 209 | 6.461 | 12.386 | 10.137 | 1.00 | 78.24 | B |
| ATOM | 1310 | C5′ | ADE | B | 209 | 6.714 | 13.641 | 9.507 | 1.00 | 75.75 | B |
| ATOM | 1311 | C4′ | ADE | B | 209 | 5.411 | 14.354 | 9.221 | 1.00 | 73.21 | B |
| ATOM | 1312 | O4′ | ADE | B | 209 | 4.873 | 14.899 | 10.460 | 1.00 | 73.62 | B |
| ATOM | 1313 | C1′ | ADE | B | 209 | 4.502 | 16.256 | 10.270 | 1.00 | 72.82 | B |
| ATOM | 1314 | N9 | ADE | B | 209 | 5.575 | 17.095 | 10.804 | 1.00 | 72.74 | B |
| ATOM | 1315 | C4 | ADE | B | 209 | 5.493 | 18.434 | 11.097 | 1.00 | 71.79 | B |
| ATOM | 1316 | N3 | ADE | B | 209 | 4.414 | 19.226 | 10.987 | 1.00 | 70.98 | B |
| ATOM | 1317 | C2 | ADE | B | 209 | 4.715 | 20.472 | 11.336 | 1.00 | 71.75 | B |
| ATOM | 1318 | N1 | ADE | B | 209 | 5.885 | 20.981 | 11.746 | 1.00 | 72.10 | B |
| ATOM | 1319 | C6 | ADE | B | 209 | 6.952 | 20.154 | 11.837 | 1.00 | 72.26 | B |
| ATOM | 1320 | N6 | ADE | B | 209 | 8.129 | 20.658 | 12.223 | 1.00 | 71.78 | B |
| ATOM | 1321 | C5 | ADE | B | 209 | 6.760 | 18.807 | 11.508 | 1.00 | 71.85 | B |
| ATOM | 1322 | N7 | ADE | B | 209 | 7.621 | 17.720 | 11.501 | 1.00 | 72.48 | B |
| ATOM | 1323 | C8 | ADE | B | 209 | 6.872 | 16.730 | 11.085 | 1.00 | 72.58 | B |
| ATOM | 1324 | C2′ | ADE | B | 209 | 4.371 | 16.428 | 8.758 | 1.00 | 72.33 | B |
| ATOM | 1325 | O2′ | ADE | B | 209 | 3.104 | 16.003 | 8.293 | 1.00 | 72.90 | B |
| ATOM | 1326 | C3′ | ADE | B | 209 | 5.512 | 15.544 | 8.279 | 1.00 | 71.38 | B |
| ATOM | 1327 | O3′ | ADE | B | 209 | 5.308 | 15.134 | 6.943 | 1.00 | 69.22 | B |
| ATOM | 1328 | P | URI | B | 210 | 5.948 | 15.987 | 5.751 | 1.00 | 68.62 | B |
| ATOM | 1329 | O1P | URI | B | 210 | 7.386 | 16.234 | 6.026 | 1.00 | 66.35 | B |
| ATOM | 1330 | O2P | URI | B | 210 | 5.552 | 15.310 | 4.493 | 1.00 | 70.59 | B |
| ATOM | 1331 | O5′ | URI | B | 210 | 5.184 | 17.379 | 5.847 | 1.00 | 67.97 | B |
| ATOM | 1332 | C5′ | URI | B | 210 | 3.784 | 17.451 | 5.619 | 1.00 | 65.59 | B |
| ATOM | 1333 | C4′ | URI | B | 210 | 3.285 | 18.867 | 5.806 | 1.00 | 64.54 | B |
| ATOM | 1334 | O4′ | URI | B | 210 | 3.438 | 19.289 | 7.190 | 1.00 | 63.95 | B |
| ATOM | 1335 | C1′ | URI | B | 210 | 3.513 | 20.706 | 7.236 | 1.00 | 63.40 | B |
| ATOM | 1336 | N1 | URI | B | 210 | 4.820 | 21.090 | 7.786 | 1.00 | 62.33 | B |
| ATOM | 1337 | C6 | URI | B | 210 | 5.885 | 20.236 | 7.750 | 1.00 | 60.71 | B |
| ATOM | 1338 | C2 | URI | B | 210 | 4.952 | 22.366 | 8.301 | 1.00 | 61.50 | B |
| ATOM | 1339 | O2 | URI | B | 210 | 4.010 | 23.134 | 8.428 | 1.00 | 62.05 | B |
| ATOM | 1340 | N3 | URI | B | 210 | 6.224 | 22.709 | 8.673 | 1.00 | 59.69 | B |
| ATOM | 1341 | C4 | URI | B | 210 | 7.342 | 21.919 | 8.613 | 1.00 | 59.43 | B |
| ATOM | 1342 | O4 | URI | B | 210 | 8.440 | 22.411 | 8.868 | 1.00 | 60.44 | B |
| ATOM | 1343 | C5 | URI | B | 210 | 7.109 | 20.596 | 8.138 | 1.00 | 60.21 | B |
| ATOM | 1344 | C2′ | URI | B | 210 | 3.400 | 21.181 | 5.782 | 1.00 | 63.67 | B |
| ATOM | 1345 | O2′ | URI | B | 210 | 2.047 | 21.387 | 5.433 | 1.00 | 64.63 | B |
| ATOM | 1346 | C3′ | URI | B | 210 | 3.980 | 19.986 | 5.050 | 1.00 | 63.00 | B |
| ATOM | 1347 | O3′ | URI | B | 210 | 3.613 | 20.025 | 3.674 | 1.00 | 61.87 | B |
| ATOM | 1348 | P | URI | B | 211 | 4.552 | 20.791 | 2.619 | 1.00 | 62.14 | B |
| ATOM | 1349 | O1P | URI | B | 211 | 5.953 | 20.288 | 2.709 | 1.00 | 62.97 | B |
| ATOM | 1350 | O2P | URI | B | 211 | 3.861 | 20.762 | 1.316 | 1.00 | 64.45 | B |
| ATOM | 1351 | O5′ | URI | B | 211 | 4.552 | 22.292 | 3.144 | 1.00 | 64.09 | B |
| ATOM | 1352 | C5′ | URI | B | 211 | 3.352 | 23.055 | 3.188 | 1.00 | 63.77 | B |
| ATOM | 1353 | C4′ | URI | B | 211 | 3.648 | 24.477 | 3.606 | 1.00 | 64.74 | B |
| ATOM | 1354 | O4′ | URI | B | 211 | 4.134 | 24.496 | 4.975 | 1.00 | 64.83 | B |
| ATOM | 1355 | C1′ | URI | B | 211 | 5.030 | 25.586 | 5.145 | 1.00 | 66.00 | B |
| ATOM | 1356 | N1 | URI | B | 211 | 6.350 | 25.066 | 5.527 | 1.00 | 64.36 | B |
| ATOM | 1357 | C6 | URI | B | 211 | 6.708 | 23.768 | 5.295 | 1.00 | 63.97 | B |
| ATOM | 1358 | C2 | URI | B | 211 | 7.224 | 25.947 | 6.111 | 1.00 | 63.57 | B |
| ATOM | 1359 | O2 | URI | B | 211 | 6.921 | 27.098 | 6.359 | 1.00 | 65.32 | B |
| ATOM | 1360 | N3 | URI | B | 211 | 8.463 | 25.434 | 6.399 | 1.00 | 62.40 | B |
| ATOM | 1361 | C4 | URI | B | 211 | 8.896 | 24.151 | 6.173 | 1.00 | 62.58 | B |
| ATOM | 1362 | O4 | URI | B | 211 | 10.061 | 23.851 | 6.424 | 1.00 | 61.65 | B |
| ATOM | 1363 | C5 | URI | B | 211 | 7.920 | 23.291 | 5.591 | 1.00 | 64.22 | B |
| ATOM | 1364 | C2′ | URI | B | 211 | 5.096 | 26.322 | 3.806 | 1.00 | 66.91 | B |
| ATOM | 1365 | O2′ | URI | B | 211 | 4.175 | 27.396 | 3.821 | 1.00 | 67.58 | B |
| ATOM | 1366 | C3′ | URI | B | 211 | 4.746 | 25.196 | 2.836 | 1.00 | 66.05 | B |
| ATOM | 1367 | O3′ | URI | B | 211 | 4.266 | 25.680 | 1.582 | 1.00 | 67.10 | B |
| ATOM | 1368 | P | URI | B | 212 | 5.296 | 25.943 | 0.371 | 1.00 | 68.24 | B |
| ATOM | 1369 | O1P | URI | B | 212 | 6.124 | 24.732 | 0.127 | 1.00 | 66.86 | B |
| ATOM | 1370 | O2P | URI | B | 212 | 4.515 | 26.535 | −0.748 | 1.00 | 67.34 | B |
| ATOM | 1371 | O5′ | URI | B | 212 | 6.262 | 27.074 | 0.935 | 1.00 | 66.78 | B |
| ATOM | 1372 | C5′ | URI | B | 212 | 5.753 | 28.356 | 1.279 | 1.00 | 63.87 | B |
| ATOM | 1373 | C4′ | URI | B | 212 | 6.880 | 29.264 | 1.683 | 1.00 | 61.99 | B |
| ATOM | 1374 | O4′ | URI | B | 212 | 7.431 | 28.817 | 2.944 | 1.00 | 61.09 | B |
| ATOM | 1375 | C1′ | URI | B | 212 | 8.789 | 29.201 | 3.012 | 1.00 | 60.91 | B |
| ATOM | 1376 | N1 | URI | B | 212 | 9.596 | 28.017 | 3.306 | 1.00 | 62.64 | B |
| ATOM | 1377 | C6 | URI | B | 212 | 9.116 | 26.742 | 3.141 | 1.00 | 63.07 | B |
| ATOM | 1378 | C2 | URI | B | 212 | 10.872 | 28.244 | 3.744 | 1.00 | 64.10 | B |
| ATOM | 1379 | O2 | URI | B | 212 | 11.315 | 29.368 | 3.896 | 1.00 | 65.97 | B |
| ATOM | 1380 | N3 | URI | B | 212 | 11.613 | 27.116 | 3.992 | 1.00 | 64.01 | B |
| ATOM | 1381 | C4 | URI | B | 212 | 11.204 | 25.814 | 3.841 | 1.00 | 62.96 | B |
| ATOM | 1382 | O4 | URI | B | 212 | 11.991 | 24.909 | 4.086 | 1.00 | 62.78 | B |
| ATOM | 1383 | C5 | URI | B | 212 | 9.858 | 25.659 | 3.384 | 1.00 | 62.88 | B |
| ATOM | 1384 | C2′ | URI | B | 212 | 9.179 | 29.779 | 1.655 | 1.00 | 60.05 | B |
| ATOM | 1385 | O2′ | URI | B | 212 | 9.195 | 31.180 | 1.725 | 1.00 | 60.00 | B |
| ATOM | 1386 | C3′ | URI | B | 212 | 8.079 | 29.232 | 0.760 | 1.00 | 60.82 | B |
| ATOM | 1387 | O3′ | URI | B | 212 | 7.861 | 30.082 | −0.345 | 1.00 | 62.32 | B |
| ATOM | 1388 | P | ADE | B | 213 | 8.786 | 29.945 | −1.642 | 1.00 | 64.19 | B |
| ATOM | 1389 | O1P | ADE | B | 213 | 8.514 | 31.129 | −2.486 | 1.00 | 62.11 | B |
| ATOM | 1390 | O2P | ADE | B | 213 | 8.592 | 28.580 | −2.199 | 1.00 | 64.56 | B |
| ATOM | 1391 | O5′ | ADE | B | 213 | 10.270 | 30.084 | −1.086 | 1.00 | 64.30 | B |
| ATOM | 1392 | C5′ | ADE | B | 213 | 10.903 | 31.352 | −1.037 | 1.00 | 66.56 | B |
| ATOM | 1393 | C4′ | ADE | B | 213 | 11.967 | 31.424 | −2.091 | 1.00 | 68.08 | B |
| ATOM | 1394 | O4′ | ADE | B | 213 | 12.985 | 30.491 | −1.693 | 1.00 | 67.73 | B |
| ATOM | 1395 | C1′ | ADE | B | 213 | 13.593 | 29.941 | −2.834 | 1.00 | 69.20 | B |
| ATOM | 1396 | N9 | ADE | B | 213 | 13.824 | 28.534 | −2.557 | 1.00 | 68.20 | B |
| ATOM | 1397 | C4 | ADE | B | 213 | 15.013 | 28.009 | −2.132 | 1.00 | 68.22 | B |
| ATOM | 1398 | N3 | ADE | B | 213 | 16.161 | 28.672 | −1.919 | 1.00 | 67.53 | B |
| ATOM | 1399 | C2 | ADE | B | 213 | 17.102 | 27.846 | −1.484 | 1.00 | 67.81 | B |
| ATOM | 1400 | N1 | ADE | B | 213 | 17.034 | 26.532 | −1.260 | 1.00 | 67.62 | B |
| ATOM | 1401 | C6 | ADE | B | 213 | 15.868 | 25.897 | −1.495 | 1.00 | 67.62 | B |
| ATOM | 1402 | N6 | ADE | B | 213 | 15.808 | 24.586 | −1.284 | 1.00 | 68.78 | B |
| ATOM | 1403 | C5 | ADE | B | 213 | 14.788 | 26.663 | −1.952 | 1.00 | 67.89 | B |
| ATOM | 1404 | N7 | ADE | B | 213 | 13.478 | 26.340 | −2.274 | 1.00 | 68.10 | B |
| ATOM | 1405 | C8 | ADE | B | 213 | 12.952 | 27.485 | −2.631 | 1.00 | 68.29 | B |
| ATOM | 1406 | C2′ | ADE | B | 213 | 12.708 | 30.196 | −4.054 | 1.00 | 70.12 | B |
| ATOM | 1407 | O2′ | ADE | B | 213 | 13.432 | 30.890 | −5.056 | 1.00 | 72.73 | B |
| ATOM | 1408 | C3′ | ADE | B | 213 | 11.508 | 30.945 | −3.462 | 1.00 | 69.63 | B |
| ATOM | 1409 | O3′ | ADE | B | 213 | 10.942 | 31.982 | −4.285 | 1.00 | 70.47 | B |
| ATOM | 1410 | P | ADE | B | 214 | 11.773 | 33.329 | −4.649 | 1.00 | 69.36 | B |
| ATOM | 1411 | O1P | ADE | B | 214 | 13.241 | 33.164 | −4.465 | 1.00 | 68.13 | B |
| ATOM | 1412 | O2P | ADE | B | 214 | 11.068 | 34.439 | −3.965 | 1.00 | 67.30 | B |
| ATOM | 1413 | O5′ | ADE | B | 214 | 11.486 | 33.476 | −6.204 | 1.00 | 67.59 | B |
| ATOM | 1414 | C5′ | ADE | B | 214 | 10.417 | 32.740 | −6.774 | 1.00 | 69.16 | B |
| ATOM | 1415 | C4′ | ADE | B | 214 | 10.411 | 32.875 | −8.268 | 1.00 | 70.01 | B |
| ATOM | 1416 | O4′ | ADE | B | 214 | 10.089 | 34.245 | −8.615 | 1.00 | 69.71 | B |
| ATOM | 1417 | C1′ | ADE | B | 214 | 10.711 | 34.566 | −9.849 | 1.00 | 69.81 | B |
| ATOM | 1418 | N9 | ADE | B | 214 | 11.758 | 35.547 | −9.587 | 1.00 | 67.72 | B |
| ATOM | 1419 | C4 | ADE | B | 214 | 11.965 | 36.718 | −10.265 | 1.00 | 66.16 | B |
| ATOM | 1420 | N3 | ADE | B | 214 | 11.225 | 37.218 | −11.267 | 1.00 | 63.36 | B |
| ATOM | 1421 | C2 | ADE | B | 214 | 11.738 | 38.363 | −11.693 | 1.00 | 64.58 | B |
| ATOM | 1422 | N1 | ADE | B | 214 | 12.832 | 39.016 | −11.266 | 1.00 | 66.51 | B |
| ATOM | 1423 | C6 | ADE | B | 214 | 13.554 | 38.478 | −10.257 | 1.00 | 66.22 | B |
| ATOM | 1424 | N6 | ADE | B | 214 | 14.648 | 39.116 | −9.833 | 1.00 | 65.04 | B |
| ATOM | 1425 | C5 | ADE | B | 214 | 13.107 | 37.272 | −9.715 | 1.00 | 66.42 | B |
| ATOM | 1426 | N7 | ADE | B | 214 | 13.594 | 36.479 | −8.691 | 1.00 | 67.11 | B |
| ATOM | 1427 | C8 | ADE | B | 214 | 12.756 | 35.475 | −8.649 | 1.00 | 67.96 | B |
| ATOM | 1428 | C2′ | ADE | B | 214 | 11.324 | 33.259 | −10.351 | 1.00 | 70.77 | B |
| ATOM | 1429 | O2′ | ADE | B | 214 | 10.339 | 32.494 | −11.025 | 1.00 | 70.42 | B |
| ATOM | 1430 | C3′ | ADE | B | 214 | 11.708 | 32.637 | −9.024 | 1.00 | 71.72 | B |
| ATOM | 1431 | O3′ | ADE | B | 214 | 12.024 | 31.255 | −9.166 | 1.00 | 76.39 | B |
| ATOM | 1432 | P | CYT | B | 215 | 13.418 | 30.800 | −9.864 | 1.00 | 79.37 | B |
| ATOM | 1433 | O1P | CYT | B | 215 | 14.341 | 30.359 | −8.769 | 1.00 | 77.34 | B |
| ATOM | 1434 | O2P | CYT | B | 215 | 13.884 | 31.829 | −10.846 | 1.00 | 79.33 | B |
| ATOM | 1435 | O5′ | CYT | B | 215 | 12.976 | 29.515 | −10.704 | 1.00 | 76.94 | B |
| ATOM | 1436 | C5′ | CYT | B | 215 | 11.607 | 29.327 | −11.086 | 1.00 | 71.33 | B |
| ATOM | 1437 | C4′ | CYT | B | 215 | 11.448 | 28.061 | −11.897 | 1.00 | 67.39 | B |
| ATOM | 1438 | O4′ | CYT | B | 215 | 12.392 | 28.049 | −13.007 | 1.00 | 65.08 | B |
| ATOM | 1439 | C1′ | CYT | B | 215 | 13.043 | 26.801 | −13.057 | 1.00 | 61.70 | B |
| ATOM | 1440 | N1 | CYT | B | 215 | 14.359 | 26.971 | −12.449 | 1.00 | 59.95 | B |
| ATOM | 1441 | C6 | CYT | B | 215 | 14.792 | 28.206 | −12.062 | 1.00 | 58.65 | B |
| ATOM | 1442 | C2 | CYT | B | 215 | 15.160 | 25.851 | −12.258 | 1.00 | 60.14 | B |
| ATOM | 1443 | O2 | CYT | B | 215 | 14.750 | 24.747 | −12.652 | 1.00 | 60.89 | B |
| ATOM | 1444 | N3 | CYT | B | 215 | 16.362 | 25.995 | −11.656 | 1.00 | 58.69 | B |
| ATOM | 1445 | C4 | CYT | B | 215 | 16.768 | 27.205 | −11.264 | 1.00 | 57.84 | B |
| ATOM | 1446 | N4 | CYT | B | 215 | 17.953 | 27.302 | −10.663 | 1.00 | 57.51 | B |
| ATOM | 1447 | C5 | CYT | B | 215 | 15.975 | 28.367 | −11.469 | 1.00 | 57.82 | B |
| ATOM | 1448 | C2′ | CYT | B | 215 | 12.156 | 25.844 | −12.265 | 1.00 | 63.48 | B |
| ATOM | 1449 | O2′ | CYT | B | 215 | 11.095 | 25.406 | −13.089 | 1.00 | 64.98 | B |
| ATOM | 1450 | C3′ | CYT | B | 215 | 11.667 | 26.754 | −11.151 | 1.00 | 64.05 | B |
| ATOM | 1451 | O3′ | CYT | B | 215 | 10.410 | 26.296 | −10.690 | 1.00 | 63.08 | B |
| ATOM | 1452 | P | CYT | B | 216 | 10.197 | 25.977 | −9.135 | 1.00 | 64.12 | B |
| ATOM | 1453 | O1P | CYT | B | 216 | 11.047 | 26.891 | −8.322 | 1.00 | 63.64 | B |
| ATOM | 1454 | O2P | CYT | B | 216 | 8.729 | 25.920 | −8.889 | 1.00 | 62.19 | B |
| ATOM | 1455 | O5′ | CYT | B | 216 | 10.802 | 24.517 | −8.988 | 1.00 | 61.93 | B |
| ATOM | 1456 | C5′ | CYT | B | 216 | 10.525 | 23.517 | −9.957 | 1.00 | 58.67 | B |
| ATOM | 1457 | C4′ | CYT | B | 216 | 11.344 | 22.282 | −9.675 | 1.00 | 56.94 | B |
| ATOM | 1458 | O4′ | CYT | B | 216 | 12.646 | 22.445 | −10.283 | 1.00 | 58.42 | B |
| ATOM | 1459 | C1′ | CYT | B | 216 | 13.623 | 21.798 | −9.496 | 1.00 | 55.98 | B |
| ATOM | 1460 | N1 | CYT | B | 216 | 14.576 | 22.800 | −9.063 | 1.00 | 56.15 | B |
| ATOM | 1461 | C6 | CYT | B | 216 | 14.295 | 24.130 | −9.178 | 1.00 | 56.50 | B |
| ATOM | 1462 | C2 | CYT | B | 216 | 15.772 | 22.374 | −8.527 | 1.00 | 57.23 | B |
| ATOM | 1463 | O2 | CYT | B | 216 | 15.970 | 21.157 | −8.420 | 1.00 | 56.87 | B |
| ATOM | 1464 | N3 | CYT | B | 216 | 16.682 | 23.290 | −8.130 | 1.00 | 57.99 | B |
| ATOM | 1465 | C4 | CYT | B | 216 | 16.406 | 24.591 | −8.247 | 1.00 | 58.03 | B |
| ATOM | 1466 | N4 | CYT | B | 216 | 17.326 | 25.466 | −7.844 | 1.00 | 58.87 | B |
| ATOM | 1467 | C5 | CYT | B | 216 | 15.172 | 25.051 | −8.784 | 1.00 | 58.58 | B |
| ATOM | 1468 | C2′ | CYT | B | 216 | 12.920 | 21.181 | −8.296 | 1.00 | 55.14 | B |
| ATOM | 1469 | O2′ | CYT | B | 216 | 12.635 | 19.837 | −8.575 | 1.00 | 58.96 | B |
| ATOM | 1470 | C3′ | CYT | B | 216 | 11.653 | 22.012 | −8.212 | 1.00 | 54.62 | B |
| ATOM | 1471 | O3′ | CYT | B | 216 | 10.641 | 21.211 | −7.651 | 1.00 | 51.22 | B |
| ATOM | 1472 | P | GUA | B | 217 | 10.195 | 21.454 | −6.141 | 1.00 | 51.46 | B |
| ATOM | 1473 | O1P | GUA | B | 217 | 9.903 | 22.898 | −6.028 | 1.00 | 51.75 | B |
| ATOM | 1474 | O2P | GUA | B | 217 | 9.140 | 20.468 | −5.804 | 1.00 | 52.33 | B |
| ATOM | 1475 | O5′ | GUA | B | 217 | 11.506 | 21.148 | −5.295 | 1.00 | 50.74 | B |
| ATOM | 1476 | C5′ | GUA | B | 217 | 12.249 | 19.952 | −5.488 | 1.00 | 53.55 | B |
| ATOM | 1477 | C4′ | GUA | B | 217 | 13.474 | 19.959 | −4.605 | 1.00 | 56.25 | B |
| ATOM | 1478 | O4′ | GUA | B | 217 | 14.391 | 20.990 | −5.056 | 1.00 | 59.75 | B |
| ATOM | 1479 | C1′ | GUA | B | 217 | 14.472 | 22.021 | −4.090 | 1.00 | 61.77 | B |
| ATOM | 1480 | N9 | GUA | B | 217 | 14.188 | 23.291 | −4.738 | 1.00 | 66.26 | B |
| ATOM | 1481 | C4 | GUA | B | 217 | 15.045 | 24.339 | −4.873 | 1.00 | 68.16 | B |
| ATOM | 1482 | N3 | GUA | B | 217 | 16.327 | 24.364 | −4.461 | 1.00 | 69.17 | B |
| ATOM | 1483 | C2 | GUA | B | 217 | 16.896 | 25.533 | −4.714 | 1.00 | 73.15 | B |
| ATOM | 1484 | N2 | GUA | B | 217 | 18.191 | 25.746 | −4.372 | 1.00 | 73.91 | B |
| ATOM | 1485 | N1 | GUA | B | 217 | 16.242 | 26.585 | −5.323 | 1.00 | 73.30 | B |
| ATOM | 1486 | C6 | GUA | B | 217 | 14.916 | 26.566 | −5.752 | 1.00 | 72.06 | B |
| ATOM | 1487 | O6 | GUA | B | 217 | 14.419 | 27.562 | −6.288 | 1.00 | 74.99 | B |
| ATOM | 1488 | C5 | GUA | B | 217 | 14.310 | 25.335 | −5.491 | 1.00 | 69.90 | B |
| ATOM | 1489 | N7 | GUA | B | 217 | 13.019 | 24.908 | −5.754 | 1.00 | 68.66 | B |
| ATOM | 1490 | C8 | GUA | B | 217 | 12.993 | 23.687 | −5.297 | 1.00 | 68.05 | B |
| ATOM | 1491 | C2′ | GUA | B | 217 | 13.388 | 21.772 | −3.047 | 1.00 | 59.42 | B |
| ATOM | 1492 | O2′ | GUA | B | 217 | 13.837 | 22.202 | −1.779 | 1.00 | 59.88 | B |
| ATOM | 1493 | C3′ | GUA | B | 217 | 13.179 | 20.270 | −3.147 | 1.00 | 56.41 | B |
| ATOM | 1494 | O3′ | GUA | B | 217 | 14.074 | 19.609 | −2.290 | 1.00 | 50.70 | B |
| ATOM | 1495 | P | URI | B | 218 | 13.502 | 18.644 | −1.157 | 1.00 | 49.79 | B |
| ATOM | 1496 | O1P | URI | B | 218 | 13.606 | 19.315 | 0.155 | 1.00 | 46.50 | B |
| ATOM | 1497 | O2P | URI | B | 218 | 12.181 | 18.143 | −1.602 | 1.00 | 49.67 | B |
| ATOM | 1498 | O5′ | URI | B | 218 | 14.553 | 17.455 | −1.218 | 1.00 | 50.09 | B |
| ATOM | 1499 | C5′ | URI | B | 218 | 15.031 | 16.855 | −0.037 | 1.00 | 54.12 | B |
| ATOM | 1500 | C4′ | URI | B | 218 | 16.493 | 16.552 | −0.168 | 1.00 | 57.01 | B |
| ATOM | 1501 | O4′ | URI | B | 218 | 17.078 | 17.324 | −1.248 | 1.00 | 58.97 | B |
| ATOM | 1502 | C1′ | URI | B | 218 | 18.380 | 17.762 | −0.867 | 1.00 | 60.00 | B |
| ATOM | 1503 | N1 | URI | B | 218 | 18.354 | 19.226 | −0.735 | 1.00 | 60.71 | B |
| ATOM | 1504 | C6 | URI | B | 218 | 17.184 | 19.929 | −0.865 | 1.00 | 60.31 | B |
| ATOM | 1505 | C2 | URI | B | 218 | 19.547 | 19.876 | −0.483 | 1.00 | 60.85 | B |
| ATOM | 1506 | O2 | URI | B | 218 | 20.601 | 19.291 | −0.346 | 1.00 | 62.32 | B |
| ATOM | 1507 | N3 | URI | B | 218 | 19.457 | 21.241 | −0.392 | 1.00 | 60.77 | B |
| ATOM | 1508 | C4 | URI | B | 218 | 18.322 | 22.006 | −0.522 | 1.00 | 60.53 | B |
| ATOM | 1509 | O4 | URI | B | 218 | 18.402 | 23.232 | −0.437 | 1.00 | 58.93 | B |
| ATOM | 1510 | C5 | URI | B | 218 | 17.128 | 21.260 | −0.769 | 1.00 | 61.10 | B |
| ATOM | 1511 | C2′ | URI | B | 218 | 18.693 | 17.057 | 0.450 | 1.00 | 59.40 | B |
| ATOM | 1512 | O2′ | URI | B | 218 | 19.214 | 15.781 | 0.165 | 1.00 | 61.50 | B |
| ATOM | 1513 | C3′ | URI | B | 218 | 17.301 | 16.966 | 1.040 | 1.00 | 59.18 | B |
| ATOM | 1514 | O3′ | URI | B | 218 | 17.185 | 15.965 | 2.016 | 1.00 | 60.42 | B |
| ATOM | 1515 | P | ADE | B | 219 | 16.564 | 16.333 | 3.438 | 1.00 | 64.24 | B |
| ATOM | 1516 | O1P | ADE | B | 219 | 15.728 | 15.179 | 3.852 | 1.00 | 62.72 | B |
| ATOM | 1517 | O2P | ADE | B | 219 | 15.965 | 17.690 | 3.380 | 1.00 | 62.59 | B |
| ATOM | 1518 | O5′ | ADE | B | 219 | 17.862 | 16.379 | 4.350 | 1.00 | 66.08 | B |
| ATOM | 1519 | C5′ | ADE | B | 219 | 19.022 | 15.656 | 3.956 | 1.00 | 68.19 | B |
| ATOM | 1520 | C4′ | ADE | B | 219 | 20.261 | 16.406 | 4.352 | 1.00 | 69.24 | B |
| ATOM | 1521 | O4′ | ADE | B | 219 | 20.575 | 17.422 | 3.373 | 1.00 | 70.00 | B |
| ATOM | 1522 | C1′ | ADE | B | 219 | 21.308 | 18.458 | 4.006 | 1.00 | 71.78 | B |
| ATOM | 1523 | N9 | ADE | B | 219 | 20.667 | 19.735 | 3.704 | 1.00 | 74.23 | B |
| ATOM | 1524 | C4 | ADE | B | 219 | 21.235 | 20.970 | 3.866 | 1.00 | 74.89 | B |
| ATOM | 1525 | N3 | ADE | B | 219 | 22.460 | 21.238 | 4.335 | 1.00 | 75.81 | B |
| ATOM | 1526 | C2 | ADE | B | 219 | 22.677 | 22.547 | 4.342 | 1.00 | 76.38 | B |
| ATOM | 1527 | N1 | ADE | B | 219 | 21.871 | 23.542 | 3.959 | 1.00 | 76.67 | B |
| ATOM | 1528 | C6 | ADE | B | 219 | 20.643 | 23.237 | 3.491 | 1.00 | 76.42 | B |
| ATOM | 1529 | N6 | ADE | B | 219 | 19.839 | 24.234 | 3.103 | 1.00 | 77.15 | B |
| ATOM | 1530 | C5 | ADE | B | 219 | 20.288 | 21.882 | 3.439 | 1.00 | 75.79 | B |
| ATOM | 1531 | N7 | ADE | B | 219 | 19.135 | 21.235 | 3.022 | 1.00 | 75.71 | B |
| ATOM | 1532 | C8 | ADE | B | 219 | 19.408 | 19.965 | 3.201 | 1.00 | 76.30 | B |
| ATOM | 1533 | C2′ | ADE | B | 219 | 21.328 | 18.143 | 5.503 | 1.00 | 70.95 | B |
| ATOM | 1534 | O2′ | ADE | B | 219 | 22.542 | 17.496 | 5.822 | 1.00 | 71.79 | B |
| ATOM | 1535 | C3′ | ADE | B | 219 | 20.135 | 17.209 | 5.625 | 1.00 | 69.63 | B |
| ATOM | 1536 | O3′ | ADE | B | 219 | 20.258 | 16.343 | 6.735 | 1.00 | 69.76 | B |
| ATOM | 1537 | P | URI | B | 220 | 19.453 | 16.666 | 8.079 | 1.00 | 70.71 | B |
| ATOM | 1538 | O1P | URI | B | 220 | 18.008 | 16.748 | 7.750 | 1.00 | 69.47 | B |
| ATOM | 1539 | O2P | URI | B | 220 | 19.918 | 15.701 | 9.099 | 1.00 | 70.74 | B |
| ATOM | 1540 | O5′ | URI | B | 220 | 19.976 | 18.115 | 8.483 | 1.00 | 70.85 | B |
| ATOM | 1541 | C5′ | URI | B | 220 | 21.273 | 18.291 | 9.050 | 1.00 | 71.86 | B |
| ATOM | 1542 | C4′ | URI | B | 220 | 21.498 | 19.742 | 9.400 | 1.00 | 73.26 | B |
| ATOM | 1543 | O4′ | URI | B | 220 | 21.577 | 20.510 | 8.172 | 1.00 | 73.76 | B |
| ATOM | 1544 | C1′ | URI | B | 220 | 20.934 | 21.761 | 8.345 | 1.00 | 74.30 | B |
| ATOM | 1545 | N1 | URI | B | 220 | 19.789 | 21.822 | 7.429 | 1.00 | 73.87 | B |
| ATOM | 1546 | C6 | URI | B | 220 | 19.072 | 20.700 | 7.101 | 1.00 | 73.82 | B |
| ATOM | 1547 | C2 | URI | B | 220 | 19.456 | 23.051 | 6.921 | 1.00 | 74.08 | B |
| ATOM | 1548 | O2 | URI | B | 220 | 20.068 | 24.066 | 7.200 | 1.00 | 74.27 | B |
| ATOM | 1549 | N3 | URI | B | 220 | 18.372 | 23.056 | 6.083 | 1.00 | 74.62 | B |
| ATOM | 1550 | C4 | URI | B | 220 | 17.597 | 21.978 | 5.723 | 1.00 | 74.41 | B |
| ATOM | 1551 | O4 | URI | B | 220 | 16.595 | 22.158 | 5.027 | 1.00 | 74.63 | B |
| ATOM | 1552 | C5 | URI | B | 220 | 18.014 | 20.734 | 6.290 | 1.00 | 74.26 | B |
| ATOM | 1553 | C2′ | URI | B | 220 | 20.503 | 21.863 | 9.808 | 1.00 | 74.65 | B |
| ATOM | 1554 | O2′ | URI | B | 220 | 21.466 | 22.564 | 10.564 | 1.00 | 74.96 | B |
| ATOM | 1555 | C3′ | URI | B | 220 | 20.375 | 20.394 | 10.192 | 1.00 | 74.77 | B |
| ATOM | 1556 | O3′ | URI | B | 220 | 20.548 | 20.202 | 11.590 | 1.00 | 75.28 | B |
| ATOM | 1557 | P | URI | B | 221 | 19.260 | 20.164 | 12.547 | 1.00 | 76.37 | B |
| ATOM | 1558 | O1P | URI | B | 221 | 19.697 | 19.545 | 13.817 | 1.00 | 76.58 | B |
| ATOM | 1559 | O2P | URI | B | 221 | 18.112 | 19.576 | 11.804 | 1.00 | 76.32 | B |
| ATOM | 1560 | O5′ | URI | B | 221 | 18.939 | 21.702 | 12.808 | 1.00 | 75.73 | B |
| ATOM | 1561 | C5′ | URI | B | 221 | 19.977 | 22.607 | 13.156 | 1.00 | 75.43 | B |
| ATOM | 1562 | C4′ | URI | B | 221 | 19.425 | 24.000 | 13.335 | 1.00 | 76.00 | B |
| ATOM | 1563 | O4′ | URI | B | 221 | 19.468 | 24.701 | 12.064 | 1.00 | 75.92 | B |
| ATOM | 1564 | C1′ | URI | B | 221 | 18.376 | 25.602 | 11.983 | 1.00 | 77.20 | B |
| ATOM | 1565 | N1 | URI | B | 221 | 17.537 | 25.194 | 10.850 | 1.00 | 78.74 | B |
| ATOM | 1566 | C6 | URI | B | 221 | 17.445 | 23.872 | 10.481 | 1.00 | 80.55 | B |
| ATOM | 1567 | C2 | URI | B | 221 | 16.831 | 26.171 | 10.178 | 1.00 | 78.55 | B |
| ATOM | 1568 | O2 | URI | B | 221 | 16.907 | 27.352 | 10.459 | 1.00 | 78.21 | B |
| ATOM | 1569 | N3 | URI | B | 221 | 16.032 | 25.709 | 9.161 | 1.00 | 78.98 | B |
| ATOM | 1570 | C4 | URI | B | 221 | 15.880 | 24.395 | 8.754 | 1.00 | 79.57 | B |
| ATOM | 1571 | O4 | URI | B | 221 | 15.102 | 24.127 | 7.839 | 1.00 | 79.33 | B |
| ATOM | 1572 | C5 | URI | B | 221 | 16.661 | 23.451 | 9.486 | 1.00 | 80.16 | B |
| ATOM | 1573 | C2′ | URI | B | 221 | 17.616 | 25.510 | 13.311 | 1.00 | 76.85 | B |
| ATOM | 1574 | O2′ | URI | B | 221 | 18.051 | 26.492 | 14.233 | 1.00 | 76.78 | B |
| ATOM | 1575 | C3′ | URI | B | 221 | 17.972 | 24.104 | 13.770 | 1.00 | 76.38 | B |
| ATOM | 1576 | O3′ | URI | B | 221 | 17.846 | 24.017 | 15.185 | 1.00 | 76.55 | B |
| ATOM | 1577 | P | GUA | B | 222 | 16.386 | 23.883 | 15.847 | 1.00 | 76.67 | B |
| ATOM | 1578 | O1P | GUA | B | 222 | 15.693 | 22.756 | 15.175 | 1.00 | 74.54 | B |
| ATOM | 1579 | O2P | GUA | B | 222 | 16.530 | 23.877 | 17.321 | 1.00 | 76.48 | B |
| ATOM | 1580 | O5′ | GUA | B | 222 | 15.650 | 25.238 | 15.450 | 1.00 | 76.37 | B |
| ATOM | 1581 | C5′ | GUA | B | 222 | 15.906 | 26.441 | 16.164 | 1.00 | 75.09 | B |
| ATOM | 1582 | C4′ | GUA | B | 222 | 14.740 | 27.398 | 16.029 | 1.00 | 75.06 | B |
| ATOM | 1583 | O4′ | GUA | B | 222 | 14.095 | 27.260 | 14.733 | 1.00 | 74.11 | B |
| ATOM | 1584 | C1′ | GUA | B | 222 | 12.733 | 27.669 | 14.841 | 1.00 | 73.06 | B |
| ATOM | 1585 | N9 | GUA | B | 222 | 11.874 | 26.564 | 14.434 | 1.00 | 71.60 | B |
| ATOM | 1586 | C4 | GUA | B | 222 | 10.550 | 26.653 | 14.082 | 1.00 | 70.04 | B |
| ATOM | 1587 | N3 | GUA | B | 222 | 9.820 | 27.782 | 14.028 | 1.00 | 68.98 | B |
| ATOM | 1588 | C2 | GUA | B | 222 | 8.580 | 27.545 | 13.656 | 1.00 | 68.24 | B |
| ATOM | 1589 | N2 | GUA | B | 222 | 7.710 | 28.556 | 13.555 | 1.00 | 69.22 | B |
| ATOM | 1590 | N1 | GUA | B | 222 | 8.099 | 26.302 | 13.355 | 1.00 | 67.50 | B |
| ATOM | 1591 | C6 | GUA | B | 222 | 8.833 | 25.129 | 13.400 | 1.00 | 67.30 | B |
| ATOM | 1592 | O6 | GUA | B | 222 | 8.303 | 24.061 | 13.100 | 1.00 | 66.59 | B |
| ATOM | 1593 | C5 | GUA | B | 222 | 10.160 | 25.365 | 13.804 | 1.00 | 69.14 | B |
| ATOM | 1594 | N7 | GUA | B | 222 | 11.216 | 24.482 | 13.974 | 1.00 | 70.34 | B |
| ATOM | 1595 | C8 | GUA | B | 222 | 12.211 | 25.238 | 14.346 | 1.00 | 70.99 | B |
| ATOM | 1596 | C2′ | GUA | B | 222 | 12.499 | 28.021 | 16.306 | 1.00 | 74.21 | B |
| ATOM | 1597 | O2′ | GUA | B | 222 | 12.671 | 29.412 | 16.469 | 1.00 | 74.59 | B |
| ATOM | 1598 | C3′ | GUA | B | 222 | 13.578 | 27.179 | 16.975 | 1.00 | 75.13 | B |
| ATOM | 1599 | O3′ | GUA | B | 222 | 13.873 | 27.625 | 18.287 | 1.00 | 76.50 | B |
| ATOM | 1600 | P | CYT | B | 223 | 13.005 | 27.071 | 19.515 | 1.00 | 78.32 | B |
| ATOM | 1601 | O1P | CYT | B | 223 | 12.973 | 25.587 | 19.411 | 1.00 | 77.82 | B |
| ATOM | 1602 | O2P | CYT | B | 223 | 13.492 | 27.714 | 20.761 | 1.00 | 77.45 | B |
| ATOM | 1603 | O5′ | CYT | B | 223 | 11.543 | 27.615 | 19.213 | 1.00 | 76.19 | B |
| ATOM | 1604 | C5′ | CYT | B | 223 | 11.321 | 29.006 | 19.055 | 1.00 | 75.87 | B |
| ATOM | 1605 | C4′ | CYT | B | 223 | 9.864 | 29.272 | 18.814 | 1.00 | 74.71 | B |
| ATOM | 1606 | O4′ | CYT | B | 223 | 9.508 | 28.903 | 17.458 | 1.00 | 75.35 | B |
| ATOM | 1607 | C1′ | CYT | B | 223 | 8.192 | 28.376 | 17.444 | 1.00 | 73.35 | B |
| ATOM | 1608 | N1 | CYT | B | 223 | 8.273 | 26.965 | 17.054 | 1.00 | 71.88 | B |
| ATOM | 1609 | C6 | CYT | B | 223 | 9.418 | 26.242 | 17.238 | 1.00 | 70.84 | B |
| ATOM | 1610 | C2 | CYT | B | 223 | 7.148 | 26.371 | 16.507 | 1.00 | 71.26 | B |
| ATOM | 1611 | O2 | CYT | B | 223 | 6.145 | 27.071 | 16.321 | 1.00 | 71.83 | B |
| ATOM | 1612 | N3 | CYT | B | 223 | 7.178 | 25.056 | 16.189 | 1.00 | 70.51 | B |
| ATOM | 1613 | C4 | CYT | B | 223 | 8.292 | 24.350 | 16.390 | 1.00 | 70.31 | B |
| ATOM | 1614 | N4 | CYT | B | 223 | 8.276 | 23.054 | 16.075 | 1.00 | 69.46 | B |
| ATOM | 1615 | C5 | CYT | B | 223 | 9.471 | 24.943 | 16.924 | 1.00 | 70.47 | B |
| ATOM | 1616 | C2′ | CYT | B | 223 | 7.638 | 28.551 | 18.859 | 1.00 | 72.90 | B |
| ATOM | 1617 | O2′ | CYT | B | 223 | 7.013 | 29.811 | 18.962 | 1.00 | 71.35 | B |
| ATOM | 1618 | C3′ | CYT | B | 223 | 8.919 | 28.459 | 19.674 | 1.00 | 73.63 | B |
| ATOM | 1619 | O3′ | CYT | B | 223 | 8.828 | 29.085 | 20.942 | 1.00 | 73.42 | B |
| ATOM | 1620 | P | ADE | B | 224 | 7.950 | 28.421 | 22.107 | 1.00 | 72.93 | B |
| ATOM | 1621 | O1P | ADE | B | 224 | 8.161 | 29.304 | 23.283 | 1.00 | 72.84 | B |
| ATOM | 1622 | O2P | ADE | B | 224 | 8.213 | 26.961 | 22.222 | 1.00 | 71.45 | B |
| ATOM | 1623 | O5′ | ADE | B | 224 | 6.463 | 28.609 | 21.589 | 1.00 | 71.28 | B |
| ATOM | 1624 | C5′ | ADE | B | 224 | 5.421 | 27.807 | 22.102 | 1.00 | 70.45 | B |
| ATOM | 1625 | C4′ | ADE | B | 224 | 4.399 | 27.548 | 21.033 | 1.00 | 69.38 | B |
| ATOM | 1626 | O4′ | ADE | B | 224 | 5.056 | 27.306 | 19.759 | 1.00 | 69.07 | B |
| ATOM | 1627 | C1′ | ADE | B | 224 | 4.415 | 26.231 | 19.092 | 1.00 | 68.77 | B |
| ATOM | 1628 | N9 | ADE | B | 224 | 5.324 | 25.083 | 19.084 | 1.00 | 68.39 | B |
| ATOM | 1629 | C4 | ADE | B | 224 | 4.975 | 23.793 | 18.763 | 1.00 | 67.71 | B |
| ATOM | 1630 | N3 | ADE | B | 224 | 3.757 | 23.343 | 18.403 | 1.00 | 66.60 | B |
| ATOM | 1631 | C2 | ADE | B | 224 | 3.797 | 22.038 | 18.169 | 1.00 | 66.24 | B |
| ATOM | 1632 | N1 | ADE | B | 224 | 4.837 | 21.193 | 18.249 | 1.00 | 65.92 | B |
| ATOM | 1633 | C6 | ADE | B | 224 | 6.042 | 21.680 | 18.615 | 1.00 | 65.51 | B |
| ATOM | 1634 | N6 | ADE | B | 224 | 7.072 | 20.842 | 18.698 | 1.00 | 66.29 | B |
| ATOM | 1635 | C5 | ADE | B | 224 | 6.135 | 23.047 | 18.887 | 1.00 | 66.23 | B |
| ATOM | 1636 | N7 | ADE | B | 224 | 7.198 | 23.848 | 19.278 | 1.00 | 65.86 | B |
| ATOM | 1637 | C8 | ADE | B | 224 | 6.667 | 25.043 | 19.383 | 1.00 | 67.75 | B |
| ATOM | 1638 | C2′ | ADE | B | 224 | 3.134 | 25.942 | 19.871 | 1.00 | 69.52 | B |
| ATOM | 1639 | O2′ | ADE | B | 224 | 2.086 | 26.764 | 19.407 | 1.00 | 71.88 | B |
| ATOM | 1640 | C3′ | ADE | B | 224 | 3.571 | 26.305 | 21.278 | 1.00 | 69.39 | B |
| ATOM | 1641 | O3′ | ADE | B | 224 | 2.483 | 26.645 | 22.114 | 1.00 | 68.87 | B |
| ATOM | 1642 | P | GUA | B | 225 | 2.437 | 26.080 | 23.611 | 1.00 | 67.84 | B |
| ATOM | 1643 | O1P | GUA | B | 225 | 3.847 | 25.967 | 24.070 | 1.00 | 65.04 | B |
| ATOM | 1644 | O2P | GUA | B | 225 | 1.460 | 26.894 | 24.385 | 1.00 | 66.71 | B |
| ATOM | 1645 | O5′ | GUA | B | 225 | 1.822 | 24.626 | 23.424 | 1.00 | 66.33 | B |
| ATOM | 1646 | C5′ | GUA | B | 225 | 0.601 | 24.455 | 22.717 | 1.00 | 65.13 | B |
| ATOM | 1647 | C4′ | GUA | B | 225 | 0.505 | 23.057 | 22.173 | 1.00 | 63.34 | B |
| ATOM | 1648 | O4′ | GUA | B | 225 | 1.584 | 22.835 | 21.228 | 1.00 | 63.06 | B |
| ATOM | 1649 | C1′ | GUA | B | 225 | 1.949 | 21.463 | 21.246 | 1.00 | 62.23 | B |
| ATOM | 1650 | N9 | GUA | B | 225 | 3.374 | 21.380 | 21.544 | 1.00 | 61.82 | B |
| ATOM | 1651 | C4 | GUA | B | 225 | 4.182 | 20.265 | 21.447 | 1.00 | 61.57 | B |
| ATOM | 1652 | N3 | GUA | B | 225 | 3.794 | 19.022 | 21.072 | 1.00 | 60.26 | B |
| ATOM | 1653 | C2 | GUA | B | 225 | 4.812 | 18.170 | 21.062 | 1.00 | 60.24 | B |
| ATOM | 1654 | N2 | GUA | B | 225 | 4.624 | 16.893 | 20.707 | 1.00 | 59.34 | B |
| ATOM | 1655 | N1 | GUA | B | 225 | 6.097 | 18.509 | 21.402 | 1.00 | 61.02 | B |
| ATOM | 1656 | C6 | GUA | B | 225 | 6.512 | 19.773 | 21.806 | 1.00 | 61.71 | B |
| ATOM | 1657 | O6 | GUA | B | 225 | 7.694 | 19.962 | 22.120 | 1.00 | 65.20 | B |
| ATOM | 1658 | C5 | GUA | B | 225 | 5.441 | 20.699 | 21.806 | 1.00 | 61.15 | B |
| ATOM | 1659 | N7 | GUA | B | 225 | 5.423 | 22.045 | 22.135 | 1.00 | 61.04 | B |
| ATOM | 1660 | C8 | GUA | B | 225 | 4.181 | 22.406 | 21.967 | 1.00 | 61.37 | B |
| ATOM | 1661 | C2′ | GUA | B | 225 | 1.058 | 20.787 | 22.291 | 1.00 | 62.66 | B |
| ATOM | 1662 | O2′ | GUA | B | 225 | −0.092 | 20.267 | 21.662 | 1.00 | 63.62 | B |
| ATOM | 1663 | C3′ | GUA | B | 225 | 0.733 | 21.964 | 23.192 | 1.00 | 62.60 | B |
| ATOM | 1664 | O3′ | GUA | B | 225 | −0.413 | 21.752 | 23.998 | 1.00 | 61.67 | B |
| ATOM | 1665 | P | URI | B | 226 | −0.221 | 21.412 | 25.560 | 1.00 | 64.35 | B |
| ATOM | 1666 | O1P | URI | B | 226 | 0.847 | 22.297 | 26.105 | 1.00 | 62.52 | B |
| ATOM | 1667 | O2P | URI | B | 226 | −1.550 | 21.373 | 26.227 | 1.00 | 62.13 | B |
| ATOM | 1668 | O5′ | URI | B | 226 | 0.377 | 19.940 | 25.549 | 1.00 | 61.29 | B |
| ATOM | 1669 | C5′ | URI | B | 226 | −0.341 | 18.874 | 24.963 | 1.00 | 59.62 | B |
| ATOM | 1670 | C4′ | URI | B | 226 | 0.582 | 17.718 | 24.726 | 1.00 | 59.37 | B |
| ATOM | 1671 | O4′ | URI | B | 226 | 1.675 | 18.167 | 23.892 | 1.00 | 60.60 | B |
| ATOM | 1672 | C1′ | URI | B | 226 | 2.834 | 17.419 | 24.200 | 1.00 | 59.54 | B |
| ATOM | 1673 | N1 | URI | B | 226 | 3.915 | 18.354 | 24.521 | 1.00 | 59.11 | B |
| ATOM | 1674 | C6 | URI | B | 226 | 3.694 | 19.697 | 24.700 | 1.00 | 59.62 | B |
| ATOM | 1675 | C2 | URI | B | 226 | 5.168 | 17.823 | 24.616 | 1.00 | 59.24 | B |
| ATOM | 1676 | O2 | URI | B | 226 | 5.371 | 16.632 | 24.511 | 1.00 | 59.59 | B |
| ATOM | 1677 | N3 | URI | B | 226 | 6.175 | 18.728 | 24.844 | 1.00 | 60.64 | B |
| ATOM | 1678 | C4 | URI | B | 226 | 6.044 | 20.091 | 25.000 | 1.00 | 60.95 | B |
| ATOM | 1679 | O4 | URI | B | 226 | 7.060 | 20.794 | 25.038 | 1.00 | 61.49 | B |
| ATOM | 1680 | C5 | URI | B | 226 | 4.689 | 20.565 | 24.935 | 1.00 | 61.22 | B |
| ATOM | 1681 | C2′ | URI | B | 226 | 2.488 | 16.492 | 25.363 | 1.00 | 59.58 | B |
| ATOM | 1682 | O2′ | URI | B | 226 | 2.192 | 15.200 | 24.876 | 1.00 | 58.65 | B |
| ATOM | 1683 | C3′ | URI | B | 226 | 1.290 | 17.209 | 25.964 | 1.00 | 57.85 | B |
| ATOM | 1684 | O3′ | URI | B | 226 | 0.465 | 16.313 | 26.675 | 1.00 | 56.69 | B |
| ATOM | 1685 | P | CYT | B | 227 | 0.658 | 16.159 | 28.251 | 1.00 | 57.65 | B |
| ATOM | 1686 | O1P | CYT | B | 227 | 0.953 | 17.503 | 28.816 | 1.00 | 56.13 | B |
| ATOM | 1687 | O2P | CYT | B | 227 | −0.457 | 15.353 | 28.811 | 1.00 | 56.22 | B |
| ATOM | 1688 | O5′ | CYT | B | 227 | 1.971 | 15.279 | 28.349 | 1.00 | 57.28 | B |
| ATOM | 1689 | C5′ | CYT | B | 227 | 1.877 | 13.870 | 28.337 | 1.00 | 55.41 | B |
| ATOM | 1690 | C4′ | CYT | B | 227 | 3.244 | 13.264 | 28.279 | 1.00 | 53.68 | B |
| ATOM | 1691 | O4′ | CYT | B | 227 | 4.029 | 13.948 | 27.268 | 1.00 | 52.41 | B |
| ATOM | 1692 | C1′ | CYT | B | 227 | 5.398 | 13.840 | 27.599 | 1.00 | 50.88 | B |
| ATOM | 1693 | N1 | CYT | B | 227 | 5.955 | 15.185 | 27.709 | 1.00 | 50.59 | B |
| ATOM | 1694 | C6 | CYT | B | 227 | 5.158 | 16.293 | 27.758 | 1.00 | 51.57 | B |
| ATOM | 1695 | C2 | CYT | B | 227 | 7.318 | 15.307 | 27.771 | 1.00 | 51.15 | B |
| ATOM | 1696 | O2 | CYT | B | 227 | 7.992 | 14.281 | 27.735 | 1.00 | 54.89 | B |
| ATOM | 1697 | N3 | CYT | B | 227 | 7.881 | 16.526 | 27.873 | 1.00 | 50.46 | B |
| ATOM | 1698 | C4 | CYT | B | 227 | 7.113 | 17.605 | 27.915 | 1.00 | 49.35 | B |
| ATOM | 1699 | N4 | CYT | B | 227 | 7.714 | 18.782 | 28.001 | 1.00 | 48.12 | B |
| ATOM | 1700 | C5 | CYT | B | 227 | 5.692 | 17.518 | 27.865 | 1.00 | 51.06 | B |
| ATOM | 1701 | C2′ | CYT | B | 227 | 5.469 | 13.096 | 28.934 | 1.00 | 52.88 | B |
| ATOM | 1702 | O2′ | CYT | B | 227 | 5.667 | 11.713 | 28.694 | 1.00 | 50.84 | B |
| ATOM | 1703 | C3′ | CYT | B | 227 | 4.101 | 13.428 | 29.516 | 1.00 | 52.37 | B |
| ATOM | 1704 | O3′ | CYT | B | 227 | 3.707 | 12.532 | 30.547 | 1.00 | 53.39 | B |
| ATOM | 1705 | P | GUA | B | 228 | 2.977 | 13.107 | 31.856 | 1.00 | 58.66 | B |
| ATOM | 1706 | O1P | GUA | B | 228 | 2.593 | 14.499 | 31.527 | 1.00 | 58.56 | B |
| ATOM | 1707 | O2P | GUA | B | 228 | 1.946 | 12.152 | 32.350 | 1.00 | 57.37 | B |
| ATOM | 1708 | O5′ | GUA | B | 228 | 4.121 | 13.179 | 32.958 | 1.00 | 57.02 | B |
| ATOM | 1709 | C5′ | GUA | B | 228 | 4.727 | 11.996 | 33.453 | 1.00 | 55.04 | B |
| ATOM | 1710 | C4′ | GUA | B | 228 | 6.155 | 11.907 | 32.973 | 1.00 | 53.26 | B |
| ATOM | 1711 | O4′ | GUA | B | 228 | 6.327 | 12.734 | 31.799 | 1.00 | 53.28 | B |
| ATOM | 1712 | C1′ | GUA | B | 228 | 7.670 | 13.187 | 31.733 | 1.00 | 53.19 | B |
| ATOM | 1713 | N9 | GUA | B | 228 | 7.665 | 14.646 | 31.617 | 1.00 | 53.81 | B |
| ATOM | 1714 | C4 | GUA | B | 228 | 8.769 | 15.465 | 31.480 | 1.00 | 53.76 | B |
| ATOM | 1715 | N3 | GUA | B | 228 | 10.056 | 15.062 | 31.408 | 1.00 | 53.36 | B |
| ATOM | 1716 | C2 | GUA | B | 228 | 10.888 | 16.083 | 31.303 | 1.00 | 51.84 | B |
| ATOM | 1717 | N2 | GUA | B | 228 | 12.197 | 15.867 | 31.210 | 1.00 | 48.75 | B |
| ATOM | 1718 | N1 | GUA | B | 228 | 10.494 | 17.391 | 31.283 | 1.00 | 52.85 | B |
| ATOM | 1719 | C6 | GUA | B | 228 | 9.181 | 17.832 | 31.371 | 1.00 | 53.04 | B |
| ATOM | 1720 | O6 | GUA | B | 228 | 8.941 | 19.044 | 31.379 | 1.00 | 54.64 | B |
| ATOM | 1721 | C5 | GUA | B | 228 | 8.270 | 16.748 | 31.461 | 1.00 | 53.49 | B |
| ATOM | 1722 | N7 | GUA | B | 228 | 6.884 | 16.741 | 31.554 | 1.00 | 54.44 | B |
| ATOM | 1723 | C8 | GUA | B | 228 | 6.571 | 15.475 | 31.643 | 1.00 | 53.87 | B |
| ATOM | 1724 | C2′ | GUA | B | 228 | 8.376 | 12.685 | 32.995 | 1.00 | 51.78 | B |
| ATOM | 1725 | O2′ | GUA | B | 228 | 9.059 | 11.488 | 32.715 | 1.00 | 50.97 | B |
| ATOM | 1726 | C3′ | GUA | B | 228 | 7.203 | 12.443 | 33.928 | 1.00 | 51.60 | B |
| ATOM | 1727 | O3′ | GUA | B | 228 | 7.537 | 11.436 | 34.862 | 1.00 | 49.14 | B |
| ATOM | 1728 | P | CYT | B | 229 | 7.620 | 11.796 | 36.417 | 1.00 | 50.14 | B |
| ATOM | 1729 | O1P | CYT | B | 229 | 6.710 | 12.935 | 36.705 | 1.00 | 47.87 | B |
| ATOM | 1730 | O2P | CYT | B | 229 | 7.440 | 10.515 | 37.145 | 1.00 | 46.49 | B |
| ATOM | 1731 | O5′ | CYT | B | 229 | 9.129 | 12.286 | 36.571 | 1.00 | 49.59 | B |
| ATOM | 1732 | C5′ | CYT | B | 229 | 10.191 | 11.415 | 36.193 | 1.00 | 49.36 | B |
| ATOM | 1733 | C4′ | CYT | B | 229 | 11.493 | 12.164 | 36.075 | 1.00 | 48.50 | B |
| ATOM | 1734 | O4′ | CYT | B | 229 | 11.513 | 12.942 | 34.849 | 1.00 | 49.47 | B |
| ATOM | 1735 | C1′ | CYT | B | 229 | 12.233 | 14.144 | 35.061 | 1.00 | 48.55 | B |
| ATOM | 1736 | N1 | CYT | B | 229 | 11.286 | 15.263 | 34.997 | 1.00 | 49.12 | B |
| ATOM | 1737 | C6 | CYT | B | 229 | 9.957 | 15.064 | 35.225 | 1.00 | 49.46 | B |
| ATOM | 1738 | C2 | CYT | B | 229 | 11.767 | 16.539 | 34.704 | 1.00 | 48.95 | B |
| ATOM | 1739 | O2 | CYT | B | 229 | 12.978 | 16.692 | 34.508 | 1.00 | 49.05 | B |
| ATOM | 1740 | N3 | CYT | B | 229 | 10.904 | 17.571 | 34.639 | 1.00 | 48.91 | B |
| ATOM | 1741 | C4 | CYT | B | 229 | 9.607 | 17.366 | 34.849 | 1.00 | 49.64 | B |
| ATOM | 1742 | N4 | CYT | B | 229 | 8.782 | 18.413 | 34.749 | 1.00 | 50.11 | B |
| ATOM | 1743 | C5 | CYT | B | 229 | 9.091 | 16.078 | 35.164 | 1.00 | 50.66 | B |
| ATOM | 1744 | C2′ | CYT | B | 229 | 12.851 | 14.011 | 36.449 | 1.00 | 48.95 | B |
| ATOM | 1745 | O2′ | CYT | B | 229 | 14.059 | 13.294 | 36.360 | 1.00 | 50.48 | B |
| ATOM | 1746 | C3′ | CYT | B | 229 | 11.785 | 13.190 | 37.145 | 1.00 | 48.46 | B |
| ATOM | 1747 | O3′ | CYT | B | 229 | 12.322 | 12.584 | 38.306 | 1.00 | 48.74 | B |
| ATOM | 1748 | P | GUA | B | 230 | 12.075 | 13.265 | 39.739 | 1.00 | 49.49 | B |
| ATOM | 1749 | O1P | GUA | B | 230 | 10.667 | 13.756 | 39.761 | 1.00 | 50.64 | B |
| ATOM | 1750 | O2P | GUA | B | 230 | 12.531 | 12.304 | 40.772 | 1.00 | 50.34 | B |
| ATOM | 1751 | O5′ | GUA | B | 230 | 13.046 | 14.531 | 39.748 | 1.00 | 49.69 | B |
| ATOM | 1752 | C5′ | GUA | B | 230 | 14.427 | 14.394 | 39.426 | 1.00 | 49.57 | B |
| ATOM | 1753 | C4′ | GUA | B | 230 | 15.145 | 15.706 | 39.621 | 1.00 | 51.51 | B |
| ATOM | 1754 | O4′ | GUA | B | 230 | 14.571 | 16.710 | 38.741 | 1.00 | 50.73 | B |
| ATOM | 1755 | C1′ | GUA | B | 230 | 14.206 | 17.841 | 39.496 | 1.00 | 51.22 | B |
| ATOM | 1756 | N9 | GUA | B | 230 | 12.995 | 18.399 | 38.911 | 1.00 | 52.23 | B |
| ATOM | 1757 | C4 | GUA | B | 230 | 12.803 | 19.701 | 38.511 | 1.00 | 52.74 | B |
| ATOM | 1758 | N3 | GUA | B | 230 | 13.715 | 20.692 | 38.570 | 1.00 | 52.74 | B |
| ATOM | 1759 | C2 | GUA | B | 230 | 13.224 | 21.838 | 38.141 | 1.00 | 52.25 | B |
| ATOM | 1760 | N2 | GUA | B | 230 | 13.984 | 22.930 | 38.134 | 1.00 | 52.25 | B |
| ATOM | 1761 | N1 | GUA | B | 230 | 11.947 | 22.000 | 37.689 | 1.00 | 54.49 | B |
| ATOM | 1762 | C6 | GUA | B | 230 | 10.988 | 21.001 | 37.622 | 1.00 | 53.15 | B |
| ATOM | 1763 | O6 | GUA | B | 230 | 9.858 | 21.269 | 37.217 | 1.00 | 54.25 | B |
| ATOM | 1764 | C5 | GUA | B | 230 | 11.498 | 19.759 | 38.071 | 1.00 | 53.53 | B |
| ATOM | 1765 | N7 | GUA | B | 230 | 10.889 | 18.515 | 38.169 | 1.00 | 54.01 | B |
| ATOM | 1766 | C8 | GUA | B | 230 | 11.816 | 17.740 | 38.669 | 1.00 | 53.29 | B |
| ATOM | 1767 | C2′ | GUA | B | 230 | 13.988 | 17.331 | 40.921 | 1.00 | 52.69 | B |
| ATOM | 1768 | O2′ | GUA | B | 230 | 14.157 | 18.372 | 41.864 | 1.00 | 50.69 | B |
| ATOM | 1769 | C3′ | GUA | B | 230 | 15.087 | 16.285 | 41.031 | 1.00 | 52.14 | B |
| ATOM | 1770 | O3′ | GUA | B | 230 | 16.296 | 16.965 | 41.337 | 1.00 | 54.91 | B |
| ATOM | 1771 | P | URI | B | 231 | 17.564 | 16.146 | 41.868 | 1.00 | 55.02 | B |
| ATOM | 1772 | O1P | URI | B | 231 | 18.266 | 17.052 | 42.816 | 1.00 | 52.02 | B |
| ATOM | 1773 | O2P | URI | B | 231 | 17.113 | 14.812 | 42.330 | 1.00 | 53.85 | B |
| ATOM | 1774 | O5′ | URI | B | 231 | 18.437 | 15.930 | 40.553 | 1.00 | 55.40 | B |
| ATOM | 1775 | C5′ | URI | B | 231 | 18.892 | 17.033 | 39.779 | 1.00 | 58.23 | B |
| ATOM | 1776 | C4′ | URI | B | 231 | 20.114 | 16.640 | 38.978 | 1.00 | 62.66 | B |
| ATOM | 1777 | O4′ | URI | B | 231 | 21.297 | 16.816 | 39.804 | 1.00 | 62.63 | B |
| ATOM | 1778 | C1′ | URI | B | 231 | 21.910 | 15.562 | 40.032 | 1.00 | 62.26 | B |
| ATOM | 1779 | N1 | URI | B | 231 | 22.357 | 15.523 | 41.428 | 1.00 | 61.38 | B |
| ATOM | 1780 | C6 | URI | B | 231 | 21.550 | 15.957 | 42.452 | 1.00 | 60.83 | B |
| ATOM | 1781 | C2 | URI | B | 231 | 23.620 | 15.042 | 41.669 | 1.00 | 61.54 | B |
| ATOM | 1782 | O2 | URI | B | 231 | 24.344 | 14.639 | 40.770 | 1.00 | 62.29 | B |
| ATOM | 1783 | N3 | URI | B | 231 | 24.004 | 15.046 | 42.994 | 1.00 | 61.42 | B |
| ATOM | 1784 | C4 | URI | B | 231 | 23.245 | 15.466 | 44.082 | 1.00 | 62.36 | B |
| ATOM | 1785 | O4 | URI | B | 231 | 23.705 | 15.380 | 45.230 | 1.00 | 61.50 | B |
| ATOM | 1786 | C5 | URI | B | 231 | 21.938 | 15.944 | 43.737 | 1.00 | 62.52 | B |
| ATOM | 1787 | C2′ | URI | B | 231 | 20.865 | 14.510 | 39.681 | 1.00 | 63.79 | B |
| ATOM | 1788 | O2′ | URI | B | 231 | 21.499 | 13.302 | 39.319 | 1.00 | 63.87 | B |
| ATOM | 1789 | C3′ | URI | B | 231 | 20.174 | 15.189 | 38.510 | 1.00 | 65.22 | B |
| ATOM | 1790 | O3′ | URI | B | 231 | 21.013 | 15.093 | 37.361 | 1.00 | 70.38 | B |
| ATOM | 1791 | P | GUA | B | 232 | 20.580 | 14.153 | 36.129 | 1.00 | 74.73 | B |
| ATOM | 1792 | O1P | GUA | B | 232 | 21.816 | 13.664 | 35.458 | 1.00 | 74.60 | B |
| ATOM | 1793 | O2P | GUA | B | 232 | 19.583 | 13.168 | 36.633 | 1.00 | 74.73 | B |
| ATOM | 1794 | O5′ | GUA | B | 232 | 19.843 | 15.167 | 35.143 | 1.00 | 74.06 | B |
| ATOM | 1795 | C5′ | GUA | B | 232 | 20.363 | 15.429 | 33.850 | 1.00 | 72.97 | B |
| ATOM | 1796 | C4′ | GUA | B | 232 | 20.387 | 16.911 | 33.587 | 1.00 | 72.59 | B |
| ATOM | 1797 | O4′ | GUA | B | 232 | 20.097 | 17.653 | 34.800 | 1.00 | 71.37 | B |
| ATOM | 1798 | C1′ | GUA | B | 232 | 20.742 | 18.918 | 34.738 | 1.00 | 72.27 | B |
| ATOM | 1799 | N9 | GUA | B | 232 | 21.585 | 19.057 | 35.920 | 1.00 | 74.07 | B |
| ATOM | 1800 | C4 | GUA | B | 232 | 21.247 | 19.648 | 37.115 | 1.00 | 74.59 | B |
| ATOM | 1801 | N3 | GUA | B | 232 | 20.058 | 20.216 | 37.415 | 1.00 | 74.20 | B |
| ATOM | 1802 | C2 | GUA | B | 232 | 20.036 | 20.686 | 38.651 | 1.00 | 73.75 | B |
| ATOM | 1803 | N2 | GUA | B | 232 | 18.936 | 21.287 | 39.118 | 1.00 | 74.62 | B |
| ATOM | 1804 | N1 | GUA | B | 232 | 21.093 | 20.604 | 39.522 | 1.00 | 74.18 | B |
| ATOM | 1805 | C6 | GUA | B | 232 | 22.326 | 20.027 | 39.236 | 1.00 | 73.75 | B |
| ATOM | 1806 | O6 | GUA | B | 232 | 23.213 | 20.004 | 40.097 | 1.00 | 72.28 | B |
| ATOM | 1807 | C5 | GUA | B | 232 | 22.366 | 19.522 | 37.914 | 1.00 | 74.36 | B |
| ATOM | 1808 | N7 | GUA | B | 232 | 23.388 | 18.877 | 37.231 | 1.00 | 74.62 | B |
| ATOM | 1809 | C8 | GUA | B | 232 | 22.880 | 18.621 | 36.057 | 1.00 | 74.00 | B |
| ATOM | 1810 | C2′ | GUA | B | 232 | 21.583 | 18.938 | 33.459 | 1.00 | 71.92 | B |
| ATOM | 1811 | O2′ | GUA | B | 232 | 20.918 | 19.654 | 32.445 | 1.00 | 69.55 | B |
| ATOM | 1812 | C3′ | GUA | B | 232 | 21.741 | 17.448 | 33.177 | 1.00 | 72.97 | B |
| ATOM | 1813 | O3′ | GUA | B | 232 | 21.999 | 17.156 | 31.808 | 1.00 | 75.27 | B |
| ATOM | 1814 | P | ADE | B | 233 | 23.030 | 15.978 | 31.426 | 1.00 | 76.87 | B |
| ATOM | 1815 | O1P | ADE | B | 233 | 23.450 | 16.242 | 30.030 | 1.00 | 74.87 | B |
| ATOM | 1816 | O2P | ADE | B | 233 | 24.054 | 15.871 | 32.501 | 1.00 | 75.85 | B |
| ATOM | 1817 | O5′ | ADE | B | 233 | 22.153 | 14.648 | 31.448 | 1.00 | 76.78 | B |
| ATOM | 1818 | C5′ | ADE | B | 233 | 22.695 | 13.427 | 31.937 | 1.00 | 79.21 | B |
| ATOM | 1819 | C4′ | ADE | B | 233 | 21.916 | 12.252 | 31.394 | 1.00 | 81.95 | B |
| ATOM | 1820 | O4′ | ADE | B | 233 | 20.571 | 12.264 | 31.942 | 1.00 | 82.72 | B |
| ATOM | 1821 | C1′ | ADE | B | 233 | 19.624 | 12.348 | 30.893 | 1.00 | 83.95 | B |
| ATOM | 1822 | N9 | ADE | B | 233 | 18.556 | 13.252 | 31.322 | 1.00 | 84.20 | B |
| ATOM | 1823 | C4 | ADE | B | 233 | 17.675 | 13.035 | 32.354 | 1.00 | 84.30 | B |
| ATOM | 1824 | N3 | ADE | B | 233 | 17.600 | 11.956 | 33.152 | 1.00 | 84.48 | B |
| ATOM | 1825 | C2 | ADE | B | 233 | 16.645 | 12.111 | 34.060 | 1.00 | 82.93 | B |
| ATOM | 1826 | N1 | ADE | B | 233 | 15.824 | 13.142 | 34.252 | 1.00 | 82.81 | B |
| ATOM | 1827 | C6 | ADE | B | 233 | 15.928 | 14.213 | 33.439 | 1.00 | 83.48 | B |
| ATOM | 1828 | N6 | ADE | B | 233 | 15.124 | 15.256 | 33.649 | 1.00 | 83.96 | B |
| ATOM | 1829 | C5 | ADE | B | 233 | 16.891 | 14.169 | 32.424 | 1.00 | 83.94 | B |
| ATOM | 1830 | N7 | ADE | B | 233 | 17.246 | 15.074 | 31.434 | 1.00 | 84.28 | B |
| ATOM | 1831 | C8 | ADE | B | 233 | 18.234 | 14.484 | 30.809 | 1.00 | 84.16 | B |
| ATOM | 1832 | C2′ | ADE | B | 233 | 20.386 | 12.855 | 29.669 | 1.00 | 84.22 | B |
| ATOM | 1833 | O2′ | ADE | B | 233 | 19.747 | 12.438 | 28.476 | 1.00 | 85.53 | B |
| ATOM | 1834 | C3′ | ADE | B | 233 | 21.752 | 12.215 | 29.881 | 1.00 | 83.72 | B |
| ATOM | 1835 | O3′ | ADE | B | 233 | 21.764 | 10.871 | 29.415 | 1.00 | 84.83 | B |
| ATOM | 1836 | P | URI | B | 234 | 22.914 | 10.397 | 28.401 | 1.00 | 86.34 | B |
| ATOM | 1837 | O1P | URI | B | 234 | 23.017 | 11.423 | 27.324 | 1.00 | 84.26 | B |
| ATOM | 1838 | O2P | URI | B | 234 | 24.114 | 10.047 | 29.213 | 1.00 | 85.14 | B |
| ATOM | 1839 | O5′ | URI | B | 234 | 22.334 | 9.072 | 27.744 | 1.00 | 85.10 | B |
| ATOM | 1840 | C5′ | URI | B | 234 | 21.216 | 9.128 | 26.873 | 1.00 | 86.55 | B |
| ATOM | 1841 | C4′ | URI | B | 234 | 20.265 | 8.015 | 27.203 | 1.00 | 87.02 | B |
| ATOM | 1842 | O4′ | URI | B | 234 | 20.923 | 6.767 | 26.879 | 1.00 | 88.52 | B |
| ATOM | 1843 | C1′ | URI | B | 234 | 21.153 | 6.018 | 28.050 | 1.00 | 89.34 | B |
| ATOM | 1844 | N1 | URI | B | 234 | 22.532 | 5.514 | 28.019 | 1.00 | 90.55 | B |
| ATOM | 1845 | C6 | URI | B | 234 | 23.609 | 6.374 | 28.072 | 1.00 | 91.32 | B |
| ATOM | 1846 | C2 | URI | B | 234 | 22.717 | 4.140 | 27.963 | 1.00 | 91.37 | B |
| ATOM | 1847 | O2 | URI | B | 234 | 21.793 | 3.343 | 27.862 | 1.00 | 91.49 | B |
| ATOM | 1848 | N3 | URI | B | 234 | 24.027 | 3.732 | 28.025 | 1.00 | 91.61 | B |
| ATOM | 1849 | C4 | URI | B | 234 | 25.146 | 4.536 | 28.125 | 1.00 | 91.41 | B |
| ATOM | 1850 | O4 | URI | B | 234 | 26.232 | 4.014 | 28.352 | 1.00 | 89.82 | B |
| ATOM | 1851 | C5 | URI | B | 234 | 24.876 | 5.944 | 28.124 | 1.00 | 91.21 | B |
| ATOM | 1852 | C2′ | URI | B | 234 | 20.862 | 6.909 | 29.257 | 1.00 | 89.26 | B |
| ATOM | 1853 | O2′ | URI | B | 234 | 20.314 | 6.159 | 30.326 | 1.00 | 90.70 | B |
| ATOM | 1854 | C3′ | URI | B | 234 | 19.908 | 7.950 | 28.677 | 1.00 | 87.78 | B |
| ATOM | 1855 | O3′ | URI | B | 234 | 18.518 | 7.722 | 28.903 | 1.00 | 86.95 | B |
| ATOM | 1856 | P | ADE | B | 235 | 17.817 | 6.342 | 28.445 | 1.00 | 85.44 | B |
| ATOM | 1857 | O1P | ADE | B | 235 | 16.896 | 6.684 | 27.332 | 1.00 | 85.69 | B |
| ATOM | 1858 | O2P | ADE | B | 235 | 18.801 | 5.257 | 28.260 | 1.00 | 85.00 | B |
| ATOM | 1859 | O5′ | ADE | B | 235 | 16.940 | 5.955 | 29.723 | 1.00 | 83.93 | B |
| ATOM | 1860 | C5′ | ADE | B | 235 | 17.569 | 5.536 | 30.939 | 1.00 | 78.89 | B |
| ATOM | 1861 | C4′ | ADE | B | 235 | 17.097 | 6.374 | 32.112 | 1.00 | 75.23 | B |
| ATOM | 1862 | O4′ | ADE | B | 235 | 17.610 | 7.722 | 32.010 | 1.00 | 73.86 | B |
| ATOM | 1863 | C1′ | ADE | B | 235 | 16.680 | 8.625 | 32.585 | 1.00 | 72.80 | B |
| ATOM | 1864 | N9 | ADE | B | 235 | 16.308 | 9.611 | 31.570 | 1.00 | 72.57 | B |
| ATOM | 1865 | C4 | ADE | B | 235 | 15.277 | 10.511 | 31.650 | 1.00 | 70.87 | B |
| ATOM | 1866 | N3 | ADE | B | 235 | 14.391 | 10.645 | 32.650 | 1.00 | 70.49 | B |
| ATOM | 1867 | C2 | ADE | B | 235 | 13.561 | 11.645 | 32.402 | 1.00 | 69.46 | B |
| ATOM | 1868 | N1 | ADE | B | 235 | 13.518 | 12.466 | 31.347 | 1.00 | 69.42 | B |
| ATOM | 1869 | C6 | ADE | B | 235 | 14.423 | 12.301 | 30.360 | 1.00 | 70.02 | B |
| ATOM | 1870 | N6 | ADE | B | 235 | 14.388 | 13.125 | 29.309 | 1.00 | 69.14 | B |
| ATOM | 1871 | C5 | ADE | B | 235 | 15.351 | 11.271 | 30.500 | 1.00 | 70.73 | B |
| ATOM | 1872 | N7 | ADE | B | 235 | 16.388 | 10.837 | 29.687 | 1.00 | 72.05 | B |
| ATOM | 1873 | C8 | ADE | B | 235 | 16.920 | 9.851 | 30.362 | 1.00 | 73.00 | B |
| ATOM | 1874 | C2′ | ADE | B | 235 | 15.490 | 7.812 | 33.092 | 1.00 | 72.76 | B |
| ATOM | 1875 | O2′ | ADE | B | 235 | 15.621 | 7.556 | 34.472 | 1.00 | 72.46 | B |
| ATOM | 1876 | C3′ | ADE | B | 235 | 15.598 | 6.553 | 32.249 | 1.00 | 73.17 | B |
| ATOM | 1877 | O3′ | ADE | B | 235 | 15.059 | 5.455 | 32.949 | 1.00 | 72.86 | B |
| ATOM | 1878 | P | ADE | B | 236 | 13.960 | 4.541 | 32.236 | 1.00 | 74.90 | B |
| ATOM | 1879 | O1P | ADE | B | 236 | 13.971 | 3.192 | 32.860 | 1.00 | 74.45 | B |
| ATOM | 1880 | O2P | ADE | B | 236 | 14.181 | 4.677 | 30.771 | 1.00 | 75.24 | B |
| ATOM | 1881 | O5′ | ADE | B | 236 | 12.590 | 5.251 | 32.625 | 1.00 | 72.45 | B |
| ATOM | 1882 | C5′ | ADE | B | 236 | 12.365 | 5.699 | 33.953 | 1.00 | 68.86 | B |
| ATOM | 1883 | C4′ | ADE | B | 236 | 11.428 | 6.869 | 33.942 | 1.00 | 66.95 | B |
| ATOM | 1884 | O4′ | ADE | B | 236 | 12.138 | 8.049 | 33.482 | 1.00 | 66.04 | B |
| ATOM | 1885 | C1′ | ADE | B | 236 | 11.263 | 8.863 | 32.724 | 1.00 | 64.52 | B |
| ATOM | 1886 | N9 | ADE | B | 236 | 11.860 | 9.062 | 31.406 | 1.00 | 63.45 | B |
| ATOM | 1887 | C4 | ADE | B | 236 | 11.669 | 10.123 | 30.554 | 1.00 | 62.72 | B |
| ATOM | 1888 | N3 | ADE | B | 236 | 10.874 | 11.186 | 30.739 | 1.00 | 62.70 | B |
| ATOM | 1889 | C2 | ADE | B | 236 | 10.977 | 12.031 | 29.717 | 1.00 | 62.13 | B |
| ATOM | 1890 | N1 | ADE | B | 236 | 11.730 | 11.942 | 28.615 | 1.00 | 61.58 | B |
| ATOM | 1891 | C6 | ADE | B | 236 | 12.519 | 10.860 | 28.464 | 1.00 | 62.43 | B |
| ATOM | 1892 | N6 | ADE | B | 236 | 13.283 | 10.775 | 27.376 | 1.00 | 62.70 | B |
| ATOM | 1893 | C5 | ADE | B | 236 | 12.494 | 9.890 | 29.472 | 1.00 | 62.23 | B |
| ATOM | 1894 | N7 | ADE | B | 236 | 13.169 | 8.695 | 29.623 | 1.00 | 64.68 | B |
| ATOM | 1895 | C8 | ADE | B | 236 | 12.754 | 8.242 | 30.781 | 1.00 | 64.90 | B |
| ATOM | 1896 | C2′ | ADE | B | 236 | 9.897 | 8.178 | 32.720 | 1.00 | 65.12 | B |
| ATOM | 1897 | O2′ | ADE | B | 236 | 9.114 | 8.717 | 33.769 | 1.00 | 64.29 | B |
| ATOM | 1898 | C3′ | ADE | B | 236 | 10.287 | 6.724 | 32.959 | 1.00 | 66.25 | B |
| ATOM | 1899 | O3′ | ADE | B | 236 | 9.240 | 5.963 | 33.556 | 1.00 | 67.09 | B |
| ATOM | 1900 | P | ADE | B | 237 | 8.247 | 5.102 | 32.626 | 1.00 | 67.47 | B |
| ATOM | 1901 | O1P | ADE | B | 237 | 7.456 | 4.214 | 33.517 | 1.00 | 66.06 | B |
| ATOM | 1902 | O2P | ADE | B | 237 | 9.027 | 4.525 | 31.498 | 1.00 | 66.74 | B |
| ATOM | 1903 | O5′ | ADE | B | 237 | 7.246 | 6.171 | 32.000 | 1.00 | 68.64 | B |
| ATOM | 1904 | C5′ | ADE | B | 237 | 6.265 | 6.819 | 32.800 | 1.00 | 67.38 | B |
| ATOM | 1905 | C4′ | ADE | B | 237 | 5.743 | 8.032 | 32.078 | 1.00 | 68.03 | B |
| ATOM | 1906 | O4′ | ADE | B | 237 | 6.855 | 8.925 | 31.800 | 1.00 | 66.67 | B |
| ATOM | 1907 | C1′ | ADE | B | 237 | 6.721 | 9.468 | 30.501 | 1.00 | 66.14 | B |
| ATOM | 1908 | N9 | ADE | B | 237 | 7.805 | 8.942 | 29.684 | 1.00 | 63.47 | B |
| ATOM | 1909 | C4 | ADE | B | 237 | 8.462 | 9.597 | 28.671 | 1.00 | 63.64 | B |
| ATOM | 1910 | N3 | ADE | B | 237 | 8.243 | 10.847 | 28.229 | 1.00 | 62.98 | B |
| ATOM | 1911 | C2 | ADE | B | 237 | 9.062 | 11.139 | 27.214 | 1.00 | 63.10 | B |
| ATOM | 1912 | N1 | ADE | B | 237 | 10.009 | 10.380 | 26.642 | 1.00 | 63.59 | B |
| ATOM | 1913 | C6 | ADE | B | 237 | 10.208 | 9.129 | 27.121 | 1.00 | 64.56 | B |
| ATOM | 1914 | N6 | ADE | B | 237 | 11.163 | 8.372 | 26.566 | 1.00 | 65.06 | B |
| ATOM | 1915 | C5 | ADE | B | 237 | 9.395 | 8.698 | 28.187 | 1.00 | 64.20 | B |
| ATOM | 1916 | N7 | ADE | B | 237 | 9.328 | 7.501 | 28.884 | 1.00 | 64.99 | B |
| ATOM | 1917 | C8 | ADE | B | 237 | 8.373 | 7.700 | 29.759 | 1.00 | 63.91 | B |
| ATOM | 1918 | C2′ | ADE | B | 237 | 5.343 | 9.059 | 29.982 | 1.00 | 67.57 | B |
| ATOM | 1919 | O2′ | ADE | B | 237 | 4.385 | 10.037 | 30.334 | 1.00 | 67.19 | B |
| ATOM | 1920 | C3′ | ADE | B | 237 | 5.121 | 7.745 | 30.719 | 1.00 | 68.79 | B |
| ATOM | 1921 | O3′ | ADE | B | 237 | 3.729 | 7.493 | 30.869 | 1.00 | 71.20 | B |
| ATOM | 1922 | P | URI | B | 238 | 2.956 | 6.603 | 29.778 | 1.00 | 74.04 | B |
| ATOM | 1923 | O1P | URI | B | 238 | 3.699 | 5.327 | 29.602 | 1.00 | 71.72 | B |
| ATOM | 1924 | O2P | URI | B | 238 | 1.516 | 6.576 | 30.161 | 1.00 | 73.12 | B |
| ATOM | 1925 | O5′ | URI | B | 238 | 3.110 | 7.429 | 28.426 | 1.00 | 74.17 | B |
| ATOM | 1926 | C5′ | URI | B | 238 | 2.071 | 8.280 | 27.968 | 1.00 | 75.65 | B |
| ATOM | 1927 | C4′ | URI | B | 238 | 2.636 | 9.338 | 27.061 | 1.00 | 77.79 | B |
| ATOM | 1928 | O4′ | URI | B | 238 | 4.078 | 9.354 | 27.176 | 1.00 | 78.47 | B |
| ATOM | 1929 | C1′ | URI | B | 238 | 4.599 | 10.049 | 26.060 | 1.00 | 78.56 | B |
| ATOM | 1930 | N1 | URI | B | 238 | 5.849 | 9.413 | 25.623 | 1.00 | 77.53 | B |
| ATOM | 1931 | C6 | URI | B | 238 | 6.286 | 8.214 | 26.139 | 1.00 | 77.02 | B |
| ATOM | 1932 | C2 | URI | B | 238 | 6.582 | 10.081 | 24.656 | 1.00 | 77.77 | B |
| ATOM | 1933 | O2 | URI | B | 238 | 6.228 | 11.146 | 24.181 | 1.00 | 77.80 | B |
| ATOM | 1934 | N3 | URI | B | 238 | 7.743 | 9.461 | 24.267 | 1.00 | 77.99 | B |
| ATOM | 1935 | C4 | URI | B | 238 | 8.242 | 8.267 | 24.736 | 1.00 | 78.47 | B |
| ATOM | 1936 | O4 | URI | B | 238 | 9.287 | 7.818 | 24.248 | 1.00 | 79.15 | B |
| ATOM | 1937 | C5 | URI | B | 238 | 7.430 | 7.632 | 25.744 | 1.00 | 77.52 | B |
| ATOM | 1938 | C2′ | URI | B | 238 | 3.504 | 10.061 | 24.988 | 1.00 | 79.64 | B |
| ATOM | 1939 | O2′ | URI | B | 238 | 2.993 | 11.371 | 24.840 | 1.00 | 79.89 | B |
| ATOM | 1940 | C3′ | URI | B | 238 | 2.483 | 9.088 | 25.574 | 1.00 | 80.64 | B |
| ATOM | 1941 | O3′ | URI | B | 238 | 1.159 | 9.356 | 25.120 | 1.00 | 83.61 | B |
| ATOM | 1942 | P | GUA | B | 239 | 0.479 | 8.386 | 24.021 | 1.00 | 85.15 | B |
| ATOM | 1943 | O1P | GUA | B | 239 | 0.691 | 6.974 | 24.448 | 1.00 | 83.25 | B |
| ATOM | 1944 | O2P | GUA | B | 239 | −0.903 | 8.895 | 23.802 | 1.00 | 83.34 | B |
| ATOM | 1945 | O5′ | GUA | B | 239 | 1.346 | 8.601 | 22.697 | 1.00 | 84.02 | B |
| ATOM | 1946 | C5′ | GUA | B | 239 | 0.951 | 9.532 | 21.690 | 1.00 | 83.77 | B |
| ATOM | 1947 | C4′ | GUA | B | 239 | 2.166 | 10.049 | 20.965 | 1.00 | 84.35 | B |
| ATOM | 1948 | O4′ | GUA | B | 239 | 3.330 | 9.793 | 21.785 | 1.00 | 84.90 | B |
| ATOM | 1949 | C1′ | GUA | B | 239 | 4.285 | 9.050 | 21.062 | 1.00 | 85.92 | B |
| ATOM | 1950 | N9 | GUA | B | 239 | 4.778 | 7.985 | 21.933 | 1.00 | 87.13 | B |
| ATOM | 1951 | C4 | GUA | B | 239 | 6.051 | 7.456 | 21.941 | 1.00 | 87.25 | B |
| ATOM | 1952 | N3 | GUA | B | 239 | 7.077 | 7.839 | 21.144 | 1.00 | 87.13 | B |
| ATOM | 1953 | C2 | GUA | B | 239 | 8.180 | 7.132 | 21.381 | 1.00 | 88.19 | B |
| ATOM | 1954 | N2 | GUA | B | 239 | 9.298 | 7.367 | 20.668 | 1.00 | 88.31 | B |
| ATOM | 1955 | N1 | GUA | B | 239 | 8.272 | 6.139 | 22.331 | 1.00 | 87.85 | B |
| ATOM | 1956 | C6 | GUA | B | 239 | 7.235 | 5.731 | 23.168 | 1.00 | 87.29 | B |
| ATOM | 1957 | O6 | GUA | B | 239 | 7.434 | 4.834 | 24.001 | 1.00 | 86.46 | B |
| ATOM | 1958 | C5 | GUA | B | 239 | 6.033 | 6.473 | 22.918 | 1.00 | 87.33 | B |
| ATOM | 1959 | N7 | GUA | B | 239 | 4.775 | 6.391 | 23.514 | 1.00 | 85.92 | B |
| ATOM | 1960 | C8 | GUA | B | 239 | 4.067 | 7.304 | 22.903 | 1.00 | 86.41 | B |
| ATOM | 1961 | C2′ | GUA | B | 239 | 3.623 | 8.504 | 19.797 | 1.00 | 85.49 | B |
| ATOM | 1962 | O2′ | GUA | B | 239 | 4.541 | 8.533 | 18.723 | 1.00 | 84.99 | B |
| ATOM | 1963 | C3′ | GUA | B | 239 | 2.427 | 9.436 | 19.596 | 1.00 | 85.70 | B |
| ATOM | 1964 | O3′ | GUA | B | 239 | 2.688 | 10.452 | 18.636 | 1.00 | 87.60 | B |
| ATOM | 1965 | P | URI | B | 240 | 1.613 | 10.740 | 17.474 | 1.00 | 88.74 | B |
| ATOM | 1966 | O1P | URI | B | 240 | 2.153 | 10.101 | 16.246 | 1.00 | 88.37 | B |
| ATOM | 1967 | O2P | URI | B | 240 | 0.261 | 10.358 | 17.977 | 1.00 | 87.64 | B |
| ATOM | 1968 | O5′ | URI | B | 240 | 1.646 | 12.324 | 17.273 | 1.00 | 87.55 | B |
| ATOM | 1969 | C5′ | URI | B | 240 | 0.919 | 13.196 | 18.146 | 1.00 | 86.34 | B |
| ATOM | 1970 | C4′ | URI | B | 240 | 0.733 | 14.555 | 17.503 | 1.00 | 84.94 | B |
| ATOM | 1971 | O4′ | URI | B | 240 | 1.698 | 15.501 | 18.037 | 1.00 | 82.31 | B |
| ATOM | 1972 | C1′ | URI | B | 240 | 2.250 | 16.239 | 16.974 | 1.00 | 80.29 | B |
| ATOM | 1973 | N1 | URI | B | 240 | 3.592 | 16.675 | 17.364 | 1.00 | 77.74 | B |
| ATOM | 1974 | C6 | URI | B | 240 | 4.588 | 15.774 | 17.632 | 1.00 | 76.62 | B |
| ATOM | 1975 | C2 | URI | B | 240 | 3.815 | 18.027 | 17.440 | 1.00 | 76.37 | B |
| ATOM | 1976 | O2 | URI | B | 240 | 2.945 | 18.846 | 17.226 | 1.00 | 75.50 | B |
| ATOM | 1977 | N3 | URI | B | 240 | 5.092 | 18.384 | 17.778 | 1.00 | 75.78 | B |
| ATOM | 1978 | C4 | URI | B | 240 | 6.145 | 17.536 | 18.044 | 1.00 | 76.60 | B |
| ATOM | 1979 | O4 | URI | B | 240 | 7.260 | 18.008 | 18.270 | 1.00 | 76.78 | B |
| ATOM | 1980 | C5 | URI | B | 240 | 5.824 | 16.146 | 17.961 | 1.00 | 76.69 | B |
| ATOM | 1981 | C2′ | URI | B | 240 | 2.248 | 15.272 | 15.797 | 1.00 | 82.64 | B |
| ATOM | 1982 | O2′ | URI | B | 240 | 2.361 | 15.966 | 14.569 | 1.00 | 80.34 | B |
| ATOM | 1983 | C3′ | URI | B | 240 | 0.893 | 14.605 | 15.985 | 1.00 | 85.43 | B |
| ATOM | 1984 | O3′ | URI | B | 240 | −0.116 | 15.438 | 15.448 | 1.00 | 90.12 | B |
| ATOM | 1985 | P | ADE | B | 241 | −1.384 | 14.778 | 14.732 | 1.00 | 93.02 | B |
| ATOM | 1986 | O1P | ADE | B | 241 | −1.786 | 13.583 | 15.518 | 1.00 | 92.58 | B |
| ATOM | 1987 | O2P | ADE | B | 241 | −1.052 | 14.636 | 13.293 | 1.00 | 93.42 | B |
| ATOM | 1988 | O5′ | ADE | B | 241 | −2.504 | 15.892 | 14.882 | 1.00 | 94.43 | B |
| ATOM | 1989 | C5′ | ADE | B | 241 | −2.321 | 17.164 | 14.290 | 1.00 | 98.62 | B |
| ATOM | 1990 | C4′ | ADE | B | 241 | −3.623 | 17.918 | 14.270 | 1.00 | 101.48 | B |
| ATOM | 1991 | O4′ | ADE | B | 241 | −4.073 | 18.103 | 15.643 | 1.00 | 104.39 | B |
| ATOM | 1992 | C1′ | ADE | B | 241 | −4.272 | 19.481 | 15.893 | 1.00 | 106.54 | B |
| ATOM | 1993 | N9 | ADE | B | 241 | −3.081 | 19.989 | 16.588 | 1.00 | 110.86 | B |
| ATOM | 1994 | C4 | ADE | B | 241 | −3.062 | 20.942 | 17.584 | 1.00 | 113.37 | B |
| ATOM | 1995 | N3 | ADE | B | 241 | −4.118 | 21.576 | 18.131 | 1.00 | 113.96 | B |
| ATOM | 1996 | C2 | ADE | B | 241 | −3.717 | 22.446 | 19.062 | 1.00 | 114.64 | B |
| ATOM | 1997 | N1 | ADE | B | 241 | −2.472 | 22.739 | 19.474 | 1.00 | 115.18 | B |
| ATOM | 1998 | C6 | ADE | B | 241 | −1.430 | 22.093 | 18.899 | 1.00 | 115.28 | B |
| ATOM | 1999 | N6 | ADE | B | 241 | −0.191 | 22.407 | 19.290 | 1.00 | 115.87 | B |
| ATOM | 2000 | C5 | ADE | B | 241 | −1.722 | 21.128 | 17.905 | 1.00 | 114.44 | B |
| ATOM | 2001 | N7 | ADE | B | 241 | −0.912 | 20.294 | 17.142 | 1.00 | 113.34 | B |
| ATOM | 2002 | C8 | ADE | B | 241 | −1.760 | 19.639 | 16.383 | 1.00 | 111.93 | B |
| ATOM | 2003 | C2′ | ADE | B | 241 | −4.500 | 20.115 | 14.516 | 1.00 | 103.51 | B |
| ATOM | 2004 | O2′ | ADE | B | 241 | −5.835 | 19.954 | 14.076 | 1.00 | 102.72 | B |
| ATOM | 2005 | C3′ | ADE | B | 241 | −3.522 | 19.316 | 13.673 | 1.00 | 101.05 | B |
| ATOM | 2006 | O3′ | ADE | B | 241 | −3.853 | 19.293 | 12.295 | 1.00 | 96.50 | B |
| ATOM | 2007 | P | GUA | B | 242 | −2.673 | 19.182 | 11.212 | 1.00 | 94.24 | B |
| ATOM | 2008 | O1P | GUA | B | 242 | −1.663 | 18.220 | 11.733 | 1.00 | 93.59 | B |
| ATOM | 2009 | O2P | GUA | B | 242 | −3.265 | 18.957 | 9.870 | 1.00 | 95.12 | B |
| ATOM | 2010 | O5′ | GUA | B | 242 | −2.028 | 20.638 | 11.239 | 1.00 | 91.69 | B |
| ATOM | 2011 | C5′ | GUA | B | 242 | −1.297 | 21.089 | 12.377 | 1.00 | 86.38 | B |
| ATOM | 2012 | C4′ | GUA | B | 242 | −1.773 | 22.460 | 12.811 | 1.00 | 81.63 | B |
| ATOM | 2013 | O4′ | GUA | B | 242 | −1.631 | 22.555 | 14.259 | 1.00 | 80.15 | B |
| ATOM | 2014 | C1′ | GUA | B | 242 | −0.711 | 23.581 | 14.575 | 1.00 | 78.46 | B |
| ATOM | 2015 | N9 | GUA | B | 242 | 0.627 | 22.994 | 14.628 | 1.00 | 77.67 | B |
| ATOM | 2016 | C4 | GUA | B | 242 | 1.808 | 23.664 | 14.862 | 1.00 | 77.32 | B |
| ATOM | 2017 | N3 | GUA | B | 242 | 1.936 | 24.983 | 15.111 | 1.00 | 77.22 | B |
| ATOM | 2018 | C2 | GUA | B | 242 | 3.201 | 25.332 | 15.287 | 1.00 | 76.26 | B |
| ATOM | 2019 | N2 | GUA | B | 242 | 3.508 | 26.613 | 15.545 | 1.00 | 76.47 | B |
| ATOM | 2020 | N1 | GUA | B | 242 | 4.255 | 24.455 | 15.223 | 1.00 | 74.74 | B |
| ATOM | 2021 | C6 | GUA | B | 242 | 4.147 | 23.096 | 14.967 | 1.00 | 74.67 | B |
| ATOM | 2022 | O6 | GUA | B | 242 | 5.159 | 22.401 | 14.926 | 1.00 | 73.56 | B |
| ATOM | 2023 | C5 | GUA | B | 242 | 2.795 | 22.705 | 14.778 | 1.00 | 76.45 | B |
| ATOM | 2024 | N7 | GUA | B | 242 | 2.248 | 21.457 | 14.511 | 1.00 | 76.62 | B |
| ATOM | 2025 | C8 | GUA | B | 242 | 0.963 | 21.675 | 14.437 | 1.00 | 77.01 | B |
| ATOM | 2026 | C2′ | GUA | B | 242 | −0.855 | 24.575 | 13.428 | 1.00 | 78.62 | B |
| ATOM | 2027 | O2′ | GUA | B | 242 | −2.017 | 25.361 | 13.596 | 1.00 | 78.37 | B |
| ATOM | 2028 | C3′ | GUA | B | 242 | −0.963 | 23.625 | 12.241 | 1.00 | 78.98 | B |
| ATOM | 2029 | O3′ | GUA | B | 242 | −1.642 | 24.238 | 11.153 | 1.00 | 75.65 | B |
| ATOM | 2030 | P | CYT | B | 243 | −0.826 | 25.160 | 10.121 | 1.00 | 73.97 | B |
| ATOM | 2031 | O1P | CYT | B | 243 | 0.183 | 24.313 | 9.425 | 1.00 | 71.15 | B |
| ATOM | 2032 | O2P | CYT | B | 243 | −1.840 | 25.894 | 9.323 | 1.00 | 73.99 | B |
| ATOM | 2033 | O5′ | CYT | B | 243 | −0.071 | 26.213 | 11.050 | 1.00 | 70.50 | B |
| ATOM | 2034 | C5′ | CYT | B | 243 | −0.772 | 27.305 | 11.636 | 1.00 | 67.12 | B |
| ATOM | 2035 | C4′ | CYT | B | 243 | 0.185 | 28.422 | 11.972 | 1.00 | 65.60 | B |
| ATOM | 2036 | O4′ | CYT | B | 243 | 1.082 | 27.971 | 13.020 | 1.00 | 65.34 | B |
| ATOM | 2037 | C1′ | CYT | B | 243 | 2.391 | 28.452 | 12.778 | 1.00 | 62.94 | B |
| ATOM | 2038 | N1 | CYT | B | 243 | 3.265 | 27.302 | 12.537 | 1.00 | 59.80 | B |
| ATOM | 2039 | C6 | CYT | B | 243 | 2.747 | 26.090 | 12.183 | 1.00 | 58.51 | B |
| ATOM | 2040 | C2 | CYT | B | 243 | 4.637 | 27.466 | 12.679 | 1.00 | 58.89 | B |
| ATOM | 2041 | O2 | CYT | B | 243 | 5.072 | 28.582 | 12.996 | 1.00 | 59.23 | B |
| ATOM | 2042 | N3 | CYT | B | 243 | 5.454 | 26.410 | 12.471 | 1.00 | 57.13 | B |
| ATOM | 2043 | C4 | CYT | B | 243 | 4.937 | 25.226 | 12.144 | 1.00 | 56.84 | B |
| ATOM | 2044 | N4 | CYT | B | 243 | 5.771 | 24.202 | 11.982 | 1.00 | 55.44 | B |
| ATOM | 2045 | C5 | CYT | B | 243 | 3.539 | 25.036 | 11.978 | 1.00 | 57.53 | B |
| ATOM | 2046 | C2′ | CYT | B | 243 | 2.319 | 29.390 | 11.577 | 1.00 | 65.14 | B |
| ATOM | 2047 | O2′ | CYT | B | 243 | 2.176 | 30.720 | 12.026 | 1.00 | 67.49 | B |
| ATOM | 2048 | C3′ | CYT | B | 243 | 1.096 | 28.858 | 10.838 | 1.00 | 65.51 | B |
| ATOM | 2049 | O3′ | CYT | B | 243 | 0.485 | 29.886 | 10.070 | 1.00 | 66.47 | B |
| ATOM | 2050 | P | URI | B | 244 | 1.011 | 30.180 | 8.578 | 1.00 | 67.88 | B |
| ATOM | 2051 | O1P | URI | B | 244 | 1.033 | 28.866 | 7.888 | 1.00 | 68.52 | B |
| ATOM | 2052 | O2P | URI | B | 244 | 0.249 | 31.306 | 7.986 | 1.00 | 67.85 | B |
| ATOM | 2053 | O5′ | URI | B | 244 | 2.512 | 30.663 | 8.781 | 1.00 | 65.45 | B |
| ATOM | 2054 | C5′ | URI | B | 244 | 2.797 | 32.001 | 9.150 | 1.00 | 64.15 | B |
| ATOM | 2055 | C4′ | URI | B | 244 | 4.286 | 32.210 | 9.234 | 1.00 | 63.22 | B |
| ATOM | 2056 | O4′ | URI | B | 244 | 4.831 | 31.277 | 10.202 | 1.00 | 63.70 | B |
| ATOM | 2057 | C1′ | URI | B | 244 | 6.142 | 30.901 | 9.812 | 1.00 | 64.16 | B |
| ATOM | 2058 | N1 | URI | B | 244 | 6.167 | 29.445 | 9.642 | 1.00 | 65.13 | B |
| ATOM | 2059 | C6 | URI | B | 244 | 5.021 | 28.735 | 9.364 | 1.00 | 65.67 | B |
| ATOM | 2060 | C2 | URI | B | 244 | 7.386 | 28.811 | 9.762 | 1.00 | 65.43 | B |
| ATOM | 2061 | O2 | URI | B | 244 | 8.413 | 29.407 | 10.018 | 1.00 | 64.90 | B |
| ATOM | 2062 | N3 | URI | B | 244 | 7.357 | 27.452 | 9.571 | 1.00 | 65.48 | B |
| ATOM | 2063 | C4 | URI | B | 244 | 6.254 | 26.680 | 9.283 | 1.00 | 66.10 | B |
| ATOM | 2064 | O4 | URI | B | 244 | 6.395 | 25.473 | 9.111 | 1.00 | 66.74 | B |
| ATOM | 2065 | C5 | URI | B | 244 | 5.022 | 27.408 | 9.184 | 1.00 | 66.64 | B |
| ATOM | 2066 | C2′ | URI | B | 244 | 6.460 | 31.632 | 8.507 | 1.00 | 63.91 | B |
| ATOM | 2067 | O2′ | URI | B | 244 | 7.219 | 32.795 | 8.762 | 1.00 | 63.56 | B |
| ATOM | 2068 | C3′ | URI | B | 244 | 5.058 | 31.895 | 7.965 | 1.00 | 63.41 | B |
| ATOM | 2069 | O3′ | URI | B | 244 | 5.008 | 32.959 | 7.018 | 1.00 | 61.34 | B |
| ATOM | 2070 | P | ADE | B | 245 | 5.762 | 32.802 | 5.605 | 1.00 | 60.75 | B |
| ATOM | 2071 | O1P | ADE | B | 245 | 5.276 | 33.892 | 4.731 | 1.00 | 60.89 | B |
| ATOM | 2072 | O2P | ADE | B | 245 | 5.658 | 31.404 | 5.142 | 1.00 | 61.86 | B |
| ATOM | 2073 | O5′ | ADE | B | 245 | 7.284 | 33.065 | 5.989 | 1.00 | 61.11 | B |
| ATOM | 2074 | C5′ | ADE | B | 245 | 8.355 | 32.596 | 5.182 | 1.00 | 61.47 | B |
| ATOM | 2075 | C4′ | ADE | B | 245 | 9.642 | 32.754 | 5.941 | 1.00 | 63.53 | B |
| ATOM | 2076 | O4′ | ADE | B | 245 | 9.548 | 31.926 | 7.129 | 1.00 | 64.13 | B |
| ATOM | 2077 | C1′ | ADE | B | 245 | 10.835 | 31.458 | 7.482 | 1.00 | 64.97 | B |
| ATOM | 2078 | N9 | ADE | B | 245 | 10.828 | 30.001 | 7.449 | 1.00 | 65.17 | B |
| ATOM | 2079 | C4 | ADE | B | 245 | 11.902 | 29.220 | 7.783 | 1.00 | 66.29 | B |
| ATOM | 2080 | N3 | ADE | B | 245 | 13.100 | 29.638 | 8.220 | 1.00 | 67.68 | B |
| ATOM | 2081 | C2 | ADE | B | 245 | 13.910 | 28.603 | 8.430 | 1.00 | 68.28 | B |
| ATOM | 2082 | N1 | ADE | B | 245 | 13.678 | 27.298 | 8.259 | 1.00 | 67.73 | B |
| ATOM | 2083 | C6 | ADE | B | 245 | 12.463 | 26.911 | 7.812 | 1.00 | 67.75 | B |
| ATOM | 2084 | N6 | ADE | B | 245 | 12.240 | 25.606 | 7.633 | 1.00 | 68.72 | B |
| ATOM | 2085 | C5 | ADE | B | 245 | 11.506 | 27.918 | 7.561 | 1.00 | 66.45 | B |
| ATOM | 2086 | N7 | ADE | B | 245 | 10.191 | 27.876 | 7.118 | 1.00 | 64.62 | B |
| ATOM | 2087 | C8 | ADE | B | 245 | 9.835 | 29.136 | 7.073 | 1.00 | 64.32 | B |
| ATOM | 2088 | C2′ | ADE | B | 245 | 11.829 | 32.017 | 6.462 | 1.00 | 65.52 | B |
| ATOM | 2089 | O2′ | ADE | B | 245 | 12.446 | 33.165 | 6.996 | 1.00 | 66.76 | B |
| ATOM | 2090 | C3′ | ADE | B | 245 | 10.929 | 32.305 | 5.262 | 1.00 | 65.07 | B |
| ATOM | 2091 | O3′ | ADE | B | 245 | 11.483 | 33.339 | 4.441 | 1.00 | 65.59 | B |
| ATOM | 2092 | P | ADE | B | 246 | 12.649 | 32.985 | 3.381 | 1.00 | 68.47 | B |
| ATOM | 2093 | O1P | ADE | B | 246 | 13.185 | 34.225 | 2.771 | 1.00 | 66.95 | B |
| ATOM | 2094 | O2P | ADE | B | 246 | 12.164 | 31.892 | 2.496 | 1.00 | 68.82 | B |
| ATOM | 2095 | O5′ | ADE | B | 246 | 13.818 | 32.421 | 4.292 | 1.00 | 66.81 | B |
| ATOM | 2096 | C5′ | ADE | B | 246 | 14.494 | 33.274 | 5.189 | 1.00 | 66.99 | B |
| ATOM | 2097 | C4′ | ADE | B | 246 | 15.813 | 32.676 | 5.539 | 1.00 | 68.35 | B |
| ATOM | 2098 | O4′ | ADE | B | 246 | 15.615 | 31.599 | 6.489 | 1.00 | 68.88 | B |
| ATOM | 2099 | C1′ | ADE | B | 246 | 16.508 | 30.539 | 6.198 | 1.00 | 69.64 | B |
| ATOM | 2100 | N9 | ADE | B | 246 | 15.709 | 29.386 | 5.804 | 1.00 | 70.53 | B |
| ATOM | 2101 | C4 | ADE | B | 246 | 16.123 | 28.081 | 5.802 | 1.00 | 70.88 | B |
| ATOM | 2102 | N3 | ADE | B | 246 | 17.327 | 27.613 | 6.165 | 1.00 | 70.57 | B |
| ATOM | 2103 | C2 | ADE | B | 246 | 17.369 | 26.292 | 6.037 | 1.00 | 72.08 | B |
| ATOM | 2104 | N1 | ADE | B | 246 | 16.417 | 25.452 | 5.616 | 1.00 | 73.37 | B |
| ATOM | 2105 | C6 | ADE | B | 246 | 15.219 | 25.966 | 5.250 | 1.00 | 73.57 | B |
| ATOM | 2106 | N6 | ADE | B | 246 | 14.269 | 25.139 | 4.810 | 1.00 | 75.70 | B |
| ATOM | 2107 | C5 | ADE | B | 246 | 15.047 | 27.345 | 5.348 | 1.00 | 71.37 | B |
| ATOM | 2108 | N7 | ADE | B | 246 | 13.969 | 28.168 | 5.071 | 1.00 | 70.32 | B |
| ATOM | 2109 | C8 | ADE | B | 246 | 14.412 | 29.368 | 5.356 | 1.00 | 70.83 | B |
| ATOM | 2110 | C2′ | ADE | B | 246 | 17.394 | 31.015 | 5.046 | 1.00 | 69.74 | B |
| ATOM | 2111 | O2′ | ADE | B | 246 | 18.583 | 31.628 | 5.501 | 1.00 | 68.86 | B |
| ATOM | 2112 | C3′ | ADE | B | 246 | 16.463 | 31.993 | 4.360 | 1.00 | 69.45 | B |
| ATOM | 2113 | O3′ | ADE | B | 246 | 17.170 | 32.940 | 3.595 | 1.00 | 71.42 | B |
| ATOM | 2114 | P | ADE | B | 247 | 17.302 | 32.725 | 2.018 | 1.00 | 74.08 | B |
| ATOM | 2115 | O1P | ADE | B | 247 | 18.211 | 33.790 | 1.522 | 1.00 | 72.22 | B |
| ATOM | 2116 | O2P | ADE | B | 247 | 15.927 | 32.594 | 1.449 | 1.00 | 71.77 | B |
| ATOM | 2117 | O5′ | ADE | B | 247 | 18.077 | 31.338 | 1.916 | 1.00 | 73.67 | B |
| ATOM | 2118 | C5′ | ADE | B | 247 | 19.313 | 31.159 | 2.597 | 1.00 | 73.69 | B |
| ATOM | 2119 | C4′ | ADE | B | 247 | 19.699 | 29.702 | 2.626 | 1.00 | 74.19 | B |
| ATOM | 2120 | O4′ | ADE | B | 247 | 18.730 | 28.929 | 3.380 | 1.00 | 74.62 | B |
| ATOM | 2121 | C1′ | ADE | B | 247 | 18.643 | 27.623 | 2.836 | 1.00 | 72.69 | B |
| ATOM | 2122 | N9 | ADE | B | 247 | 17.276 | 27.432 | 2.363 | 1.00 | 70.73 | B |
| ATOM | 2123 | C4 | ADE | B | 247 | 16.635 | 26.234 | 2.170 | 1.00 | 69.33 | B |
| ATOM | 2124 | N3 | ADE | B | 247 | 17.137 | 25.003 | 2.361 | 1.00 | 68.40 | B |
| ATOM | 2125 | C2 | ADE | B | 247 | 16.219 | 24.079 | 2.093 | 1.00 | 67.91 | B |
| ATOM | 2126 | N1 | ADE | B | 247 | 14.950 | 24.236 | 1.690 | 1.00 | 67.46 | B |
| ATOM | 2127 | C6 | ADE | B | 247 | 14.478 | 25.487 | 1.511 | 1.00 | 67.48 | B |
| ATOM | 2128 | N6 | ADE | B | 247 | 13.212 | 25.643 | 1.122 | 1.00 | 66.56 | B |
| ATOM | 2129 | C5 | ADE | B | 247 | 15.356 | 26.554 | 1.753 | 1.00 | 68.54 | B |
| ATOM | 2130 | N7 | ADE | B | 247 | 15.199 | 27.929 | 1.665 | 1.00 | 69.46 | B |
| ATOM | 2131 | C8 | ADE | B | 247 | 16.363 | 28.402 | 2.033 | 1.00 | 69.88 | B |
| ATOM | 2132 | C2′ | ADE | B | 247 | 19.687 | 27.545 | 1.721 | 1.00 | 74.57 | B |
| ATOM | 2133 | O2′ | ADE | B | 247 | 20.918 | 27.104 | 2.257 | 1.00 | 75.29 | B |
| ATOM | 2134 | C3′ | ADE | B | 247 | 19.759 | 29.000 | 1.285 | 1.00 | 74.55 | B |
| ATOM | 2135 | O3′ | ADE | B | 247 | 21.008 | 29.282 | 0.681 | 1.00 | 76.68 | B |
| ATOM | 2136 | P | ADE | B | 248 | 21.113 | 29.431 | −0.913 | 1.00 | 78.64 | B |
| ATOM | 2137 | O1P | ADE | B | 248 | 22.305 | 30.276 | −1.169 | 1.00 | 79.02 | B |
| ATOM | 2138 | O2P | ADE | B | 248 | 19.800 | 29.822 | −1.487 | 1.00 | 77.28 | B |
| ATOM | 2139 | O5′ | ADE | B | 248 | 21.459 | 27.953 | −1.387 | 1.00 | 80.68 | B |
| ATOM | 2140 | C5′ | ADE | B | 248 | 22.772 | 27.608 | −1.819 | 1.00 | 81.26 | B |
| ATOM | 2141 | C4′ | ADE | B | 248 | 23.671 | 27.365 | −0.630 | 1.00 | 81.73 | B |
| ATOM | 2142 | O4′ | ADE | B | 248 | 22.939 | 26.704 | 0.436 | 1.00 | 81.21 | B |
| ATOM | 2143 | C1′ | ADE | B | 248 | 23.827 | 25.861 | 1.159 | 1.00 | 82.59 | B |
| ATOM | 2144 | N9 | ADE | B | 248 | 23.358 | 24.484 | 1.048 | 1.00 | 82.41 | B |
| ATOM | 2145 | C4 | ADE | B | 248 | 24.067 | 23.360 | 1.389 | 1.00 | 82.27 | B |
| ATOM | 2146 | N3 | ADE | B | 248 | 25.319 | 23.302 | 1.863 | 1.00 | 82.48 | B |
| ATOM | 2147 | C2 | ADE | B | 248 | 25.670 | 22.041 | 2.111 | 1.00 | 82.79 | B |
| ATOM | 2148 | N1 | ADE | B | 248 | 24.964 | 20.917 | 1.954 | 1.00 | 83.05 | B |
| ATOM | 2149 | C6 | ADE | B | 248 | 23.706 | 21.011 | 1.475 | 1.00 | 83.89 | B |
| ATOM | 2150 | N6 | ADE | B | 248 | 22.992 | 19.890 | 1.329 | 1.00 | 83.78 | B |
| ATOM | 2151 | C5 | ADE | B | 248 | 23.219 | 22.295 | 1.164 | 1.00 | 83.22 | B |
| ATOM | 2152 | N7 | ADE | B | 248 | 22.003 | 22.737 | 0.668 | 1.00 | 83.80 | B |
| ATOM | 2153 | C8 | ADE | B | 248 | 22.140 | 24.039 | 0.613 | 1.00 | 83.41 | B |
| ATOM | 2154 | C2′ | ADE | B | 248 | 25.209 | 26.036 | 0.536 | 1.00 | 83.45 | B |
| ATOM | 2155 | O2′ | ADE | B | 248 | 25.901 | 27.065 | 1.212 | 1.00 | 85.26 | B |
| ATOM | 2156 | C3′ | ADE | B | 248 | 24.827 | 26.415 | −0.882 | 1.00 | 83.04 | B |
| ATOM | 2157 | O3′ | ADE | B | 248 | 25.890 | 27.072 | −1.550 | 1.00 | 84.14 | B |
| ATOM | 2158 | P | ADE | B | 249 | 26.230 | 26.683 | −3.069 | 1.00 | 85.50 | B |
| ATOM | 2159 | O1P | ADE | B | 249 | 27.452 | 27.452 | −3.406 | 1.00 | 84.72 | B |
| ATOM | 2160 | O2P | ADE | B | 249 | 25.009 | 26.835 | −3.911 | 1.00 | 84.17 | B |
| ATOM | 2161 | O5′ | ADE | B | 249 | 26.583 | 25.132 | −2.986 | 1.00 | 85.53 | B |
| ATOM | 2162 | C5′ | ADE | B | 249 | 27.427 | 24.630 | −1.956 | 1.00 | 87.01 | B |
| ATOM | 2163 | C4′ | ADE | B | 249 | 27.413 | 23.117 | −1.958 | 1.00 | 89.05 | B |
| ATOM | 2164 | O4′ | ADE | B | 249 | 26.309 | 22.642 | −1.135 | 1.00 | 89.34 | B |
| ATOM | 2165 | C1′ | ADE | B | 249 | 25.689 | 21.529 | −1.761 | 1.00 | 87.86 | B |
| ATOM | 2166 | N9 | ADE | B | 249 | 24.405 | 21.978 | −2.284 | 1.00 | 86.17 | B |
| ATOM | 2167 | C4 | ADE | B | 249 | 23.339 | 21.182 | −2.615 | 1.00 | 85.23 | B |
| ATOM | 2168 | N3 | ADE | B | 249 | 23.254 | 19.847 | −2.499 | 1.00 | 84.97 | B |
| ATOM | 2169 | C2 | ADE | B | 249 | 22.076 | 19.418 | −2.932 | 1.00 | 84.39 | B |
| ATOM | 2170 | N1 | ADE | B | 249 | 21.049 | 20.117 | −3.427 | 1.00 | 83.82 | B |
| ATOM | 2171 | C6 | ADE | B | 249 | 21.166 | 21.455 | −3.527 | 1.00 | 83.86 | B |
| ATOM | 2172 | N6 | ADE | B | 249 | 20.139 | 22.150 | −4.016 | 1.00 | 83.30 | B |
| ATOM | 2173 | C5 | ADE | B | 249 | 22.371 | 22.035 | −3.105 | 1.00 | 84.70 | B |
| ATOM | 2174 | N7 | ADE | B | 249 | 22.815 | 23.349 | −3.073 | 1.00 | 85.43 | B |
| ATOM | 2175 | C8 | ADE | B | 249 | 24.020 | 23.261 | −2.572 | 1.00 | 85.15 | B |
| ATOM | 2176 | C2′ | ADE | B | 249 | 26.616 | 21.102 | −2.899 | 1.00 | 88.41 | B |
| ATOM | 2177 | O2′ | ADE | B | 249 | 27.588 | 20.176 | −2.454 | 1.00 | 87.62 | B |
| ATOM | 2178 | C3′ | ADE | B | 249 | 27.192 | 22.450 | −3.309 | 1.00 | 89.31 | B |
| ATOM | 2179 | O3′ | ADE | B | 249 | 28.405 | 22.304 | −4.033 | 1.00 | 91.31 | B |
| ATOM | 2180 | P | GUA | B | 250 | 28.374 | 22.328 | −5.640 | 1.00 | 92.94 | B |
| ATOM | 2181 | O1P | GUA | B | 250 | 27.440 | 23.404 | −6.075 | 1.00 | 91.54 | B |
| ATOM | 2182 | O2P | GUA | B | 250 | 29.774 | 22.320 | −6.136 | 1.00 | 93.37 | B |
| ATOM | 2183 | O5′ | GUA | B | 250 | 27.712 | 20.935 | −6.024 | 1.00 | 91.73 | B |
| ATOM | 2184 | C5′ | GUA | B | 250 | 26.822 | 20.839 | −7.121 | 1.00 | 89.58 | B |
| ATOM | 2185 | C4′ | GUA | B | 250 | 25.917 | 19.655 | −6.941 | 1.00 | 88.31 | B |
| ATOM | 2186 | O4′ | GUA | B | 250 | 25.089 | 19.860 | −5.762 | 1.00 | 87.08 | B |
| ATOM | 2187 | C1′ | GUA | B | 250 | 23.726 | 19.682 | −6.102 | 1.00 | 86.14 | B |
| ATOM | 2188 | N9 | GUA | B | 250 | 23.146 | 21.005 | −6.315 | 1.00 | 84.76 | B |
| ATOM | 2189 | C4 | GUA | B | 250 | 21.856 | 21.278 | −6.690 | 1.00 | 83.37 | B |
| ATOM | 2190 | N3 | GUA | B | 250 | 20.888 | 20.366 | −6.902 | 1.00 | 82.60 | B |
| ATOM | 2191 | C2 | GUA | B | 250 | 19.756 | 20.929 | −7.278 | 1.00 | 81.86 | B |
| ATOM | 2192 | N2 | GUA | B | 250 | 18.689 | 20.160 | −7.531 | 1.00 | 81.52 | B |
| ATOM | 2193 | N1 | GUA | B | 250 | 19.588 | 22.286 | −7.433 | 1.00 | 81.35 | B |
| ATOM | 2194 | C6 | GUA | B | 250 | 20.574 | 23.244 | −7.219 | 1.00 | 81.91 | B |
| ATOM | 2195 | O6 | GUA | B | 250 | 20.322 | 24.442 | −7.397 | 1.00 | 81.19 | B |
| ATOM | 2196 | C5 | GUA | B | 250 | 21.791 | 22.651 | −6.811 | 1.00 | 82.53 | B |
| ATOM | 2197 | N7 | GUA | B | 250 | 23.010 | 23.231 | −6.498 | 1.00 | 82.75 | B |
| ATOM | 2198 | C8 | GUA | B | 250 | 23.781 | 22.219 | −6.205 | 1.00 | 84.50 | B |
| ATOM | 2199 | C2′ | GUA | B | 250 | 23.740 | 18.841 | −7.380 | 1.00 | 87.17 | B |
| ATOM | 2200 | O2′ | GUA | B | 250 | 23.898 | 17.468 | −7.079 | 1.00 | 86.01 | B |
| ATOM | 2201 | C3′ | GUA | B | 250 | 24.948 | 19.437 | −8.089 | 1.00 | 88.35 | B |
| ATOM | 2202 | O3′ | GUA | B | 250 | 25.535 | 18.561 | −9.039 | 1.00 | 89.61 | B |
| ATOM | 2203 | P | GUA | B | 251 | 25.721 | 19.050 | −10.558 | 1.00 | 90.34 | B |
| ATOM | 2204 | O1P | GUA | B | 251 | 25.813 | 20.535 | −10.570 | 1.00 | 88.07 | B |
| ATOM | 2205 | O2P | GUA | B | 251 | 26.809 | 18.240 | −11.162 | 1.00 | 88.48 | B |
| ATOM | 2206 | O5′ | GUA | B | 251 | 24.340 | 18.632 | −11.230 | 1.00 | 89.75 | B |
| ATOM | 2207 | C5′ | GUA | B | 251 | 23.603 | 17.524 | −10.724 | 1.00 | 88.88 | B |
| ATOM | 2208 | C4′ | GUA | B | 251 | 22.141 | 17.681 | −11.048 | 1.00 | 88.40 | B |
| ATOM | 2209 | O4′ | GUA | B | 251 | 21.513 | 18.539 | −10.062 | 1.00 | 87.77 | B |
| ATOM | 2210 | C1′ | GUA | B | 251 | 20.486 | 19.303 | −10.681 | 1.00 | 86.45 | B |
| ATOM | 2211 | N9 | GUA | B | 251 | 20.798 | 20.717 | −10.503 | 1.00 | 83.86 | B |
| ATOM | 2212 | C4 | GUA | B | 251 | 19.947 | 21.778 | −10.691 | 1.00 | 81.12 | B |
| ATOM | 2213 | N3 | GUA | B | 251 | 18.662 | 21.702 | −11.096 | 1.00 | 78.40 | B |
| ATOM | 2214 | C2 | GUA | B | 251 | 18.095 | 22.898 | −11.166 | 1.00 | 78.93 | B |
| ATOM | 2215 | N2 | GUA | B | 251 | 16.813 | 23.013 | −11.541 | 1.00 | 76.86 | B |
| ATOM | 2216 | N1 | GUA | B | 251 | 18.742 | 24.075 | −10.867 | 1.00 | 79.94 | B |
| ATOM | 2217 | C6 | GUA | B | 251 | 20.067 | 24.177 | −10.441 | 1.00 | 81.36 | B |
| ATOM | 2218 | O6 | GUA | B | 251 | 20.556 | 25.296 | −10.172 | 1.00 | 80.76 | B |
| ATOM | 2219 | C5 | GUA | B | 251 | 20.685 | 22.900 | −10.367 | 1.00 | 81.39 | B |
| ATOM | 2220 | N7 | GUA | B | 251 | 21.976 | 22.551 | −9.999 | 1.00 | 82.18 | B |
| ATOM | 2221 | C8 | GUA | B | 251 | 22.000 | 21.250 | −10.098 | 1.00 | 83.36 | B |
| ATOM | 2222 | C2′ | GUA | B | 251 | 20.428 | 18.880 | −12.149 | 1.00 | 87.55 | B |
| ATOM | 2223 | O2′ | GUA | B | 251 | 19.444 | 17.881 | −12.316 | 1.00 | 87.74 | B |
| ATOM | 2224 | C3′ | GUA | B | 251 | 21.848 | 18.371 | −12.367 | 1.00 | 88.81 | B |
| ATOM | 2225 | O3′ | GUA | B | 251 | 21.920 | 17.441 | −13.436 | 1.00 | 90.30 | B |
| ATOM | 2226 | P | ADE | B | 252 | 22.780 | 17.800 | −14.739 | 1.00 | 91.05 | B |
| ATOM | 2227 | O1P | ADE | B | 252 | 23.910 | 18.660 | −14.296 | 1.00 | 90.39 | B |
| ATOM | 2228 | O2P | ADE | B | 252 | 23.054 | 16.533 | −15.459 | 1.00 | 89.96 | B |
| ATOM | 2229 | O5′ | ADE | B | 252 | 21.781 | 18.671 | −15.620 | 1.00 | 91.95 | B |
| ATOM | 2230 | PC | ADE | B | 252 | 20.987 | 18.578 | −20.533 | 1.00 | 104.90 | B |
| ATOM | 2231 | O1C | ADE | B | 252 | 22.147 | 19.251 | −21.189 | 1.00 | 103.28 | B |
| ATOM | 2232 | O2C | ADE | B | 252 | 20.449 | 17.555 | −21.467 | 1.00 | 104.27 | B |
| ATOM | 2233 | O3′ | ADE | B | 252 | 21.484 | 17.850 | −19.180 | 1.00 | 101.71 | B |
| ATOM | 2234 | C5′ | ADE | B | 252 | 20.538 | 18.128 | −16.046 | 1.00 | 94.48 | B |
| ATOM | 2235 | C4′ | ADE | B | 252 | 20.126 | 18.735 | −17.362 | 1.00 | 96.88 | B |
| ATOM | 2236 | O4′ | ADE | B | 252 | 19.568 | 20.051 | −17.110 | 1.00 | 97.25 | B |
| ATOM | 2237 | C1′ | ADE | B | 252 | 19.932 | 20.933 | −18.158 | 1.00 | 97.83 | B |
| ATOM | 2238 | N9 | ADE | B | 252 | 20.812 | 21.964 | −17.615 | 1.00 | 96.09 | B |
| ATOM | 2239 | C4 | ADE | B | 252 | 20.556 | 23.312 | −17.556 | 1.00 | 94.96 | B |
| ATOM | 2240 | N3 | ADE | B | 252 | 19.437 | 23.947 | −17.947 | 1.00 | 94.36 | B |
| ATOM | 2241 | C2 | ADE | B | 252 | 19.555 | 25.259 | −17.758 | 1.00 | 93.41 | B |
| ATOM | 2242 | N1 | ADE | B | 252 | 20.587 | 25.953 | −17.262 | 1.00 | 93.74 | B |
| ATOM | 2243 | C6 | ADE | B | 252 | 21.697 | 25.278 | −16.878 | 1.00 | 94.05 | B |
| ATOM | 2244 | N6 | ADE | B | 252 | 22.733 | 25.964 | −16.384 | 1.00 | 93.71 | B |
| ATOM | 2245 | C5 | ADE | B | 252 | 21.696 | 23.887 | −17.025 | 1.00 | 94.40 | B |
| ATOM | 2246 | N7 | ADE | B | 252 | 22.647 | 22.920 | −16.734 | 1.00 | 95.22 | B |
| ATOM | 2247 | C8 | ADE | B | 252 | 22.074 | 21.799 | −17.094 | 1.00 | 95.90 | B |
| ATOM | 2248 | C2′ | ADE | B | 252 | 20.677 | 20.106 | −19.204 | 1.00 | 99.30 | B |
| ATOM | 2249 | O2′ | ADE | B | 252 | 19.816 | 19.648 | −20.230 | 1.00 | 101.99 | B |
| ATOM | 2250 | C3′ | ADE | B | 252 | 21.251 | 18.984 | −18.355 | 1.00 | 98.41 | B |
| TER | | | | | | | | | | | |
| ATOM | 2251 | O6P | GUA | C | 401 | 19.129 | 13.958 | 103.041 | 1.00 | 82.82 | C |
| ATOM | 2252 | O3P | GUA | C | 401 | 19.403 | 15.512 | 101.028 | 1.00 | 79.10 | C |
| ATOM | 2253 | O4P | GUA | C | 401 | 21.349 | 15.070 | 102.651 | 1.00 | 84.74 | C |
| ATOM | 2254 | O5P | GUA | C | 401 | 20.623 | 13.217 | 101.168 | 1.00 | 81.84 | C |
| ATOM | 2255 | P2 | GUA | C | 401 | 20.137 | 14.411 | 101.990 | 1.00 | 84.24 | C |
| ATOM | 2256 | P | GUA | C | 401 | 20.421 | 16.053 | 99.862 | 1.00 | 74.41 | C |
| ATOM | 2257 | O1P | GUA | C | 401 | 20.862 | 14.926 | 98.946 | 1.00 | 74.15 | C |
| ATOM | 2258 | O2P | GUA | C | 401 | 21.628 | 16.722 | 100.495 | 1.00 | 73.92 | C |
| ATOM | 2259 | O5′ | GUA | C | 401 | 19.661 | 17.160 | 98.943 | 1.00 | 70.87 | C |
| ATOM | 2260 | C5′ | GUA | C | 401 | 18.262 | 17.037 | 98.648 | 1.00 | 65.12 | C |
| ATOM | 2261 | C4′ | GUA | C | 401 | 17.543 | 18.329 | 98.955 | 1.00 | 61.77 | C |
| ATOM | 2262 | O4′ | GUA | C | 401 | 17.378 | 18.484 | 100.385 | 1.00 | 61.70 | C |
| ATOM | 2263 | C1′ | GUA | C | 401 | 17.296 | 19.867 | 100.708 | 1.00 | 61.13 | C |
| ATOM | 2264 | N9 | GUA | C | 401 | 18.352 | 20.172 | 101.671 | 1.00 | 61.89 | C |
| ATOM | 2265 | C4 | GUA | C | 401 | 18.674 | 21.411 | 102.173 | 1.00 | 62.05 | C |
| ATOM | 2266 | N3 | GUA | C | 401 | 18.048 | 22.569 | 101.884 | 1.00 | 63.41 | C |
| ATOM | 2267 | C2 | GUA | C | 401 | 18.613 | 23.598 | 102.490 | 1.00 | 63.99 | C |
| ATOM | 2268 | N2 | GUA | C | 401 | 18.118 | 24.834 | 102.308 | 1.00 | 65.06 | C |
| ATOM | 2269 | N1 | GUA | C | 401 | 19.703 | 23.495 | 103.315 | 1.00 | 62.94 | C |
| ATOM | 2270 | C6 | GUA | C | 401 | 20.363 | 22.313 | 103.626 | 1.00 | 62.01 | C |
| ATOM | 2271 | O6 | GUA | C | 401 | 21.352 | 22.334 | 104.371 | 1.00 | 61.22 | C |
| ATOM | 2272 | C5 | GUA | C | 401 | 19.766 | 21.203 | 102.988 | 1.00 | 61.68 | C |
| ATOM | 2273 | N7 | GUA | C | 401 | 20.106 | 19.860 | 103.023 | 1.00 | 60.80 | C |
| ATOM | 2274 | C8 | GUA | C | 401 | 19.241 | 19.287 | 102.233 | 1.00 | 61.30 | C |
| ATOM | 2275 | C2′ | GUA | C | 401 | 17.485 | 20.633 | 99.396 | 1.00 | 61.41 | C |
| ATOM | 2276 | O2′ | GUA | C | 401 | 16.246 | 20.987 | 98.818 | 1.00 | 60.56 | C |
| ATOM | 2277 | C3′ | GUA | C | 401 | 18.242 | 19.614 | 98.560 | 1.00 | 60.57 | C |
| ATOM | 2278 | O3′ | GUA | C | 401 | 18.078 | 19.901 | 97.186 | 1.00 | 60.34 | C |
| ATOM | 2279 | P | CYT | C | 402 | 19.241 | 20.679 | 96.403 | 1.00 | 61.86 | C |
| ATOM | 2280 | O1P | CYT | C | 402 | 20.524 | 20.130 | 96.912 | 1.00 | 59.62 | C |
| ATOM | 2281 | O2P | CYT | C | 402 | 18.953 | 20.634 | 94.946 | 1.00 | 59.04 | C |
| ATOM | 2282 | O5′ | CYT | C | 402 | 19.105 | 22.187 | 96.914 | 1.00 | 60.52 | C |
| ATOM | 2283 | C5′ | CYT | C | 402 | 17.883 | 22.915 | 96.767 | 1.00 | 58.50 | C |
| ATOM | 2284 | C4′ | CYT | C | 402 | 18.073 | 24.367 | 97.163 | 1.00 | 57.08 | C |
| ATOM | 2285 | O4′ | CYT | C | 402 | 18.105 | 24.471 | 98.613 | 1.00 | 56.35 | C |
| ATOM | 2286 | C1′ | CYT | C | 402 | 19.046 | 25.456 | 99.004 | 1.00 | 56.26 | C |
| ATOM | 2287 | N1 | CYT | C | 402 | 20.152 | 24.804 | 99.712 | 1.00 | 54.77 | C |
| ATOM | 2288 | C6 | CYT | C | 402 | 20.387 | 23.472 | 99.565 | 1.00 | 55.24 | C |
| ATOM | 2289 | C2 | CYT | C | 402 | 20.968 | 25.572 | 100.529 | 1.00 | 55.02 | C |
| ATOM | 2290 | O2 | CYT | C | 402 | 20.734 | 26.778 | 100.644 | 1.00 | 55.36 | C |
| ATOM | 2291 | N3 | CYT | C | 402 | 21.999 | 24.986 | 101.173 | 1.00 | 56.15 | C |
| ATOM | 2292 | C4 | CYT | C | 402 | 22.227 | 23.685 | 101.017 | 1.00 | 54.69 | C |
| ATOM | 2293 | N4 | CYT | C | 402 | 23.260 | 23.146 | 101.663 | 1.00 | 53.49 | C |
| ATOM | 2294 | C5 | CYT | C | 402 | 21.407 | 22.879 | 100.191 | 1.00 | 55.59 | C |
| ATOM | 2295 | C2′ | CYT | C | 402 | 19.544 | 26.136 | 97.733 | 1.00 | 57.33 | C |
| ATOM | 2296 | O2′ | CYT | C | 402 | 18.766 | 27.279 | 97.489 | 1.00 | 58.09 | C |
| ATOM | 2297 | C3′ | CYT | C | 402 | 19.362 | 25.031 | 96.697 | 1.00 | 57.73 | C |
| ATOM | 2298 | O3′ | CYT | C | 402 | 19.225 | 25.584 | 95.387 | 1.00 | 58.74 | C |
| ATOM | 2299 | P | GUA | C | 403 | 20.522 | 25.744 | 94.435 | 1.00 | 61.06 | C |
| ATOM | 2300 | O1P | GUA | C | 403 | 21.422 | 24.578 | 94.631 | 1.00 | 58.35 | C |
| ATOM | 2301 | O2P | GUA | C | 403 | 20.042 | 26.077 | 93.069 | 1.00 | 59.89 | C |
| ATOM | 2302 | O5′ | GUA | C | 403 | 21.291 | 27.014 | 95.018 | 1.00 | 60.71 | C |
| ATOM | 2303 | C5′ | GUA | C | 403 | 20.609 | 28.245 | 95.250 | 1.00 | 59.30 | C |
| ATOM | 2304 | C4′ | GUA | C | 403 | 21.497 | 29.205 | 96.019 | 1.00 | 58.89 | C |
| ATOM | 2305 | O4′ | GUA | C | 403 | 21.602 | 28.788 | 97.407 | 1.00 | 56.96 | C |
| ATOM | 2306 | C1′ | GUA | C | 403 | 22.882 | 29.133 | 97.907 | 1.00 | 57.03 | C |
| ATOM | 2307 | N9 | GUA | C | 403 | 23.580 | 27.909 | 98.280 | 1.00 | 56.30 | C |
| ATOM | 2308 | C4 | GUA | C | 403 | 24.724 | 27.815 | 99.036 | 1.00 | 56.42 | C |
| ATOM | 2309 | N3 | GUA | C | 403 | 25.397 | 28.845 | 99.594 | 1.00 | 57.34 | C |
| ATOM | 2310 | C2 | GUA | C | 403 | 26.467 | 28.435 | 100.254 | 1.00 | 57.44 | C |
| ATOM | 2311 | N2 | GUA | C | 403 | 27.242 | 29.308 | 100.887 | 1.00 | 57.79 | C |
| ATOM | 2312 | N1 | GUA | C | 403 | 26.852 | 27.129 | 100.346 | 1.00 | 58.64 | C |
| ATOM | 2313 | C6 | GUA | C | 403 | 26.177 | 26.054 | 99.782 | 1.00 | 58.04 | C |
| ATOM | 2314 | O6 | GUA | C | 403 | 26.616 | 24.907 | 99.929 | 1.00 | 59.05 | C |
| ATOM | 2315 | C5 | GUA | C | 403 | 25.020 | 26.474 | 99.083 | 1.00 | 56.53 | C |
| ATOM | 2316 | N7 | GUA | C | 403 | 24.074 | 25.736 | 98.390 | 1.00 | 56.87 | C |
| ATOM | 2317 | C8 | GUA | C | 403 | 23.240 | 26.628 | 97.934 | 1.00 | 56.90 | C |
| ATOM | 2318 | C2′ | GUA | C | 403 | 23.602 | 29.888 | 96.793 | 1.00 | 57.48 | C |
| ATOM | 2319 | O2′ | GUA | C | 403 | 23.309 | 31.256 | 96.911 | 1.00 | 58.00 | C |
| ATOM | 2320 | C3′ | GUA | C | 403 | 22.943 | 29.297 | 95.563 | 1.00 | 58.20 | C |
| ATOM | 2321 | O3′ | GUA | C | 403 | 23.064 | 30.206 | 94.483 | 1.00 | 59.54 | C |
| ATOM | 2322 | P | CYT | C | 404 | 24.252 | 30.020 | 93.414 | 1.00 | 62.17 | C |
| ATOM | 2323 | O1P | CYT | C | 404 | 24.328 | 28.585 | 93.021 | 1.00 | 60.43 | C |
| ATOM | 2324 | O2P | CYT | C | 404 | 24.071 | 31.055 | 92.367 | 1.00 | 60.24 | C |
| ATOM | 2325 | O5′ | CYT | C | 404 | 25.561 | 30.375 | 94.245 | 1.00 | 59.69 | C |
| ATOM | 2326 | C5′ | CYT | C | 404 | 25.812 | 31.702 | 94.657 | 1.00 | 58.57 | C |
| ATOM | 2327 | C4′ | CYT | C | 404 | 27.087 | 31.757 | 95.437 | 1.00 | 58.59 | C |
| ATOM | 2328 | O4′ | CYT | C | 404 | 26.953 | 30.922 | 96.612 | 1.00 | 58.87 | C |
| ATOM | 2329 | C1′ | CYT | C | 404 | 28.217 | 30.366 | 96.943 | 1.00 | 60.79 | C |
| ATOM | 2330 | N1 | CYT | C | 404 | 28.099 | 28.897 | 96.917 | 1.00 | 63.43 | C |
| ATOM | 2331 | C6 | CYT | C | 404 | 26.989 | 28.297 | 96.391 | 1.00 | 64.17 | C |
| ATOM | 2332 | C2 | CYT | C | 404 | 29.140 | 28.113 | 97.449 | 1.00 | 64.26 | C |
| ATOM | 2333 | O2 | CYT | C | 404 | 30.148 | 28.671 | 97.898 | 1.00 | 65.75 | C |
| ATOM | 2334 | N3 | CYT | C | 404 | 29.017 | 26.768 | 97.454 | 1.00 | 63.85 | C |
| ATOM | 2335 | C4 | CYT | C | 404 | 27.921 | 26.198 | 96.948 | 1.00 | 63.56 | C |
| ATOM | 2336 | N4 | CYT | C | 404 | 27.837 | 24.872 | 96.985 | 1.00 | 63.44 | C |
| ATOM | 2337 | C5 | CYT | C | 404 | 26.861 | 26.964 | 96.384 | 1.00 | 63.47 | C |
| ATOM | 2338 | C2′ | CYT | C | 404 | 29.213 | 30.917 | 95.922 | 1.00 | 60.30 | C |
| ATOM | 2339 | O2′ | CYT | C | 404 | 29.788 | 32.110 | 96.419 | 1.00 | 59.22 | C |
| ATOM | 2340 | C3′ | CYT | C | 404 | 28.298 | 31.176 | 94.737 | 1.00 | 59.12 | C |
| ATOM | 2341 | O3′ | CYT | C | 404 | 28.874 | 32.138 | 93.870 | 1.00 | 60.41 | C |
| ATOM | 2342 | P | GUA | C | 405 | 29.058 | 31.800 | 92.313 | 1.00 | 61.74 | C |
| ATOM | 2343 | O1P | GUA | C | 405 | 27.858 | 31.061 | 91.852 | 1.00 | 62.51 | C |
| ATOM | 2344 | O2P | GUA | C | 405 | 29.451 | 33.057 | 91.636 | 1.00 | 62.92 | C |
| ATOM | 2345 | O5′ | GUA | C | 405 | 30.300 | 30.809 | 92.261 | 1.00 | 59.83 | C |
| ATOM | 2346 | C5′ | GUA | C | 405 | 31.594 | 31.286 | 91.938 | 1.00 | 55.90 | C |
| ATOM | 2347 | C4′ | GUA | C | 405 | 32.500 | 31.127 | 93.120 | 1.00 | 55.47 | C |
| ATOM | 2348 | O4′ | GUA | C | 405 | 31.704 | 30.707 | 94.256 | 1.00 | 56.80 | C |
| ATOM | 2349 | C1′ | GUA | C | 405 | 32.410 | 29.728 | 95.004 | 1.00 | 57.76 | C |
| ATOM | 2350 | N9 | GUA | C | 405 | 31.719 | 28.450 | 94.860 | 1.00 | 58.84 | C |
| ATOM | 2351 | C4 | GUA | C | 405 | 32.102 | 27.246 | 95.395 | 1.00 | 58.87 | C |
| ATOM | 2352 | N3 | GUA | C | 405 | 33.195 | 27.026 | 96.142 | 1.00 | 59.93 | C |
| ATOM | 2353 | C2 | GUA | C | 405 | 33.291 | 25.767 | 96.511 | 1.00 | 59.79 | C |
| ATOM | 2354 | N2 | GUA | C | 405 | 34.328 | 25.368 | 97.258 | 1.00 | 59.82 | C |
| ATOM | 2355 | N1 | GUA | C | 405 | 32.381 | 24.806 | 96.176 | 1.00 | 59.90 | C |
| ATOM | 2356 | C6 | GUA | C | 405 | 31.255 | 25.014 | 95.400 | 1.00 | 59.29 | C |
| ATOM | 2357 | O6 | GUA | C | 405 | 30.510 | 24.076 | 95.143 | 1.00 | 61.07 | C |
| ATOM | 2358 | C5 | GUA | C | 405 | 31.142 | 26.349 | 95.000 | 1.00 | 59.13 | C |
| ATOM | 2359 | N7 | GUA | C | 405 | 30.178 | 26.969 | 94.225 | 1.00 | 60.31 | C |
| ATOM | 2360 | C8 | GUA | C | 405 | 30.562 | 28.213 | 94.168 | 1.00 | 60.29 | C |
| ATOM | 2361 | C2′ | GUA | C | 405 | 33.822 | 29.694 | 94.434 | 1.00 | 56.55 | C |
| ATOM | 2362 | O2′ | GUA | C | 405 | 34.605 | 30.659 | 95.099 | 1.00 | 56.61 | C |
| ATOM | 2363 | C3′ | GUA | C | 405 | 33.543 | 30.040 | 92.984 | 1.00 | 55.45 | C |
| ATOM | 2364 | O3′ | GUA | C | 405 | 34.691 | 30.557 | 92.361 | 1.00 | 55.55 | C |
| ATOM | 2365 | P | GUA | C | 406 | 35.407 | 29.710 | 91.213 | 1.00 | 59.15 | C |
| ATOM | 2366 | O1P | GUA | C | 406 | 34.348 | 28.968 | 90.477 | 1.00 | 58.47 | C |
| ATOM | 2367 | O2P | GUA | C | 406 | 36.287 | 30.642 | 90.475 | 1.00 | 59.19 | C |
| ATOM | 2368 | O5′ | GUA | C | 406 | 36.327 | 28.690 | 92.023 | 1.00 | 59.02 | C |
| ATOM | 2369 | C5′ | GUA | C | 406 | 37.191 | 29.170 | 93.049 | 1.00 | 60.68 | C |
| ATOM | 2370 | C4′ | GUA | C | 406 | 37.996 | 28.040 | 93.648 | 1.00 | 61.84 | C |
| ATOM | 2371 | O4′ | GUA | C | 406 | 37.229 | 27.387 | 94.701 | 1.00 | 63.42 | C |
| ATOM | 2372 | C1′ | GUA | C | 406 | 37.474 | 25.985 | 94.671 | 1.00 | 62.97 | C |
| ATOM | 2373 | N9 | GUA | C | 406 | 36.247 | 25.298 | 94.264 | 1.00 | 63.61 | C |
| ATOM | 2374 | C4 | GUA | C | 406 | 35.986 | 23.955 | 94.391 | 1.00 | 62.31 | C |
| ATOM | 2375 | N3 | GUA | C | 406 | 36.819 | 23.036 | 94.915 | 1.00 | 62.46 | C |
| ATOM | 2376 | C2 | GUA | C | 406 | 36.282 | 21.828 | 94.914 | 1.00 | 63.04 | C |
| ATOM | 2377 | N2 | GUA | C | 406 | 36.972 | 20.790 | 95.410 | 1.00 | 63.23 | C |
| ATOM | 2378 | N1 | GUA | C | 406 | 35.028 | 21.545 | 94.431 | 1.00 | 61.67 | C |
| ATOM | 2379 | C6 | GUA | C | 406 | 34.152 | 22.475 | 93.891 | 1.00 | 60.40 | C |
| ATOM | 2380 | O6 | GUA | C | 406 | 33.040 | 22.110 | 93.494 | 1.00 | 58.01 | C |
| ATOM | 2381 | C5 | GUA | C | 406 | 34.714 | 23.778 | 93.887 | 1.00 | 61.21 | C |
| ATOM | 2382 | N7 | GUA | C | 406 | 34.185 | 24.985 | 93.452 | 1.00 | 61.85 | C |
| ATOM | 2383 | C8 | GUA | C | 406 | 35.127 | 25.855 | 93.690 | 1.00 | 63.00 | C |
| ATOM | 2384 | C2′ | GUA | C | 406 | 38.643 | 25.785 | 93.709 | 1.00 | 61.67 | C |
| ATOM | 2385 | O2′ | GUA | C | 406 | 39.842 | 25.986 | 94.423 | 1.00 | 61.44 | C |
| ATOM | 2386 | C3′ | GUA | C | 406 | 38.391 | 26.908 | 92.714 | 1.00 | 61.32 | C |
| ATOM | 2387 | O3′ | GUA | C | 406 | 39.575 | 27.226 | 91.992 | 1.00 | 61.04 | C |
| ATOM | 2388 | P | CYT | C | 407 | 39.696 | 26.813 | 90.441 | 1.00 | 62.09 | C |
| ATOM | 2389 | O1P | CYT | C | 407 | 38.390 | 27.100 | 89.786 | 1.00 | 60.46 | C |
| ATOM | 2390 | O2P | CYT | C | 407 | 40.938 | 27.411 | 89.900 | 1.00 | 61.54 | C |
| ATOM | 2391 | O5′ | CYT | C | 407 | 39.905 | 25.237 | 90.475 | 1.00 | 62.16 | C |
| ATOM | 2392 | C5′ | CYT | C | 407 | 40.898 | 24.665 | 91.310 | 1.00 | 64.28 | C |
| ATOM | 2393 | C4′ | CYT | C | 407 | 40.632 | 23.197 | 91.503 | 1.00 | 65.55 | C |
| ATOM | 2394 | O4′ | CYT | C | 407 | 39.418 | 23.007 | 92.269 | 1.00 | 65.83 | C |
| ATOM | 2395 | C1′ | CYT | C | 407 | 38.826 | 21.770 | 91.907 | 1.00 | 65.87 | C |
| ATOM | 2396 | N1 | CYT | C | 407 | 37.451 | 22.028 | 91.465 | 1.00 | 65.12 | C |
| ATOM | 2397 | C6 | CYT | C | 407 | 37.117 | 23.212 | 90.878 | 1.00 | 65.22 | C |
| ATOM | 2398 | C2 | CYT | C | 407 | 36.490 | 21.042 | 91.653 | 1.00 | 64.19 | C |
| ATOM | 2399 | O2 | CYT | C | 407 | 36.823 | 19.981 | 92.188 | 1.00 | 64.69 | C |
| ATOM | 2400 | N3 | CYT | C | 407 | 35.225 | 21.267 | 91.252 | 1.00 | 63.31 | C |
| ATOM | 2401 | C4 | CYT | C | 407 | 34.906 | 22.425 | 90.680 | 1.00 | 63.47 | C |
| ATOM | 2402 | N4 | CYT | C | 407 | 33.645 | 22.608 | 90.301 | 1.00 | 63.66 | C |
| ATOM | 2403 | C5 | CYT | C | 407 | 35.866 | 23.449 | 90.473 | 1.00 | 64.34 | C |
| ATOM | 2404 | C2′ | CYT | C | 407 | 39.701 | 21.163 | 90.810 | 1.00 | 67.10 | C |
| ATOM | 2405 | O2′ | CYT | C | 407 | 40.644 | 20.291 | 91.403 | 1.00 | 68.84 | C |
| ATOM | 2406 | C3′ | CYT | C | 407 | 40.360 | 22.410 | 90.238 | 1.00 | 66.94 | C |
| ATOM | 2407 | O3′ | CYT | C | 407 | 41.586 | 22.095 | 89.599 | 1.00 | 68.23 | C |
| ATOM | 2408 | P | GUA | C | 408 | 41.689 | 22.166 | 88.002 | 1.00 | 70.32 | C |
| ATOM | 2409 | O1P | GUA | C | 408 | 40.649 | 23.080 | 87.472 | 1.00 | 68.95 | C |
| ATOM | 2410 | O2P | GUA | C | 408 | 43.111 | 22.392 | 87.664 | 1.00 | 70.47 | C |
| ATOM | 2411 | O5′ | GUA | C | 408 | 41.345 | 20.690 | 87.551 | 1.00 | 70.99 | C |
| ATOM | 2412 | C5′ | GUA | C | 408 | 42.147 | 19.625 | 88.008 | 1.00 | 73.91 | C |
| ATOM | 2413 | C4′ | GUA | C | 408 | 41.779 | 18.368 | 87.287 | 1.00 | 75.11 | C |
| ATOM | 2414 | O4′ | GUA | C | 408 | 40.379 | 18.115 | 87.519 | 1.00 | 75.66 | C |
| ATOM | 2415 | C1′ | GUA | C | 408 | 39.857 | 17.389 | 86.431 | 1.00 | 74.76 | C |
| ATOM | 2416 | N9 | GUA | C | 408 | 38.630 | 18.047 | 85.997 | 1.00 | 72.57 | C |
| ATOM | 2417 | C4 | GUA | C | 408 | 37.432 | 17.423 | 85.774 | 1.00 | 71.47 | C |
| ATOM | 2418 | N3 | GUA | C | 408 | 37.199 | 16.098 | 85.901 | 1.00 | 70.90 | C |
| ATOM | 2419 | C2 | GUA | C | 408 | 35.948 | 15.786 | 85.611 | 1.00 | 70.55 | C |
| ATOM | 2420 | N2 | GUA | C | 408 | 35.556 | 14.509 | 85.662 | 1.00 | 69.17 | C |
| ATOM | 2421 | N1 | GUA | C | 408 | 34.994 | 16.703 | 85.241 | 1.00 | 70.87 | C |
| ATOM | 2422 | C6 | GUA | C | 408 | 35.208 | 18.072 | 85.110 | 1.00 | 71.62 | C |
| ATOM | 2423 | O6 | GUA | C | 408 | 34.271 | 18.811 | 84.776 | 1.00 | 73.46 | C |
| ATOM | 2424 | C5 | GUA | C | 408 | 36.559 | 18.423 | 85.405 | 1.00 | 71.18 | C |
| ATOM | 2425 | N7 | GUA | C | 408 | 37.200 | 19.656 | 85.391 | 1.00 | 70.33 | C |
| ATOM | 2426 | C8 | GUA | C | 408 | 38.427 | 19.384 | 85.746 | 1.00 | 71.47 | C |
| ATOM | 2427 | C2′ | GUA | C | 408 | 40.966 | 17.271 | 85.378 | 1.00 | 76.55 | C |
| ATOM | 2428 | O2′ | GUA | C | 408 | 41.615 | 16.012 | 85.508 | 1.00 | 76.60 | C |
| ATOM | 2429 | C3′ | GUA | C | 408 | 41.889 | 18.415 | 85.774 | 1.00 | 75.72 | C |
| ATOM | 2430 | O3′ | GUA | C | 408 | 43.239 | 18.127 | 85.411 | 1.00 | 77.71 | C |
| ATOM | 2431 | P | ADE | C | 409 | 43.631 | 17.905 | 83.865 | 1.00 | 78.05 | C |
| ATOM | 2432 | O1P | ADE | C | 409 | 45.116 | 17.841 | 83.822 | 1.00 | 77.62 | C |
| ATOM | 2433 | O2P | ADE | C | 409 | 42.909 | 18.918 | 83.043 | 1.00 | 77.11 | C |
| ATOM | 2434 | O5′ | ADE | C | 409 | 43.084 | 16.445 | 83.528 | 1.00 | 75.49 | C |
| ATOM | 2435 | C5′ | ADE | C | 409 | 42.285 | 16.201 | 82.374 | 1.00 | 71.72 | C |
| ATOM | 2436 | C4′ | ADE | C | 409 | 41.693 | 14.813 | 82.439 | 1.00 | 70.03 | C |
| ATOM | 2437 | O4′ | ADE | C | 409 | 40.734 | 14.749 | 83.535 | 1.00 | 68.77 | C |
| ATOM | 2438 | C1′ | ADE | C | 409 | 39.546 | 14.109 | 83.095 | 1.00 | 67.15 | C |
| ATOM | 2439 | N9 | ADE | C | 409 | 38.538 | 15.134 | 82.827 | 1.00 | 63.77 | C |
| ATOM | 2440 | C4 | ADE | C | 409 | 37.201 | 14.910 | 82.610 | 1.00 | 61.48 | C |
| ATOM | 2441 | N3 | ADE | C | 409 | 36.566 | 13.727 | 82.597 | 1.00 | 60.86 | C |
| ATOM | 2442 | C2 | ADE | C | 409 | 35.272 | 13.896 | 82.350 | 1.00 | 60.34 | C |
| ATOM | 2443 | N1 | ADE | C | 409 | 34.590 | 15.026 | 82.133 | 1.00 | 59.77 | C |
| ATOM | 2444 | C6 | ADE | C | 409 | 35.261 | 16.196 | 82.150 | 1.00 | 60.33 | C |
| ATOM | 2445 | N6 | ADE | C | 409 | 34.584 | 17.323 | 81.924 | 1.00 | 60.45 | C |
| ATOM | 2446 | C5 | ADE | C | 409 | 36.640 | 16.153 | 82.402 | 1.00 | 60.68 | C |
| ATOM | 2447 | N7 | ADE | C | 409 | 37.604 | 17.146 | 82.484 | 1.00 | 61.47 | C |
| ATOM | 2448 | C8 | ADE | C | 409 | 38.710 | 16.490 | 82.735 | 1.00 | 62.04 | C |
| ATOM | 2449 | C2′ | ADE | C | 409 | 39.935 | 13.373 | 81.821 | 1.00 | 68.85 | C |
| ATOM | 2450 | O2′ | ADE | C | 409 | 40.547 | 12.152 | 82.173 | 1.00 | 69.62 | C |
| ATOM | 2451 | C3′ | ADE | C | 409 | 40.900 | 14.379 | 81.216 | 1.00 | 69.51 | C |
| ATOM | 2452 | O3′ | ADE | C | 409 | 41.738 | 13.788 | 80.238 | 1.00 | 69.60 | C |
| ATOM | 2453 | P | URI | C | 410 | 41.499 | 14.124 | 78.685 | 1.00 | 71.45 | C |
| ATOM | 2454 | O1P | URI | C | 410 | 41.183 | 15.569 | 78.541 | 1.00 | 69.04 | C |
| ATOM | 2455 | O2P | URI | C | 410 | 42.648 | 13.542 | 77.940 | 1.00 | 70.39 | C |
| ATOM | 2456 | O5′ | URI | C | 410 | 40.172 | 13.320 | 78.324 | 1.00 | 70.02 | C |
| ATOM | 2457 | C5′ | URI | C | 410 | 40.097 | 11.929 | 78.575 | 1.00 | 70.01 | C |
| ATOM | 2458 | C4′ | URI | C | 410 | 38.670 | 11.458 | 78.515 | 1.00 | 69.54 | C |
| ATOM | 2459 | O4′ | URI | C | 410 | 37.849 | 12.157 | 79.484 | 1.00 | 69.30 | C |
| ATOM | 2460 | C1′ | URI | C | 410 | 36.490 | 12.014 | 79.104 | 1.00 | 69.48 | C |
| ATOM | 2461 | N1 | URI | C | 410 | 35.864 | 13.337 | 79.046 | 1.00 | 68.66 | C |
| ATOM | 2462 | C6 | URI | C | 410 | 36.592 | 14.498 | 79.080 | 1.00 | 68.60 | C |
| ATOM | 2463 | C2 | URI | C | 410 | 34.498 | 13.359 | 78.915 | 1.00 | 69.40 | C |
| ATOM | 2464 | O2 | URI | C | 410 | 33.828 | 12.342 | 78.918 | 1.00 | 69.84 | C |
| ATOM | 2465 | N3 | URI | C | 410 | 33.944 | 14.606 | 78.776 | 1.00 | 69.35 | C |
| ATOM | 2466 | C4 | URI | C | 410 | 34.610 | 15.808 | 78.765 | 1.00 | 69.40 | C |
| ATOM | 2467 | O4 | URI | C | 410 | 33.980 | 16.842 | 78.552 | 1.00 | 70.68 | C |
| ATOM | 2468 | C5 | URI | C | 410 | 36.028 | 15.705 | 78.944 | 1.00 | 68.74 | C |
| ATOM | 2469 | C2′ | URI | C | 410 | 36.492 | 11.412 | 77.694 | 1.00 | 70.35 | C |
| ATOM | 2470 | O2′ | URI | C | 410 | 36.224 | 10.025 | 77.741 | 1.00 | 72.52 | C |
| ATOM | 2471 | C3′ | URI | C | 410 | 37.907 | 11.736 | 77.240 | 1.00 | 68.80 | C |
| ATOM | 2472 | O3′ | URI | C | 410 | 38.307 | 10.895 | 76.171 | 1.00 | 66.35 | C |
| ATOM | 2473 | P | URI | C | 411 | 37.802 | 11.224 | 74.683 | 1.00 | 66.37 | C |
| ATOM | 2474 | O1P | URI | C | 411 | 38.216 | 12.601 | 74.317 | 1.00 | 65.70 | C |
| ATOM | 2475 | O2P | URI | C | 411 | 38.191 | 10.095 | 73.816 | 1.00 | 67.37 | C |
| ATOM | 2476 | O5′ | URI | C | 411 | 36.216 | 11.211 | 74.803 | 1.00 | 66.35 | C |
| ATOM | 2477 | C5′ | URI | C | 411 | 35.502 | 9.992 | 74.705 | 1.00 | 65.04 | C |
| ATOM | 2478 | C4′ | URI | C | 411 | 34.034 | 10.229 | 74.921 | 1.00 | 65.29 | C |
| ATOM | 2479 | O4′ | URI | C | 411 | 33.822 | 11.266 | 75.909 | 1.00 | 64.72 | C |
| ATOM | 2480 | C1′ | URI | C | 411 | 32.551 | 11.861 | 75.680 | 1.00 | 64.83 | C |
| ATOM | 2481 | N1 | URI | C | 411 | 32.744 | 13.300 | 75.497 | 1.00 | 64.62 | C |
| ATOM | 2482 | C6 | URI | C | 411 | 33.986 | 13.869 | 75.569 | 1.00 | 65.33 | C |
| ATOM | 2483 | C2 | URI | C | 411 | 31.626 | 14.058 | 75.254 | 1.00 | 64.72 | C |
| ATOM | 2484 | O2 | URI | C | 411 | 30.517 | 13.575 | 75.169 | 1.00 | 65.12 | C |
| ATOM | 2485 | N3 | URI | C | 411 | 31.857 | 15.403 | 75.112 | 1.00 | 65.38 | C |
| ATOM | 2486 | C4 | URI | C | 411 | 33.077 | 16.046 | 75.186 | 1.00 | 66.26 | C |
| ATOM | 2487 | O4 | URI | C | 411 | 33.127 | 17.274 | 75.088 | 1.00 | 67.41 | C |
| ATOM | 2488 | C5 | URI | C | 411 | 34.188 | 15.182 | 75.425 | 1.00 | 65.97 | C |
| ATOM | 2489 | C2′ | URI | C | 411 | 31.982 | 11.214 | 74.419 | 1.00 | 65.19 | C |
| ATOM | 2490 | O2′ | URI | C | 411 | 31.146 | 10.139 | 74.799 | 1.00 | 65.30 | C |
| ATOM | 2491 | C3′ | URI | C | 411 | 33.264 | 10.769 | 73.737 | 1.00 | 65.47 | C |
| ATOM | 2492 | O3′ | URI | C | 411 | 33.036 | 9.746 | 72.786 | 1.00 | 67.90 | C |
| ATOM | 2493 | P | URI | C | 412 | 33.458 | 9.981 | 71.256 | 1.00 | 69.75 | C |
| ATOM | 2494 | O1P | URI | C | 412 | 34.775 | 10.668 | 71.271 | 1.00 | 67.86 | C |
| ATOM | 2495 | O2P | URI | C | 412 | 33.316 | 8.688 | 70.542 | 1.00 | 69.18 | C |
| ATOM | 2496 | O5′ | URI | C | 412 | 32.325 | 10.965 | 70.717 | 1.00 | 69.11 | C |
| ATOM | 2497 | C5′ | URI | C | 412 | 30.975 | 10.520 | 70.646 | 1.00 | 67.01 | C |
| ATOM | 2498 | C4′ | URI | C | 412 | 30.031 | 11.686 | 70.489 | 1.00 | 66.73 | C |
| ATOM | 2499 | O4′ | URI | C | 412 | 30.184 | 12.623 | 71.575 | 1.00 | 65.01 | C |
| ATOM | 2500 | C1′ | URI | C | 412 | 29.687 | 13.882 | 71.158 | 1.00 | 63.86 | C |
| ATOM | 2501 | N1 | URI | C | 412 | 30.717 | 14.892 | 71.399 | 1.00 | 62.20 | C |
| ATOM | 2502 | C6 | URI | C | 412 | 32.002 | 14.558 | 71.731 | 1.00 | 61.49 | C |
| ATOM | 2503 | C2 | URI | C | 412 | 30.332 | 16.196 | 71.274 | 1.00 | 61.27 | C |
| ATOM | 2504 | O2 | URI | C | 412 | 29.202 | 16.515 | 70.962 | 1.00 | 62.77 | C |
| ATOM | 2505 | N3 | URI | C | 412 | 31.312 | 17.115 | 71.518 | 1.00 | 59.78 | C |
| ATOM | 2506 | C4 | URI | C | 412 | 32.611 | 16.861 | 71.861 | 1.00 | 59.79 | C |
| ATOM | 2507 | O4 | URI | C | 412 | 33.374 | 17.804 | 72.049 | 1.00 | 59.40 | C |
| ATOM | 2508 | C5 | URI | C | 412 | 32.941 | 15.475 | 71.962 | 1.00 | 60.45 | C |
| ATOM | 2509 | C2′ | URI | C | 412 | 29.380 | 13.779 | 69.665 | 1.00 | 66.11 | C |
| ATOM | 2510 | O2′ | URI | C | 412 | 27.995 | 13.585 | 69.466 | 1.00 | 67.05 | C |
| ATOM | 2511 | C3′ | URI | C | 412 | 30.221 | 12.574 | 69.276 | 1.00 | 67.25 | C |
| ATOM | 2512 | O3′ | URI | C | 412 | 29.704 | 11.954 | 68.111 | 1.00 | 71.00 | C |
| ATOM | 2513 | P | ADE | C | 413 | 30.718 | 11.338 | 67.033 | 1.00 | 73.23 | C |
| ATOM | 2514 | O1P | ADE | C | 413 | 30.203 | 10.003 | 66.618 | 1.00 | 73.04 | C |
| ATOM | 2515 | O2P | ADE | C | 413 | 32.098 | 11.461 | 67.573 | 1.00 | 72.20 | C |
| ATOM | 2516 | O5′ | ADE | C | 413 | 30.585 | 12.334 | 65.810 | 1.00 | 73.28 | C |
| ATOM | 2517 | C5′ | ADE | C | 413 | 30.645 | 13.727 | 66.017 | 1.00 | 73.61 | C |
| ATOM | 2518 | C4′ | ADE | C | 413 | 29.751 | 14.418 | 65.038 | 1.00 | 74.87 | C |
| ATOM | 2519 | O4′ | ADE | C | 413 | 29.847 | 15.831 | 65.290 | 1.00 | 75.30 | C |
| ATOM | 2520 | C1′ | ADE | C | 413 | 30.475 | 16.458 | 64.205 | 1.00 | 75.52 | C |
| ATOM | 2521 | N9 | ADE | C | 413 | 31.805 | 16.884 | 64.637 | 1.00 | 72.95 | C |
| ATOM | 2522 | C4 | ADE | C | 413 | 32.223 | 18.189 | 64.754 | 1.00 | 71.28 | C |
| ATOM | 2523 | N3 | ADE | C | 413 | 31.532 | 19.298 | 64.439 | 1.00 | 69.94 | C |
| ATOM | 2524 | C2 | ADE | C | 413 | 32.242 | 20.389 | 64.724 | 1.00 | 70.10 | C |
| ATOM | 2525 | N1 | ADE | C | 413 | 33.467 | 20.488 | 65.256 | 1.00 | 69.99 | C |
| ATOM | 2526 | C6 | ADE | C | 413 | 34.128 | 19.351 | 65.564 | 1.00 | 69.99 | C |
| ATOM | 2527 | N6 | ADE | C | 413 | 35.337 | 19.444 | 66.113 | 1.00 | 69.89 | C |
| ATOM | 2528 | C5 | ADE | C | 413 | 33.492 | 18.130 | 65.296 | 1.00 | 70.40 | C |
| ATOM | 2529 | N7 | ADE | C | 413 | 33.887 | 16.814 | 65.474 | 1.00 | 70.94 | C |
| ATOM | 2530 | C8 | ADE | C | 413 | 32.857 | 16.116 | 65.058 | 1.00 | 72.68 | C |
| ATOM | 2531 | C2′ | ADE | C | 413 | 30.364 | 15.552 | 62.973 | 1.00 | 76.97 | C |
| ATOM | 2532 | O2′ | ADE | C | 413 | 29.313 | 16.035 | 62.146 | 1.00 | 80.11 | C |
| ATOM | 2533 | C3′ | ADE | C | 413 | 30.064 | 14.164 | 63.561 | 1.00 | 75.75 | C |
| ATOM | 2534 | O3′ | ADE | C | 413 | 28.930 | 13.511 | 62.951 | 1.00 | 75.06 | C |
| ATOM | 2535 | P | ADE | C | 414 | 28.765 | 13.461 | 61.338 | 1.00 | 76.96 | C |
| ATOM | 2536 | O1P | ADE | C | 414 | 27.335 | 13.728 | 61.042 | 1.00 | 76.26 | C |
| ATOM | 2537 | O2P | ADE | C | 414 | 29.811 | 14.270 | 60.658 | 1.00 | 78.09 | C |
| ATOM | 2538 | O5′ | ADE | C | 414 | 29.092 | 11.954 | 60.949 | 1.00 | 74.22 | C |
| ATOM | 2539 | C5′ | ADE | C | 414 | 30.427 | 11.471 | 61.047 | 1.00 | 70.82 | C |
| ATOM | 2540 | C4′ | ADE | C | 414 | 30.851 | 10.780 | 59.771 | 1.00 | 66.77 | C |
| ATOM | 2541 | O4′ | ADE | C | 414 | 29.773 | 9.960 | 59.251 | 1.00 | 65.37 | C |
| ATOM | 2542 | C1′ | ADE | C | 414 | 29.876 | 9.887 | 57.841 | 1.00 | 63.05 | C |
| ATOM | 2543 | N9 | ADE | C | 414 | 28.710 | 10.543 | 57.255 | 1.00 | 61.54 | C |
| ATOM | 2544 | C4 | ADE | C | 414 | 28.003 | 10.100 | 56.168 | 1.00 | 59.49 | C |
| ATOM | 2545 | N3 | ADE | C | 414 | 28.217 | 8.979 | 55.466 | 1.00 | 58.27 | C |
| ATOM | 2546 | C2 | ADE | C | 414 | 27.363 | 8.889 | 54.459 | 1.00 | 58.36 | C |
| ATOM | 2547 | N1 | ADE | C | 414 | 26.395 | 9.731 | 54.090 | 1.00 | 60.36 | C |
| ATOM | 2548 | C6 | ADE | C | 414 | 26.211 | 10.858 | 54.810 | 1.00 | 61.88 | C |
| ATOM | 2549 | N6 | ADE | C | 414 | 25.261 | 11.719 | 54.421 | 1.00 | 62.50 | C |
| ATOM | 2550 | C5 | ADE | C | 414 | 27.045 | 11.062 | 55.922 | 1.00 | 60.84 | C |
| ATOM | 2551 | N7 | ADE | C | 414 | 27.123 | 12.083 | 56.858 | 1.00 | 61.16 | C |
| ATOM | 2552 | C8 | ADE | C | 414 | 28.119 | 11.723 | 57.630 | 1.00 | 61.66 | C |
| ATOM | 2553 | C2′ | ADE | C | 414 | 31.169 | 10.612 | 57.475 | 1.00 | 63.77 | C |
| ATOM | 2554 | O2′ | ADE | C | 414 | 32.225 | 9.683 | 57.550 | 1.00 | 63.04 | C |
| ATOM | 2555 | C3′ | ADE | C | 414 | 31.237 | 11.646 | 58.587 | 1.00 | 64.95 | C |
| ATOM | 2556 | O3′ | ADE | C | 414 | 32.562 | 12.156 | 58.741 | 1.00 | 65.26 | C |
| ATOM | 2557 | P | CYT | C | 415 | 33.103 | 13.307 | 57.750 | 1.00 | 64.49 | C |
| ATOM | 2558 | O1P | CYT | C | 415 | 32.556 | 13.058 | 56.397 | 1.00 | 64.22 | C |
| ATOM | 2559 | O2P | CYT | C | 415 | 32.929 | 14.645 | 58.378 | 1.00 | 63.51 | C |
| ATOM | 2560 | O5′ | CYT | C | 415 | 34.657 | 13.010 | 57.669 | 1.00 | 63.60 | C |
| ATOM | 2561 | C5′ | CYT | C | 415 | 35.129 | 11.714 | 57.340 | 1.00 | 61.44 | C |
| ATOM | 2562 | C4′ | CYT | C | 415 | 36.619 | 11.758 | 57.148 | 1.00 | 60.98 | C |
| ATOM | 2563 | O4′ | CYT | C | 415 | 36.914 | 12.600 | 55.995 | 1.00 | 61.26 | C |
| ATOM | 2564 | C1′ | CYT | C | 415 | 38.068 | 13.381 | 56.256 | 1.00 | 58.35 | C |
| ATOM | 2565 | N1 | CYT | C | 415 | 37.666 | 14.787 | 56.333 | 1.00 | 57.21 | C |
| ATOM | 2566 | C6 | CYT | C | 415 | 36.358 | 15.155 | 56.196 | 1.00 | 57.26 | C |
| ATOM | 2567 | C2 | CYT | C | 415 | 38.647 | 15.747 | 56.549 | 1.00 | 57.02 | C |
| ATOM | 2568 | O2 | CYT | C | 415 | 39.823 | 15.383 | 56.668 | 1.00 | 59.07 | C |
| ATOM | 2569 | N3 | CYT | C | 415 | 38.298 | 17.046 | 56.625 | 1.00 | 55.70 | C |
| ATOM | 2570 | C4 | CYT | C | 415 | 37.024 | 17.399 | 56.496 | 1.00 | 55.51 | C |
| ATOM | 2571 | N4 | CYT | C | 415 | 36.730 | 18.693 | 56.588 | 1.00 | 56.00 | C |
| ATOM | 2572 | C5 | CYT | C | 415 | 35.995 | 16.440 | 56.270 | 1.00 | 56.63 | C |
| ATOM | 2573 | C2′ | CYT | C | 415 | 38.651 | 12.861 | 57.566 | 1.00 | 58.28 | C |
| ATOM | 2574 | O2′ | CYT | C | 415 | 39.501 | 11.771 | 57.275 | 1.00 | 58.67 | C |
| ATOM | 2575 | C3′ | CYT | C | 415 | 37.386 | 12.423 | 58.280 | 1.00 | 59.17 | C |
| ATOM | 2576 | O3′ | CYT | C | 415 | 37.659 | 11.498 | 59.322 | 1.00 | 57.74 | C |
| ATOM | 2577 | P | CYT | C | 416 | 37.493 | 11.963 | 60.851 | 1.00 | 59.52 | C |
| ATOM | 2578 | O1P | CYT | C | 416 | 36.259 | 12.797 | 60.912 | 1.00 | 58.54 | C |
| ATOM | 2579 | O2P | CYT | C | 416 | 37.627 | 10.769 | 61.730 | 1.00 | 54.71 | C |
| ATOM | 2580 | O5′ | CYT | C | 416 | 38.731 | 12.941 | 61.096 | 1.00 | 58.54 | C |
| ATOM | 2581 | C5′ | CYT | C | 416 | 40.063 | 12.480 | 60.905 | 1.00 | 56.26 | C |
| ATOM | 2582 | C4′ | CYT | C | 416 | 41.046 | 13.602 | 61.094 | 1.00 | 53.74 | C |
| ATOM | 2583 | O4′ | CYT | C | 416 | 40.937 | 14.552 | 60.009 | 1.00 | 54.50 | C |
| ATOM | 2584 | C1′ | CYT | C | 416 | 41.313 | 15.837 | 60.466 | 1.00 | 52.76 | C |
| ATOM | 2585 | N1 | CYT | C | 416 | 40.191 | 16.738 | 60.245 | 1.00 | 52.47 | C |
| ATOM | 2586 | C6 | CYT | C | 416 | 38.959 | 16.263 | 59.895 | 1.00 | 53.65 | C |
| ATOM | 2587 | C2 | CYT | C | 416 | 40.398 | 18.085 | 60.416 | 1.00 | 52.84 | C |
| ATOM | 2588 | O2 | CYT | C | 416 | 41.525 | 18.463 | 60.740 | 1.00 | 53.51 | C |
| ATOM | 2589 | N3 | CYT | C | 416 | 39.366 | 18.949 | 60.236 | 1.00 | 53.73 | C |
| ATOM | 2590 | C4 | CYT | C | 416 | 38.160 | 18.482 | 59.906 | 1.00 | 53.74 | C |
| ATOM | 2591 | N4 | CYT | C | 416 | 37.164 | 19.358 | 59.753 | 1.00 | 53.22 | C |
| ATOM | 2592 | C5 | CYT | C | 416 | 37.921 | 17.094 | 59.721 | 1.00 | 54.01 | C |
| ATOM | 2593 | C2′ | CYT | C | 416 | 41.598 | 15.715 | 61.962 | 1.00 | 53.86 | C |
| ATOM | 2594 | O2′ | CYT | C | 416 | 42.981 | 15.591 | 62.179 | 1.00 | 56.70 | C |
| ATOM | 2595 | C3′ | CYT | C | 416 | 40.826 | 14.451 | 62.322 | 1.00 | 54.09 | C |
| ATOM | 2596 | O3′ | CYT | C | 416 | 41.376 | 13.796 | 63.453 | 1.00 | 52.07 | C |
| ATOM | 2597 | P | GUA | C | 417 | 40.666 | 13.954 | 64.877 | 1.00 | 50.57 | C |
| ATOM | 2598 | O1P | GUA | C | 417 | 39.279 | 13.475 | 64.689 | 1.00 | 50.99 | C |
| ATOM | 2599 | O2P | GUA | C | 417 | 41.515 | 13.315 | 65.909 | 1.00 | 49.71 | C |
| ATOM | 2600 | O5′ | GUA | C | 417 | 40.676 | 15.533 | 65.098 | 1.00 | 49.38 | C |
| ATOM | 2601 | C5′ | GUA | C | 417 | 41.879 | 16.271 | 64.953 | 1.00 | 51.97 | C |
| ATOM | 2602 | C4′ | GUA | C | 417 | 41.622 | 17.748 | 65.133 | 1.00 | 57.26 | C |
| ATOM | 2603 | O4′ | GUA | C | 417 | 41.047 | 18.318 | 63.925 | 1.00 | 57.83 | C |
| ATOM | 2604 | C1′ | GUA | C | 417 | 40.017 | 19.231 | 64.270 | 1.00 | 59.99 | C |
| ATOM | 2605 | N9 | GUA | C | 417 | 38.746 | 18.618 | 63.896 | 1.00 | 61.24 | C |
| ATOM | 2606 | C4 | GUA | C | 417 | 37.612 | 19.249 | 63.444 | 1.00 | 59.92 | C |
| ATOM | 2607 | N3 | GUA | C | 417 | 37.455 | 20.576 | 63.273 | 1.00 | 59.88 | C |
| ATOM | 2608 | C2 | GUA | C | 417 | 36.240 | 20.875 | 62.829 | 1.00 | 61.24 | C |
| ATOM | 2609 | N2 | GUA | C | 417 | 35.894 | 22.158 | 62.613 | 1.00 | 59.68 | C |
| ATOM | 2610 | N1 | GUA | C | 417 | 35.268 | 19.939 | 62.568 | 1.00 | 61.42 | C |
| ATOM | 2611 | C6 | GUA | C | 417 | 35.416 | 18.565 | 62.736 | 1.00 | 62.16 | C |
| ATOM | 2612 | O6 | GUA | C | 417 | 34.475 | 17.800 | 62.475 | 1.00 | 63.58 | C |
| ATOM | 2613 | C5 | GUA | C | 417 | 36.703 | 18.237 | 63.214 | 1.00 | 61.70 | C |
| ATOM | 2614 | N7 | GUA | C | 417 | 37.250 | 16.999 | 63.519 | 1.00 | 61.89 | C |
| ATOM | 2615 | C8 | GUA | C | 417 | 38.457 | 17.272 | 63.921 | 1.00 | 62.05 | C |
| ATOM | 2616 | C2′ | GUA | C | 417 | 40.144 | 19.478 | 65.776 | 1.00 | 60.59 | C |
| ATOM | 2617 | O2′ | GUA | C | 417 | 41.060 | 20.524 | 66.020 | 1.00 | 60.70 | C |
| ATOM | 2618 | C3′ | GUA | C | 417 | 40.675 | 18.134 | 66.259 | 1.00 | 60.19 | C |
| ATOM | 2619 | O3′ | GUA | C | 417 | 41.418 | 18.282 | 67.467 | 1.00 | 63.36 | C |
| ATOM | 2620 | P | URI | C | 418 | 40.649 | 18.448 | 68.868 | 1.00 | 64.75 | C |
| ATOM | 2621 | O1P | URI | C | 418 | 39.195 | 18.308 | 68.586 | 1.00 | 64.68 | C |
| ATOM | 2622 | O2P | URI | C | 418 | 41.292 | 17.551 | 69.870 | 1.00 | 60.42 | C |
| ATOM | 2623 | O5′ | URI | C | 418 | 40.941 | 19.967 | 69.252 | 1.00 | 63.59 | C |
| ATOM | 2624 | C5′ | URI | C | 418 | 42.277 | 20.447 | 69.381 | 1.00 | 63.57 | C |
| ATOM | 2625 | C4′ | URI | C | 418 | 42.293 | 21.952 | 69.332 | 1.00 | 64.03 | C |
| ATOM | 2626 | O4′ | URI | C | 418 | 41.790 | 22.373 | 68.040 | 1.00 | 64.53 | C |
| ATOM | 2627 | C1′ | URI | C | 418 | 41.029 | 23.558 | 68.183 | 1.00 | 64.27 | C |
| ATOM | 2628 | N1 | URI | C | 418 | 39.654 | 23.276 | 67.757 | 1.00 | 64.25 | C |
| ATOM | 2629 | C6 | URI | C | 418 | 39.136 | 21.998 | 67.775 | 1.00 | 64.17 | C |
| ATOM | 2630 | C2 | URI | C | 418 | 38.900 | 24.342 | 67.338 | 1.00 | 63.52 | C |
| ATOM | 2631 | O2 | URI | C | 418 | 39.319 | 25.477 | 67.342 | 1.00 | 63.70 | C |
| ATOM | 2632 | N3 | URI | C | 418 | 37.630 | 24.032 | 66.922 | 1.00 | 63.65 | C |
| ATOM | 2633 | C4 | URI | C | 418 | 37.048 | 22.782 | 66.896 | 1.00 | 63.02 | C |
| ATOM | 2634 | O4 | URI | C | 418 | 35.904 | 22.659 | 66.450 | 1.00 | 62.11 | C |
| ATOM | 2635 | C5 | URI | C | 418 | 37.892 | 21.721 | 67.372 | 1.00 | 63.38 | C |
| ATOM | 2636 | C2′ | URI | C | 418 | 41.084 | 23.960 | 69.651 | 1.00 | 65.55 | C |
| ATOM | 2637 | O2′ | URI | C | 418 | 42.113 | 24.892 | 69.868 | 1.00 | 67.22 | C |
| ATOM | 2638 | C3′ | URI | C | 418 | 41.362 | 22.625 | 70.320 | 1.00 | 66.18 | C |
| ATOM | 2639 | O3′ | URI | C | 418 | 42.000 | 22.835 | 71.564 | 1.00 | 68.22 | C |
| ATOM | 2640 | P | ADE | C | 419 | 41.108 | 23.135 | 72.859 | 1.00 | 69.94 | C |
| ATOM | 2641 | O1P | ADE | C | 419 | 42.038 | 23.210 | 74.017 | 1.00 | 69.40 | C |
| ATOM | 2642 | O2P | ADE | C | 419 | 39.974 | 22.169 | 72.868 | 1.00 | 68.19 | C |
| ATOM | 2643 | O5′ | ADE | C | 419 | 40.535 | 24.595 | 72.609 | 1.00 | 68.67 | C |
| ATOM | 2644 | C5′ | ADE | C | 419 | 41.334 | 25.731 | 72.889 | 1.00 | 69.84 | C |
| ATOM | 2645 | C4′ | ADE | C | 419 | 40.592 | 26.984 | 72.518 | 1.00 | 71.36 | C |
| ATOM | 2646 | O4′ | ADE | C | 419 | 39.995 | 26.802 | 71.212 | 1.00 | 71.02 | C |
| ATOM | 2647 | C1′ | ADE | C | 419 | 38.859 | 27.640 | 71.099 | 1.00 | 71.24 | C |
| ATOM | 2648 | N9 | ADE | C | 419 | 37.749 | 26.831 | 70.600 | 1.00 | 71.50 | C |
| ATOM | 2649 | C4 | ADE | C | 419 | 36.570 | 27.309 | 70.086 | 1.00 | 71.60 | C |
| ATOM | 2650 | N3 | ADE | C | 419 | 36.211 | 28.596 | 69.936 | 1.00 | 71.24 | C |
| ATOM | 2651 | C2 | ADE | C | 419 | 34.998 | 28.680 | 69.398 | 1.00 | 71.83 | C |
| ATOM | 2652 | N1 | ADE | C | 419 | 34.163 | 27.698 | 69.022 | 1.00 | 71.68 | C |
| ATOM | 2653 | C6 | ADE | C | 419 | 34.554 | 26.417 | 69.193 | 1.00 | 71.81 | C |
| ATOM | 2654 | N6 | ADE | C | 419 | 33.719 | 25.440 | 68.829 | 1.00 | 72.19 | C |
| ATOM | 2655 | C5 | ADE | C | 419 | 35.826 | 26.192 | 69.751 | 1.00 | 71.46 | C |
| ATOM | 2656 | N7 | ADE | C | 419 | 36.525 | 25.031 | 70.047 | 1.00 | 70.90 | C |
| ATOM | 2657 | C8 | ADE | C | 419 | 37.657 | 25.463 | 70.544 | 1.00 | 71.01 | C |
| ATOM | 2658 | C2′ | ADE | C | 419 | 38.597 | 28.251 | 72.480 | 1.00 | 71.58 | C |
| ATOM | 2659 | O2′ | ADE | C | 419 | 39.053 | 29.588 | 72.523 | 1.00 | 70.81 | C |
| ATOM | 2660 | C3′ | ADE | C | 419 | 39.391 | 27.314 | 73.384 | 1.00 | 71.75 | C |
| ATOM | 2661 | O3′ | ADE | C | 419 | 39.783 | 27.926 | 74.606 | 1.00 | 72.07 | C |
| ATOM | 2662 | P | URI | C | 420 | 39.153 | 27.397 | 75.984 | 1.00 | 73.09 | C |
| ATOM | 2663 | O1P | URI | C | 420 | 39.251 | 25.913 | 75.945 | 1.00 | 72.17 | C |
| ATOM | 2664 | O2P | URI | C | 420 | 39.755 | 28.136 | 77.116 | 1.00 | 72.78 | C |
| ATOM | 2665 | O5′ | URI | C | 420 | 37.623 | 27.829 | 75.875 | 1.00 | 72.35 | C |
| ATOM | 2666 | C5′ | URI | C | 420 | 37.270 | 29.189 | 75.637 | 1.00 | 71.51 | C |
| ATOM | 2667 | C4′ | URI | C | 420 | 35.856 | 29.286 | 75.113 | 1.00 | 71.76 | C |
| ATOM | 2668 | O4′ | URI | C | 420 | 35.791 | 28.756 | 73.764 | 1.00 | 72.59 | C |
| ATOM | 2669 | C1′ | URI | C | 420 | 34.534 | 28.135 | 73.556 | 1.00 | 72.66 | C |
| ATOM | 2670 | N1 | URI | C | 420 | 34.769 | 26.717 | 73.253 | 1.00 | 73.83 | C |
| ATOM | 2671 | C6 | URI | C | 420 | 35.983 | 26.127 | 73.498 | 1.00 | 73.89 | C |
| ATOM | 2672 | C2 | URI | C | 420 | 33.722 | 25.997 | 72.708 | 1.00 | 74.56 | C |
| ATOM | 2673 | O2 | URI | C | 420 | 32.618 | 26.482 | 72.506 | 1.00 | 74.87 | C |
| ATOM | 2674 | N3 | URI | C | 420 | 34.012 | 24.685 | 72.420 | 1.00 | 74.69 | C |
| ATOM | 2675 | C4 | URI | C | 420 | 35.209 | 24.033 | 72.635 | 1.00 | 75.13 | C |
| ATOM | 2676 | O4 | URI | C | 420 | 35.320 | 22.846 | 72.320 | 1.00 | 75.68 | C |
| ATOM | 2677 | C5 | URI | C | 420 | 36.231 | 24.844 | 73.221 | 1.00 | 75.12 | C |
| ATOM | 2678 | C2′ | URI | C | 420 | 33.715 | 28.336 | 74.828 | 1.00 | 72.30 | C |
| ATOM | 2679 | O2′ | URI | C | 420 | 32.894 | 29.473 | 74.680 | 1.00 | 71.57 | C |
| ATOM | 2680 | C3′ | URI | C | 420 | 34.817 | 28.485 | 75.870 | 1.00 | 71.73 | C |
| ATOM | 2681 | O3′ | URI | C | 420 | 34.385 | 29.203 | 77.013 | 1.00 | 72.72 | C |
| ATOM | 2682 | P | URI | C | 421 | 34.258 | 28.445 | 78.421 | 1.00 | 73.56 | C |
| ATOM | 2683 | O1P | URI | C | 421 | 34.493 | 29.419 | 79.518 | 1.00 | 71.63 | C |
| ATOM | 2684 | O2P | URI | C | 421 | 35.102 | 27.234 | 78.331 | 1.00 | 73.93 | C |
| ATOM | 2685 | O5′ | URI | C | 421 | 32.734 | 27.986 | 78.454 | 1.00 | 72.57 | C |
| ATOM | 2686 | C5′ | URI | C | 421 | 31.714 | 28.809 | 77.898 | 1.00 | 70.50 | C |
| ATOM | 2687 | C4′ | URI | C | 421 | 30.450 | 28.009 | 77.697 | 1.00 | 70.66 | C |
| ATOM | 2688 | O4′ | URI | C | 421 | 30.495 | 27.386 | 76.389 | 1.00 | 70.40 | C |
| ATOM | 2689 | C1′ | URI | C | 421 | 29.731 | 26.190 | 76.413 | 1.00 | 70.60 | C |
| ATOM | 2690 | N1 | URI | C | 421 | 30.585 | 25.074 | 75.982 | 1.00 | 69.64 | C |
| ATOM | 2691 | C6 | URI | C | 421 | 31.945 | 25.095 | 76.161 | 1.00 | 69.33 | C |
| ATOM | 2692 | C2 | URI | C | 421 | 29.966 | 23.990 | 75.404 | 1.00 | 69.60 | C |
| ATOM | 2693 | O2 | URI | C | 421 | 28.765 | 23.939 | 75.223 | 1.00 | 70.36 | C |
| ATOM | 2694 | N3 | URI | C | 421 | 30.802 | 22.963 | 75.053 | 1.00 | 69.02 | C |
| ATOM | 2695 | C4 | URI | C | 421 | 32.165 | 22.911 | 75.222 | 1.00 | 68.95 | C |
| ATOM | 2696 | O4 | URI | C | 421 | 32.772 | 21.881 | 74.924 | 1.00 | 69.29 | C |
| ATOM | 2697 | C5 | URI | C | 421 | 32.734 | 24.077 | 75.813 | 1.00 | 68.62 | C |
| ATOM | 2698 | C2′ | URI | C | 421 | 29.220 | 26.007 | 77.845 | 1.00 | 71.32 | C |
| ATOM | 2699 | O2′ | URI | C | 421 | 27.907 | 26.514 | 77.977 | 1.00 | 72.51 | C |
| ATOM | 2700 | C3′ | URI | C | 421 | 30.224 | 26.831 | 78.635 | 1.00 | 70.79 | C |
| ATOM | 2701 | O3′ | URI | C | 421 | 29.629 | 27.235 | 79.860 | 1.00 | 70.56 | C |
| ATOM | 2702 | P | GUA | C | 422 | 29.852 | 26.353 | 81.188 | 1.00 | 72.90 | C |
| ATOM | 2703 | O1P | GUA | C | 422 | 31.224 | 25.778 | 81.143 | 1.00 | 71.16 | C |
| ATOM | 2704 | O2P | GUA | C | 422 | 29.449 | 27.203 | 82.343 | 1.00 | 71.02 | C |
| ATOM | 2705 | O5′ | GUA | C | 422 | 28.807 | 25.155 | 81.054 | 1.00 | 68.77 | C |
| ATOM | 2706 | C5′ | GUA | C | 422 | 27.420 | 25.393 | 81.250 | 1.00 | 66.08 | C |
| ATOM | 2707 | C4′ | GUA | C | 422 | 26.633 | 24.104 | 81.165 | 1.00 | 64.53 | C |
| ATOM | 2708 | O4′ | GUA | C | 422 | 27.229 | 23.176 | 80.219 | 1.00 | 64.44 | C |
| ATOM | 2709 | C1′ | GUA | C | 422 | 26.859 | 21.846 | 80.570 | 1.00 | 62.29 | C |
| ATOM | 2710 | N9 | GUA | C | 422 | 28.079 | 21.089 | 80.834 | 1.00 | 62.20 | C |
| ATOM | 2711 | C4 | GUA | C | 422 | 28.208 | 19.719 | 80.893 | 1.00 | 61.30 | C |
| ATOM | 2712 | N3 | GUA | C | 422 | 27.229 | 18.815 | 80.671 | 1.00 | 61.22 | C |
| ATOM | 2713 | C2 | GUA | C | 422 | 27.663 | 17.569 | 80.819 | 1.00 | 60.68 | C |
| ATOM | 2714 | N2 | GUA | C | 422 | 26.832 | 16.537 | 80.629 | 1.00 | 59.27 | C |
| ATOM | 2715 | N1 | GUA | C | 422 | 28.947 | 17.241 | 81.161 | 1.00 | 60.17 | C |
| ATOM | 2716 | C6 | GUA | C | 422 | 29.969 | 18.149 | 81.394 | 1.00 | 59.68 | C |
| ATOM | 2717 | O6 | GUA | C | 422 | 31.092 | 17.738 | 81.696 | 1.00 | 59.79 | C |
| ATOM | 2718 | C5 | GUA | C | 422 | 29.524 | 19.491 | 81.234 | 1.00 | 60.33 | C |
| ATOM | 2719 | N7 | GUA | C | 422 | 30.214 | 20.688 | 81.359 | 1.00 | 61.18 | C |
| ATOM | 2720 | C8 | GUA | C | 422 | 29.322 | 21.606 | 81.108 | 1.00 | 61.74 | C |
| ATOM | 2721 | C2′ | GUA | C | 422 | 25.987 | 21.974 | 81.823 | 1.00 | 62.23 | C |
| ATOM | 2722 | O2′ | GUA | C | 422 | 24.633 | 22.114 | 81.447 | 1.00 | 59.81 | C |
| ATOM | 2723 | C3′ | GUA | C | 422 | 26.531 | 23.260 | 82.419 | 1.00 | 63.24 | C |
| ATOM | 2724 | O3′ | GUA | C | 422 | 25.614 | 23.826 | 83.350 | 1.00 | 63.42 | C |
| ATOM | 2725 | P | CYT | C | 423 | 25.701 | 23.419 | 84.903 | 1.00 | 63.44 | C |
| ATOM | 2726 | O1P | CYT | C | 423 | 27.112 | 23.605 | 85.339 | 1.00 | 62.60 | C |
| ATOM | 2727 | O2P | CYT | C | 423 | 24.612 | 24.108 | 85.639 | 1.00 | 63.14 | C |
| ATOM | 2728 | O5′ | CYT | C | 423 | 25.367 | 21.859 | 84.903 | 1.00 | 62.50 | C |
| ATOM | 2729 | C5′ | CYT | C | 423 | 24.200 | 21.356 | 84.249 | 1.00 | 59.63 | C |
| ATOM | 2730 | C4′ | CYT | C | 423 | 24.091 | 19.861 | 84.448 | 1.00 | 57.75 | C |
| ATOM | 2731 | O4′ | CYT | C | 423 | 24.956 | 19.159 | 83.503 | 1.00 | 54.89 | C |
| ATOM | 2732 | C1′ | CYT | C | 423 | 25.509 | 18.008 | 84.127 | 1.00 | 54.29 | C |
| ATOM | 2733 | N1 | CYT | C | 423 | 26.964 | 18.198 | 84.267 | 1.00 | 53.29 | C |
| ATOM | 2734 | C6 | CYT | C | 423 | 27.507 | 19.448 | 84.335 | 1.00 | 53.60 | C |
| ATOM | 2735 | C2 | CYT | C | 423 | 27.786 | 17.075 | 84.331 | 1.00 | 53.88 | C |
| ATOM | 2736 | O2 | CYT | C | 423 | 27.268 | 15.953 | 84.273 | 1.00 | 57.13 | C |
| ATOM | 2737 | N3 | CYT | C | 423 | 29.122 | 17.235 | 84.456 | 1.00 | 52.34 | C |
| ATOM | 2738 | C4 | CYT | C | 423 | 29.641 | 18.456 | 84.517 | 1.00 | 51.34 | C |
| ATOM | 2739 | N4 | CYT | C | 423 | 30.960 | 18.566 | 84.632 | 1.00 | 51.69 | C |
| ATOM | 2740 | C5 | CYT | C | 423 | 28.831 | 19.620 | 84.459 | 1.00 | 52.45 | C |
| ATOM | 2741 | C2′ | CYT | C | 423 | 24.816 | 17.892 | 85.481 | 1.00 | 56.07 | C |
| ATOM | 2742 | O2′ | CYT | C | 423 | 23.620 | 17.170 | 85.325 | 1.00 | 54.32 | C |
| ATOM | 2743 | C3′ | CYT | C | 423 | 24.580 | 19.361 | 85.793 | 1.00 | 57.83 | C |
| ATOM | 2744 | O3′ | CYT | C | 423 | 23.641 | 19.584 | 86.842 | 1.00 | 59.37 | C |
| ATOM | 2745 | P | ADE | C | 424 | 23.978 | 19.061 | 88.330 | 1.00 | 63.43 | C |
| ATOM | 2746 | O1P | ADE | C | 424 | 22.818 | 19.387 | 89.198 | 1.00 | 61.43 | C |
| ATOM | 2747 | O2P | ADE | C | 424 | 25.346 | 19.517 | 88.742 | 1.00 | 61.11 | C |
| ATOM | 2748 | O5′ | ADE | C | 424 | 24.014 | 17.483 | 88.115 | 1.00 | 60.07 | C |
| ATOM | 2749 | C5′ | ADE | C | 424 | 24.210 | 16.607 | 89.205 | 1.00 | 58.07 | C |
| ATOM | 2750 | C4′ | ADE | C | 424 | 25.091 | 15.462 | 88.795 | 1.00 | 55.88 | C |
| ATOM | 2751 | O4′ | ADE | C | 424 | 25.801 | 15.776 | 87.572 | 1.00 | 56.48 | C |
| ATOM | 2752 | C1′ | ADE | C | 424 | 27.120 | 15.250 | 87.647 | 1.00 | 57.15 | C |
| ATOM | 2753 | N9 | ADE | C | 424 | 28.055 | 16.370 | 87.707 | 1.00 | 59.22 | C |
| ATOM | 2754 | C4 | ADE | C | 424 | 29.412 | 16.286 | 87.902 | 1.00 | 58.46 | C |
| ATOM | 2755 | N3 | ADE | C | 424 | 30.138 | 15.179 | 88.114 | 1.00 | 57.20 | C |
| ATOM | 2756 | C2 | ADE | C | 424 | 31.420 | 15.482 | 88.231 | 1.00 | 57.56 | C |
| ATOM | 2757 | N1 | ADE | C | 424 | 32.018 | 16.672 | 88.157 | 1.00 | 57.82 | C |
| ATOM | 2758 | C6 | ADE | C | 424 | 31.258 | 17.762 | 87.941 | 1.00 | 58.97 | C |
| ATOM | 2759 | N6 | ADE | C | 424 | 31.852 | 18.953 | 87.845 | 1.00 | 60.54 | C |
| ATOM | 2760 | C5 | ADE | C | 424 | 29.883 | 17.578 | 87.819 | 1.00 | 58.76 | C |
| ATOM | 2761 | N7 | ADE | C | 424 | 28.841 | 18.468 | 87.613 | 1.00 | 61.16 | C |
| ATOM | 2762 | C8 | ADE | C | 424 | 27.779 | 17.704 | 87.561 | 1.00 | 60.44 | C |
| ATOM | 2763 | C2′ | ADE | C | 424 | 27.184 | 14.421 | 88.923 | 1.00 | 55.92 | C |
| ATOM | 2764 | O2′ | ADE | C | 424 | 26.784 | 13.099 | 88.655 | 1.00 | 56.30 | C |
| ATOM | 2765 | C3′ | ADE | C | 424 | 26.199 | 15.182 | 89.780 | 1.00 | 54.65 | C |
| ATOM | 2766 | O3′ | ADE | C | 424 | 25.681 | 14.415 | 90.827 | 1.00 | 54.00 | C |
| ATOM | 2767 | P | GUA | C | 425 | 25.671 | 15.023 | 92.297 | 1.00 | 55.14 | C |
| ATOM | 2768 | O1P | GUA | C | 425 | 25.540 | 16.492 | 92.171 | 1.00 | 54.82 | C |
| ATOM | 2769 | O2P | GUA | C | 425 | 24.679 | 14.267 | 93.096 | 1.00 | 55.19 | C |
| ATOM | 2770 | O5′ | GUA | C | 425 | 27.130 | 14.653 | 92.800 | 1.00 | 54.58 | C |
| ATOM | 2771 | C5′ | GUA | C | 425 | 27.602 | 13.329 | 92.630 | 1.00 | 53.72 | C |
| ATOM | 2772 | C4′ | GUA | C | 425 | 29.087 | 13.320 | 92.505 | 1.00 | 52.36 | C |
| ATOM | 2773 | O4′ | GUA | C | 425 | 29.485 | 14.125 | 91.377 | 1.00 | 53.75 | C |
| ATOM | 2774 | C1′ | GUA | C | 425 | 30.791 | 14.626 | 91.607 | 1.00 | 55.50 | C |
| ATOM | 2775 | N9 | GUA | C | 425 | 30.774 | 16.076 | 91.399 | 1.00 | 57.33 | C |
| ATOM | 2776 | C4 | GUA | C | 425 | 31.849 | 16.935 | 91.500 | 1.00 | 57.15 | C |
| ATOM | 2777 | N3 | GUA | C | 425 | 33.115 | 16.592 | 91.812 | 1.00 | 57.47 | C |
| ATOM | 2778 | C2 | GUA | C | 425 | 33.925 | 17.635 | 91.819 | 1.00 | 56.02 | C |
| ATOM | 2779 | N2 | GUA | C | 425 | 35.219 | 17.469 | 92.092 | 1.00 | 55.54 | C |
| ATOM | 2780 | N1 | GUA | C | 425 | 33.528 | 18.917 | 91.559 | 1.00 | 56.41 | C |
| ATOM | 2781 | C6 | GUA | C | 425 | 32.232 | 19.303 | 91.247 | 1.00 | 57.54 | C |
| ATOM | 2782 | O6 | GUA | C | 425 | 31.981 | 20.499 | 91.048 | 1.00 | 59.51 | C |
| ATOM | 2783 | C5 | GUA | C | 425 | 31.345 | 18.187 | 91.216 | 1.00 | 57.41 | C |
| ATOM | 2784 | N7 | GUA | C | 425 | 29.985 | 18.121 | 90.943 | 1.00 | 56.45 | C |
| ATOM | 2785 | C8 | GUA | C | 425 | 29.690 | 16.854 | 91.065 | 1.00 | 57.56 | C |
| ATOM | 2786 | C2′ | GUA | C | 425 | 31.178 | 14.194 | 93.027 | 1.00 | 54.96 | C |
| ATOM | 2787 | O2′ | GUA | C | 425 | 31.884 | 12.977 | 92.972 | 1.00 | 55.11 | C |
| ATOM | 2788 | C3′ | GUA | C | 425 | 29.814 | 13.975 | 93.652 | 1.00 | 53.91 | C |
| ATOM | 2789 | O3′ | GUA | C | 425 | 29.879 | 13.068 | 94.743 | 1.00 | 55.10 | C |
| ATOM | 2790 | P | URI | C | 426 | 29.644 | 13.602 | 96.243 | 1.00 | 55.46 | C |
| ATOM | 2791 | O1P | URI | C | 426 | 28.622 | 14.689 | 96.184 | 1.00 | 54.09 | C |
| ATOM | 2792 | O2P | URI | C | 426 | 29.429 | 12.439 | 97.148 | 1.00 | 52.76 | C |
| ATOM | 2793 | O5′ | URI | C | 426 | 31.064 | 14.214 | 96.588 | 1.00 | 53.38 | C |
| ATOM | 2794 | C5′ | URI | C | 426 | 32.232 | 13.489 | 96.251 | 1.00 | 51.95 | C |
| ATOM | 2795 | C4′ | URI | C | 426 | 33.440 | 14.328 | 96.507 | 1.00 | 51.33 | C |
| ATOM | 2796 | O4′ | URI | C | 426 | 33.752 | 15.073 | 95.305 | 1.00 | 50.28 | C |
| ATOM | 2797 | C1′ | URI | C | 426 | 34.131 | 16.385 | 95.647 | 1.00 | 49.45 | C |
| ATOM | 2798 | N1 | URI | C | 426 | 33.084 | 17.280 | 95.148 | 1.00 | 48.32 | C |
| ATOM | 2799 | C6 | URI | C | 426 | 31.803 | 16.839 | 95.002 | 1.00 | 46.72 | C |
| ATOM | 2800 | C2 | URI | C | 426 | 33.435 | 18.569 | 94.830 | 1.00 | 47.83 | C |
| ATOM | 2801 | O2 | URI | C | 426 | 34.564 | 19.001 | 94.955 | 1.00 | 47.66 | C |
| ATOM | 2802 | N3 | URI | C | 426 | 32.413 | 19.340 | 94.358 | 1.00 | 47.12 | C |
| ATOM | 2803 | C4 | URI | C | 426 | 31.117 | 18.960 | 94.182 | 1.00 | 47.17 | C |
| ATOM | 2804 | O4 | URI | C | 426 | 30.342 | 19.736 | 93.635 | 1.00 | 50.31 | C |
| ATOM | 2805 | C5 | URI | C | 426 | 30.833 | 17.614 | 94.547 | 1.00 | 47.19 | C |
| ATOM | 2806 | C2′ | URI | C | 426 | 34.275 | 16.420 | 97.170 | 1.00 | 51.69 | C |
| ATOM | 2807 | O2′ | URI | C | 426 | 35.583 | 16.059 | 97.573 | 1.00 | 52.95 | C |
| ATOM | 2808 | C3′ | URI | C | 426 | 33.259 | 15.375 | 97.590 | 1.00 | 51.04 | C |
| ATOM | 2809 | O3′ | URI | C | 426 | 33.625 | 14.817 | 98.839 | 1.00 | 52.74 | C |
| ATOM | 2810 | P | CYT | C | 427 | 33.377 | 15.647 | 100.187 | 1.00 | 55.80 | C |
| ATOM | 2811 | O1P | CYT | C | 427 | 31.930 | 15.990 | 100.242 | 1.00 | 53.06 | C |
| ATOM | 2812 | O2P | CYT | C | 427 | 34.005 | 14.909 | 101.317 | 1.00 | 54.75 | C |
| ATOM | 2813 | O5′ | CYT | C | 427 | 34.224 | 16.977 | 99.972 | 1.00 | 54.34 | C |
| ATOM | 2814 | C5′ | CYT | C | 427 | 35.518 | 17.108 | 100.541 | 1.00 | 53.79 | C |
| ATOM | 2815 | C4′ | CYT | C | 427 | 36.034 | 18.508 | 100.340 | 1.00 | 52.68 | C |
| ATOM | 2816 | O4′ | CYT | C | 427 | 35.823 | 18.895 | 98.961 | 1.00 | 51.71 | C |
| ATOM | 2817 | C1′ | CYT | C | 427 | 35.651 | 20.299 | 98.886 | 1.00 | 52.92 | C |
| ATOM | 2818 | N1 | CYT | C | 427 | 34.338 | 20.573 | 98.292 | 1.00 | 54.24 | C |
| ATOM | 2819 | C6 | CYT | C | 427 | 33.402 | 19.583 | 98.172 | 1.00 | 55.08 | C |
| ATOM | 2820 | C2 | CYT | C | 427 | 34.049 | 21.874 | 97.865 | 1.00 | 54.20 | C |
| ATOM | 2821 | O2 | CYT | C | 427 | 34.929 | 22.740 | 97.950 | 1.00 | 53.16 | C |
| ATOM | 2822 | N3 | CYT | C | 427 | 32.819 | 22.148 | 97.365 | 1.00 | 53.57 | C |
| ATOM | 2823 | C4 | CYT | C | 427 | 31.909 | 21.178 | 97.272 | 1.00 | 54.42 | C |
| ATOM | 2824 | N4 | CYT | C | 427 | 30.714 | 21.491 | 96.794 | 1.00 | 55.39 | C |
| ATOM | 2825 | C5 | CYT | C | 427 | 32.186 | 19.839 | 97.671 | 1.00 | 55.03 | C |
| ATOM | 2826 | C2′ | CYT | C | 427 | 35.748 | 20.827 | 100.317 | 1.00 | 53.24 | C |
| ATOM | 2827 | O2′ | CYT | C | 427 | 37.085 | 21.179 | 100.595 | 1.00 | 55.47 | C |
| ATOM | 2828 | C3′ | CYT | C | 427 | 35.320 | 19.605 | 101.106 | 1.00 | 52.83 | C |
| ATOM | 2829 | O3′ | CYT | C | 427 | 35.781 | 19.690 | 102.445 | 1.00 | 53.08 | C |
| ATOM | 2830 | P | GUA | C | 428 | 34.819 | 19.224 | 103.637 | 1.00 | 56.16 | C |
| ATOM | 2831 | O1P | GUA | C | 428 | 33.772 | 18.364 | 103.013 | 1.00 | 56.05 | C |
| ATOM | 2832 | O2P | GUA | C | 428 | 35.649 | 18.690 | 104.747 | 1.00 | 56.57 | C |
| ATOM | 2833 | O5′ | GUA | C | 428 | 34.122 | 20.566 | 104.140 | 1.00 | 56.07 | C |
| ATOM | 2834 | C5′ | GUA | C | 428 | 34.815 | 21.494 | 104.971 | 1.00 | 55.18 | C |
| ATOM | 2835 | C4′ | GUA | C | 428 | 35.171 | 22.736 | 104.186 | 1.00 | 54.66 | C |
| ATOM | 2836 | O4′ | GUA | C | 428 | 34.976 | 22.483 | 102.774 | 1.00 | 55.76 | C |
| ATOM | 2837 | C1′ | GUA | C | 428 | 34.557 | 23.666 | 102.128 | 1.00 | 55.37 | C |
| ATOM | 2838 | N9 | GUA | C | 428 | 33.277 | 23.407 | 101.484 | 1.00 | 56.56 | C |
| ATOM | 2839 | C4 | GUA | C | 428 | 32.555 | 24.276 | 100.713 | 1.00 | 57.44 | C |
| ATOM | 2840 | N3 | GUA | C | 428 | 32.898 | 25.541 | 100.414 | 1.00 | 59.24 | C |
| ATOM | 2841 | C2 | GUA | C | 428 | 32.000 | 26.121 | 99.635 | 1.00 | 60.77 | C |
| ATOM | 2842 | N2 | GUA | C | 428 | 32.188 | 27.382 | 99.216 | 1.00 | 61.88 | C |
| ATOM | 2843 | N1 | GUA | C | 428 | 30.855 | 25.507 | 99.203 | 1.00 | 59.95 | C |
| ATOM | 2844 | C6 | GUA | C | 428 | 30.487 | 24.206 | 99.509 | 1.00 | 58.62 | C |
| ATOM | 2845 | O6 | GUA | C | 428 | 29.436 | 23.747 | 99.075 | 1.00 | 61.20 | C |
| ATOM | 2846 | C5 | GUA | C | 428 | 31.436 | 23.576 | 100.324 | 1.00 | 57.79 | C |
| ATOM | 2847 | N7 | GUA | C | 428 | 31.452 | 22.295 | 100.842 | 1.00 | 58.66 | C |
| ATOM | 2848 | C8 | GUA | C | 428 | 32.562 | 22.240 | 101.525 | 1.00 | 58.08 | C |
| ATOM | 2849 | C2′ | GUA | C | 428 | 34.510 | 24.768 | 103.182 | 1.00 | 54.88 | C |
| ATOM | 2850 | O2′ | GUA | C | 428 | 35.741 | 25.444 | 103.138 | 1.00 | 56.37 | C |
| ATOM | 2851 | C3′ | GUA | C | 428 | 34.340 | 23.975 | 104.471 | 1.00 | 54.17 | C |
| ATOM | 2852 | O3′ | GUA | C | 428 | 34.938 | 24.693 | 105.543 | 1.00 | 53.83 | C |
| ATOM | 2853 | P | CYT | C | 429 | 34.036 | 25.227 | 106.760 | 1.00 | 55.71 | C |
| ATOM | 2854 | O1P | CYT | C | 429 | 32.936 | 24.258 | 107.022 | 1.00 | 54.57 | C |
| ATOM | 2855 | O2P | CYT | C | 429 | 34.965 | 25.601 | 107.852 | 1.00 | 52.48 | C |
| ATOM | 2856 | O5′ | CYT | C | 429 | 33.373 | 26.557 | 106.191 | 1.00 | 55.60 | C |
| ATOM | 2857 | C5′ | CYT | C | 429 | 34.183 | 27.609 | 105.690 | 1.00 | 54.46 | C |
| ATOM | 2858 | C4′ | CYT | C | 429 | 33.340 | 28.611 | 104.941 | 1.00 | 54.38 | C |
| ATOM | 2859 | O4′ | CYT | C | 429 | 33.174 | 28.184 | 103.562 | 1.00 | 54.38 | C |
| ATOM | 2860 | C1′ | CYT | C | 429 | 31.881 | 28.547 | 103.102 | 1.00 | 54.82 | C |
| ATOM | 2861 | N1 | CYT | C | 429 | 31.095 | 27.318 | 102.934 | 1.00 | 55.41 | C |
| ATOM | 2862 | C6 | CYT | C | 429 | 31.401 | 26.195 | 103.646 | 1.00 | 56.89 | C |
| ATOM | 2863 | C2 | CYT | C | 429 | 30.018 | 27.318 | 102.047 | 1.00 | 55.74 | C |
| ATOM | 2864 | O2 | CYT | C | 429 | 29.769 | 28.337 | 101.409 | 1.00 | 56.97 | C |
| ATOM | 2865 | N3 | CYT | C | 429 | 29.273 | 26.207 | 101.910 | 1.00 | 57.40 | C |
| ATOM | 2866 | C4 | CYT | C | 429 | 29.567 | 25.118 | 102.620 | 1.00 | 59.54 | C |
| ATOM | 2867 | N4 | CYT | C | 429 | 28.789 | 24.038 | 102.465 | 1.00 | 59.89 | C |
| ATOM | 2868 | C5 | CYT | C | 429 | 30.670 | 25.084 | 103.525 | 1.00 | 59.76 | C |
| ATOM | 2869 | C2′ | CYT | C | 429 | 31.292 | 29.436 | 104.194 | 1.00 | 54.49 | C |
| ATOM | 2870 | O2′ | CYT | C | 429 | 31.715 | 30.769 | 104.009 | 1.00 | 54.42 | C |
| ATOM | 2871 | C3′ | CYT | C | 429 | 31.916 | 28.809 | 105.427 | 1.00 | 54.17 | C |
| ATOM | 2872 | O3′ | CYT | C | 429 | 31.868 | 29.702 | 106.530 | 1.00 | 55.30 | C |
| ATOM | 2873 | P | GUA | C | 430 | 30.474 | 29.975 | 107.280 | 1.00 | 55.18 | C |
| ATOM | 2874 | O1P | GUA | C | 430 | 29.896 | 28.696 | 107.762 | 1.00 | 54.20 | C |
| ATOM | 2875 | O2P | GUA | C | 430 | 30.703 | 31.072 | 108.240 | 1.00 | 55.34 | C |
| ATOM | 2876 | O5′ | GUA | C | 430 | 29.533 | 30.517 | 106.120 | 1.00 | 56.60 | C |
| ATOM | 2877 | C5′ | GUA | C | 430 | 29.584 | 31.875 | 105.702 | 1.00 | 58.01 | C |
| ATOM | 2878 | C4′ | GUA | C | 430 | 28.227 | 32.297 | 105.204 | 1.00 | 60.07 | C |
| ATOM | 2879 | O4′ | GUA | C | 430 | 27.824 | 31.334 | 104.211 | 1.00 | 59.91 | C |
| ATOM | 2880 | C1′ | GUA | C | 430 | 26.494 | 30.933 | 104.428 | 1.00 | 60.74 | C |
| ATOM | 2881 | N9 | GUA | C | 430 | 26.421 | 29.482 | 104.266 | 1.00 | 60.14 | C |
| ATOM | 2882 | C4 | GUA | C | 430 | 25.465 | 28.782 | 103.566 | 1.00 | 59.13 | C |
| ATOM | 2883 | N3 | GUA | C | 430 | 24.429 | 29.315 | 102.889 | 1.00 | 59.30 | C |
| ATOM | 2884 | C2 | GUA | C | 430 | 23.658 | 28.384 | 102.353 | 1.00 | 60.04 | C |
| ATOM | 2885 | N2 | GUA | C | 430 | 22.564 | 28.731 | 101.664 | 1.00 | 60.31 | C |
| ATOM | 2886 | N1 | GUA | C | 430 | 23.891 | 27.043 | 102.458 | 1.00 | 59.51 | C |
| ATOM | 2887 | C6 | GUA | C | 430 | 24.948 | 26.470 | 103.141 | 1.00 | 59.46 | C |
| ATOM | 2888 | O6 | GUA | C | 430 | 25.052 | 25.244 | 103.178 | 1.00 | 60.37 | C |
| ATOM | 2889 | C5 | GUA | C | 430 | 25.788 | 27.456 | 103.730 | 1.00 | 59.42 | C |
| ATOM | 2890 | N7 | GUA | C | 430 | 26.939 | 27.321 | 104.493 | 1.00 | 59.34 | C |
| ATOM | 2891 | C8 | GUA | C | 430 | 27.279 | 28.547 | 104.787 | 1.00 | 59.75 | C |
| ATOM | 2892 | C2′ | GUA | C | 430 | 26.012 | 31.435 | 105.789 | 1.00 | 60.81 | C |
| ATOM | 2893 | O2′ | GUA | C | 430 | 24.775 | 32.101 | 105.631 | 1.00 | 63.49 | C |
| ATOM | 2894 | C3′ | GUA | C | 430 | 27.162 | 32.318 | 106.291 | 1.00 | 61.67 | C |
| ATOM | 2895 | O3′ | GUA | C | 430 | 26.835 | 33.650 | 106.732 | 1.00 | 66.15 | C |
| ATOM | 2896 | P | URI | C | 431 | 26.269 | 34.760 | 105.696 | 1.00 | 68.52 | C |
| ATOM | 2897 | O1P | URI | C | 431 | 25.470 | 34.098 | 104.634 | 1.00 | 71.17 | C |
| ATOM | 2898 | O2P | URI | C | 431 | 25.641 | 35.811 | 106.523 | 1.00 | 69.16 | C |
| ATOM | 2899 | O5′ | URI | C | 431 | 27.553 | 35.410 | 105.010 | 1.00 | 71.54 | C |
| ATOM | 2900 | C5′ | URI | C | 431 | 27.731 | 35.343 | 103.595 | 1.00 | 77.99 | C |
| ATOM | 2901 | C4′ | URI | C | 431 | 27.963 | 36.722 | 103.013 | 1.00 | 82.63 | C |
| ATOM | 2902 | O4′ | URI | C | 431 | 26.978 | 37.659 | 103.522 | 1.00 | 84.74 | C |
| ATOM | 2903 | C1′ | URI | C | 431 | 27.617 | 38.881 | 103.806 | 1.00 | 84.92 | C |
| ATOM | 2904 | N1 | URI | C | 431 | 26.808 | 39.608 | 104.793 | 1.00 | 84.31 | C |
| ATOM | 2905 | C6 | URI | C | 431 | 26.189 | 38.950 | 105.829 | 1.00 | 84.61 | C |
| ATOM | 2906 | C2 | URI | C | 431 | 26.681 | 40.977 | 104.640 | 1.00 | 84.06 | C |
| ATOM | 2907 | O2 | URI | C | 431 | 27.224 | 41.598 | 103.736 | 1.00 | 83.22 | C |
| ATOM | 2908 | N3 | URI | C | 431 | 25.894 | 41.593 | 105.583 | 1.00 | 84.06 | C |
| ATOM | 2909 | C4 | URI | C | 431 | 25.238 | 40.990 | 106.641 | 1.00 | 84.44 | C |
| ATOM | 2910 | O4 | URI | C | 431 | 24.541 | 41.674 | 107.393 | 1.00 | 84.73 | C |
| ATOM | 2911 | C5 | URI | C | 431 | 25.428 | 39.576 | 106.733 | 1.00 | 84.34 | C |
| ATOM | 2912 | C2′ | URI | C | 431 | 29.005 | 38.467 | 104.283 | 1.00 | 85.04 | C |
| ATOM | 2913 | O2′ | URI | C | 431 | 29.904 | 39.548 | 104.148 | 1.00 | 87.61 | C |
| ATOM | 2914 | C3′ | URI | C | 431 | 29.324 | 37.354 | 103.291 | 1.00 | 84.77 | C |
| ATOM | 2915 | O3′ | URI | C | 431 | 29.813 | 37.934 | 102.097 | 1.00 | 86.28 | C |
| ATOM | 2916 | P | GUA | C | 432 | 31.210 | 37.441 | 101.497 | 1.00 | 90.42 | C |
| ATOM | 2917 | O1P | GUA | C | 432 | 31.952 | 38.646 | 101.053 | 1.00 | 90.72 | C |
| ATOM | 2918 | O2P | GUA | C | 432 | 31.839 | 36.515 | 102.472 | 1.00 | 89.74 | C |
| ATOM | 2919 | O5′ | GUA | C | 432 | 30.782 | 36.625 | 100.200 | 1.00 | 90.83 | C |
| ATOM | 2920 | C5′ | GUA | C | 432 | 30.313 | 37.303 | 99.043 | 1.00 | 92.43 | C |
| ATOM | 2921 | C4′ | GUA | C | 432 | 29.470 | 36.381 | 98.201 | 1.00 | 94.35 | C |
| ATOM | 2922 | O4′ | GUA | C | 432 | 28.357 | 35.893 | 99.001 | 1.00 | 95.57 | C |
| ATOM | 2923 | C1′ | GUA | C | 432 | 27.137 | 36.278 | 98.401 | 1.00 | 97.09 | C |
| ATOM | 2924 | N9 | GUA | C | 432 | 26.682 | 37.501 | 99.056 | 1.00 | 99.52 | C |
| ATOM | 2925 | C4 | GUA | C | 432 | 25.840 | 37.572 | 100.135 | 1.00 | 100.59 | C |
| ATOM | 2926 | N3 | GUA | C | 432 | 25.272 | 36.523 | 100.757 | 1.00 | 101.44 | C |
| ATOM | 2927 | C2 | GUA | C | 432 | 24.518 | 36.902 | 101.774 | 1.00 | 103.28 | C |
| ATOM | 2928 | N2 | GUA | C | 432 | 23.868 | 35.971 | 102.501 | 1.00 | 104.52 | C |
| ATOM | 2929 | N1 | GUA | C | 432 | 24.340 | 38.217 | 102.155 | 1.00 | 102.87 | C |
| ATOM | 2930 | C6 | GUA | C | 432 | 24.916 | 39.317 | 101.527 | 1.00 | 101.33 | C |
| ATOM | 2931 | O6 | GUA | C | 432 | 24.694 | 40.461 | 101.953 | 1.00 | 99.83 | C |
| ATOM | 2932 | C5 | GUA | C | 432 | 25.725 | 38.919 | 100.426 | 1.00 | 101.00 | C |
| ATOM | 2933 | N7 | GUA | C | 432 | 26.467 | 39.680 | 99.533 | 1.00 | 100.89 | C |
| ATOM | 2934 | C8 | GUA | C | 432 | 27.014 | 38.800 | 98.738 | 1.00 | 100.42 | C |
| ATOM | 2935 | C2′ | GUA | C | 432 | 27.453 | 36.453 | 96.918 | 1.00 | 96.55 | C |
| ATOM | 2936 | O2′ | GUA | C | 432 | 27.415 | 35.208 | 96.248 | 1.00 | 97.29 | C |
| ATOM | 2937 | C3′ | GUA | C | 432 | 28.852 | 37.056 | 96.983 | 1.00 | 95.20 | C |
| ATOM | 2938 | O3′ | GUA | C | 432 | 29.616 | 36.775 | 95.821 | 1.00 | 94.28 | C |
| ATOM | 2939 | P | ADE | C | 433 | 30.860 | 37.719 | 95.454 | 1.00 | 93.89 | C |
| ATOM | 2940 | O1P | ADE | C | 433 | 31.273 | 37.434 | 94.053 | 1.00 | 93.02 | C |
| ATOM | 2941 | O2P | ADE | C | 433 | 30.498 | 39.108 | 95.848 | 1.00 | 92.98 | C |
| ATOM | 2942 | O5′ | ADE | C | 433 | 32.011 | 37.212 | 96.427 | 1.00 | 91.05 | C |
| ATOM | 2943 | C5′ | ADE | C | 433 | 32.764 | 38.132 | 97.209 | 1.00 | 85.17 | C |
| ATOM | 2944 | C4′ | ADE | C | 433 | 34.017 | 37.471 | 97.711 | 1.00 | 80.49 | C |
| ATOM | 2945 | O4′ | ADE | C | 433 | 33.648 | 36.316 | 98.506 | 1.00 | 77.74 | C |
| ATOM | 2946 | C1′ | ADE | C | 433 | 34.344 | 35.184 | 98.048 | 1.00 | 74.83 | C |
| ATOM | 2947 | N9 | ADE | C | 433 | 33.454 | 34.036 | 98.172 | 1.00 | 71.45 | C |
| ATOM | 2948 | C4 | ADE | C | 433 | 33.173 | 33.345 | 99.322 | 1.00 | 69.90 | C |
| ATOM | 2949 | N3 | ADE | C | 433 | 33.654 | 33.582 | 100.554 | 1.00 | 70.14 | C |
| ATOM | 2950 | C2 | ADE | C | 433 | 33.152 | 32.710 | 101.426 | 1.00 | 69.09 | C |
| ATOM | 2951 | N1 | ADE | C | 433 | 32.295 | 31.706 | 101.220 | 1.00 | 67.52 | C |
| ATOM | 2952 | C6 | ADE | C | 433 | 31.836 | 31.498 | 99.969 | 1.00 | 67.36 | C |
| ATOM | 2953 | N6 | ADE | C | 433 | 30.986 | 30.500 | 99.759 | 1.00 | 66.58 | C |
| ATOM | 2954 | C5 | ADE | C | 433 | 32.286 | 32.353 | 98.957 | 1.00 | 68.74 | C |
| ATOM | 2955 | N7 | ADE | C | 433 | 32.013 | 32.414 | 97.599 | 1.00 | 69.59 | C |
| ATOM | 2956 | C8 | ADE | C | 433 | 32.728 | 33.426 | 97.181 | 1.00 | 70.82 | C |
| ATOM | 2957 | C2′ | ADE | C | 433 | 34.731 | 35.467 | 96.600 | 1.00 | 76.41 | C |
| ATOM | 2958 | O2′ | ADE | C | 433 | 35.919 | 34.775 | 96.295 | 1.00 | 77.66 | C |
| ATOM | 2959 | C3′ | ADE | C | 433 | 34.946 | 36.973 | 96.613 | 1.00 | 77.91 | C |
| ATOM | 2960 | O3′ | ADE | C | 433 | 36.287 | 37.288 | 96.938 | 1.00 | 78.38 | C |
| ATOM | 2961 | P | URI | C | 434 | 36.861 | 38.737 | 96.573 | 1.00 | 79.09 | C |
| ATOM | 2962 | O1P | URI | C | 434 | 36.931 | 38.805 | 95.093 | 1.00 | 79.39 | C |
| ATOM | 2963 | O2P | URI | C | 434 | 36.083 | 39.759 | 97.329 | 1.00 | 76.85 | C |
| ATOM | 2964 | O5′ | URI | C | 434 | 38.352 | 38.733 | 97.122 | 1.00 | 77.52 | C |
| ATOM | 2965 | C5′ | URI | C | 434 | 39.306 | 37.780 | 96.677 | 1.00 | 75.37 | C |
| ATOM | 2966 | C4′ | URI | C | 434 | 40.437 | 37.713 | 97.670 | 1.00 | 76.23 | C |
| ATOM | 2967 | O4′ | URI | C | 434 | 41.106 | 38.997 | 97.657 | 1.00 | 77.08 | C |
| ATOM | 2968 | C1′ | URI | C | 434 | 41.570 | 39.311 | 98.957 | 1.00 | 77.57 | C |
| ATOM | 2969 | N1 | URI | C | 434 | 41.269 | 40.727 | 99.224 | 1.00 | 78.74 | C |
| ATOM | 2970 | C6 | URI | C | 434 | 40.067 | 41.285 | 98.853 | 1.00 | 79.58 | C |
| ATOM | 2971 | C2 | URI | C | 434 | 42.238 | 41.486 | 99.858 | 1.00 | 78.34 | C |
| ATOM | 2972 | O2 | URI | C | 434 | 43.312 | 41.027 | 100.204 | 1.00 | 79.04 | C |
| ATOM | 2973 | N3 | URI | C | 434 | 41.899 | 42.804 | 100.067 | 1.00 | 77.66 | C |
| ATOM | 2974 | C4 | URI | C | 434 | 40.716 | 43.428 | 99.715 | 1.00 | 78.36 | C |
| ATOM | 2975 | O4 | URI | C | 434 | 40.557 | 44.627 | 99.965 | 1.00 | 79.03 | C |
| ATOM | 2976 | C5 | URI | C | 434 | 39.766 | 42.576 | 99.070 | 1.00 | 79.25 | C |
| ATOM | 2977 | C2′ | URI | C | 434 | 40.942 | 38.338 | 99.963 | 1.00 | 75.79 | C |
| ATOM | 2978 | O2′ | URI | C | 434 | 41.926 | 37.552 | 100.609 | 1.00 | 75.86 | C |
| ATOM | 2979 | C3′ | URI | C | 434 | 39.957 | 37.538 | 99.106 | 1.00 | 75.37 | C |
| ATOM | 2980 | O3′ | URI | C | 434 | 39.822 | 36.165 | 99.498 | 1.00 | 73.16 | C |
| ATOM | 2981 | P | ADE | C | 435 | 40.896 | 35.055 | 99.030 | 1.00 | 70.07 | C |
| ATOM | 2982 | O1P | ADE | C | 435 | 40.489 | 34.541 | 97.697 | 1.00 | 67.84 | C |
| ATOM | 2983 | O2P | ADE | C | 435 | 42.289 | 35.524 | 99.248 | 1.00 | 69.36 | C |
| ATOM | 2984 | O5′ | ADE | C | 435 | 40.663 | 33.927 | 100.113 | 1.00 | 69.65 | C |
| ATOM | 2985 | C5′ | ADE | C | 435 | 40.828 | 34.244 | 101.483 | 1.00 | 70.14 | C |
| ATOM | 2986 | C4′ | ADE | C | 435 | 39.571 | 33.936 | 102.246 | 1.00 | 69.70 | C |
| ATOM | 2987 | O4′ | ADE | C | 435 | 38.403 | 34.134 | 101.418 | 1.00 | 71.18 | C |
| ATOM | 2988 | C1′ | ADE | C | 435 | 37.383 | 33.234 | 101.823 | 1.00 | 70.22 | C |
| ATOM | 2989 | N9 | ADE | C | 435 | 36.925 | 32.513 | 100.636 | 1.00 | 70.43 | C |
| ATOM | 2990 | C4 | ADE | C | 435 | 36.083 | 31.428 | 100.598 | 1.00 | 69.40 | C |
| ATOM | 2991 | N3 | ADE | C | 435 | 35.515 | 30.801 | 101.639 | 1.00 | 69.03 | C |
| ATOM | 2992 | C2 | ADE | C | 435 | 34.747 | 29.800 | 101.219 | 1.00 | 68.55 | C |
| ATOM | 2993 | N1 | ADE | C | 435 | 34.500 | 29.389 | 99.969 | 1.00 | 68.15 | C |
| ATOM | 2994 | C6 | ADE | C | 435 | 35.083 | 30.044 | 98.947 | 1.00 | 68.35 | C |
| ATOM | 2995 | N6 | ADE | C | 435 | 34.828 | 29.641 | 97.703 | 1.00 | 67.46 | C |
| ATOM | 2996 | C5 | ADE | C | 435 | 35.925 | 31.121 | 99.259 | 1.00 | 69.18 | C |
| ATOM | 2997 | N7 | ADE | C | 435 | 36.661 | 31.988 | 98.465 | 1.00 | 70.13 | C |
| ATOM | 2998 | C8 | ADE | C | 435 | 37.237 | 32.790 | 99.327 | 1.00 | 70.31 | C |
| ATOM | 2999 | C2′ | ADE | C | 435 | 37.973 | 32.339 | 102.916 | 1.00 | 69.38 | C |
| ATOM | 3000 | O2′ | ADE | C | 435 | 37.591 | 32.838 | 104.176 | 1.00 | 68.95 | C |
| ATOM | 3001 | C3′ | ADE | C | 435 | 39.465 | 32.487 | 102.646 | 1.00 | 69.55 | C |
| ATOM | 3002 | O3′ | ADE | C | 435 | 40.295 | 32.301 | 103.774 | 1.00 | 68.77 | C |
| ATOM | 3003 | P | ADE | C | 436 | 41.552 | 31.314 | 103.666 | 1.00 | 69.41 | C |
| ATOM | 3004 | O1P | ADE | C | 436 | 42.557 | 31.779 | 104.656 | 1.00 | 67.75 | C |
| ATOM | 3005 | O2P | ADE | C | 436 | 41.938 | 31.161 | 102.236 | 1.00 | 66.39 | C |
| ATOM | 3006 | O5′ | ADE | C | 436 | 40.930 | 29.948 | 104.192 | 1.00 | 68.62 | C |
| ATOM | 3007 | C5′ | ADE | C | 436 | 39.961 | 29.978 | 105.237 | 1.00 | 67.50 | C |
| ATOM | 3008 | C4′ | ADE | C | 436 | 39.062 | 28.778 | 105.153 | 1.00 | 66.76 | C |
| ATOM | 3009 | O4′ | ADE | C | 436 | 37.955 | 29.042 | 104.253 | 1.00 | 65.84 | C |
| ATOM | 3010 | C1′ | ADE | C | 436 | 37.673 | 27.878 | 103.500 | 1.00 | 64.84 | C |
| ATOM | 3011 | N9 | ADE | C | 436 | 37.994 | 28.169 | 102.107 | 1.00 | 63.84 | C |
| ATOM | 3012 | C4 | ADE | C | 436 | 37.339 | 27.711 | 100.988 | 1.00 | 63.55 | C |
| ATOM | 3013 | N3 | ADE | C | 436 | 36.295 | 26.869 | 100.940 | 1.00 | 62.20 | C |
| ATOM | 3014 | C2 | ADE | C | 436 | 35.910 | 26.673 | 99.678 | 1.00 | 63.29 | C |
| ATOM | 3015 | N1 | ADE | C | 436 | 36.410 | 27.187 | 98.546 | 1.00 | 62.42 | C |
| ATOM | 3016 | C6 | ADE | C | 436 | 37.456 | 28.030 | 98.631 | 1.00 | 62.84 | C |
| ATOM | 3017 | N6 | ADE | C | 436 | 37.944 | 28.550 | 97.505 | 1.00 | 62.83 | C |
| ATOM | 3018 | C5 | ADE | C | 436 | 37.964 | 28.314 | 99.912 | 1.00 | 63.58 | C |
| ATOM | 3019 | N7 | ADE | C | 436 | 39.010 | 29.113 | 100.343 | 1.00 | 65.53 | C |
| ATOM | 3020 | C8 | ADE | C | 436 | 38.989 | 28.986 | 101.649 | 1.00 | 64.55 | C |
| ATOM | 3021 | C2′ | ADE | C | 436 | 38.503 | 26.756 | 104.120 | 1.00 | 65.24 | C |
| ATOM | 3022 | O2′ | ADE | C | 436 | 37.804 | 26.224 | 105.227 | 1.00 | 66.40 | C |
| ATOM | 3023 | C3′ | ADE | C | 436 | 39.715 | 27.531 | 104.595 | 1.00 | 66.18 | C |
| ATOM | 3024 | O3′ | ADE | C | 436 | 40.368 | 26.843 | 105.646 | 1.00 | 66.58 | C |
| ATOM | 3025 | P | ADE | C | 437 | 41.651 | 25.943 | 105.318 | 1.00 | 67.89 | C |
| ATOM | 3026 | O1P | ADE | C | 437 | 42.351 | 25.674 | 106.598 | 1.00 | 67.81 | C |
| ATOM | 3027 | O2P | ADE | C | 437 | 42.384 | 26.565 | 104.182 | 1.00 | 67.40 | C |
| ATOM | 3028 | O5′ | ADE | C | 437 | 41.023 | 24.572 | 104.831 | 1.00 | 66.73 | C |
| ATOM | 3029 | C5′ | ADE | C | 437 | 40.025 | 23.933 | 105.603 | 1.00 | 67.55 | C |
| ATOM | 3030 | C4′ | ADE | C | 437 | 39.313 | 22.918 | 104.762 | 1.00 | 68.47 | C |
| ATOM | 3031 | O4′ | ADE | C | 437 | 38.419 | 23.610 | 103.845 | 1.00 | 68.30 | C |
| ATOM | 3032 | C1′ | ADE | C | 437 | 38.485 | 23.002 | 102.567 | 1.00 | 67.40 | C |
| ATOM | 3033 | N9 | ADE | C | 437 | 39.111 | 23.954 | 101.651 | 1.00 | 65.38 | C |
| ATOM | 3034 | C4 | ADE | C | 437 | 38.823 | 24.112 | 100.320 | 1.00 | 64.64 | C |
| ATOM | 3035 | N3 | ADE | C | 437 | 37.923 | 23.432 | 99.598 | 1.00 | 64.22 | C |
| ATOM | 3036 | C2 | ADE | C | 437 | 37.920 | 23.852 | 98.335 | 1.00 | 65.30 | C |
| ATOM | 3037 | N1 | ADE | C | 437 | 38.660 | 24.801 | 97.756 | 1.00 | 65.94 | C |
| ATOM | 3038 | C6 | ADE | C | 437 | 39.558 | 25.467 | 98.512 | 1.00 | 65.81 | C |
| ATOM | 3039 | N6 | ADE | C | 437 | 40.298 | 26.411 | 97.934 | 1.00 | 66.54 | C |
| ATOM | 3040 | C5 | ADE | C | 437 | 39.656 | 25.118 | 99.870 | 1.00 | 65.25 | C |
| ATOM | 3041 | N7 | ADE | C | 437 | 40.454 | 25.591 | 100.900 | 1.00 | 66.13 | C |
| ATOM | 3042 | C8 | ADE | C | 437 | 40.092 | 24.869 | 101.932 | 1.00 | 66.12 | C |
| ATOM | 3043 | C2′ | ADE | C | 437 | 39.272 | 21.703 | 102.755 | 1.00 | 67.83 | C |
| ATOM | 3044 | O2′ | ADE | C | 437 | 38.406 | 20.672 | 103.193 | 1.00 | 66.91 | C |
| ATOM | 3045 | C3′ | ADE | C | 437 | 40.231 | 22.118 | 103.857 | 1.00 | 68.95 | C |
| ATOM | 3046 | O3′ | ADE | C | 437 | 40.751 | 21.005 | 104.565 | 1.00 | 71.12 | C |
| ATOM | 3047 | P | URI | C | 438 | 41.863 | 20.075 | 103.879 | 1.00 | 73.66 | C |
| ATOM | 3048 | O1P | URI | C | 438 | 42.506 | 20.826 | 102.769 | 1.00 | 71.57 | C |
| ATOM | 3049 | O2P | URI | C | 438 | 42.702 | 19.539 | 104.981 | 1.00 | 73.16 | C |
| ATOM | 3050 | O5′ | URI | C | 438 | 41.002 | 18.886 | 103.258 | 1.00 | 74.03 | C |
| ATOM | 3051 | C5′ | URI | C | 438 | 41.468 | 18.148 | 102.138 | 1.00 | 76.70 | C |
| ATOM | 3052 | C4′ | URI | C | 438 | 40.353 | 17.964 | 101.141 | 1.00 | 78.88 | C |
| ATOM | 3053 | O4′ | URI | C | 438 | 39.685 | 19.237 | 100.955 | 1.00 | 79.02 | C |
| ATOM | 3054 | C1′ | URI | C | 438 | 39.506 | 19.493 | 99.575 | 1.00 | 78.40 | C |
| ATOM | 3055 | N1 | URI | C | 438 | 40.353 | 20.638 | 99.206 | 1.00 | 76.81 | C |
| ATOM | 3056 | C6 | URI | C | 438 | 41.213 | 21.205 | 100.120 | 1.00 | 77.28 | C |
| ATOM | 3057 | C2 | URI | C | 438 | 40.249 | 21.142 | 97.920 | 1.00 | 75.94 | C |
| ATOM | 3058 | O2 | URI | C | 438 | 39.522 | 20.654 | 97.072 | 1.00 | 75.16 | C |
| ATOM | 3059 | N3 | URI | C | 438 | 41.033 | 22.243 | 97.666 | 1.00 | 75.22 | C |
| ATOM | 3060 | C4 | URI | C | 438 | 41.892 | 22.877 | 98.545 | 1.00 | 75.03 | C |
| ATOM | 3061 | O4 | URI | C | 438 | 42.431 | 23.931 | 98.208 | 1.00 | 73.84 | C |
| ATOM | 3062 | C5 | URI | C | 438 | 41.966 | 22.278 | 99.840 | 1.00 | 76.07 | C |
| ATOM | 3063 | C2′ | URI | C | 438 | 39.817 | 18.199 | 98.826 | 1.00 | 79.68 | C |
| ATOM | 3064 | O2′ | URI | C | 438 | 38.650 | 17.426 | 98.641 | 1.00 | 80.05 | C |
| ATOM | 3065 | C3′ | URI | C | 438 | 40.810 | 17.522 | 99.759 | 1.00 | 81.02 | C |
| ATOM | 3066 | O3′ | URI | C | 438 | 40.687 | 16.114 | 99.641 | 1.00 | 85.73 | C |
| ATOM | 3067 | P | GUA | C | 439 | 41.958 | 15.243 | 99.201 | 1.00 | 89.61 | C |
| ATOM | 3068 | O1P | GUA | C | 439 | 43.063 | 15.595 | 100.137 | 1.00 | 86.65 | C |
| ATOM | 3069 | O2P | GUA | C | 439 | 41.487 | 13.839 | 99.104 | 1.00 | 88.33 | C |
| ATOM | 3070 | O5′ | GUA | C | 439 | 42.317 | 15.776 | 97.734 | 1.00 | 94.00 | C |
| ATOM | 3071 | C5′ | GUA | C | 439 | 41.454 | 15.520 | 96.617 | 1.00 | 101.35 | C |
| ATOM | 3072 | C4′ | GUA | C | 439 | 41.385 | 16.731 | 95.698 | 1.00 | 106.66 | C |
| ATOM | 3073 | O4′ | GUA | C | 439 | 41.296 | 17.935 | 96.498 | 1.00 | 109.59 | C |
| ATOM | 3074 | C1′ | GUA | C | 439 | 42.080 | 18.963 | 95.920 | 1.00 | 111.32 | C |
| ATOM | 3075 | N9 | GUA | C | 439 | 43.115 | 19.344 | 96.874 | 1.00 | 113.82 | C |
| ATOM | 3076 | C4 | GUA | C | 439 | 43.905 | 20.462 | 96.810 | 1.00 | 115.05 | C |
| ATOM | 3077 | N3 | GUA | C | 439 | 43.864 | 21.402 | 95.844 | 1.00 | 115.22 | C |
| ATOM | 3078 | C2 | GUA | C | 439 | 44.748 | 22.363 | 96.054 | 1.00 | 116.16 | C |
| ATOM | 3079 | N2 | GUA | C | 439 | 44.845 | 23.380 | 95.188 | 1.00 | 116.49 | C |
| ATOM | 3080 | N1 | GUA | C | 439 | 45.605 | 22.397 | 97.130 | 1.00 | 117.15 | C |
| ATOM | 3081 | C6 | GUA | C | 439 | 45.665 | 21.437 | 98.139 | 1.00 | 117.34 | C |
| ATOM | 3082 | O6 | GUA | C | 439 | 46.480 | 21.561 | 99.069 | 1.00 | 118.17 | C |
| ATOM | 3083 | C5 | GUA | C | 439 | 44.720 | 20.400 | 97.924 | 1.00 | 116.29 | C |
| ATOM | 3084 | N7 | GUA | C | 439 | 44.448 | 19.263 | 98.672 | 1.00 | 115.53 | C |
| ATOM | 3085 | C8 | GUA | C | 439 | 43.492 | 18.666 | 98.012 | 1.00 | 115.04 | C |
| ATOM | 3086 | C2′ | GUA | C | 439 | 42.648 | 18.457 | 94.595 | 1.00 | 110.41 | C |
| ATOM | 3087 | O2′ | GUA | C | 439 | 41.899 | 18.960 | 93.505 | 1.00 | 109.71 | C |
| ATOM | 3088 | C3′ | GUA | C | 439 | 42.567 | 16.938 | 94.756 | 1.00 | 109.48 | C |
| ATOM | 3089 | O3′ | GUA | C | 439 | 42.291 | 16.365 | 93.486 | 1.00 | 111.37 | C |
| ATOM | 3090 | P | URI | C | 440 | 41.819 | 14.834 | 93.390 | 1.00 | 113.61 | C |
| ATOM | 3091 | O1P | URI | C | 440 | 42.876 | 14.008 | 94.033 | 1.00 | 113.47 | C |
| ATOM | 3092 | O2P | URI | C | 440 | 40.423 | 14.748 | 93.891 | 1.00 | 113.46 | C |
| ATOM | 3093 | O5′ | URI | C | 440 | 41.824 | 14.541 | 91.820 | 1.00 | 113.15 | C |
| ATOM | 3094 | C5′ | URI | C | 440 | 41.126 | 15.393 | 90.908 | 1.00 | 112.76 | C |
| ATOM | 3095 | C4′ | URI | C | 440 | 39.673 | 14.991 | 90.843 | 1.00 | 112.75 | C |
| ATOM | 3096 | O4′ | URI | C | 440 | 38.835 | 16.101 | 90.479 | 1.00 | 112.32 | C |
| ATOM | 3097 | C1′ | URI | C | 440 | 37.587 | 15.567 | 90.130 | 1.00 | 111.52 | C |
| ATOM | 3098 | N1 | URI | C | 440 | 36.778 | 16.612 | 89.482 | 1.00 | 109.39 | C |
| ATOM | 3099 | C6 | URI | C | 440 | 37.282 | 17.876 | 89.285 | 1.00 | 109.63 | C |
| ATOM | 3100 | C2 | URI | C | 440 | 35.484 | 16.296 | 89.108 | 1.00 | 107.64 | C |
| ATOM | 3101 | O2 | URI | C | 440 | 35.011 | 15.182 | 89.229 | 1.00 | 105.80 | C |
| ATOM | 3102 | N3 | URI | C | 440 | 34.764 | 17.338 | 88.584 | 1.00 | 107.65 | C |
| ATOM | 3103 | C4 | URI | C | 440 | 35.198 | 18.634 | 88.389 | 1.00 | 108.72 | C |
| ATOM | 3104 | O4 | URI | C | 440 | 34.416 | 19.477 | 87.948 | 1.00 | 109.01 | C |
| ATOM | 3105 | C5 | URI | C | 440 | 36.556 | 18.871 | 88.768 | 1.00 | 109.19 | C |
| ATOM | 3106 | C2′ | URI | C | 440 | 37.908 | 14.313 | 89.313 | 1.00 | 112.54 | C |
| ATOM | 3107 | O2′ | URI | C | 440 | 36.925 | 13.323 | 89.561 | 1.00 | 112.81 | C |
| ATOM | 3108 | C3′ | URI | C | 440 | 39.304 | 13.908 | 89.834 | 1.00 | 113.01 | C |
| ATOM | 3109 | O3′ | URI | C | 440 | 39.263 | 12.650 | 90.501 | 1.00 | 113.21 | C |
| ATOM | 3110 | P | ADE | C | 441 | 40.229 | 11.459 | 90.017 | 1.00 | 113.40 | C |
| ATOM | 3111 | O1P | ADE | C | 441 | 40.156 | 10.387 | 91.047 | 1.00 | 113.55 | C |
| ATOM | 3112 | O2P | ADE | C | 441 | 41.553 | 12.033 | 89.643 | 1.00 | 112.92 | C |
| ATOM | 3113 | O5′ | ADE | C | 441 | 39.519 | 10.921 | 88.696 | 1.00 | 110.64 | C |
| ATOM | 3114 | C5′ | ADE | C | 441 | 38.116 | 11.095 | 88.499 | 1.00 | 104.96 | C |
| ATOM | 3115 | C4′ | ADE | C | 441 | 37.362 | 9.926 | 89.076 | 1.00 | 100.67 | C |
| ATOM | 3116 | O4′ | ADE | C | 441 | 36.506 | 10.381 | 90.160 | 1.00 | 99.35 | C |
| ATOM | 3117 | C1′ | ADE | C | 441 | 35.350 | 9.551 | 90.222 | 1.00 | 99.29 | C |
| ATOM | 3118 | N9 | ADE | C | 441 | 34.178 | 10.370 | 89.921 | 1.00 | 99.63 | C |
| ATOM | 3119 | C4 | ADE | C | 441 | 32.866 | 9.986 | 90.077 | 1.00 | 100.33 | C |
| ATOM | 3120 | N3 | ADE | C | 441 | 32.409 | 8.815 | 90.560 | 1.00 | 100.94 | C |
| ATOM | 3121 | C2 | ADE | C | 441 | 31.076 | 8.787 | 90.543 | 1.00 | 100.93 | C |
| ATOM | 3122 | N1 | ADE | C | 441 | 30.214 | 9.724 | 90.127 | 1.00 | 100.56 | C |
| ATOM | 3123 | C6 | ADE | C | 441 | 30.706 | 10.889 | 89.647 | 1.00 | 100.33 | C |
| ATOM | 3124 | N6 | ADE | C | 441 | 29.847 | 11.819 | 89.224 | 1.00 | 100.95 | C |
| ATOM | 3125 | C5 | ADE | C | 441 | 32.106 | 11.047 | 89.618 | 1.00 | 100.21 | C |
| ATOM | 3126 | N7 | ADE | C | 441 | 32.922 | 12.092 | 89.202 | 1.00 | 99.64 | C |
| ATOM | 3127 | C8 | ADE | C | 441 | 34.138 | 11.644 | 89.407 | 1.00 | 99.73 | C |
| ATOM | 3128 | C2′ | ADE | C | 441 | 35.535 | 8.476 | 89.149 | 1.00 | 98.46 | C |
| ATOM | 3129 | O2′ | ADE | C | 441 | 36.198 | 7.350 | 89.691 | 1.00 | 98.08 | C |
| ATOM | 3130 | C3′ | ADE | C | 441 | 36.378 | 9.242 | 88.143 | 1.00 | 98.19 | C |
| ATOM | 3131 | O3′ | ADE | C | 441 | 37.018 | 8.396 | 87.189 | 1.00 | 93.51 | C |
| ATOM | 3132 | P | GUA | C | 442 | 36.821 | 8.678 | 85.612 | 1.00 | 89.51 | C |
| ATOM | 3133 | O1P | GUA | C | 442 | 37.440 | 9.991 | 85.294 | 1.00 | 89.28 | C |
| ATOM | 3134 | O2P | GUA | C | 442 | 37.273 | 7.462 | 84.887 | 1.00 | 89.23 | C |
| ATOM | 3135 | O5′ | GUA | C | 442 | 35.241 | 8.828 | 85.427 | 1.00 | 84.16 | C |
| ATOM | 3136 | C5′ | GUA | C | 442 | 34.414 | 7.677 | 85.295 | 1.00 | 76.13 | C |
| ATOM | 3137 | C4′ | GUA | C | 442 | 32.964 | 8.028 | 85.534 | 1.00 | 70.89 | C |
| ATOM | 3138 | O4′ | GUA | C | 442 | 32.854 | 9.114 | 86.483 | 1.00 | 69.42 | C |
| ATOM | 3139 | C1′ | GUA | C | 442 | 31.710 | 9.895 | 86.176 | 1.00 | 69.07 | C |
| ATOM | 3140 | N9 | GUA | C | 442 | 32.148 | 11.239 | 85.830 | 1.00 | 69.99 | C |
| ATOM | 3141 | C4 | GUA | C | 442 | 31.341 | 12.276 | 85.441 | 1.00 | 70.26 | C |
| ATOM | 3142 | N3 | GUA | C | 442 | 30.001 | 12.225 | 85.304 | 1.00 | 70.70 | C |
| ATOM | 3143 | C2 | GUA | C | 442 | 29.500 | 13.389 | 84.932 | 1.00 | 71.20 | C |
| ATOM | 3144 | N2 | GUA | C | 442 | 28.181 | 13.513 | 84.739 | 1.00 | 72.00 | C |
| ATOM | 3145 | N1 | GUA | C | 442 | 30.255 | 14.518 | 84.720 | 1.00 | 71.27 | C |
| ATOM | 3146 | C6 | GUA | C | 442 | 31.638 | 14.595 | 84.866 | 1.00 | 71.01 | C |
| ATOM | 3147 | O6 | GUA | C | 442 | 32.224 | 15.671 | 84.676 | 1.00 | 71.49 | C |
| ATOM | 3148 | C5 | GUA | C | 442 | 32.188 | 13.348 | 85.248 | 1.00 | 70.44 | C |
| ATOM | 3149 | N7 | GUA | C | 442 | 33.504 | 12.984 | 85.492 | 1.00 | 69.90 | C |
| ATOM | 3150 | C8 | GUA | C | 442 | 33.433 | 11.726 | 85.833 | 1.00 | 70.15 | C |
| ATOM | 3151 | C2′ | GUA | C | 442 | 31.029 | 9.239 | 84.983 | 1.00 | 68.98 | C |
| ATOM | 3152 | O2′ | GUA | C | 442 | 30.049 | 8.342 | 85.438 | 1.00 | 69.32 | C |
| ATOM | 3153 | C3′ | GUA | C | 442 | 32.207 | 8.541 | 84.327 | 1.00 | 69.32 | C |
| ATOM | 3154 | O3′ | GUA | C | 442 | 31.757 | 7.461 | 83.538 | 1.00 | 67.22 | C |
| ATOM | 3155 | P | CYT | C | 443 | 31.675 | 7.628 | 81.951 | 1.00 | 66.82 | C |
| ATOM | 3156 | O1P | CYT | C | 443 | 32.780 | 8.543 | 81.568 | 1.00 | 65.40 | C |
| ATOM | 3157 | O2P | CYT | C | 443 | 31.602 | 6.273 | 81.351 | 1.00 | 66.64 | C |
| ATOM | 3158 | O5′ | CYT | C | 443 | 30.280 | 8.362 | 81.719 | 1.00 | 64.23 | C |
| ATOM | 3159 | C5′ | CYT | C | 443 | 29.212 | 8.179 | 82.632 | 1.00 | 60.44 | C |
| ATOM | 3160 | C4′ | CYT | C | 443 | 28.034 | 9.028 | 82.237 | 1.00 | 60.42 | C |
| ATOM | 3161 | O4′ | CYT | C | 443 | 28.120 | 10.311 | 82.901 | 1.00 | 60.75 | C |
| ATOM | 3162 | C1′ | CYT | C | 443 | 27.632 | 11.325 | 82.043 | 1.00 | 59.94 | C |
| ATOM | 3163 | N1 | CYT | C | 443 | 28.741 | 12.233 | 81.752 | 1.00 | 60.55 | C |
| ATOM | 3164 | C6 | CYT | C | 443 | 30.034 | 11.807 | 81.851 | 1.00 | 61.41 | C |
| ATOM | 3165 | C2 | CYT | C | 443 | 28.458 | 13.543 | 81.398 | 1.00 | 61.61 | C |
| ATOM | 3166 | O2 | CYT | C | 443 | 27.281 | 13.879 | 81.264 | 1.00 | 62.93 | C |
| ATOM | 3167 | N3 | CYT | C | 443 | 29.473 | 14.411 | 81.203 | 1.00 | 62.99 | C |
| ATOM | 3168 | C4 | CYT | C | 443 | 30.736 | 13.998 | 81.338 | 1.00 | 63.16 | C |
| ATOM | 3169 | N4 | CYT | C | 443 | 31.714 | 14.898 | 81.176 | 1.00 | 63.54 | C |
| ATOM | 3170 | C5 | CYT | C | 443 | 31.054 | 12.648 | 81.655 | 1.00 | 62.50 | C |
| ATOM | 3171 | C2′ | CYT | C | 443 | 27.071 | 10.626 | 80.811 | 1.00 | 60.08 | C |
| ATOM | 3172 | O2′ | CYT | C | 443 | 25.713 | 10.318 | 81.032 | 1.00 | 58.31 | C |
| ATOM | 3173 | C3′ | CYT | C | 443 | 27.939 | 9.382 | 80.770 | 1.00 | 60.24 | C |
| ATOM | 3174 | O3′ | CYT | C | 443 | 27.290 | 8.353 | 80.052 | 1.00 | 61.43 | C |
| ATOM | 3175 | P | URI | C | 444 | 27.651 | 8.130 | 78.509 | 1.00 | 62.89 | C |
| ATOM | 3176 | O1P | URI | C | 444 | 29.135 | 8.146 | 78.411 | 1.00 | 62.11 | C |
| ATOM | 3177 | O2P | URI | C | 444 | 26.902 | 6.940 | 78.051 | 1.00 | 62.24 | C |
| ATOM | 3178 | O5′ | URI | C | 444 | 27.049 | 9.422 | 77.789 | 1.00 | 62.10 | C |
| ATOM | 3179 | C5′ | URI | C | 444 | 25.641 | 9.652 | 77.769 | 1.00 | 60.87 | C |
| ATOM | 3180 | C4′ | URI | C | 444 | 25.324 | 11.023 | 77.207 | 1.00 | 61.87 | C |
| ATOM | 3181 | O4′ | URI | C | 444 | 25.733 | 12.063 | 78.133 | 1.00 | 63.01 | C |
| ATOM | 3182 | C1′ | URI | C | 444 | 26.046 | 13.248 | 77.410 | 1.00 | 62.90 | C |
| ATOM | 3183 | N1 | URI | C | 444 | 27.444 | 13.612 | 77.690 | 1.00 | 64.77 | C |
| ATOM | 3184 | C6 | URI | C | 444 | 28.354 | 12.674 | 78.113 | 1.00 | 65.25 | C |
| ATOM | 3185 | C2 | URI | C | 444 | 27.826 | 14.936 | 77.505 | 1.00 | 64.97 | C |
| ATOM | 3186 | O2 | URI | C | 444 | 27.060 | 15.803 | 77.139 | 1.00 | 66.12 | C |
| ATOM | 3187 | N3 | URI | C | 444 | 29.144 | 15.204 | 77.763 | 1.00 | 65.12 | C |
| ATOM | 3188 | C4 | URI | C | 444 | 30.105 | 14.311 | 78.170 | 1.00 | 65.52 | C |
| ATOM | 3189 | O4 | URI | C | 444 | 31.270 | 14.694 | 78.287 | 1.00 | 66.32 | C |
| ATOM | 3190 | C5 | URI | C | 444 | 29.637 | 12.970 | 78.351 | 1.00 | 66.21 | C |
| ATOM | 3191 | C2′ | URI | C | 444 | 25.805 | 12.934 | 75.933 | 1.00 | 62.39 | C |
| ATOM | 3192 | O2′ | URI | C | 444 | 24.491 | 13.306 | 75.581 | 1.00 | 60.13 | C |
| ATOM | 3193 | C3′ | URI | C | 444 | 26.013 | 11.425 | 75.918 | 1.00 | 62.63 | C |
| ATOM | 3194 | O3′ | URI | C | 444 | 25.383 | 10.827 | 74.793 | 1.00 | 64.64 | C |
| ATOM | 3195 | P | ADE | C | 445 | 25.916 | 11.155 | 73.308 | 1.00 | 66.28 | C |
| ATOM | 3196 | O1P | ADE | C | 445 | 25.219 | 10.211 | 72.397 | 1.00 | 64.73 | C |
| ATOM | 3197 | O2P | ADE | C | 445 | 27.409 | 11.194 | 73.325 | 1.00 | 66.33 | C |
| ATOM | 3198 | O5′ | ADE | C | 445 | 25.378 | 12.631 | 73.036 | 1.00 | 62.89 | C |
| ATOM | 3199 | C5′ | ADE | C | 445 | 26.059 | 13.503 | 72.143 | 1.00 | 62.04 | C |
| ATOM | 3200 | C4′ | ADE | C | 445 | 25.576 | 14.918 | 72.337 | 1.00 | 61.54 | C |
| ATOM | 3201 | O4′ | ADE | C | 445 | 26.035 | 15.392 | 73.628 | 1.00 | 61.02 | C |
| ATOM | 3202 | C1′ | ADE | C | 445 | 26.237 | 16.797 | 73.573 | 1.00 | 59.77 | C |
| ATOM | 3203 | N9 | ADE | C | 445 | 27.620 | 17.092 | 73.952 | 1.00 | 57.68 | C |
| ATOM | 3204 | C4 | ADE | C | 445 | 28.145 | 18.350 | 74.089 | 1.00 | 57.02 | C |
| ATOM | 3205 | N3 | ADE | C | 445 | 27.499 | 19.518 | 73.939 | 1.00 | 58.61 | C |
| ATOM | 3206 | C2 | ADE | C | 445 | 28.333 | 20.541 | 74.126 | 1.00 | 60.44 | C |
| ATOM | 3207 | N1 | ADE | C | 445 | 29.641 | 20.529 | 74.422 | 1.00 | 58.87 | C |
| ATOM | 3208 | C6 | ADE | C | 445 | 30.256 | 19.337 | 74.571 | 1.00 | 57.61 | C |
| ATOM | 3209 | N6 | ADE | C | 445 | 31.557 | 19.330 | 74.874 | 1.00 | 58.29 | C |
| ATOM | 3210 | C5 | ADE | C | 445 | 29.477 | 18.172 | 74.399 | 1.00 | 56.54 | C |
| ATOM | 3211 | N7 | ADE | C | 445 | 29.783 | 16.821 | 74.481 | 1.00 | 56.12 | C |
| ATOM | 3212 | C8 | ADE | C | 445 | 28.649 | 16.225 | 74.213 | 1.00 | 56.67 | C |
| ATOM | 3213 | C2′ | ADE | C | 445 | 25.940 | 17.243 | 72.139 | 1.00 | 60.70 | C |
| ATOM | 3214 | O2′ | ADE | C | 445 | 24.631 | 17.755 | 72.031 | 1.00 | 58.79 | C |
| ATOM | 3215 | C3′ | ADE | C | 445 | 26.135 | 15.946 | 71.368 | 1.00 | 61.36 | C |
| ATOM | 3216 | O3′ | ADE | C | 445 | 25.406 | 15.987 | 70.150 | 1.00 | 63.54 | C |
| ATOM | 3217 | P | ADE | C | 446 | 26.082 | 16.646 | 68.855 | 1.00 | 65.43 | C |
| ATOM | 3218 | O1P | ADE | C | 446 | 25.161 | 16.539 | 67.699 | 1.00 | 64.30 | C |
| ATOM | 3219 | O2P | ADE | C | 446 | 27.449 | 16.070 | 68.762 | 1.00 | 64.89 | C |
| ATOM | 3220 | O5′ | ADE | C | 446 | 26.212 | 18.185 | 69.236 | 1.00 | 65.58 | C |
| ATOM | 3221 | C5′ | ADE | C | 446 | 25.061 | 18.980 | 69.484 | 1.00 | 67.19 | C |
| ATOM | 3222 | C4′ | ADE | C | 446 | 25.449 | 20.438 | 69.609 | 1.00 | 69.70 | C |
| ATOM | 3223 | O4′ | ADE | C | 446 | 26.117 | 20.673 | 70.881 | 1.00 | 69.91 | C |
| ATOM | 3224 | C1′ | ADE | C | 446 | 27.070 | 21.719 | 70.732 | 1.00 | 68.88 | C |
| ATOM | 3225 | N9 | ADE | C | 446 | 28.390 | 21.162 | 71.001 | 1.00 | 67.70 | C |
| ATOM | 3226 | C4 | ADE | C | 446 | 29.535 | 21.876 | 71.246 | 1.00 | 66.81 | C |
| ATOM | 3227 | N3 | ADE | C | 446 | 29.667 | 23.211 | 71.291 | 1.00 | 66.09 | C |
| ATOM | 3228 | C2 | ADE | C | 446 | 30.929 | 23.545 | 71.544 | 1.00 | 65.77 | C |
| ATOM | 3229 | N1 | ADE | C | 446 | 31.991 | 22.758 | 71.741 | 1.00 | 64.82 | C |
| ATOM | 3230 | C6 | ADE | C | 446 | 31.821 | 21.422 | 71.690 | 1.00 | 65.47 | C |
| ATOM | 3231 | N6 | ADE | C | 446 | 32.882 | 20.636 | 71.883 | 1.00 | 66.43 | C |
| ATOM | 3232 | C5 | ADE | C | 446 | 30.532 | 20.938 | 71.431 | 1.00 | 66.11 | C |
| ATOM | 3233 | N7 | ADE | C | 446 | 30.025 | 19.653 | 71.306 | 1.00 | 67.03 | C |
| ATOM | 3234 | C8 | ADE | C | 446 | 28.753 | 19.840 | 71.051 | 1.00 | 67.08 | C |
| ATOM | 3235 | C2′ | ADE | C | 446 | 26.974 | 22.201 | 69.284 | 1.00 | 70.69 | C |
| ATOM | 3236 | O2′ | ADE | C | 446 | 26.100 | 23.309 | 69.172 | 1.00 | 71.01 | C |
| ATOM | 3237 | C3′ | ADE | C | 446 | 26.462 | 20.944 | 68.599 | 1.00 | 70.64 | C |
| ATOM | 3238 | O3′ | ADE | C | 446 | 25.863 | 21.222 | 67.346 | 1.00 | 72.10 | C |
| ATOM | 3239 | P | ADE | C | 447 | 26.629 | 20.801 | 66.002 | 1.00 | 74.80 | C |
| ATOM | 3240 | O1P | ADE | C | 447 | 25.726 | 21.123 | 64.867 | 1.00 | 74.26 | C |
| ATOM | 3241 | O2P | ADE | C | 447 | 27.136 | 19.409 | 66.164 | 1.00 | 73.67 | C |
| ATOM | 3242 | O5′ | ADE | C | 447 | 27.877 | 21.791 | 65.965 | 1.00 | 73.64 | C |
| ATOM | 3243 | C5′ | ADE | C | 447 | 27.703 | 23.182 | 66.193 | 1.00 | 73.27 | C |
| ATOM | 3244 | C4′ | ADE | C | 447 | 29.010 | 23.806 | 66.603 | 1.00 | 73.72 | C |
| ATOM | 3245 | O4′ | ADE | C | 447 | 29.479 | 23.193 | 67.826 | 1.00 | 72.33 | C |
| ATOM | 3246 | C1′ | ADE | C | 447 | 30.894 | 23.188 | 67.843 | 1.00 | 72.82 | C |
| ATOM | 3247 | N9 | ADE | C | 447 | 31.335 | 21.803 | 67.995 | 1.00 | 73.10 | C |
| ATOM | 3248 | C4 | ADE | C | 447 | 32.552 | 21.369 | 68.464 | 1.00 | 72.15 | C |
| ATOM | 3249 | N3 | ADE | C | 447 | 33.582 | 22.124 | 68.880 | 1.00 | 72.73 | C |
| ATOM | 3250 | C2 | ADE | C | 447 | 34.600 | 21.352 | 69.276 | 1.00 | 72.40 | C |
| ATOM | 3251 | N1 | ADE | C | 447 | 34.695 | 20.015 | 69.296 | 1.00 | 70.81 | C |
| ATOM | 3252 | C6 | ADE | C | 447 | 33.642 | 19.290 | 68.867 | 1.00 | 70.89 | C |
| ATOM | 3253 | N6 | ADE | C | 447 | 33.738 | 17.961 | 68.877 | 1.00 | 69.94 | C |
| ATOM | 3254 | C5 | ADE | C | 447 | 32.500 | 19.987 | 68.428 | 1.00 | 71.53 | C |
| ATOM | 3255 | N7 | ADE | C | 447 | 31.271 | 19.555 | 67.949 | 1.00 | 72.07 | C |
| ATOM | 3256 | C8 | ADE | C | 447 | 30.620 | 20.667 | 67.706 | 1.00 | 72.94 | C |
| ATOM | 3257 | C2′ | ADE | C | 447 | 31.359 | 23.840 | 66.541 | 1.00 | 73.60 | C |
| ATOM | 3258 | O2′ | ADE | C | 447 | 31.584 | 25.213 | 66.757 | 1.00 | 72.67 | C |
| ATOM | 3259 | C3′ | ADE | C | 447 | 30.158 | 23.597 | 65.639 | 1.00 | 74.90 | C |
| ATOM | 3260 | O3′ | ADE | C | 447 | 30.084 | 24.566 | 64.603 | 1.00 | 79.87 | C |
| ATOM | 3261 | P | ADE | C | 448 | 30.492 | 24.163 | 63.098 | 1.00 | 83.67 | C |
| ATOM | 3262 | O1P | ADE | C | 448 | 29.899 | 25.203 | 62.213 | 1.00 | 82.49 | C |
| ATOM | 3263 | O2P | ADE | C | 448 | 30.178 | 22.725 | 62.860 | 1.00 | 80.82 | C |
| ATOM | 3264 | O5′ | ADE | C | 448 | 32.077 | 24.339 | 63.091 | 1.00 | 84.13 | C |
| ATOM | 3265 | C5′ | ADE | C | 448 | 32.699 | 25.406 | 62.374 | 1.00 | 84.08 | C |
| ATOM | 3266 | C4′ | ADE | C | 448 | 32.454 | 26.728 | 63.066 | 1.00 | 83.97 | C |
| ATOM | 3267 | O4′ | ADE | C | 448 | 32.636 | 26.595 | 64.504 | 1.00 | 83.19 | C |
| ATOM | 3268 | C1′ | ADE | C | 448 | 33.134 | 27.821 | 65.033 | 1.00 | 83.31 | C |
| ATOM | 3269 | N9 | ADE | C | 448 | 34.454 | 27.575 | 65.611 | 1.00 | 82.50 | C |
| ATOM | 3270 | C4 | ADE | C | 448 | 35.292 | 28.540 | 66.112 | 1.00 | 81.82 | C |
| ATOM | 3271 | N3 | ADE | C | 448 | 35.047 | 29.857 | 66.208 | 1.00 | 82.11 | C |
| ATOM | 3272 | C2 | ADE | C | 448 | 36.095 | 30.491 | 66.727 | 1.00 | 82.15 | C |
| ATOM | 3273 | N1 | ADE | C | 448 | 37.275 | 29.996 | 67.122 | 1.00 | 81.66 | C |
| ATOM | 3274 | C6 | ADE | C | 448 | 37.492 | 28.671 | 67.003 | 1.00 | 80.57 | C |
| ATOM | 3275 | N6 | ADE | C | 448 | 38.676 | 28.191 | 67.369 | 1.00 | 78.90 | C |
| ATOM | 3276 | C5 | ADE | C | 448 | 36.449 | 27.882 | 66.484 | 1.00 | 81.08 | C |
| ATOM | 3277 | N7 | ADE | C | 448 | 36.333 | 26.520 | 66.253 | 1.00 | 81.17 | C |
| ATOM | 3278 | C8 | ADE | C | 448 | 35.131 | 26.388 | 65.742 | 1.00 | 82.13 | C |
| ATOM | 3279 | C2′ | ADE | C | 448 | 33.242 | 28.796 | 63.858 | 1.00 | 83.95 | C |
| ATOM | 3280 | O2′ | ADE | C | 448 | 32.075 | 29.588 | 63.742 | 1.00 | 84.45 | C |
| ATOM | 3281 | C3′ | ADE | C | 448 | 33.436 | 27.827 | 62.704 | 1.00 | 84.49 | C |
| ATOM | 3282 | O3′ | ADE | C | 448 | 33.142 | 28.405 | 61.440 | 1.00 | 86.19 | C |
| ATOM | 3283 | P | ADE | C | 449 | 34.210 | 28.275 | 60.245 | 1.00 | 87.99 | C |
| ATOM | 3284 | O1P | ADE | C | 449 | 33.853 | 29.283 | 59.215 | 1.00 | 87.00 | C |
| ATOM | 3285 | O2P | ADE | C | 449 | 34.307 | 26.840 | 59.863 | 1.00 | 86.93 | C |
| ATOM | 3286 | O5′ | ADE | C | 449 | 35.588 | 28.715 | 60.919 | 1.00 | 86.55 | C |
| ATOM | 3287 | C5′ | ADE | C | 449 | 35.750 | 30.027 | 61.447 | 1.00 | 85.31 | C |
| ATOM | 3288 | C4′ | ADE | C | 449 | 37.078 | 30.154 | 62.154 | 1.00 | 85.28 | C |
| ATOM | 3289 | O4′ | ADE | C | 449 | 37.082 | 29.328 | 63.350 | 1.00 | 85.85 | C |
| ATOM | 3290 | C1′ | ADE | C | 449 | 38.410 | 28.880 | 63.611 | 1.00 | 84.64 | C |
| ATOM | 3291 | N9 | ADE | C | 449 | 38.424 | 27.420 | 63.534 | 1.00 | 82.84 | C |
| ATOM | 3292 | C4 | ADE | C | 449 | 39.426 | 26.589 | 63.976 | 1.00 | 81.56 | C |
| ATOM | 3293 | N3 | ADE | C | 449 | 40.587 | 26.942 | 64.555 | 1.00 | 80.63 | C |
| ATOM | 3294 | C2 | ADE | C | 449 | 41.310 | 25.864 | 64.858 | 1.00 | 79.78 | C |
| ATOM | 3295 | N1 | ADE | C | 449 | 41.030 | 24.571 | 64.665 | 1.00 | 79.45 | C |
| ATOM | 3296 | C6 | ADE | C | 449 | 39.856 | 24.249 | 64.080 | 1.00 | 81.05 | C |
| ATOM | 3297 | N6 | ADE | C | 449 | 39.571 | 22.957 | 63.888 | 1.00 | 81.21 | C |
| ATOM | 3298 | C5 | ADE | C | 449 | 38.996 | 25.305 | 63.706 | 1.00 | 81.23 | C |
| ATOM | 3299 | N7 | ADE | C | 449 | 37.749 | 25.326 | 63.096 | 1.00 | 81.07 | C |
| ATOM | 3300 | C8 | ADE | C | 449 | 37.456 | 26.600 | 63.017 | 1.00 | 80.87 | C |
| ATOM | 3301 | C2′ | ADE | C | 449 | 39.302 | 29.513 | 62.544 | 1.00 | 84.95 | C |
| ATOM | 3302 | O2′ | ADE | C | 449 | 39.817 | 30.743 | 63.022 | 1.00 | 84.38 | C |
| ATOM | 3303 | C3′ | ADE | C | 449 | 38.305 | 29.666 | 61.403 | 1.00 | 85.19 | C |
| ATOM | 3304 | O3′ | ADE | C | 449 | 38.751 | 30.611 | 60.443 | 1.00 | 84.91 | C |
| ATOM | 3305 | P | GUA | C | 450 | 39.413 | 30.095 | 59.074 | 1.00 | 84.58 | C |
| ATOM | 3306 | O1P | GUA | C | 450 | 38.369 | 29.339 | 58.332 | 1.00 | 83.17 | C |
| ATOM | 3307 | O2P | GUA | C | 450 | 40.085 | 31.251 | 58.427 | 1.00 | 84.98 | C |
| ATOM | 3308 | O5′ | GUA | C | 450 | 40.556 | 29.094 | 59.551 | 1.00 | 83.16 | C |
| ATOM | 3309 | C5′ | GUA | C | 450 | 41.783 | 29.595 | 60.073 | 1.00 | 81.52 | C |
| ATOM | 3310 | C4′ | GUA | C | 450 | 42.628 | 28.459 | 60.594 | 1.00 | 81.00 | C |
| ATOM | 3311 | O4′ | GUA | C | 450 | 41.834 | 27.654 | 61.508 | 1.00 | 80.06 | C |
| ATOM | 3312 | C1′ | GUA | C | 450 | 42.197 | 26.286 | 61.380 | 1.00 | 79.33 | C |
| ATOM | 3313 | N9 | GUA | C | 450 | 41.032 | 25.541 | 60.904 | 1.00 | 77.56 | C |
| ATOM | 3314 | C4 | GUA | C | 450 | 40.926 | 24.175 | 60.809 | 1.00 | 74.96 | C |
| ATOM | 3315 | N3 | GUA | C | 450 | 41.891 | 23.285 | 61.115 | 1.00 | 72.47 | C |
| ATOM | 3316 | C2 | GUA | C | 450 | 41.486 | 22.042 | 60.933 | 1.00 | 72.36 | C |
| ATOM | 3317 | N2 | GUA | C | 450 | 42.324 | 21.023 | 61.174 | 1.00 | 71.30 | C |
| ATOM | 3318 | N1 | GUA | C | 450 | 40.227 | 21.701 | 60.498 | 1.00 | 72.99 | C |
| ATOM | 3319 | C6 | GUA | C | 450 | 39.216 | 22.600 | 60.187 | 1.00 | 72.97 | C |
| ATOM | 3320 | C6 | GUA | C | 450 | 38.113 | 22.183 | 59.828 | 1.00 | 72.47 | C |
| ATOM | 3321 | C5 | GUA | C | 450 | 39.641 | 23.937 | 60.357 | 1.00 | 74.31 | C |
| ATOM | 3322 | N7 | GUA | C | 450 | 38.963 | 25.129 | 60.146 | 1.00 | 75.07 | C |
| ATOM | 3323 | C8 | GUA | C | 450 | 39.825 | 26.054 | 60.481 | 1.00 | 77.45 | C |
| ATOM | 3324 | C2′ | GUA | C | 450 | 43.375 | 26.233 | 60.410 | 1.00 | 80.25 | C |
| ATOM | 3325 | O2′ | GUA | C | 450 | 44.596 | 26.322 | 61.117 | 1.00 | 78.73 | C |
| ATOM | 3326 | C3′ | GUA | C | 450 | 43.084 | 27.454 | 59.551 | 1.00 | 80.96 | C |
| ATOM | 3327 | O3′ | GUA | C | 450 | 44.236 | 27.901 | 58.853 | 1.00 | 81.13 | C |
| ATOM | 3328 | P | GUA | C | 451 | 44.420 | 27.508 | 57.305 | 1.00 | 81.46 | C |
| ATOM | 3329 | O1P | GUA | C | 451 | 43.133 | 27.796 | 56.616 | 1.00 | 80.36 | C |
| ATOM | 3330 | O2P | GUA | C | 451 | 45.673 | 28.147 | 56.825 | 1.00 | 80.48 | C |
| ATOM | 3331 | O5′ | GUA | C | 451 | 44.631 | 25.927 | 57.341 | 1.00 | 79.28 | C |
| ATOM | 3332 | C5′ | GUA | C | 451 | 45.580 | 25.349 | 58.229 | 1.00 | 76.29 | C |
| ATOM | 3333 | C4′ | GUA | C | 451 | 45.730 | 23.875 | 57.957 | 1.00 | 74.88 | C |
| ATOM | 3334 | O4′ | GUA | C | 451 | 44.758 | 23.130 | 58.728 | 1.00 | 73.92 | C |
| ATOM | 3335 | C1′ | GUA | C | 451 | 44.363 | 21.975 | 58.003 | 1.00 | 72.37 | C |
| ATOM | 3336 | N9 | GUA | C | 451 | 42.947 | 22.106 | 57.687 | 1.00 | 69.07 | C |
| ATOM | 3337 | C4 | GUA | C | 451 | 42.051 | 21.085 | 57.486 | 1.00 | 66.32 | C |
| ATOM | 3338 | N3 | GUA | C | 451 | 42.325 | 19.763 | 57.546 | 1.00 | 63.40 | C |
| ATOM | 3339 | C2 | GUA | C | 451 | 41.246 | 19.027 | 57.317 | 1.00 | 63.39 | C |
| ATOM | 3340 | N2 | GUA | C | 451 | 41.323 | 17.683 | 57.356 | 1.00 | 60.65 | C |
| ATOM | 3341 | N1 | GUA | C | 451 | 40.004 | 19.554 | 57.040 | 1.00 | 64.12 | C |
| ATOM | 3342 | C6 | GUA | C | 451 | 39.703 | 20.914 | 56.975 | 1.00 | 64.33 | C |
| ATOM | 3343 | O6 | GUA | C | 451 | 38.544 | 21.287 | 56.729 | 1.00 | 62.58 | C |
| ATOM | 3344 | C5 | GUA | C | 451 | 40.846 | 21.707 | 57.222 | 1.00 | 65.72 | C |
| ATOM | 3345 | N7 | GUA | C | 451 | 40.987 | 23.086 | 57.252 | 1.00 | 67.31 | C |
| ATOM | 3346 | C8 | GUA | C | 451 | 42.247 | 23.277 | 57.530 | 1.00 | 67.97 | C |
| ATOM | 3347 | C2′ | GUA | C | 451 | 45.215 | 21.935 | 56.736 | 1.00 | 73.66 | C |
| ATOM | 3348 | O2′ | GUA | C | 451 | 46.375 | 21.162 | 56.966 | 1.00 | 72.93 | C |
| ATOM | 3349 | C3′ | GUA | C | 451 | 45.501 | 23.414 | 56.529 | 1.00 | 74.10 | C |
| ATOM | 3350 | O3′ | GUA | C | 451 | 46.695 | 23.571 | 55.791 | 1.00 | 76.92 | C |
| ATOM | 3351 | P | ADE | C | 452 | 46.646 | 23.622 | 54.191 | 1.00 | 79.92 | C |
| ATOM | 3352 | O1P | ADE | C | 452 | 45.749 | 24.737 | 53.778 | 1.00 | 77.97 | C |
| ATOM | 3353 | O2P | ADE | C | 452 | 48.062 | 23.622 | 53.752 | 1.00 | 80.27 | C |
| ATOM | 3354 | O5′ | ADE | C | 452 | 46.002 | 22.225 | 53.768 | 1.00 | 81.04 | C |
| ATOM | 3355 | PC | ADE | C | 452 | 47.403 | 20.187 | 49.052 | 1.00 | 94.69 | C |
| ATOM | 3356 | O1C | ADE | C | 452 | 48.478 | 20.648 | 48.135 | 1.00 | 92.74 | C |
| ATOM | 3357 | O2C | ADE | C | 452 | 46.873 | 18.888 | 48.562 | 1.00 | 93.16 | C |
| ATOM | 3358 | O3′ | ADE | C | 452 | 47.997 | 19.949 | 50.531 | 1.00 | 92.25 | C |
| ATOM | 3359 | C5′ | ADE | C | 452 | 46.769 | 21.023 | 53.811 | 1.00 | 84.82 | C |
| ATOM | 3360 | C4′ | ADE | C | 452 | 46.607 | 20.249 | 52.521 | 1.00 | 88.58 | C |
| ATOM | 3361 | O4′ | ADE | C | 452 | 45.193 | 20.079 | 52.251 | 1.00 | 89.88 | C |
| ATOM | 3362 | C1′ | ADE | C | 452 | 44.954 | 20.314 | 50.882 | 1.00 | 90.96 | C |
| ATOM | 3363 | N9 | ADE | C | 452 | 43.567 | 20.751 | 50.722 | 1.00 | 90.40 | C |
| ATOM | 3364 | C4 | ADE | C | 452 | 42.474 | 19.924 | 50.615 | 1.00 | 89.68 | C |
| ATOM | 3365 | N3 | ADE | C | 452 | 42.462 | 18.579 | 50.648 | 1.00 | 89.15 | C |
| ATOM | 3366 | C2 | ADE | C | 452 | 41.222 | 18.118 | 50.497 | 1.00 | 89.12 | C |
| ATOM | 3367 | N1 | ADE | C | 452 | 40.076 | 18.797 | 50.332 | 1.00 | 88.58 | C |
| ATOM | 3368 | C6 | ADE | C | 452 | 40.123 | 20.148 | 50.308 | 1.00 | 89.15 | C |
| ATOM | 3369 | N6 | ADE | C | 452 | 38.982 | 20.827 | 50.148 | 1.00 | 88.59 | C |
| ATOM | 3370 | C5 | ADE | C | 452 | 41.383 | 20.761 | 50.455 | 1.00 | 89.44 | C |
| ATOM | 3371 | N7 | ADE | C | 452 | 41.779 | 22.092 | 50.468 | 1.00 | 89.57 | C |
| ATOM | 3372 | C8 | ADE | C | 452 | 43.079 | 22.033 | 50.633 | 1.00 | 90.05 | C |
| ATOM | 3373 | C2′ | ADE | C | 452 | 46.001 | 21.345 | 50.459 | 1.00 | 91.00 | C |
| ATOM | 3374 | O2′ | ADE | C | 452 | 46.225 | 21.291 | 49.060 | 1.00 | 92.14 | C |
| ATOM | 3375 | C3′ | ADE | C | 452 | 47.216 | 20.892 | 51.270 | 1.00 | 90.65 | C |
| TER | | | | | | | | | | | |
| HETATM | 3376 | CS + 1 | CS1 | | 101 | 2.853 | 25.764 | 50.394 | 1.00 | 98.04 | D |
| HETATM | 3377 | CS + 1 | CS1 | | 501 | 30.101 | 22.718 | 92.220 | 1.00 | 108.11 | F |
| HETATM | 3378 | MG + 2 | MG2 | | 102 | 2.352 | 24.068 | 36.432 | 1.00 | 50.96 | E |
| HETATM | 3379 | N | SAM | | 100 | −8.439 | 27.146 | 69.630 | 1.00 | 67.99 | G |
| HETATM | 3380 | CA | SAM | | 100 | −9.271 | 27.703 | 68.534 | 1.00 | 70.40 | G |
| HETATM | 3381 | C | SAM | | 100 | −10.749 | 27.414 | 68.789 | 1.00 | 71.30 | G |
| HETATM | 3382 | O | SAM | | 100 | −11.601 | 27.834 | 68.013 | 1.00 | 71.72 | G |
| HETATM | 3383 | OXT | SAM | | 100 | −11.093 | 26.708 | 69.727 | 1.00 | 72.44 | G |
| HETATM | 3384 | CB | SAM | | 100 | −8.870 | 27.061 | 67.190 | 1.00 | 70.62 | G |
| HETATM | 3385 | CG | SAM | | 100 | −7.645 | 27.736 | 66.544 | 1.00 | 71.18 | G |
| HETATM | 3386 | SD | SAM | | 100 | −6.617 | 26.445 | 65.764 | 1.00 | 69.00 | G |
| HETATM | 3387 | CE | SAM | | 100 | −5.148 | 26.604 | 66.812 | 1.00 | 70.58 | G |
| HETATM | 3388 | C5′ | SAM | | 100 | −6.000 | 27.274 | 64.272 | 1.00 | 68.56 | G |
| HETATM | 3389 | C4′ | SAM | | 100 | −5.124 | 26.274 | 63.522 | 1.00 | 67.11 | G |
| HETATM | 3390 | O4′ | SAM | | 100 | −5.882 | 25.101 | 63.332 | 1.00 | 68.09 | G |
| HETATM | 3391 | C3′ | SAM | | 100 | −4.748 | 26.782 | 62.126 | 1.00 | 67.21 | G |
| HETATM | 3392 | O3′ | SAM | | 100 | −3.403 | 27.274 | 62.073 | 1.00 | 66.59 | G |
| HETATM | 3393 | C2′ | SAM | | 100 | −4.914 | 25.524 | 61.221 | 1.00 | 68.03 | G |
| HETATM | 3394 | O2′ | SAM | | 100 | −3.763 | 25.403 | 60.376 | 1.00 | 68.41 | G |
| HETATM | 3395 | C1′ | SAM | | 100 | −5.287 | 24.402 | 62.250 | 1.00 | 67.94 | G |
| HETATM | 3396 | N9 | SAM | | 100 | −6.236 | 23.412 | 61.722 | 1.00 | 68.51 | G |
| HETATM | 3397 | C8 | SAM | | 100 | −7.480 | 23.661 | 61.200 | 1.00 | 69.21 | G |
| HETATM | 3398 | N7 | SAM | | 100 | −8.049 | 22.542 | 60.827 | 1.00 | 70.09 | G |
| HETATM | 3399 | C5 | SAM | | 100 | −7.217 | 21.507 | 61.086 | 1.00 | 69.15 | G |
| HETATM | 3400 | C6 | SAM | | 100 | −7.270 | 20.099 | 60.911 | 1.00 | 68.58 | G |
| HETATM | 3401 | N6 | SAM | | 100 | −8.373 | 19.508 | 60.350 | 1.00 | 69.87 | G |
| HETATM | 3402 | N1 | SAM | | 100 | −6.223 | 19.349 | 61.301 | 1.00 | 68.53 | G |
| HETATM | 3403 | C2 | SAM | | 100 | −5.144 | 19.895 | 61.859 | 1.00 | 69.32 | G |
| HETATM | 3404 | N3 | SAM | | 100 | −5.039 | 21.218 | 62.023 | 1.00 | 69.30 | G |
| HETATM | 3405 | C4 | SAM | | 100 | −6.039 | 22.051 | 61.667 | 1.00 | 69.21 | G |
| HETATM | 3406 | N | SAM | | 300 | 10.237 | 21.648 | 2.816 | 1.00 | 60.97 | H |
| HETATM | 3407 | CA | SAM | | 300 | 11.651 | 22.047 | 3.005 | 1.00 | 62.32 | H |
| HETATM | 3408 | C | SAM | | 300 | 12.362 | 21.942 | 1.712 | 1.00 | 62.83 | H |
| HETATM | 3409 | O | SAM | | 300 | 13.587 | 21.876 | 1.687 | 1.00 | 64.00 | H |
| HETATM | 3410 | OXT | SAM | | 300 | 11.716 | 21.890 | 0.688 | 1.00 | 64.42 | H |
| HETATM | 3411 | CB | SAM | | 300 | 12.377 | 21.174 | 4.031 | 1.00 | 60.97 | H |
| HETATM | 3412 | CG | SAM | | 300 | 11.577 | 21.118 | 5.324 | 1.00 | 63.92 | H |
| HETATM | 3413 | SD | SAM | | 300 | 12.498 | 21.859 | 6.693 | 1.00 | 63.71 | H |
| HETATM | 3414 | CE | SAM | | 300 | 14.101 | 21.012 | 6.547 | 1.00 | 64.61 | H |
| HETATM | 3415 | C5′ | SAM | | 300 | 11.741 | 20.937 | 8.074 | 1.00 | 64.51 | H |
| HETATM | 3416 | C4′ | SAM | | 300 | 12.212 | 21.514 | 9.408 | 1.00 | 64.50 | H |
| HETATM | 3417 | O4′ | SAM | | 300 | 12.014 | 22.911 | 9.367 | 1.00 | 64.16 | H |
| HETATM | 3418 | C3′ | SAM | | 300 | 11.355 | 20.977 | 10.578 | 1.00 | 64.84 | H |
| HETATM | 3419 | O3′ | SAM | | 300 | 12.242 | 20.357 | 11.519 | 1.00 | 63.50 | H |
| HETATM | 3420 | C2′ | SAM | | 300 | 10.763 | 22.266 | 11.209 | 1.00 | 64.14 | H |
| HETATM | 3421 | O2′ | SAM | | 300 | 10.507 | 22.213 | 12.604 | 1.00 | 65.81 | H |
| HETATM | 3422 | C1′ | SAM | | 300 | 11.770 | 23.312 | 10.702 | 1.00 | 64.78 | H |
| HETATM | 3423 | N9 | SAM | | 300 | 11.276 | 24.678 | 10.695 | 1.00 | 66.74 | H |
| HETATM | 3424 | C8 | SAM | | 300 | 10.025 | 25.110 | 10.342 | 1.00 | 67.03 | H |
| HETATM | 3425 | N7 | SAM | | 300 | 9.961 | 26.407 | 10.437 | 1.00 | 68.42 | H |
| HETATM | 3426 | C5 | SAM | | 300 | 11.160 | 26.888 | 10.851 | 1.00 | 68.11 | H |
| HETATM | 3427 | C6 | SAM | | 300 | 11.695 | 28.174 | 11.120 | 1.00 | 68.47 | H |
| HETATM | 3428 | N6 | SAM | | 300 | 10.914 | 29.302 | 10.963 | 1.00 | 68.71 | H |
| HETATM | 3429 | N1 | SAM | | 300 | 12.984 | 28.276 | 11.521 | 1.00 | 68.18 | H |
| HETATM | 3430 | C2 | SAM | | 300 | 13.765 | 27.201 | 11.655 | 1.00 | 67.60 | H |
| HETATM | 3431 | N3 | SAM | | 300 | 13.296 | 25.977 | 11.432 | 1.00 | 67.44 | H |
| HETATM | 3432 | C4 | SAM | | 300 | 12.020 | 25.779 | 11.024 | 1.00 | 67.46 | H |
| HETATM | 3433 | N | SAM | | 500 | 37.476 | 19.907 | 71.774 | 1.00 | 68.92 | I |
| HETATM | 3434 | CA | SAM | | 500 | 37.189 | 18.545 | 72.257 | 1.00 | 67.92 | I |
| HETATM | 3435 | C | SAM | | 500 | 36.755 | 17.664 | 71.104 | 1.00 | 68.27 | I |
| HETATM | 3436 | O | SAM | | 500 | 36.488 | 16.474 | 71.300 | 1.00 | 67.81 | I |
| HETATM | 3437 | OXT | SAM | | 500 | 36.602 | 18.151 | 69.994 | 1.00 | 68.25 | I |
| HETATM | 3438 | CB | SAM | | 500 | 36.063 | 18.606 | 73.284 | 1.00 | 66.91 | I |
| HETATM | 3439 | CG | SAM | | 500 | 36.585 | 19.106 | 74.614 | 1.00 | 65.20 | I |
| HETATM | 3440 | SD | SAM | | 500 | 35.226 | 19.924 | 75.468 | 1.00 | 66.03 | I |
| HETATM | 3441 | CE | SAM | | 500 | 35.742 | 21.645 | 75.216 | 1.00 | 64.78 | I |
| HETATM | 3442 | C5′ | SAM | | 500 | 35.723 | 19.678 | 77.207 | 1.00 | 63.31 | I |
| HETATM | 3443 | C4′ | SAM | | 500 | 34.701 | 20.362 | 78.111 | 1.00 | 61.48 | I |
| HETATM | 3444 | O4′ | SAM | | 500 | 33.419 | 20.080 | 77.600 | 1.00 | 60.13 | I |
| HETATM | 3445 | C3′ | SAM | | 500 | 34.729 | 19.784 | 79.530 | 1.00 | 61.39 | I |
| HETATM | 3446 | O3′ | SAM | | 500 | 35.604 | 20.343 | 80.522 | 1.00 | 60.89 | I |
| HETATM | 3447 | C2′ | SAM | | 500 | 33.217 | 19.729 | 79.929 | 1.00 | 61.21 | I |
| HETATM | 3448 | O2′ | SAM | | 500 | 33.076 | 20.298 | 81.242 | 1.00 | 62.97 | I |
| HETATM | 3449 | C1′ | SAM | | 500 | 32.495 | 20.295 | 78.655 | 1.00 | 59.83 | I |
| HETATM | 3450 | N9 | SAM | | 500 | 31.254 | 19.595 | 78.306 | 1.00 | 59.64 | I |
| HETATM | 3451 | C8 | SAM | | 500 | 31.073 | 18.238 | 78.197 | 1.00 | 59.28 | I |
| HETATM | 3452 | N7 | SAM | | 500 | 29.849 | 17.970 | 77.845 | 1.00 | 59.16 | I |
| HETATM | 3453 | C5 | SAM | | 500 | 29.164 | 19.128 | 77.702 | 1.00 | 59.32 | I |
| HETATM | 3454 | C6 | SAM | | 500 | 27.830 | 19.479 | 77.339 | 1.00 | 59.18 | I |
| HETATM | 3455 | N6 | SAM | | 500 | 26.899 | 18.506 | 77.027 | 1.00 | 58.09 | I |
| HETATM | 3456 | N1 | SAM | | 500 | 27.493 | 20.782 | 77.296 | 1.00 | 59.09 | I |
| HETATM | 3457 | C2 | SAM | | 500 | 28.380 | 21.745 | 77.571 | 1.00 | 58.95 | I |
| HETATM | 3458 | N3 | SAM | | 500 | 29.630 | 21.460 | 77.925 | 1.00 | 58.97 | I |
| HETATM | 3459 | C4 | SAM | | 500 | 30.062 | 20.188 | 77.999 | 1.00 | 59.11 | I |
| HETATM | 3460 | O | HOH | | 1000 | 1.710 | 38.862 | 57.073 | 1.00 | 6.38 | S |
| HETATM | 3461 | O | HOH | | 1002 | 9.109 | 30.706 | −10.011 | 1.00 | 41.55 | S |
| HETATM | 3462 | O | HOH | | 1003 | 10.672 | 39.117 | 54.293 | 1.00 | 13.45 | S |
| HETATM | 3463 | O | HOH | | 1004 | −1.136 | 27.508 | 33.730 | 1.00 | 37.86 | S |
| HETATM | 3464 | O | HOH | | 1005 | 9.642 | 20.371 | 24.593 | 1.00 | 23.91 | S |
| HETATM | 3465 | O | HOH | | 1006 | 24.182 | 12.968 | 37.659 | 1.00 | 29.49 | S |
| HETATM | 3466 | O | HOH | | 1007 | 23.014 | 43.756 | 107.299 | 1.00 | 42.31 | S |
| HETATM | 3467 | O | HOH | | 1008 | 1.203 | 10.487 | 33.970 | 1.00 | 19.01 | S |
| HETATM | 3468 | O | HOH | | 1009 | 0.895 | 28.820 | 56.844 | 1.00 | 19.53 | S |
| HETATM | 3469 | O | HOH | | 1011 | −12.488 | 37.567 | 51.296 | 1.00 | 41.81 | S |
| HETATM | 3470 | O | HOH | | 1012 | 30.364 | 42.530 | 104.047 | 1.00 | 38.54 | S |
| HETATM | 3471 | O | HOH | | 1013 | 14.002 | 22.976 | 53.710 | 1.00 | 32.39 | S |
| HETATM | 3472 | O | HOH | | 1014 | 10.679 | 28.362 | −4.887 | 1.00 | 74.66 | S |
| HETATM | 3473 | O | HOH | | 1015 | 8.558 | 21.534 | −0.603 | 1.00 | 37.38 | S |
| HETATM | 3474 | O | HOH | | 1016 | 6.242 | 38.900 | 42.266 | 1.00 | 33.46 | S |
| HETATM | 3475 | O | HOH | | 1017 | −15.226 | 17.903 | 65.737 | 1.00 | 45.33 | S |
| HETATM | 3476 | O | HOH | | 1018 | 4.523 | 11.428 | 11.327 | 1.00 | 36.06 | S |
| HETATM | 3477 | O | HOH | | 1020 | 18.196 | 24.026 | 37.816 | 1.00 | 57.02 | S |
| HETATM | 3478 | O | HOH | | 1021 | −1.012 | 36.439 | 45.147 | 1.00 | 32.47 | S |
| HETATM | 3479 | O | HOH | | 1022 | 7.578 | 20.568 | 51.009 | 1.00 | 39.39 | S |
| HETATM | 3480 | O | HOH | | 1023 | −14.823 | 28.188 | 59.227 | 1.00 | 30.57 | S |
| HETATM | 3481 | O | HOH | | 1024 | 36.985 | 17.198 | 95.795 | 1.00 | 52.06 | S |
| HETATM | 3482 | O | HOH | | 1026 | 25.333 | 21.807 | 45.339 | 1.00 | 36.69 | S |
| HETATM | 3483 | O | HOH | | 1027 | 15.473 | 28.282 | 57.160 | 1.00 | 49.29 | S |
| HETATM | 3484 | O | HOH | | 1028 | 41.659 | 12.070 | 93.116 | 1.00 | 51.81 | S |
| HETATM | 3485 | O | HOH | | 1029 | 37.830 | 24.077 | 56.753 | 1.00 | 56.04 | S |
| HETATM | 3486 | O | HOH | | 1030 | 1.496 | 18.974 | 19.910 | 1.00 | 46.52 | S |
| HETATM | 3487 | O | HOH | | 1031 | −21.564 | 18.695 | 76.954 | 1.00 | 42.72 | S |
| HETATM | 3488 | O | HOH | | 1032 | 5.632 | 6.369 | 28.271 | 1.00 | 54.24 | S |
| HETATM | 3489 | O | HOH | | 1033 | −12.164 | 20.042 | 54.326 | 1.00 | 37.69 | S |
| HETATM | 3490 | O | HOH | | 1034 | −9.635 | 21.741 | 83.667 | 1.00 | 28.64 | S |
| HETATM | 3491 | O | HOH | | 1035 | 16.349 | 18.517 | −10.014 | 1.00 | 32.54 | S |
| HETATM | 3492 | O | HOH | | 1036 | −19.165 | 22.912 | 60.859 | 1.00 | 40.83 | S |
| HETATM | 3493 | O | HOH | | 1037 | 42.317 | 14.698 | 87.404 | 1.00 | 54.29 | S |
| HETATM | 3494 | O | HOH | | 1038 | 5.305 | 30.972 | 14.298 | 1.00 | 45.65 | S |
| HETATM | 3495 | O | HOH | | 1039 | 17.404 | 7.202 | 37.021 | 1.00 | 42.45 | S |
| HETATM | 3496 | O | HOH | | 1040 | 9.144 | 11.513 | 24.225 | 1.00 | 119.44 | S |
| HETATM | 3497 | O | HOH | | 1041 | 3.177 | 32.569 | 38.072 | 1.00 | 25.83 | S |
| HETATM | 3498 | O | HOH | | 1042 | 29.325 | 17.646 | 67.611 | 1.00 | 53.76 | S |
| HETATM | 3499 | O | HOH | | 1043 | −11.046 | 32.302 | 57.055 | 1.00 | 47.80 | S |
| HETATM | 3500 | O | HOH | | 1045 | 2.422 | 16.973 | 31.694 | 1.00 | 41.35 | S |
| HETATM | 3501 | O | HOH | | 1046 | −13.045 | 28.534 | 71.549 | 1.00 | 47.13 | S |
| HETATM | 3502 | O | HOH | | 1047 | 8.497 | 31.635 | 16.931 | 1.00 | 50.64 | S |
| HETATM | 3503 | O | HOH | | 1048 | −0.041 | 28.567 | 59.602 | 1.00 | 32.18 | S |
| HETATM | 3504 | O | HOH | | 1049 | 24.871 | 19.578 | 80.883 | 1.00 | 68.34 | S |
| HETATM | 3505 | O | HOH | | 1050 | −7.523 | 19.578 | 79.061 | 1.00 | 113.57 | S |
| HETATM | 3506 | O | HOH | | 1051 | 32.273 | 17.481 | 60.997 | 1.00 | 41.48 | S |
| HETATM | 3507 | O | HOH | | 1052 | 1.502 | 18.555 | 10.879 | 1.00 | 50.88 | S |
| HETATM | 3508 | O | HOH | | 1053 | −1.995 | 27.026 | 58.607 | 1.00 | 49.02 | S |
| HETATM | 3509 | O | HOH | | 1054 | −17.653 | 24.848 | 53.584 | 1.00 | 77.80 | S |
| HETATM | 3510 | O | HOH | | 1055 | 40.398 | 23.070 | 95.424 | 1.00 | 51.36 | S |
| HETATM | 3511 | O | HOH | | 1056 | 15.212 | 31.641 | −13.652 | 1.00 | 63.71 | S |
| HETATM | 3512 | O | HOH | | 1057 | 24.773 | 21.394 | 76.777 | 1.00 | 54.68 | S |
| HETATM | 3513 | O | HOH | | 1058 | 16.892 | 31.552 | 45.517 | 1.00 | 36.41 | S |
| HETATM | 3514 | O | HOH | | 1059 | 10.090 | 33.120 | 2.682 | 1.00 | 35.81 | S |
| HETATM | 3515 | O | HOH | | 1060 | 40.562 | 32.813 | 61.039 | 1.00 | 58.14 | S |
| HETATM | 3516 | O | HOH | | 1061 | 14.161 | 20.416 | 47.160 | 1.00 | 49.47 | S |
| HETATM | 3517 | O | HOH | | 1062 | 16.854 | 25.674 | 45.737 | 1.00 | 63.64 | S |
| HETATM | 3518 | O | HOH | | 1063 | 3.203 | 43.376 | 51.094 | 1.00 | 49.11 | S |
| HETATM | 3519 | O | HOH | | 1064 | 10.256 | 14.418 | 12.230 | 1.00 | 47.72 | S |
| HETATM | 3520 | O | HOH | | 1065 | −11.321 | 23.776 | 50.912 | 1.00 | 41.32 | S |
| HETATM | 3521 | O | HOH | | 1066 | 43.248 | 26.720 | 100.251 | 1.00 | 43.17 | S |
| HETATM | 3522 | O | HOH | | 1067 | 6.827 | 22.199 | 53.781 | 1.00 | 44.26 | S |
| HETATM | 3523 | O | HOH | | 1068 | 11.021 | 5.705 | 24.649 | 1.00 | 32.82 | S |
| HETATM | 3524 | O | HOH | | 1069 | −5.141 | 16.290 | 65.889 | 1.00 | 67.72 | S |
| HETATM | 3525 | O | HOH | | 1070 | 37.653 | 20.345 | 106.218 | 1.00 | 43.17 | S |
| HETATM | 3526 | O | HOH | | 1071 | 1.874 | 28.432 | 16.664 | 1.00 | 40.53 | S |
| HETATM | 3527 | O | HOH | | 1072 | −3.039 | 39.908 | 51.178 | 1.00 | 52.15 | S |
| HETATM | 3528 | O | HOH | | 1073 | 1.616 | 35.304 | 49.349 | 1.00 | 31.83 | S |
| HETATM | 3529 | O | HOH | | 1074 | 19.821 | 9.573 | 32.159 | 1.00 | 94.53 | S |
| HETATM | 3530 | O | HOH | | 1075 | 21.624 | 32.806 | 94.159 | 1.00 | 47.85 | S |
| HETATM | 3531 | O | HOH | | 1076 | 24.732 | 34.155 | 108.435 | 1.00 | 51.09 | S |
| HETATM | 3532 | O | HOH | | 1077 | 44.822 | 15.942 | 65.700 | 1.00 | 67.94 | S |
| HETATM | 3533 | O | HOH | | 1078 | 36.613 | 14.026 | 63.136 | 1.00 | 45.24 | S |
| HETATM | 3534 | O | HOH | | 1079 | 6.121 | 30.417 | −2.435 | 1.00 | 46.00 | S |
| HETATM | 3535 | O | HOH | | 1080 | −4.110 | 38.667 | 58.528 | 1.00 | 47.87 | S |
| HETATM | 3536 | O | HOH | | 1081 | 12.209 | 22.292 | −13.923 | 1.00 | 42.28 | S |
| HETATM | 3537 | O | HOH | | 1082 | 0.899 | 35.587 | 51.902 | 1.00 | 26.89 | S |
| HETATM | 3538 | O | HOH | | 1083 | 44.968 | 38.661 | 100.718 | 1.00 | 44.22 | S |
| HETATM | 3539 | O | HOH | | 1084 | −16.065 | 33.146 | 77.476 | 1.00 | 39.70 | S |
| HETATM | 3540 | O | HOH | | 1085 | 5.667 | 23.312 | 50.094 | 1.00 | 54.65 | S |
| HETATM | 3541 | O | HOH | | 1086 | 13.710 | 35.700 | 41.870 | 1.00 | 32.97 | S |
| HETATM | 3542 | O | HOH | | 1087 | 12.259 | 28.157 | 57.075 | 1.00 | 58.60 | S |
| HETATM | 3543 | O | HOH | | 1088 | 9.409 | 21.549 | 27.534 | 1.00 | 31.98 | S |
| HETATM | 3544 | O | HOH | | 1089 | 10.012 | 36.290 | 54.703 | 1.00 | 31.08 | S |
| HETATM | 3545 | O | HOH | | 1090 | 24.694 | 14.271 | 55.811 | 1.00 | 58.74 | S |
| HETATM | 3546 | O | HOH | | 1091 | 21.329 | 17.967 | 85.737 | 1.00 | 52.06 | S |
| HETATM | 3547 | O | HOH | | 1092 | 28.126 | 18.707 | −4.949 | 1.00 | 56.14 | S |
| HETATM | 3548 | O | HOH | | 1093 | 7.439 | 16.780 | 52.218 | 1.00 | 40.03 | S |
| HETATM | 3549 | O | HOH | | 1094 | 23.184 | 15.715 | 36.268 | 1.00 | 74.82 | S |
| HETATM | 3550 | O | HOH | | 1095 | 12.880 | 8.881 | 36.591 | 1.00 | 45.48 | S |
| HETATM | 3551 | O | HOH | | 1096 | 32.397 | 14.748 | 68.336 | 1.00 | 66.46 | S |
| HETATM | 3552 | O | HOH | | 1097 | 29.121 | 35.417 | 93.382 | 1.00 | 61.44 | S |
| HETATM | 3553 | O | HOH | | 1098 | 38.004 | 11.531 | 92.474 | 1.00 | 48.19 | S |
| HETATM | 3554 | O | HOH | | 1099 | −22.649 | 19.849 | 71.095 | 1.00 | 64.38 | S |
| HETATM | 3555 | O | HOH | | 1100 | 12.052 | 19.274 | 24.729 | 1.00 | 31.27 | S |
| HETATM | 3556 | O | HOH | | 1102 | 0.090 | 13.188 | 35.366 | 1.00 | 32.03 | S |
| HETATM | 3557 | O | HOH | | 1104 | 23.453 | 21.387 | 48.664 | 1.00 | 63.46 | S |
| HETATM | 3558 | O | HOH | | 1105 | 1.388 | 17.977 | 33.901 | 1.00 | 23.04 | S |
| HETATM | 3559 | O | HOH | | 1106 | 7.878 | 30.217 | −11.732 | 1.00 | 37.04 | S |
| HETATM | 3560 | O | HOH | | 1107 | −24.804 | 23.927 | 75.393 | 1.00 | 49.59 | S |
| HETATM | 3561 | O | HOH | | 1108 | 16.882 | 19.485 | −12.274 | 1.00 | 44.73 | S |
| HETATM | 3562 | O | HOH | | 1109 | 14.777 | 27.186 | 38.023 | 1.00 | 29.39 | S |
| HETATM | 3563 | O | HOH | | 1110 | 10.347 | 22.869 | 25.228 | 1.00 | 25.02 | S |
| HETATM | 3564 | O | HOH | | 1111 | 29.806 | 43.730 | 102.021 | 1.00 | 65.53 | S |
| HETATM | 3565 | O | HOH | | 1112 | 24.539 | 44.456 | 105.464 | 1.00 | 47.15 | S |
| HETATM | 3566 | O | HOH | | 1114 | 2.511 | 8.834 | 32.890 | 1.00 | 40.34 | S |
| HETATM | 3567 | O | HOH | | 1115 | 0.302 | 44.186 | 52.911 | 1.00 | 35.43 | S |
| HETATM | 3568 | O | HOH | | 1117 | 35.474 | 19.466 | 108.178 | 1.00 | 47.59 | S |
| HETATM | 3569 | O | HOH | | 1118 | 9.046 | 17.460 | 7.452 | 1.00 | 52.67 | S |
| HETATM | 3570 | O | HOH | | 1119 | 13.977 | 25.103 | 55.311 | 1.00 | 65.26 | S |
| HETATM | 3571 | O | HOH | | 1120 | 35.407 | 36.186 | 102.048 | 1.00 | 50.73 | S |
| HETATM | 3572 | O | HOH | | 1121 | 22.824 | 25.739 | −8.232 | 1.00 | 37.73 | S |
| HETATM | 3573 | O | HOH | | 1122 | 0.043 | 26.609 | 56.812 | 1.00 | 54.24 | S |
| HETATM | 3574 | O | HOH | | 1123 | −13.846 | 35.118 | 50.740 | 1.00 | 32.63 | S |
| HETATM | 3575 | O | HOH | | 1125 | 11.978 | 33.811 | 55.449 | 1.00 | 51.87 | S |
| HETATM | 3576 | O | HOH | | 1126 | −9.343 | 19.524 | 53.426 | 1.00 | 46.19 | S |
| HETATM | 3577 | O | HOH | | 1127 | 6.170 | 24.270 | 42.074 | 1.00 | 36.77 | S |
| HETATM | 3578 | O | HOH | | 1128 | −4.329 | 16.897 | 62.855 | 1.00 | 54.10 | S |
| HETATM | 3579 | O | HOH | | 1129 | 14.894 | 23.528 | 19.116 | 1.00 | 54.16 | S |
| HETATM | 3580 | O | HOH | | 1130 | 17.139 | 17.867 | 35.891 | 1.00 | 36.70 | S |
| HETATM | 3581 | O | HOH | | 1131 | 24.693 | 13.072 | 28.311 | 1.00 | 43.09 | S |
| HETATM | 3582 | O | HOH | | 1132 | −9.732 | 23.050 | 48.641 | 1.00 | 59.37 | S |
| HETATM | 3583 | O | HOH | | 1134 | 19.573 | 25.812 | −1.906 | 1.00 | 60.33 | S |
| HETATM | 3584 | O | HOH | | 1135 | 16.632 | 2.961 | 29.344 | 1.00 | 55.25 | S |
| HETATM | 3585 | O | HOH | | 1136 | 14.928 | 18.328 | −7.543 | 1.00 | 56.32 | S |
| HETATM | 3586 | O | HOH | | 1137 | 0.639 | 23.943 | 84.616 | 1.00 | 72.67 | S |
| HETATM | 3587 | O | HOH | | 1138 | 7.076 | 38.013 | 40.230 | 1.00 | 36.05 | S |
| HETATM | 3588 | O | HOH | | 1139 | 5.349 | 21.667 | 44.278 | 1.00 | 35.31 | S |
| HETATM | 3589 | O | HOH | | 1140 | −10.726 | 18.421 | 55.747 | 1.00 | 39.21 | S |
| HETATM | 3590 | O | HOH | | 1141 | 13.721 | 30.346 | 55.808 | 1.00 | 60.85 | S |
| HETATM | 3591 | O | HOH | | 1142 | 15.808 | 12.052 | 20.001 | 1.00 | 38.62 | S |
| HETATM | 3592 | O | HOH | | 1143 | 41.564 | 40.597 | 95.179 | 1.00 | 54.30 | S |
| HETATM | 3593 | O | HOH | | 1144 | 28.387 | 35.762 | 107.518 | 1.00 | 41.34 | S |
| HETATM | 3594 | O | HOH | | 1145 | −7.803 | 17.872 | 56.940 | 1.00 | 50.33 | S |
| HETATM | 3595 | O | HOH | | 1146 | −0.206 | 16.169 | 9.275 | 1.00 | 45.70 | S |
| HETATM | 3596 | O | HOH | | 1147 | 22.226 | 14.100 | 6.368 | 1.00 | 54.35 | S |
| HETATM | 3597 | O | HOH | | 1148 | −3.958 | 39.941 | 64.039 | 1.00 | 38.06 | S |
| HETATM | 3598 | O | HOH | | 1149 | −15.212 | 25.794 | 59.423 | 1.00 | 56.35 | S |
| HETATM | 3599 | O | HOH | | 1150 | −0.407 | 7.594 | 32.284 | 1.00 | 37.21 | S |
| HETATM | 3600 | O | HOH | | 1151 | 42.733 | 12.142 | 57.515 | 1.00 | 37.02 | S |
| HETATM | 3601 | O | HOH | | 1152 | 31.461 | 6.907 | 58.235 | 1.00 | 37.15 | S |
| HETATM | 3602 | O | HOH | | 1153 | 5.446 | 26.093 | 52.566 | 1.00 | 51.35 | S |
| HETATM | 3603 | O | HOH | | 1154 | 33.199 | 9.154 | 79.042 | 1.00 | 48.70 | S |
| HETATM | 3604 | O | HOH | | 1155 | −18.518 | 20.525 | 59.955 | 1.00 | 46.00 | S |
| HETATM | 3605 | O | HOH | | 1156 | −2.783 | 28.127 | 64.601 | 1.00 | 49.49 | S |
| HETATM | 3606 | O | HOH | | 1157 | 10.260 | 2.953 | 24.931 | 1.00 | 35.76 | S |
| HETATM | 3607 | O | HOH | | 1158 | 17.806 | 19.552 | 41.831 | 1.00 | 62.42 | S |
| HETATM | 3608 | O | HOH | | 1159 | 34.865 | 16.077 | 59.910 | 1.00 | 77.33 | S |
| HETATM | 3609 | O | HOH | | 1160 | 13.810 | 38.261 | −7.058 | 1.00 | 40.41 | S |
| HETATM | 3610 | O | HOH | | 1161 | 10.253 | 23.899 | 0.112 | 1.00 | 44.76 | S |
| HETATM | 3611 | O | HOH | | 1162 | −8.092 | 22.939 | 45.493 | 1.00 | 60.62 | S |
| HETATM | 3612 | O | HOH | | 1163 | 12.939 | 42.444 | 43.941 | 1.00 | 47.94 | S |
| HETATM | 3613 | O | HOH | | 1164 | −7.966 | 16.660 | 61.300 | 1.00 | 56.65 | S |
| END | | | | | | | | | | | |

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for identifying a compound that associates with a SAM-II riboswitch comprising the steps of:
modeling at least one portion of the SAM-II riboswitch atomic structure depicted in at least one of FIG. 4A (SEQ ID NO:6) or FIG. 4B (SEQ ID NO:7) with a test compound; and
determining an association between the test compound and the SAM-II riboswitch atomic structure.

2. The method of claim 1, further comprising determining that the test compound reduces bacterial gene expression.

3. The method of claim 1, further comprising determining that the test compound induces bacterial gene expression.

4. The method of claim 1, wherein the association determination step comprises determining at least one of a minimum interaction energy, a binding constant, a dissociation constant, or a combination thereof, for the test compound with the modeling of at least one portion of the SAM-II riboswitch atomic structure.

5. The method of claim 1, wherein the association determination step comprises determining the interaction of the test compound with one or more nucleotides of the SAM-II riboswitch comprising U10, U11 , U12, U20, U21, G22, U44, A45, A46, A47, or a combination thereof.

6. The method of claim 1, wherein the association determination step further comprises determining an interaction of the test compound with a S-adenosyl-methionine moiety comprising a ribose sugar, a methionine side chain, a sulfur atom, an adenine moiety or combination thereof.

7. The method of claim 1, wherein the association determination step further comprises determining an interaction of the test compound with a nucleotide of the SAM-II riboswitch atomic structure comprising U10, U12, U20, G22, U44, A46, A47 or a combination thereof.

8. The method of claim 1, wherein the association determination step further comprises determining an interaction of the test compound with a P2b helix region of the SAM-II riboswitch atomic structure.

9. A method of regulating gene expression in a cell by modulating an mRNA, the method comprising the steps of administering a SAM-II riboswitch modulating compound to the cell to modulate the SAM-II riboswitch activity of the mRNA.

10. The method of claim 9, wherein gene expression is stimulated.

11. The method of claim 9, wherein gene expression is inhibited.

12. The method of claim 9, wherein the SAM-II riboswitch modulating compound forms a complex with the SAM-II riboswitch decreasing the formation of an antiterminator element by the mRNA.

13. The method of claim 10, wherein the cell is a bacterial cell.

14. The method of claim 14, wherein the bacterial cell is a gram-negative bacterial cell.

15. A SAM-II riboswitch, wherein one or more of nucleotides U10, U11, U12, U20, U21, G22, U44, A45, A46, A47 are modified.

16. The method of claim 15, wherein interaction with a SAM-II riboswitch having the one or more modified nucleotide causes an increase gene expression in a cell.

17. The method of claim 15, wherein interaction with a SAM-II riboswitch having the one or more modified nucleotide causes a decrease gene expression in a cell.

18. The method of claim 15, wherein interaction with a SAM-II riboswitch having the one or more modified nucleotide causes a decreases in sulfur production in a cell.

19. A composition comprising a compound that associates with at least a portion of the SAM-II riboswitch atomic structure depicted in at least one of FIG. 4A (SEQ ID NO:6) or FIG. 4B (SEQ ID NO:7), wherein the association includes compound interaction with at least one of nucleotides U10, U11, U12, U20, U21, G22, U44, A45, A46, or A47, and wherein the composition is capable of modifying SAM-II riboswitch activity in a bacterial organism.

20. The composition of claim 19, wherein the composition further comprises a pharmaceutically acceptable excipient.

21. A composition comprising at least 80% of a conserved nucleotide sequence of a SAM-II riboswitch core depicted in FIG. 1 (SEQ ID NO:1) and 80% or more of nucleotides depicted outside of a conserved region depicted in FIG. 4A (SEQ ID NO:6) or 4B (SEQ ID NO:7).

22. The composition of claim 21, further comprising a nucleotide sequence depicted in FIG. 4A (SEQ ID NO:6) or 4B (SEQ ID NO:7).
